(19) United States
(12) Patent Application Publication
(10) Pub. No.: US 2023/0286969 A1
(43) Pub. Date: Sep. 14, 2023

(54) AROMATIC SPIRO RING AMIDE DERIVATIVES FOR THE TREATMENT AND PROPHYLAXIS OF HEPATITIS B VIRUS INFECTION

(71) Applicant: Hoffmann-La Roche Inc., Little Falls, NJ (US)

(72) Inventors: Xianfeng LIN, Shanghai (CN); Hongying YUN, Shanghai (CN); Bo ZHANG, Shanghai (CN); Xiufang ZHENG, Shanghai (CN)

(73) Assignee: Hoffmann-La Roche Inc., Little Falls, NJ (US)

(21) Appl. No.: 18/319,895

(22) Filed: May 18, 2023

Related U.S. Application Data
(63) Continuation of application No. PCT/EP2021/082593, filed on Nov. 23, 2021.

(30) Foreign Application Priority Data
Nov. 25, 2020 (WO) PCT/CN2020/131371

(51) Int. Cl. C07D 413/12; C07D 498/04

(52) U.S. Cl. CPC C07D 413/12; C07D 498/04; C07B 2200/13

(57) ABSTRACT

The present invention provides novel compounds having the general formula:

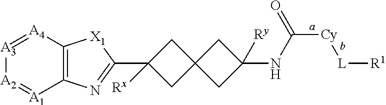

wherein A₁ to A₄, X₁, R^{x}, R^{y}, Cy, L and R¹ are as described herein, compositions including the compounds and methods of using the compounds.

### CROSS-REFERENCE TO PRIOR APPLICATIONS

This application is a continuation of International Application No. PCT/EP2021/082593 having an International Filing Date of Nov. 23, 2021 and which claims benefit under 35 U.S.C. § 119 to International Application No. PCT/CN2020/131371 having an International Filing Date of Nov. 25, 2020. The entire contents of both are incorporated herein by reference.

### FIELD OF THE INVENTION

The present invention relates to aromatic spiro ring amide derivatives useful for therapy and/or prophylaxis of HBV infection in a mammal, and in particular to HBsAg (HBV Surface antigen) and HBeAg (HBV e antigen) inhibitors as well as their manufacture and pharmaceutical compositions containing them.

The present invention relates to compounds of formula (I)

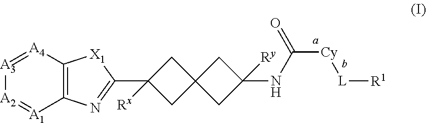

wherein A₁ to A₄, X₁, R^{x}, R^{y}, Cy, L and R¹ are as described below, or a pharmaceutically acceptable salt thereof.

### BACKGROUND OF THE INVENTION

Hepatitis B virus (HBV) is one of the most dangerous human pathogens. A safe and effective vaccine has been available for longer than two decades; however, WHO estimated that approximately 257 million people are chronically infected with HBV. Chronic Hepatitis B (CHB) infection predisposes its host to severe liver disease, including liver cirrhosis and hepatocellular carcinoma, if left untreated. HBV infection is ranked among the top unmet medical need worldwide. The currently approved drugs have contributed to substantial progress in CHB treatment; however, the cure rate remains less than 10%.

The control of viral infection needs an effective immune surveillance. Upon recognition of viral infection, the host innate immune system could respond within minutes to impede viral replication and limits the development of a chronic and persistent infection. The secretion of antiviral cytokines from infected hepatocytes and intra-hepatic immune cells is critically important for the clearance of viral infection. However, chronically infected patients only display a weak immune response due to various escape strategies adopted by the virus to counteract the host cell recognition systems and the subsequent antiviral responses.

Many observations showed that several HBV viral proteins could counteract the initial host cellular response by interfering with the viral recognition signaling system and subsequently the interferon (IFN) antiviral activity. Among these, the excessive secretion of HBV empty subviral particles (SVPs, HBsAg) may contribute to immune tolerant state observed in CHB patients. The persistent exposure to HBsAg and other viral antigens can lead to HBV-specific T-cell functional impairment and depletion (Kondo et al. Journal of Immunology (1993), 150, 4659-4671; Kondo et al. Journal of Medical Virology (2004), 74, 425-433; Fisicaro et al. Gastroenterology, (2010), 138, 682-693;). Moreover HBsAg has been reported to suppress immune cell functions, including monocytes, dendritic cells (DCs) and natural killer (NK) cells (Op den Brouw et al. Immunology, (2009b), 126, 280-289; Woltman et al. PLoS One, (2011), 6, e15324; Shi et al. J Viral Hepat. (2012), 19, e26-33; Kondo et al. ISRN Gasteroenterology, (2013), Article ID 935295).

HBsAg is an important biomarker for prognosis and treatment response in CHB. However, the achievement of HBsAg loss and seroconversion is rarely achieved in CHB patients. HBsAg loss with or without anti-HBsAg seroconversion remains the ideal clinical treatment endpoints. Current therapies, such as nucleos(t)ide analogues, are effective in suppressing HBV DNA, but are not effective in reducing HBsAg level. Nucleos(t)ide analogs, even with prolonged therapy, have demonstrated HBsAg clearance rates comparable to those observed naturally (Janssen et al. Lancet, (2005), 365, 123-129; Marcellin et al. N. Engl. J Med., (2004), 351, 1206-1217; Buster et al. Hepatology, (2007), 46, 388-394). Therefore, there is an urgent need for the development of novel therapeutic agents that could efficiently reduce HBsAg. (Wieland, S. F. & F. V. Chisari. J Virol, (2005), 79, 9369-9380; Kumar et al. J Virol, (2011), 85, 987-995; Woltman et al. PLoS One, (2011), 6, e15324; Op den Brouw et al. Immunology, (2009b), 126, 280-289).

### SUMMARY OF THE INVENTION

Objects of the present invention are novel compounds of formula (I), their manufacture, medicaments based on a compound in accordance with the invention and their production as well as the use of compounds of formula (I) as HBV inhibitors and for the treatment or prophylaxis of HBV infection. The compounds of formula (I) show superior anti-HBV activity. In addition, the compounds of formula (I) also show good safety and good PK profiles.

The present invention relates to a compound of formula (I)

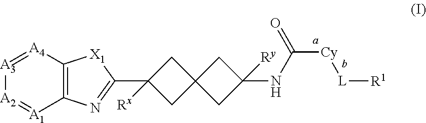

wherein
R¹ is selected from H, halogen, amino, CN, C₁₋₆alkyl, haloC₁₋₆alkyl, C₁₋₆alkoxy, C₁₋₆alkoxycarbonylC₁₋₆alkyl, C₁₋₆alkoxyC₂₋₆alkoxyC₁₋₆alkyl, C₁₋₆alkoxyC₂₋₆alkoxyC₂₋₆alkoxyC₁₋₆alkyl, C₁₋₆alkylamino, (C₁₋₆alkyl)₂amino, C₁₋₆alkoxyC₁₋₆alkyl, C₃₋₇cycloalkyl, C₃₋₇cycloalkylC₁₋₆alkyl, heterocyclyl, heterocyclylC₁₋₆alkyl, C₁₋₆alkylheterocyclylC₁₋₆alkyl and phenylC₁₋₆alkoxycarbonylheterocyclylC₁₋₆alkyl; wherein C₃₋₇cycloalkyl, C₃₋₇cycloalkylC₁₋₆alkyl, heterocyclyl, heterocyclylC₁₋₆alkyl, C₁₋₆alkylheterocyclylC₁₋₆alkyl and phenylC₁₋₆alkoxycarbonylheterocyclylC₁₋₆alkyl are unsubstituted or substituted one or two or three times by halogen;
R^{x} is selected from H, halogen, hydroxy and C₁₋₆alkyl;
R^{y} is selected from H, halogen, hydroxy and C₁₋₆alkyl;
A₁ is selected from N and CR²; wherein R² is selected from H and halogen;
A₂ is selected from N and CR³; wherein R³ is selected from H and halogen;
A₃ is selected from N and CR⁴; wherein R⁴ is selected from H and halogen;
A₄ is selected from N and CR⁵; wherein R⁵ is selected from H and halogen;
X₁ is selected from NR^{z} and O; wherein R^{z} is C₁₋₆alkyl;
Cy is a 5 membered heteroaryl; wherein the 5 membered heteroaryl is unsubstituted or substituted by one or two or three substituents independently selected from halogen, C₁₋₆alkyl, haloC₁₋₆alkyl, C₁₋₆alkoxy and C₁₋₆alkoxyC₁₋₆alkyl;
L is selected from a bond, carbonyl, sulfinyl, sulfonyl, sulfonylC₁₋₆alkyl, sulfonylaminoC₁₋₆ alkyl, sulfanyl, sulfonimidoyl, sulfonimidoylC₁₋₆alkyl and carbonylaminosulfonyl;
or a pharmaceutically acceptable salt thereof.

### DETAILED DESCRIPTION OF THE INVENTION

### Definitions

As used herein, the term “C₁₋₆alkyl” alone or in combination signifies a saturated, linear- or branched chain alkyl group containing 1 to 6, particularly 2 to 6 or 1 to 4 carbon atoms, for example methyl, ethyl, propyl, isopropyl, butyl, isobutyl, tert-butyl and the like. Particular “C₁₋₆alkyl” groups are methyl, ethyl, propyl, isopropyl, isobutyl and tert-butyl.

The term “C₁₋₆alkoxy” alone or in combination signifies a group C₁₋₆alkyl-O—, wherein the “C₁₋₆alkyl” is as defined above; for example methoxy, ethoxy, propoxy, iso-propoxy, n-butoxy, iso-butoxy, 2-butoxy, tert-butoxy, pentoxy, hexyloxy and the like. Particular “C₁₋₆alkoxy” groups are methoxy and ethoxy and propoxy.

The term “C₃₋₇cycloalkyl” denotes to a saturated carbon mono or bicyclic ring or a saturated spiro-linked bicyclic carbon ring or a bridged carbon ring, containing from 3, 4, 5, 6, or 7 carbon atoms, particularly from 3 to 6 carbon atoms, for example, cyclopropyl, cyclobutyl, cyclopentyl, cyclohexyl, cycloheptyl, bicyclo[1.1.1]pentanyl and the like. Particular “C₃₋₇ cycloalkyl” group is cyclopropyl, cyclobutyl or cyclohexyl.

The term “halogen” and “halo” are used interchangeably herein and denote fluoro, chloro, bromo, or iodo.

The term “haloC₁₋₆alkyl” denotes an alkyl group wherein at least one of the hydrogen atoms of the alkyl group is replaced by same or different halogen atoms, particularly fluoro atoms. Examples of haloC₁₋₆alkyl include monochloro-, difluoro- or trifluoro-methyl, -ethyl or -propyl, for example difluoromethyl and trifluoromethyl.

The term “heterocyclyl” refers to any mono-, bi-, tricyclic or spiro, saturated or unsaturated, aromatic (heteroaryl) or non-aromatic (e.g., heterocycloalkyl), ring system, having 3 to 20 ring atoms, where the ring atoms are carbon, and at least one atom in the ring or ring system is a heteroatom selected from nitrogen, sulfur or oxygen. If any ring atom of a cyclic system is a heteroatom, that system is a heterocyclyl, regardless of the point of attachment of the cyclic system to the rest of the molecule. In one example, heterocyclyl includes 3-11 ring atoms (“members”) and includes monocycles, bicycles, tricycles and spiro ring systems, wherein the ring atoms are carbon, where at least one atom in the ring or ring system is a heteroatom selected from nitrogen, sulfur or oxygen. In one example, heterocyclyl includes 3- to 7-membered monocycles having 1, 2, 3 or 4 heteroatoms selected from nitrogen, sulfur or oxygen. In another example, heterocyclyl includes 4-, 5- or 6-membered monocycles having 1, 2, 3 or 4 heteroatoms selected from nitrogen, sulfur or oxygen. In one example, heterocyclyl includes 8- to 12-membered bicycles having 1, 2, 3, 4, 5 or 6 heteroatoms selected from nitrogen, sulfur or oxygen. In another example, heterocyclyl includes 9- or 10-membered bicycles having 1, 2, 3, 4, 5 or 6 heteroatoms selected from nitrogen, sulfur or oxygen. Examplary heterocyclyls are furyl, pyrrolidinyl, morpholino, morpholinyl, thiazolyl, oxazolidinyl, 1,3-dioxole, 2,3-dihydrofuran, 2,3-dihydro-1,4-dioxine, 2,3-dihydro-1H-pyrrole, azetidinyl, oxetanyl, tetrahydrofuranyl, thietanyl, tetrahydrothiophenyl, tetrahydrothiopyranyl, tetrahydropyranyl, 4,5-dihydro-3H-isothiazol-1-yl, 1lambda4-thia-2-azacyclohexen-1-yl, 1,1-dioxothietanyl, 1,1-dioxothiolanyl, 1,1-dioxothianyl, 1-oxo-4,5-dihydro-3H-isothiazol-1-yl, 1-oxo-1lambda6-thia-2-azacyclohexen-1-yl. Heterocyclyl may be optionally substituted by halogen, OH, SH, cyano, NH₂, NHCH₃, N(CH₃)₂, NO₂, N₃, C(O)CH₃, COOH, CO₂CH₃, C₁₋₆alkyl, C₁₋₆alkoxy, oxo, haloC₁₋₆alkyl, phenyl or heterocyclyl.

The term “carbonyl” alone or in combination refers to the group —C(O)—.

The term “sulfanyl” alone or in combination refers to the group —S—.

The term “sulfinyl” alone or in combination refers to the group —S(O)—.

The term “sulfonyl” alone or in combination refers to the group —S(O)₂—.

The term “sulfonimidoyl” alone or in combination refers to the group —S(O)(NH)—, whose formula is

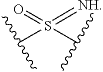 [SMILES: *S(*)(=N)=O]

The term “bond” refers to a chemical bond between two atoms, or two moieties when the atoms joined by the bond are considered to be part of larger substructure. In one aspect, when a group described herein is a bond, the referenced group is absent thereby allowing a bond to be formed between the remaining identified groups. The “a” and “b” on the bonds are used as the symbols of the bonds to indicate the connection sites.

The term “oxo” means an ═O group and may be attached to a carbon atom or a sulfur atom.

As used herein, the wavy line “ [symbol] ” that intersects a bond in a chemical structure refers to the point of attachment of the bond to which the wavy bond intersects in the chemical structure fragment to the remainder of a molecule or structural formula.

The compounds according to the present invention may exist in the form of their pharmaceutically acceptable salts. The term “pharmaceutically acceptable salt” refers to conventional acid-addition salts or base-addition salts that retain the biological effectiveness and properties of the compounds of formula (I) and are formed from suitable non-toxic organic or inorganic acids or organic or inorganic bases. Acid-addition salts include for example those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, salicylic acid, methanesulfonic acid, oxalic acid, succinic acid, citric acid, malic acid, lactic acid, fumaric acid, and the like. Base-addition salts include those derived from ammonium, potassium, sodium and, quaternary ammonium hydroxides, such as for example, tetramethyl ammonium hydroxide. The chemical modification of a pharmaceutical compound into a salt is a technique well known to pharmaceutical chemists in order to obtain improved physical and chemical stability, hygroscopicity, flowability and solubility of compounds. It is for example described in Bastin R. J., et al., Organic Process Research & Development 2000, 4, 427-435. Particular are the sodium salts of the compounds of formula (I).

Compounds of the general formula (I) which contain one or several chiral centers can either be present as racemates, diastereomeric mixtures, or optically active single isomers. The racemates can be separated according to known methods into the enantiomers. Particularly, diastereomeric salts which can be separated by crystallization are formed from the racemic mixtures by reaction with an optically active acid such as e.g. D- or L-tartaric acid, mandelic acid, malic acid, lactic acid or camphorsulfonic acid.

### HBV Inhibitors

The present invention provides (i) a compound having the general formula (I):

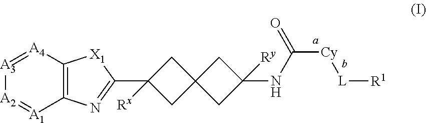

wherein
R¹ is selected from H, halogen, amino, CN, C₁₋₆alkyl, haloC₁₋₆alkyl, C₁₋₆alkoxy, C₁₋₆ alkoxycarbonylC₁₋₆alkyl, C₁₋₆alkoxyC₂₋₆alkoxyC₁₋₆alkyl, C₁₋₆alkoxyC₂₋₆alkoxyC₂₋₆alkoxyC₁₋₆alkyl, C₁₋₆alkylamino, (C₁₋₆alkyl)₂amino, C₁₋₆alkoxyC₁₋₆alkyl, C₃₋₇cycloalkyl, C₃₋₇cycloalkylC₁₋₆alkyl, heterocyclyl, heterocyclylC₁₋₆alkyl, C₁₋₆alkylheterocyclylC₁₋₆alkyl and phenylC₁₋₆alkoxycarbonylheterocyclylC₁₋₆alkyl; wherein C₃₋₇cycloalkyl, C₃₋₇cycloalkylC₁₋₆alkyl, heterocyclyl, heterocyclylC₁₋₆alkyl, C₁₋₆alkylheterocyclylC₁₋₆alkyl and phenylC₁₋₆alkoxycarbonylheterocyclylC₁₋₆alkyl are unsubstituted or substituted one or two or three times by halogen;
R^{x} is selected from H, halogen, hydroxy and C₁₋₆alkyl;
R^{y} is selected from H, halogen, hydroxy and C₁₋₆alkyl;
A₁ is selected from N and CR²; wherein R² is selected from H and halogen;
A₂ is selected from N and CR³; wherein R³ is selected from H and halogen;
A₃ is selected from N and CR⁴; wherein R⁴ is selected from H and halogen;
A₄ is selected from N and CR⁵; wherein R⁵ is selected from H and halogen;
X₁ is selected from NR^{z} and O; wherein R^{z} is C₁₋₆alkyl;
Cy is a 5 membered heteroaryl; wherein the 5 membered heteroaryl is unsubstituted or substituted by one or two or three substituents independently selected from halogen, C₁₋₆ alkyl, haloC₁₋₆alkyl, C₁₋₆alkoxy and C₁₋₆alkoxyC₁₋₆alkyl;
L is selected from a bond, carbonyl, sulfinyl, sulfonyl, sulfonylC₁₋₆alkyl, sulfonylaminoC₁₋₆alkyl, sulfanyl, sulfonimidoyl, sulfonimidoylC₁₋₆alkyl and carbonylaminosulfonyl;
or a pharmaceutically acceptable salt thereof.

A further embodiment of the present invention is (ii) a compound of formula (I) according to (i), wherein
R¹ is selected from H, halogen, amino, CN, C₁₋₆alkyl, haloC₁₋₆alkyl, C₁₋₆alkoxy, C₁₋₆alkoxycarbonylC₁₋₆alkyl, C₁₋₆alkoxyC₂₋₆alkoxyC₁₋₆alkyl, C₁₋₆alkoxyC₂₋₆alkoxyC₂₋₆alkoxyC₁₋₆alkyl, C₁₋₆alkylamino, (C₁₋₆alkyl)₂amino, C₁₋₆alkoxyC₁₋₆alkyl, C₃₋₇cycloalkyl, C₃₋₇cycloalkylC₁₋₆alkyl, oxetanyl, pyrrolidinyl, morpholino, tetrahydrofuranyl, 1,1-dioxothietanyl, 1,1-dioxothiolanyl, 1,1-dioxothianyl, tetrahydropyranyl, 1-oxo-4,5-dihydro-3H-isothiazol-1-yl, 1-oxo-1lambda6-thia-2-azacyclohexen-1-yl, oxetanylC₁₋₆alkyl, C₁₋₆alkylazetidinylC₁₋₆alkyl and phenylC₁₋₆alkoxycarbonylazetidinylC₁₋₆alkyl; wherein C₃₋₇cycloalkylC₁₋₆alkyl is unsubstituted or substituted one or two or three times by halogen;
R^{x} is H;
R^{y} is H;
A₁ is CH;
A₂ is selected from N and CR³; wherein R³ is selected from H and halogen;
A₃ is CR⁴; wherein R⁴ is selected from H and halogen;
A₄ is selected from N and CH;
X₁ is O;
Cy is formula (Cy1)

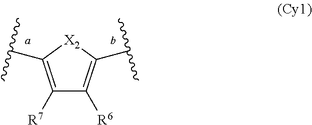

wherein
R⁶ is selected from H and halogen;
R⁷ is selected from H, halogen and C₁₋₆alkyl;
X₂ is selected from O and S;
or formula (Cy2)

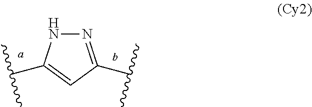 [SMILES: *c1cc(*)[nH]n1]

or formula (Cy3)

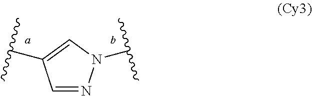 [SMILES: *c1cnn(*)c1]

L is selected from a bond, carbonyl, sulfinyl, sulfonyl, sulfonylC₁₋₆alkyl, sulfonylaminoC₁₋₆alkyl, sulfanyl, sulfonimidoyl, sulfonimidoylC₁₋₆alkyl and carbonylaminosulfonyl; or a pharmaceutically acceptable salt thereof.

A further embodiment of the present invention is (iii) a compound of formula (I) according to (i), wherein
R¹ is selected from H, Cl, Br, amino, CN, methyl, ethyl, propyl, isopropyl, isobutyl, tert-butyl, CF₃, methoxy, ethoxy, methoxycarbonylethyl, methoxyethoxymethyl, methoxyethoxyethoxymethyl, methylamino, dimethylamino, methoxymethyl, methoxyethyl, cyclopropyl, cyclobutyl, cyclopentyl, cyclohexyl, bicyclo[1.1.1]pentanyl, cyclopropylmethyl, cyclobutylmethyl, difluorocyclobutylmethyl, oxetanyl, pyrrolidinyl, morpholino, tetrahydrofuranyl, 1,1-dioxothietanyl, 1,1-dioxothiolanyl, 1,1-dioxothianyl, tetrahydropyranyl, 1-oxo-4,5-dihydro-3H-isothiazol-1-yl, 1-oxo-1lambda6-thia-2-azacyclohexen-1-yl, oxetanylmethyl, methylazetidinylmethyl and phenylmethoxycarbonylazetidinylmethyl;
R^{x} is H;
R^{y} is H;
A₁ is CH;
A₂ is selected from N and CR³; wherein R³ is selected from H and Cl;
A₃ is CR⁴; wherein R⁴ is selected from H, F and Cl;
A₄ is selected from N and CH;
X₁ is O;
Cy is formula (Cy1)

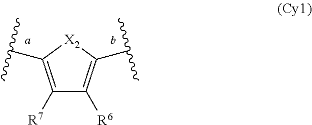

wherein
R⁶ is selected from H and Br;
R⁷ is selected from H, Br and methyl;
X₂ is selected from O and S;
or formula (Cy2)

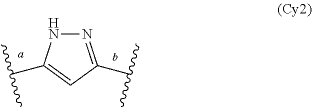 [SMILES: *c1cc(*)[nH]n1]

or formula (Cy3)

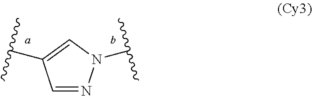 [SMILES: *c1cnn(*)c1]

L is selected from a bond, carbonyl, sulfinyl, sulfonyl, sulfonylmethyl, sulfonylethyl, sulfonylaminomethyl, sulfanyl, sulfonimidoyl, sulfonimidoylmethyl and carbonylaminosulfonyl;
or a pharmaceutically acceptable salt thereof.

A further embodiment of the present invention is (iv) a compound of formula (I) according to (i), or a pharmaceutically acceptable salt thereof, wherein R¹ is selected from amino, C₁₋₆alkyl, haloC₁₋₆alkyl, C₁₋₆alkylamino, C₁₋₆alkoxyC₁₋₆alkyl, C₃₋₇cycloalkyl, C₃₋₇cycloalkylC₁₋₆alkyl and phenylC₁₋₆alkoxycarbonylazetidinylC₁₋₆alkyl; wherein C₃₋₇cycloalkylC₁₋₆alkyl is unsubstituted or substituted one or two times independently by halogen.

A further embodiment of the present invention is (v) a compound of formula (I) according to (i), or a pharmaceutically acceptable salt thereof, wherein R¹ is selected from amino, methyl, ethyl, propyl, isopropyl, isobutyl, tert-butyl, CF₃, methylamino, methoxymethyl, methoxyethyl, cyclopropyl, cyclobutyl, cyclohexyl, cyclopropylmethyl, cyclobutylmethyl, difluorocyclobutylmethyl and phenylmethoxycarbonylazetidinylmethyl.

A further embodiment of the present invention is (vi) a compound of formula (I) according to (i), or a pharmaceutically acceptable salt thereof, wherein A₂ is CR³; wherein R³ is halogen.

A further embodiment of the present invention is (vii) a compound of formula (I) according to (i), or a pharmaceutically acceptable salt thereof, wherein A₂ is CCl.

A further embodiment of the present invention is (viii) a compound of formula (I) according to (i), or a pharmaceutically acceptable salt thereof, wherein
Cy is formula (Cy1)

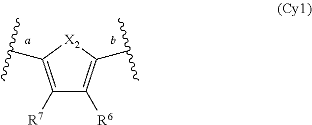

wherein
R⁶ is selected from H and halogen;
R⁷ is H;
X₂ is O.

A further embodiment of the present invention is (ix) a compound of formula (I) according to (viii), or a pharmaceutically acceptable salt thereof, wherein R⁶ is selected from H and Br.

A further embodiment of the present invention is (x) a compound of formula (I) according to (i), or a pharmaceutically acceptable salt thereof, wherein L is selected from sulfinyl, sulfonyl, sulfonylC₁₋₆alkyl, sulfonimidoyl and carbonylaminosulfonyl.

A further embodiment of the present invention is (xi) a compound of formula (I) according to (i), or a pharmaceutically acceptable salt thereof, wherein L is selected from sulfinyl, sulfonyl, sulfonylmethyl, sulfonimidoyl and carbonylaminosulfonyl.

A further embodiment of the present invention is (xii) a compound of formula (II) according to (i), or a pharmaceutically acceptable salt thereof,

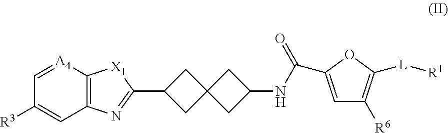

wherein
R¹ is selected from amino, C₁₋₆alkyl, haloC₁₋₆alkyl, C₁₋₆alkylamino, C₁₋₆alkoxyC₁₋₆alkyl, C₃₋₇cycloalkyl, C₃₋₇cycloalkylC₁₋₆alkyl and phenylC₁₋₆alkoxycarbonylazetidinylC₁₋₆alkyl; wherein C₃₋₇cycloalkylC₁₋₆alkyl is unsubstituted or substituted one or two times independently by halogen;
R³ is halogen;
A₄ is selected from N and CH;
X₁ is O;
R⁶ is selected from H and halogen;
L is selected from sulfinyl, sulfonyl, sulfonylC₁₋₆alkyl, sulfonimidoyl and carbonylaminosulfonyl.

A further embodiment of the present invention is (xiii) a compound of formula (II) according to (i), or a pharmaceutically acceptable salt thereof, wherein
R¹ is selected from amino, methyl, ethyl, propyl, isopropyl, isobutyl, tert-butyl, CF₃, methylamino, methoxymethyl, methoxyethyl, cyclopropyl, cyclobutyl, cyclohexyl, cyclopropylmethyl, cyclobutylmethyl, difluorocyclobutylmethyl and phenylmethoxycarbonylazetidinylmethyl;
R³ is Cl;
A₄ is selected from N and CH;
X₁ is O;
R⁶ is selected from H and Br;
L is selected from sulfinyl, sulfonyl, sulfonylmethyl, sulfonimidoyl and carbonylaminosulfonyl.

A further embodiment of the present invention is (xiv) a compound of formula (II) according to (i), or a pharmaceutically acceptable salt thereof, wherein A₄ is CH.

In another embodiment (xv) of the present invention, particular compounds of the present invention are selected from:
N-[6-(5-chloro-1,3-benzoxazol-2-yl)spiro[3.3]heptan-2-yl]-5-(trifluoromethyl)furan-2-carboxamide;
N-[6-(5-chloro-1,3-benzoxazol-2-yl)spiro[3.3]heptan-2-yl]-5-cyano-furan-2-carboxamide;
5-bromo-N-[6-(5-chloro-1,3-benzoxazol-2-yl)spiro[3.3]heptan-2-yl]furan-2-carboxamide;
4-bromo-N-[6-(5-chloro-1,3-benzoxazol-2-yl)spiro[3.3]heptan-2-yl]furan-2-carboxamide;
3-bromo-N-[6-(5-chloro-1,3-benzoxazol-2-yl)spiro[3.3]heptan-2-yl]furan-2-carboxamide;
5-chloro-N-[6-(5-chloro-1,3-benzoxazol-2-yl)spiro[3.3]heptan-2-yl]furan-2-carboxamide;
N-[6-(5-chloro-1,3-benzoxazol-2-yl)spiro[3.3]heptan-2-yl]-5-isopropyl-furan-2-carboxamide;
N-[6-(5-chloro-1,3-benzoxazol-2-yl)spiro[3.3]heptan-2-yl]-5-methoxy-furan-2-carboxamide;
N-[6-(5-chloro-1,3-benzoxazol-2-yl)spiro[3.3]heptan-2-yl]-5-(methoxymethyl)furan-2-carboxamide;
N-[6-(5-chloro-1,3-benzoxazol-2-yl)spiro[3.3]heptan-2-yl]-5-(cyclopropanecarbonyl)furan-2-carboxamide;
N2-[6-(5-chloro-1,3-benzoxazol-2-yl)spiro[3.3]heptan-2-yl]furan-2,5-dicarboxamide;
N-[6-(5-chloro-1,3-benzoxazol-2-yl)spiro[3.3]heptan-2-yl]-3-(trifluoromethyl)-1H-pyrazole-5-carboxamide;
N-[6-(5-chloro-1,3-benzoxazol-2-yl)spiro[3.3]heptan-2-yl]-1-methyl-pyrazole-4-carboxamide;
N-[6-(5-chloro-1,3-benzoxazol-2-yl)spiro[3.3]heptan-2-yl]-5-methylsulfinyl-furan-2-carboxamide;
N-[6-(5-chloro-1,3-benzoxazol-2-yl)spiro[3.3]heptan-2-yl]-5-methylsulfonyl-furan-2-carboxamide;
(Sₐ)—N-[6-(5-chloro-1,3-benzoxazol-2-yl)spiro[3.3]heptan-2-yl]-5-methylsulfonyl-furan-2-carboxamide;
(Rₐ)—N-[6-(5-chloro-1,3-benzoxazol-2-yl)spiro[3.3]heptan-2-yl]-5-methylsulfonyl-furan-2-carboxamide;
N-[6-(5-chloro-1,3-benzoxazol-2-yl)spiro[3.3]heptan-2-yl]-5-ethylsulfonyl-furan-2-carboxamide;
N-[6-(5-chloro-1,3-benzoxazol-2-yl)spiro[3.3]heptan-2-yl]-5-cyclopropylsulfonyl-furan-2-carboxamide;
N-[6-(5-chloro-1,3-benzoxazol-2-yl)spiro[3.3]heptan-2-yl]-5-(oxetan-3-ylsulfonyl)furan-2-carboxamide;
N-[6-(5-Chloro-1,3-benzoxazol-2-yl)spiro[3.3]heptan-2-yl]-5-(cyclopropylmethylsulfinyl)furan-2-carboxamide;
(Rₐ)—N-[6-(5-chloro-1,3-benzoxazol-2-yl)spiro[3.3]heptan-2-yl]-5-[(S)-cyclopropylmethylsulfinyl]furan-2-carboxamide;
(Rₐ)—N-[6-(5-chloro-1,3-benzoxazol-2-yl)spiro[3.3]heptan-2-yl]-5-[(R)-cyclopropylmethylsulfinyl]furan-2-carboxamide;
(Sₐ)—N-[6-(5-Chloro-1,3-benzoxazol-2-yl)spiro[3.3]heptan-2-yl]-5-[(S)-cyclopropylmethylsulfinyl]furan-2-carboxamide;
(Sₐ)—N-[6-(5-chloro-1,3-benzoxazol-2-yl)spiro[3.3]heptan-2-yl]-5-[(R)-cyclopropylmethylsulfinyl]furan-2-carboxamide;
N-[6-(5-chloro-1,3-benzoxazol-2-yl)spiro[3.3]heptan-2-yl]-5-(cyclopropylmethylsulfonyl)furan-2-carboxamide;
N-[6-(5-chloro-1,3-benzoxazol-2-yl)spiro[3.3]heptan-2-yl]-5-isobutylsulfinyl-furan-2-carboxamide;
N-[6-(5-chloro-1,3-benzoxazol-2-yl)spiro[3.3]heptan-2-yl]-5-isobutylsulfonyl-furan-2-carboxamide;
N-[6-(5-chloro-1,3-benzoxazol-2-yl)spiro[3.3]heptan-2-yl]-5-(cyclobutylmethylsulfonyl)furan-2-carboxamide;
N-[6-(5-chloro-1,3-benzoxazol-2-yl)spiro[3.3]heptan-2-yl]-5-(oxetan-3-ylmethylsulfonyl)furan-2-carboxamide;
N-[6-(5-chloro-1,3-benzoxazol-2-yl)spiro[3.3]heptan-2-yl]-5-[(1-methylazetidin-3-yl)methylsulfonyl]furan-2-carboxamide;
benzyl 3-[[5-[[6-(5-chloro-1,3-benzoxazol-2-yl)spiro[3.3]heptan-2-yl]carbamoyl]-2-furyl]sulfonylmethyl]azetidine-1-carboxylate;
N-[6-(5-chloro-1,3-benzoxazol-2-yl)spiro[3.3]heptan-2-yl]-5-[(3,3-difluorocyclobutyl)methylsulfonyl]furan-2-carboxamide;
N-[6-(5-chloro-1,3-benzoxazol-2-yl)spiro[3.3]heptan-2-yl]-5-(2-methoxyethylsulfonyl)furan-2-carboxamide;
N-[6-(5-chloro-1,3-benzoxazol-2-yl)spiro[3.3]heptan-2-yl]-5-sulfamoyl-furan-2-carboxamide;
(Sₐ)—N-[6-(5-chloro-1,3-benzoxazol-2-yl)spiro[3.3]heptan-2-yl]-5-sulfamoyl-furan-2-carboxamide;
(Rₐ)—N-[6-(5-chloro-1,3-benzoxazol-2-yl)spiro[3.3]heptan-2-yl]-5-sulfamoyl-furan-2-carboxamide;
N-[6-(5-chloro-1,3-benzoxazol-2-yl)spiro[3.3]heptan-2-yl]-5-(methylsulfonylmethyl)furan-2-carboxamide;
N-[6-(5-chloro-1,3-benzoxazol-2-yl)spiro[3.3]heptan-2-yl]-5-(cyclopropylsulfonylmethyl)furan-2-carboxamide;
N-[6-(5-chloro-1,3-benzoxazol-2-yl)spiro[3.3]heptan-2-yl]-5-(2-methylsulfonylethyl)furan-2-carboxamide;
N-[6-(5-chloro-1,3-benzoxazol-2-yl)spiro[3.3]heptan-2-yl]-5-(sulfamoylmethyl)furan-2-carboxamide;
N-[6-(5-chloro-1,3-benzoxazol-2-yl)spiro[3.3]heptan-2-yl]-5-(2-sulfamoylethyl)furan-2-carboxamide;
N-[6-(5-chloro-1,3-benzoxazol-2-yl)spiro[3.3]heptan-2-yl]-5-[(sulfamoylamino)methyl]furan-2-carboxamide;
4-bromo-N-[6-(5-chloro-1,3-benzoxazol-2-yl)spiro[3.3]heptan-2-yl]-5-ethylsulfonyl-furan-2-carboxamide;
N-[6-(5-chloro-1,3-benzoxazol-2-yl)spiro[3.3]heptan-2-yl]-5-ethylsulfonyl-3-methyl-furan-2-carboxamide;
5-tert-butylsulfinyl-N-[6-(5-chloro-1,3-benzoxazol-2-yl)spiro[3.3]heptan-2-yl]furan-2-carboxamide;
5-tert-butylsulfonyl-N-[6-(5-chloro-1,3-benzoxazol-2-yl)spiro[3.3]heptan-2-yl]furan-2-carboxamide;
N-[6-(5-chloro-1,3-benzoxazol-2-yl)spiro[3.3]heptan-2-yl]-5-cyclopropylsulfinyl-furan-2-carboxamide;
N-[6-(5-chloro-1,3-benzoxazol-2-yl)spiro[3.3]heptan-2-yl]-5-(trifluoromethylsulfanyl)furan-2-carboxamide;
N-[6-(5-chloro-1,3-benzoxazol-2-yl)spiro[3.3]heptan-2-yl]-5-(trifluoromethylsulfinyl)furan-2-carboxamide;
N-[6-(5-chloro-1,3-benzoxazol-2-yl)spiro[3.3]heptan-2-yl]-5-(trifluoromethylsulfonyl)furan-2-carboxamide;
N-[6-(5-chloro-1,3-benzoxazol-2-yl)spiro[3.3]heptan-2-yl]-5-(isopropylsulfonylmethyl)furan-2-carboxamide;
N-[6-(5-chloro-1,3-benzoxazol-2-yl)spiro[3.3]heptan-2-yl]-5-methylsulfonyl-thiophene-2-carboxamide;
N-[6-(6-Chloro-1,3-benzoxazol-2-yl)spiro[3.3]heptan-2-yl]-5-(trifluoromethyl)furan-2-carboxamide;
N-[6-(6-Fluoro-1,3-benzoxazol-2-yl)spiro[3.3]heptan-2-yl]-5-(trifluoromethyl)furan-2-carboxamide;
N-[6-(6-chlorooxazolo[5,4-b]pyridin-2-yl)spiro[3.3]heptan-2-yl]-5-methylsulfonyl-furan-2-carboxamide;
N-[6-(6-chlorooxazolo[5,4-b]pyridin-2-yl)spiro[3.3]heptan-2-yl]-5-cyclopropylsulfonyl-furan-2-carboxamide;
(Rₐ)—N-[6-(6-chlorooxazolo[5,4-b]pyridin-2-yl)spiro[3.3]heptan-2-yl]-5-cyclopropylsulfonyl-furan-2-carboxamide;
(Sₐ)—N-[6-(6-chlorooxazolo[5,4-b]pyridin-2-yl)spiro[3.3]heptan-2-yl]-5-cyclopropylsulfonyl-furan-2-carboxamide;
N-[6-(6-chlorooxazolo[5,4-b]pyridin-2-yl)spiro[3.3]heptan-2-yl]-5-cyclopropylsulfinyl-furan-2-carboxamide;
N-[6-(6-chlorooxazolo[4,5-c]pyridin-2-yl)spiro[3.3]heptan-2-yl]-5-cyclopropylsulfonyl-furan-2-carboxamide;
N-[6-(5-chloro-1,3-benzoxazol-2-yl)spiro[3.3]heptan-2-yl]-5-(methylsulfonimidoyl)furan-2-carboxamide;
(Rₐ)—N-[6-(5-chloro-1,3-benzoxazol-2-yl)spiro[3.3]heptan-2-yl]-5-[(S)-methylsulfonimidoyl]furan-2-carboxamide;
(Sₐ)—N-[6-(5-chloro-1,3-benzoxazol-2-yl)spiro[3.3]heptan-2-yl]-5-[(S)-methylsulfonimidoyl]furan-2-carboxamide;
(Rₐ)—N-[6-(5-chloro-1,3-benzoxazol-2-yl)spiro[3.3]heptan-2-yl]-5-[(R)-methylsulfonimidoyl]furan-2-carboxamide;
(Sₐ)—N-[6-(5-chloro-1,3-benzoxazol-2-yl)spiro[3.3]heptan-2-yl]-5-[(R)-methylsulfonimidoyl]furan-2-carboxamide;
N-[6-(5-chloro-1,3-benzoxazol-2-yl)spiro[3.3]heptan-2-yl]-5-(cyclopropylsulfonimidoyl)furan-2-carboxamide;
(Rₐ)—N-[6-(5-chloro-1,3-benzoxazol-2-yl)spiro[3.3]heptan-2-yl]-5-[(S)-cyclopropylsulfonimidoyl]furan-2-carboxamide;
(Sₐ)—N-[6-(5-chloro-1,3-benzoxazol-2-yl)spiro[3.3]heptan-2-yl]-5-[(S)-cyclopropylsulfonimidoyl]furan-2-carboxamide;
(Rₐ)—N-[6-(5-chloro-1,3-benzoxazol-2-yl)spiro[3.3]heptan-2-yl]-5-[(R)-cyclopropylsulfonimidoyl]furan-2-carboxamide;
(Sₐ)—N-[6-(5-chloro-1,3-benzoxazol-2-yl)spiro[3.3]heptan-2-yl]-5-[(R)-cyclopropylsulfonimidoyl]furan-2-carboxamide;
N-[6-(5-chloro-1,3-benzoxazol-2-yl)spiro[3.3]heptan-2-yl]-5-(cyclopropylmethylsulfonimidoyl)furan-2-carboxamide;
(Rₐ)—N-[6-(5-chloro-1,3-benzoxazol-2-yl)spiro[3.3]heptan-2-yl]-5-[(S)-cyclopropylmethylsulfonimidoyl]furan-2-carboxamide;
(Rₐ)—N-[6-(5-Chloro-1,3-benzoxazol-2-yl)spiro[3.3]heptan-2-yl]-5-[(R)-cyclopropylmethylsulfonimidoyl]furan-2-carboxamide;
(Sₐ)—N-[6-(5-Chloro-1,3-benzoxazol-2-yl)spiro[3.3]heptan-2-yl]-5-[(S)-cyclopropylmethylsulfonimidoyl]furan-2-carboxamide;
(Sₐ)—N-[6-(5-Chloro-1,3-benzoxazol-2-yl)spiro[3.3]heptan-2-yl]-5-[(R)-cyclopropylmethylsulfonimidoyl]furan-2-carboxamide;
N-[6-(5-chloro-1,3-benzoxazol-2-yl)spiro[3.3]heptan-2-yl]-5-(isobutylsulfonimidoyl)furan-2-carboxamide;
N-[6-(5-chloro-1,3-benzoxazol-2-yl)spiro[3.3]heptan-2-yl]-5-(oxetan-3-ylmethylsulfonimidoyl)furan-2-carboxamide;
N-[6-(5-chloro-1,3-benzoxazol-2-yl)spiro[3.3]heptan-2-yl]-5-[(3,3-difluorocyclobutyl)methylsulfonimidoyl]furan-2-carboxamide;
N-[6-(5-chloro-1,3-benzoxazol-2-yl)spiro[3.3]heptan-2-yl]-5-[(ethylsulfonimidoyl)methyl]furan-2-carboxamide;
N-[6-(5-chloro-1,3-benzoxazol-2-yl)spiro[3.3]heptan-2-yl]-5-[(isopropylsulfonimidoyl)methyl]furan-2-carboxamide;
N-[6-(5-chloro-1,3-benzoxazol-2-yl)spiro[3.3]heptan-2-yl]-5-[(cyclopropylsulfonimidoyl)methyl]furan-2-carboxamide;
N-[6-(5-chloro-1,3-benzoxazol-2-yl)spiro[3.3]heptan-2-yl]-5-(1-oxo-4,5-dihydro-3H-isothiazol-1-yl)furan-2-carboxamide;
N-[6-(5-chloro-1,3-benzoxazol-2-yl)spiro[3.3]heptan-2-yl]-5-(1-oxo-1lambda6-thia-2-azacyclohexen-1-yl)furan-2-carboxamide;
N-[6-(6-chlorooxazolo[5,4-b]pyridin-2-yl)spiro[3.3]heptan-2-yl]-5-(methylsulfonimidoyl)furan-2-carboxamide;
N-[6-(6-chlorooxazolo[5,4-b]pyridin-2-yl)spiro[3.3]heptan-2-yl]-5-(cyclopropylsulfonimidoyl)furan-2-carboxamide;
(Rₐ)—N-[6-(6-chlorooxazolo[5,4-b]pyridin-2-yl)spiro[3.3]heptan-2-yl]-5-[(S)-cyclopropylsulfonimidoyl]furan-2-carboxamide;
(Rₐ)—N-[6-(6-chlorooxazolo[5,4-b]pyridin-2-yl)spiro[3.3]heptan-2-yl]-5-[(R)-cyclopropylsulfonimidoyl]furan-2-carboxamide;
(Sₐ)—N-[6-(6-chlorooxazolo[5,4-b]pyridin-2-yl)spiro[3.3]heptan-2-yl]-5-[(S)-cyclopropylsulfonimidoyl]furan-2-carboxamide;
(Sₐ)—N-[6-(6-chlorooxazolo[5,4-b]pyridin-2-yl)spiro[3.3]heptan-2-yl]-5-[(R)-cyclopropylsulfonimidoyl]furan-2-carboxamide;
N-[6-(6-chlorooxazolo[4,5-c]pyridin-2-yl)spiro[3.3]heptan-2-yl]-5-(cyclopropylsulfonimidoyl)furan-2-carboxamide;
N-[6-(5-chloro-1,3-benzoxazol-2-yl)spiro[3.3]heptan-2-yl]-5-(pyrrolidin-1-ylsulfonimidoyl)furan-2-carboxamide;
N-[6-(5-chloro-1,3-benzoxazol-2-yl)spiro[3.3]heptan-2-yl]-5-(morpholinosulfonimidoyl)furan-2-carboxamide;
N-[6-(5-chloro-1,3-benzoxazol-2-yl)spiro[3.3]heptan-2-yl]-5-(propanoylsulfamoyl)furan-2-carboxamide;
(Sₐ)—N-[6-(5-chloro-1,3-benzoxazol-2-yl)spiro[3.3]heptan-2-yl]-5-(propanoylsulfamoyl)furan-2-carboxamide;
(Rₐ)—N-[6-(5-chloro-1,3-benzoxazol-2-yl)spiro[3.3]heptan-2-yl]-5-(propanoylsulfamoyl)furan-2-carboxamide;
N-(butanoylsulfamoyl)-N-[6-(5-chloro-1,3-benzoxazol-2-yl)spiro[3.3]heptan-2-yl]furan-2-carboxamide;
(Sₐ)—N-(butanoylsulfamoyl)-N-[6-(5-chloro-1,3-benzoxazol-2-yl)spiro[3.3]heptan-2-yl]furan-2-carboxamide;
(Rₐ)—N-(butanoylsulfamoyl)-N-[6-(5-chloro-1,3-benzoxazol-2-yl)spiro[3.3]heptan-2-yl]furan-2-carboxamide;
N-[6-(5-chloro-1,3-benzoxazol-2-yl)spiro[3.3]heptan-2-yl]-5-(2-methylpropanoylsulfamoyl)furan-2-carboxamide;
(Sₐ)—N-[6-(5-chloro-1,3-benzoxazol-2-yl)spiro[3.3]heptan-2-yl]-5-(2-methylpropanoylsulfamoyl)furan-2-carboxamide;
(Rₐ)—N-[6-(5-chloro-1,3-benzoxazol-2-yl)spiro[3.3]heptan-2-yl]-5-(2-methylpropanoylsulfamoyl)furan-2-carboxamide;
N-[6-(5-chloro-1,3-benzoxazol-2-yl)spiro[3.3]heptan-2-yl]-5-(cyclopropanecarbonylsulfamoyl)furan-2-carboxamide;
(Sₐ)—N-[6-(5-chloro-1,3-benzoxazol-2-yl)spiro[3.3]heptan-2-yl]-5-(cyclopropanecarbonylsulfamoyl)furan-2-carboxamide;
(Rₐ)—N-[6-(5-chloro-1,3-benzoxazol-2-yl)spiro[3.3]heptan-2-yl]-5-(cyclopropanecarbonylsulfamoyl)furan-2-carboxamide;
N-[6-(5-chloro-1,3-benzoxazol-2-yl)spiro[3.3]heptan-2-yl]-5-(cyclobutanecarbonylsulfamoyl)furan-2-carboxamide;
(Sₐ)—N-[6-(5-chloro-1,3-benzoxazol-2-yl)spiro[3.3]heptan-2-yl]-5-(cyclobutanecarbonylsulfamoyl)furan-2-carboxamide;
(Rₐ)—N-[6-(5-chloro-1,3-benzoxazol-2-yl)spiro[3.3]heptan-2-yl]-5-(cyclobutanecarbonylsulfamoyl)furan-2-carboxamide;
N-[6-(5-chloro-1,3-benzoxazol-2-yl)spiro[3.3]heptan-2-yl]-5-[(2-methoxyacetyl)sulfamoyl]furan-2-carboxamide;
(Sₐ)—N-[6-(5-chloro-1,3-benzoxazol-2-yl)spiro[3.3]heptan-2-yl]-5-[(2-methoxyacetyl)sulfamoyl]furan-2-carboxamide;
(Rₐ)—N-[6-(5-chloro-1,3-benzoxazol-2-yl)spiro[3.3]heptan-2-yl]-5-[(2-methoxyacetyl)sulfamoyl]furan-2-carboxamide;
ethyl N-[[5-[[6-(5-chloro-1,3-benzoxazol-2-yl)spiro[3.3]heptan-2-yl]carbamoyl]-2-furyl]sulfonyl]carbamate;
(Sₐ)-ethyl N-[[5-[[6-(5-chloro-1,3-benzoxazol-2-yl)spiro[3.3]heptan-2-yl]carbamoyl]-2-furyl]sulfonyl]carbamate;
(Rₐ)-ethyl N-[[5-[[6-(5-chloro-1,3-benzoxazol-2-yl)spiro[3.3]heptan-2-yl]carbamoyl]-2-furyl]sulfonyl]carbamate;
methyl 4-[[5-[[6-(5-chloro-1,3-benzoxazol-2-yl)spiro[3.3]heptan-2-yl]carbamoyl]-2-furyl]sulfonylamino]-4-oxo-butanoate;
(Sₐ)-methyl 4-[[5-[[6-(5-chloro-1,3-benzoxazol-2-yl)spiro[3.3]heptan-2-yl]carbamoyl]-2-furyl]sulfonylamino]-4-oxo-butanoate;
(Rₐ)-methyl 4-[[5-[[6-(5-chloro-1,3-benzoxazol-2-yl)spiro[3.3]heptan-2-yl]carbamoyl]-2-furyl]sulfonylamino]-4-oxo-butanoate;
5-(bicyclo[1.1.1]pentane-1-carbonylsulfamoyl)-N-[6-(5-chloro-1,3-benzoxazol-2-yl)spiro[3.3]heptan-2-yl]furan-2-carboxamide;
(Sₐ)-5-(bicyclo[1.1.1]pentane-1-carbonylsulfamoyl)-N-[6-(5-chloro-1,3-benzoxazol-2-yl)spiro[3.3]heptan-2-yl]furan-2-carboxamide;
(Rₐ)-5-(bicyclo[1.1.1]pentane-1-carbonylsulfamoyl)-N-[6-(5-chloro-1,3-benzoxazol-2-yl)spiro[3.3]heptan-2-yl]furan-2-carboxamide;
N-[6-(5-chloro-1,3-benzoxazol-2-yl)spiro[3.3]heptan-2-yl]-5-(tetrahydrofuran-3-carbonylsulfamoyl)furan-2-carboxamide;
(Sₐ)—N-[6-(5-chloro-1,3-benzoxazol-2-yl)spiro[3.3]heptan-2-yl]-5-(tetrahydrofuran-3-carbonylsulfamoyl)furan-2-carboxamide;
(Rₐ)—N-[6-(5-chloro-1,3-benzoxazol-2-yl)spiro[3.3]heptan-2-yl]-5-(tetrahydrofuran-3-carbonylsulfamoyl)furan-2-carboxamide;
N-[6-(5-chloro-1,3-benzoxazol-2-yl)spiro[3.3]heptan-2-yl]-5-[[2-(2-methoxyethoxy)acetyl]sulfamoyl]furan-2-carboxamide;
(Sₐ)—N-[6-(5-chloro-1,3-benzoxazol-2-yl)spiro[3.3]heptan-2-yl]-5-[[2-(2-methoxyethoxy)acetyl]sulfamoyl]furan-2-carboxamide;
(Rₐ)—N-[6-(5-chloro-1,3-benzoxazol-2-yl)spiro[3.3]heptan-2-yl]-5-[[2-(2-methoxyethoxy)acetyl]sulfamoyl]furan-2-carboxamide;
N-[6-(5-chloro-1,3-benzoxazol-2-yl)spiro[3.3]heptan-2-yl]-5-[[2-[2-(2-methoxyethoxy)ethoxy]acetyl]sulfamoyl]furan-2-carboxamide;
(Sₐ)—N-[6-(5-chloro-1,3-benzoxazol-2-yl)spiro[3.3]heptan-2-yl]-5-[[2-[2-(2-methoxyethoxy)ethoxy]acetyl]sulfamoyl]furan-2-carboxamide;
(Rₐ)—N-[6-(5-chloro-1,3-benzoxazol-2-yl)spiro[3.3]heptan-2-yl]-5-[[2-[2-(2-methoxyethoxy)ethoxy]acetyl]sulfamoyl]furan-2-carboxamide;
5-(acetylsulfamoyl)-N-[6-(5-chloro-1,3-benzoxazol-2-yl)spiro[3.3]heptan-2-yl]furan-2-carboxamide;
N-[6-(5-chloro-1,3-benzoxazol-2-yl)spiro[3.3]heptan-2-yl]-5-(cyclopentanecarbonylsulfamoyl)furan-2-carboxamide;
N-[6-(5-chloro-1,3-benzoxazol-2-yl)spiro[3.3]heptan-2-yl]-5-(cyclohexanecarbonylsulfamoyl)furan-2-carboxamide;
N-[6-(5-chloro-1,3-benzoxazol-2-yl)spiro[3.3]heptan-2-yl]-5-[(1,1-dioxothietane-3-carbonyl)sulfamoyl]furan-2-carboxamide;
N-[6-(5-chloro-1,3-benzoxazol-2-yl)spiro[3.3]heptan-2-yl]-5-[(1,1-dioxothiolane-3-carbonyl)sulfamoyl]furan-2-carboxamide;
N-[6-(5-chloro-1,3-benzoxazol-2-yl)spiro[3.3]heptan-2-yl]-5-[(1,1-dioxothiane-3-carbonyl)sulfamoyl]furan-2-carboxamide;
N-[6-(5-chloro-1,3-benzoxazol-2-yl)spiro[3.3]heptan-2-yl]-5-(oxetane-3-carbonylsulfamoyl)furan-2-carboxamide;
N-[[5-[[6-(5-chloro-1,3-benzoxazol-2-yl)spiro[3.3]heptan-2-yl]carbamoyl]-2-furyl]sulfonyl]tetrahydropyran-4-carboxamide;
N-[6-(5-chloro-1,3-benzoxazol-2-yl)spiro[3.3]heptan-2-yl]-5-(methylsulfamoyl)furan-2-carboxamide; and
N-[6-(5-chloro-1,3-benzoxazol-2-yl)spiro[3.3]heptan-2-yl]-5-(dimethylsulfamoyl)furan-2-carboxamide;
or a pharmaceutically acceptable salt thereof.

In another embodiment (xvi) of the present invention, particular compounds of the present invention are selected from:
(Rₐ)—N-[6-(5-chloro-1,3-benzoxazol-2-yl)spiro[3.3]heptan-2-yl]-5-methylsulfonyl-furan-2-carboxamide;
N-[6-(5-chloro-1,3-benzoxazol-2-yl)spiro[3.3]heptan-2-yl]-5-cyclopropylsulfonyl-furan-2-carboxamide;
N-[6-(5-Chloro-1,3-benzoxazol-2-yl)spiro[3.3]heptan-2-yl]-5-(cyclopropylmethylsulfinyl)furan-2-carboxamide;
(Rₐ)—N-[6-(5-chloro-1,3-benzoxazol-2-yl)spiro[3.3]heptan-2-yl]-5-[(S)-cyclopropylmethylsulfinyl]furan-2-carboxamide;
(Rₐ)—N-[6-(5-chloro-1,3-benzoxazol-2-yl)spiro[3.3]heptan-2-yl]-5-[(R)-cyclopropylmethylsulfinyl]furan-2-carboxamide;
(Sₐ)—N-[6-(5-Chloro-1,3-benzoxazol-2-yl)spiro[3.3]heptan-2-yl]-5-[(S)-cyclopropylmethylsulfinyl]furan-2-carboxamide;
(Sₐ)—N-[6-(5-chloro-1,3-benzoxazol-2-yl)spiro[3.3]heptan-2-yl]-5-[(R)-cyclopropylmethylsulfinyl]furan-2-carboxamide;
N-[6-(5-chloro-1,3-benzoxazol-2-yl)spiro[3.3]heptan-2-yl]-5-(cyclopropylmethylsulfonyl)furan-2-carboxamide;
N-[6-(5-chloro-1,3-benzoxazol-2-yl)spiro[3.3]heptan-2-yl]-5-isobutylsulfinyl-furan-2-carboxamide;
N-[6-(5-chloro-1,3-benzoxazol-2-yl)spiro[3.3]heptan-2-yl]-5-isobutylsulfonyl-furan-2-carboxamide;
N-[6-(5-chloro-1,3-benzoxazol-2-yl)spiro[3.3]heptan-2-yl]-5-(cyclobutylmethylsulfonyl)furan-2-carboxamide;
benzyl 3-[[5-[[6-(5-chloro-1,3-benzoxazol-2-yl)spiro[3.3]heptan-2-yl]carbamoyl]-2-furyl]sulfonylmethyl]azetidine-1-carboxylate;
N-[6-(5-chloro-1,3-benzoxazol-2-yl)spiro[3.3]heptan-2-yl]-5-[(3,3-difluorocyclobutyl)methylsulfonyl]furan-2-carboxamide;
N-[6-(5-chloro-1,3-benzoxazol-2-yl)spiro[3.3]heptan-2-yl]-5-(2-methoxyethylsulfonyl)furan-2-carboxamide;
N-[6-(5-chloro-1,3-benzoxazol-2-yl)spiro[3.3]heptan-2-yl]-5-sulfamoyl-furan-2-carboxamide;
(Sₐ)—N-[6-(5-chloro-1,3-benzoxazol-2-yl)spiro[3.3]heptan-2-yl]-5-sulfamoyl-furan-2-carboxamide;
(Rₐ)—N-[6-(5-chloro-1,3-benzoxazol-2-yl)spiro[3.3]heptan-2-yl]-5-sulfamoyl-furan-2-carboxamide;
N-[6-(5-chloro-1,3-benzoxazol-2-yl)spiro[3.3]heptan-2-yl]-5-(sulfamoylmethyl)furan-2-carboxamide;
4-bromo-N-[6-(5-chloro-1,3-benzoxazol-2-yl)spiro[3.3]heptan-2-yl]-5-ethylsulfonyl-furan-2-carboxamide;
5-tert-butylsulfinyl-N-[6-(5-chloro-1,3-benzoxazol-2-yl)spiro[3.3]heptan-2-yl]furan-2-carboxamide;
5-tert-butylsulfonyl-N-[6-(5-chloro-1,3-benzoxazol-2-yl)spiro[3.3]heptan-2-yl]furan-2-carboxamide;
N-[6-(5-chloro-1,3-benzoxazol-2-yl)spiro[3.3]heptan-2-yl]-5-cyclopropylsulfinyl-furan-2-carboxamide;
N-[6-(5-chloro-1,3-benzoxazol-2-yl)spiro[3.3]heptan-2-yl]-5-(trifluoromethylsulfonyl)furan-2-carboxamide;
N-[6-(6-chlorooxazolo[5,4-b]pyridin-2-yl)spiro[3.3]heptan-2-yl]-5-cyclopropylsulfonyl-furan-2-carboxamide;
(Rₐ)—N-[6-(6-chlorooxazolo[5,4-b]pyridin-2-yl)spiro[3.3]heptan-2-yl]-5-cyclopropylsulfonyl-furan-2-carboxamide;
(Sₐ)—N-[6-(6-chlorooxazolo[5,4-b]pyridin-2-yl)spiro[3.3]heptan-2-yl]-5-cyclopropylsulfonyl-furan-2-carboxamide;
N-[6-(6-chlorooxazolo[5,4-b]pyridin-2-yl)spiro[3.3]heptan-2-yl]-5-cyclopropylsulfinyl-furan-2-carboxamide;
N-[6-(5-chloro-1,3-benzoxazol-2-yl)spiro[3.3]heptan-2-yl]-5-(methylsulfonimidoyl)furan-2-carboxamide;
(Rₐ)—N-[6-(5-chloro-1,3-benzoxazol-2-yl)spiro[3.3]heptan-2-yl]-5-[(S)-methylsulfonimidoyl]furan-2-carboxamide;
(Sₐ)—N-[6-(5-chloro-1,3-benzoxazol-2-yl)spiro[3.3]heptan-2-yl]-5-[(S)-methylsulfonimidoyl]furan-2-carboxamide;
(Rₐ)—N-[6-(5-chloro-1,3-benzoxazol-2-yl)spiro[3.3]heptan-2-yl]-5-[(R)-methylsulfonimidoyl]furan-2-carboxamide;
(Sₐ)—N-[6-(5-chloro-1,3-benzoxazol-2-yl)spiro[3.3]heptan-2-yl]-5-[(R)-methylsulfonimidoyl]furan-2-carboxamide;
N-[6-(5-chloro-1,3-benzoxazol-2-yl)spiro[3.3]heptan-2-yl]-5-(cyclopropylsulfonimidoyl)furan-2-carboxamide;
(Rₐ)—N-[6-(5-chloro-1,3-benzoxazol-2-yl)spiro[3.3]heptan-2-yl]-5-[(S)-cyclopropylsulfonimidoyl]furan-2-carboxamide;
(Sₐ)—N-[6-(5-chloro-1,3-benzoxazol-2-yl)spiro[3.3]heptan-2-yl]-5-[(S)-cyclopropylsulfonimidoyl]furan-2-carboxamide;
(Rₐ)—N-[6-(5-chloro-1,3-benzoxazol-2-yl)spiro[3.3]heptan-2-yl]-5-[(R)-cyclopropylsulfonimidoyl]furan-2-carboxamide;
(Sₐ)—N-[6-(5-chloro-1,3-benzoxazol-2-yl)spiro[3.3]heptan-2-yl]-5-[(R)-cyclopropylsulfonimidoyl]furan-2-carboxamide;
N-[6-(5-chloro-1,3-benzoxazol-2-yl)spiro[3.3]heptan-2-yl]-5-(cyclopropylmethylsulfonimidoyl)furan-2-carboxamide;
(Rₐ)—N-[6-(5-chloro-1,3-benzoxazol-2-yl)spiro[3.3]heptan-2-yl]-5-[(S)-cyclopropylmethylsulfonimidoyl]furan-2-carboxamide;
(Rₐ)—N-[6-(5-Chloro-1,3-benzoxazol-2-yl)spiro[3.3]heptan-2-yl]-5-[(R)-cyclopropylmethylsulfonimidoyl]furan-2-carboxamide;
(Sₐ)—N-[6-(5-Chloro-1,3-benzoxazol-2-yl)spiro[3.3]heptan-2-yl]-5-[(S)-cyclopropylmethylsulfonimidoyl]furan-2-carboxamide;
(Sₐ)—N-[6-(5-Chloro-1,3-benzoxazol-2-yl)spiro[3.3]heptan-2-yl]-5-[(R)-cyclopropylmethylsulfonimidoyl]furan-2-carboxamide;
N-[6-(5-chloro-1,3-benzoxazol-2-yl)spiro[3.3]heptan-2-yl]-5-(isobutylsulfonimidoyl)furan-2-carboxamide;
N-[6-(5-chloro-1,3-benzoxazol-2-yl)spiro[3.3]heptan-2-yl]-5-[(3,3-difluorocyclobutyl)methylsulfonimidoyl]furan-2-carboxamide;
N-[6-(6-chlorooxazolo[5,4-b]pyridin-2-yl)spiro[3.3]heptan-2-yl]-5-(cyclopropylsulfonimidoyl)furan-2-carboxamide;
(Rₐ)—N-[6-(6-chlorooxazolo[5,4-b]pyridin-2-yl)spiro[3.3]heptan-2-yl]-5-[(S)-cyclopropylsulfonimidoyl]furan-2-carboxamide;
(Rₐ)—N-[6-(6-chlorooxazolo[5,4-b]pyridin-2-yl)spiro[3.3]heptan-2-yl]-5-[(R)-cyclopropylsulfonimidoyl]furan-2-carboxamide;
(Sₐ)—N-[6-(6-chlorooxazolo[5,4-b]pyridin-2-yl)spiro[3.3]heptan-2-yl]-5-[(S)-cyclopropylsulfonimidoyl]furan-2-carboxamide;
(Sₐ)—N-[6-(6-chlorooxazolo[5,4-b]pyridin-2-yl)spiro[3.3]heptan-2-yl]-5-[(R)-cyclopropylsulfonimidoyl]furan-2-carboxamide;
N-[6-(5-chloro-1,3-benzoxazol-2-yl)spiro[3.3]heptan-2-yl]-5-(propanoylsulfamoyl)furan-2-carboxamide;
(Sₐ)—N-[6-(5-chloro-1,3-benzoxazol-2-yl)spiro[3.3]heptan-2-yl]-5-(propanoylsulfamoyl)furan-2-carboxamide;
(Rₐ)—N-[6-(5-chloro-1,3-benzoxazol-2-yl)spiro[3.3]heptan-2-yl]-5-(propanoylsulfamoyl)furan-2-carboxamide;
N-(butanoylsulfamoyl)-N-[6-(5-chloro-1,3-benzoxazol-2-yl)spiro[3.3]heptan-2-yl]furan-2-carboxamide;
(Sₐ)—N-(butanoylsulfamoyl)-N-[6-(5-chloro-1,3-benzoxazol-2-yl)spiro[3.3]heptan-2-yl]furan-2-carboxamide;
(Rₐ)—N-(butanoylsulfamoyl)-N-[6-(5-chloro-1,3-benzoxazol-2-yl)spiro[3.3]heptan-2-yl]furan-2-carboxamide;
N-[6-(5-chloro-1,3-benzoxazol-2-yl)spiro[3.3]heptan-2-yl]-5-(2-methylpropanoylsulfamoyl)furan-2-carboxamide;
(Sₐ)—N-[6-(5-chloro-1,3-benzoxazol-2-yl)spiro[3.3]heptan-2-yl]-5-(2-methylpropanoylsulfamoyl)furan-2-carboxamide;
(Rₐ)—N-[6-(5-chloro-1,3-benzoxazol-2-yl)spiro[3.3]heptan-2-yl]-5-(2-methylpropanoylsulfamoyl)furan-2-carboxamide;
N-[6-(5-chloro-1,3-benzoxazol-2-yl)spiro[3.3]heptan-2-yl]-5-(cyclobutanecarbonylsulfamoyl)furan-2-carboxamide;
(Sₐ)—N-[6-(5-chloro-1,3-benzoxazol-2-yl)spiro[3.3]heptan-2-yl]-5-(cyclobutanecarbonylsulfamoyl)furan-2-carboxamide;
(Rₐ)—N-[6-(5-chloro-1,3-benzoxazol-2-yl)spiro[3.3]heptan-2-yl]-5-(cyclobutanecarbonylsulfamoyl)furan-2-carboxamide;
N-[6-(5-chloro-1,3-benzoxazol-2-yl)spiro[3.3]heptan-2-yl]-5-[(2-methoxyacetyl)sulfamoyl]furan-2-carboxamide;
(Sₐ)—N-[6-(5-chloro-1,3-benzoxazol-2-yl)spiro[3.3]heptan-2-yl]-5-[(2-methoxyacetyl)sulfamoyl]furan-2-carboxamide;
(Rₐ)—N-[6-(5-chloro-1,3-benzoxazol-2-yl)spiro[3.3]heptan-2-yl]-5-[(2-methoxyacetyl)sulfamoyl]furan-2-carboxamide;
N-[6-(5-chloro-1,3-benzoxazol-2-yl)spiro[3.3]heptan-2-yl]-5-(cyclohexanecarbonylsulfamoyl)furan-2-carboxamide; and
N-[6-(5-chloro-1,3-benzoxazol-2-yl)spiro[3.3]heptan-2-yl]-5-(methylsulfamoyl)furan-2-carboxamide;
or a pharmaceutically acceptable salt thereof.

### Synthesis

The compounds of the present invention can be prepared by any conventional means. Suitable processes for synthesizing these compounds as well as their starting materials are provided in the schemes below and in the examples. All substituents, in particular, A₁ to A₄, X₁, R^{x}, R^{y}, Cy, L and R¹ are as defined above unless otherwise indicated. Furthermore, and unless explicitly otherwise stated, all reactions, reaction conditions, abbreviations and symbols have the meanings well known to a person of ordinary skill in organic chemistry.

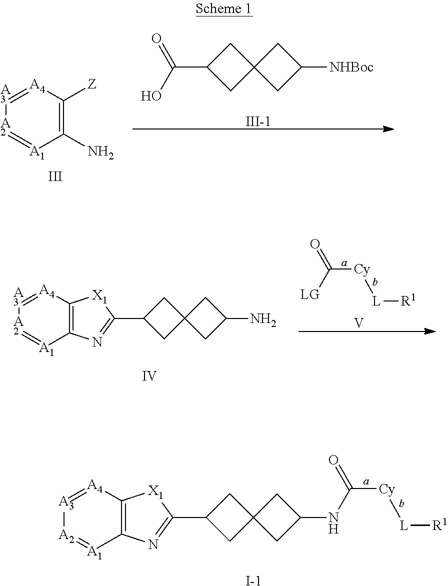

Wherein Z is halogen or OH; LG is halogen or OH.

Compound of formula III is heated with a carboxylic acid III-1 in the presence of an acid, such as polyphosphoric acid, to give compound of formula IV, which then reacts with compound of formula V in the presence of a coupling reagent, such as HATU or T₃P, and a base such as TEA or DIPEA, in a solvent such as DMF or DCM, to afford compound of formula I-1.

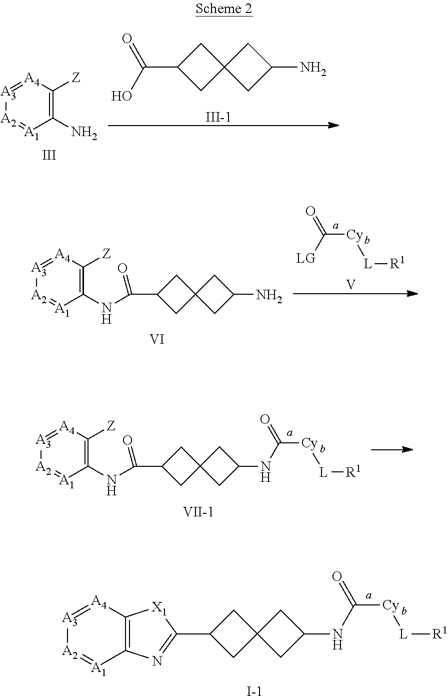

Wherein Z is halogen or OH.

Compound of formula III is heated with a carboxylic acid III-1 in the presence of an acid, such as polyphosphoric acid, to give compound of formula VI, which then reacts with compound of formula V in the presence of a coupling reagent such as HATU or T₃P, and a base such as TEA or DIPEA, in a solvent such as DMF or DCM, to afford compound of formula VII-1. Cyclization of compound of formula VII-1 with microwave irradiation in the presence of a base such as K₂CO₃, in a suitable solvent such as NMP, affords compound of formula I-1. Compound of formula I-1 can also be formed with DIAD and PPh₃, in a suitable solvent such as THF.

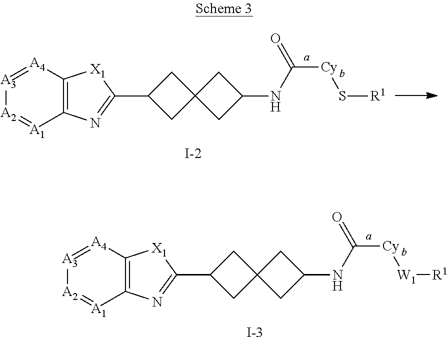

Wherein W₁ is S(O), S(O)₂ or S(O)(NH).

Oxidation of compound of formula I-2 in the presence of an oxidate, such as m-CPBA, or PhI(OAc)₂ and (NH₄)₂CO₃, in a suitable solvent, such as MeOH or DCM, affords compound of formula I-3.

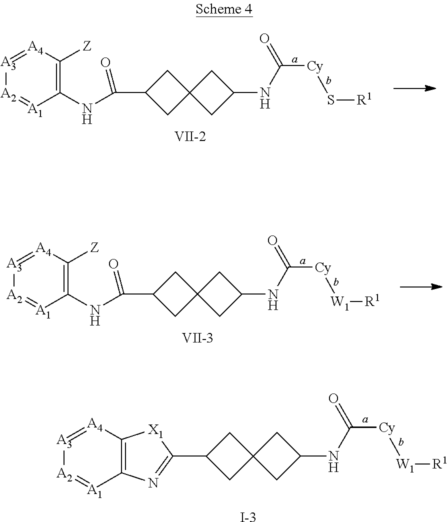

Wherein Z is halogen or OH; W₁ is S(O), S(O)₂ or S(O)(NH).

Oxidation of compound of formula VII-2 in the presence of an oxidate, such as m-CPBA, or PhI(OAc)₂ and (NH₄)₂CO₃, affords compound of formula VII-3. Cyclization of compound of formula VII-3 with microwave irradiation in the presence of a base, such as K₂CO₃, in a suitable solvent, such as NMP, affords compound of formula I-3. Compound of formula I-3 can also be formed with DIAD and PPh₃, in a suitable solvent such as THF.

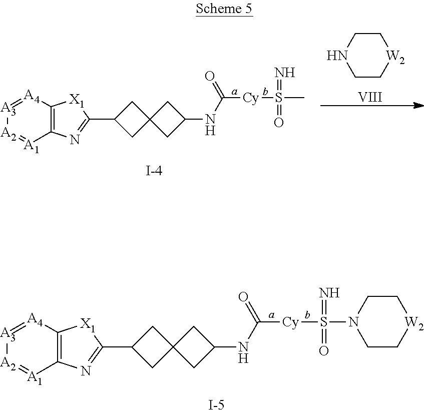

Wherein W₂ is a bond or O.

Compound of formula I-4 is heated with a cyclized amine VIII under O₂ atmosphere in the presence of a catalyst, such as thiophene-2-carbonyloxycopper, in a suitable solvent, such as toluene, affords compound of formula I-5.

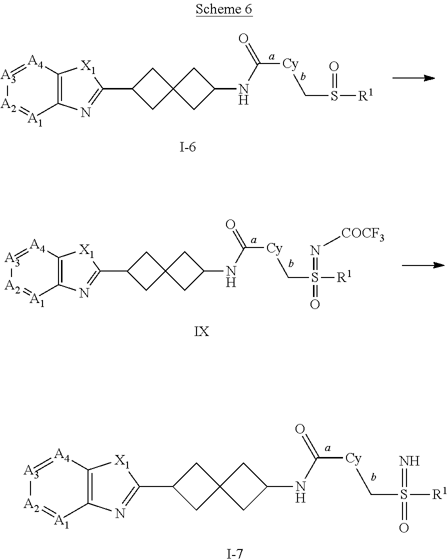

Compound of formula I-6 reacts with 2,2,2-trifluoroacetamide in the presence of magnesium oxide, rhodium(II) acetate dimer and iodobenzene diacetate, in a suitable solvent such as DCM, affords compound of formula IX, which is then deprotected with a base, such as K₂CO₃, in a suitable solvent such as MeOH, to afford compound of formula I-7.

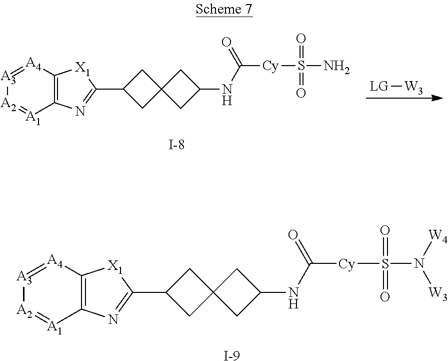

Wherein LG is halogen; W₃ is C₁₋₆alkyl or C(O)R¹; W₄ is H or W₃.

Compound of formula I-8 reacts with halide X in the presence of a base, such as K₂CO₃, DMAP or TEA, in a solvent such as DCM or MeOH, to afford compound of formula I-9.

This invention also relates to a process for the preparation of a compound of formula (I) comprising at least one of the following steps:
(a) Reaction of a compound of formula (IV),

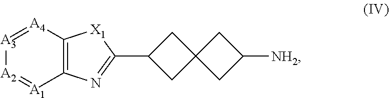

with a compound of formula (V),

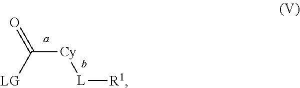

in the presence of a coupling reagent and a base;
(b) Cyclization of a compound of formula (VII-1),

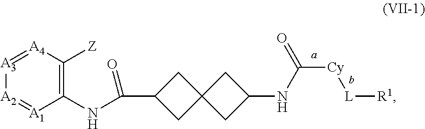

in the presence of a base;
(c) Cyclization of a compound of formula (VII-1) in the presence of DIAD and PPh₃;
(d) Oxidation of a compound of formula (I-2),

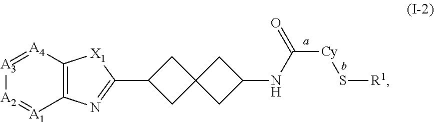

in the presence of an oxidate;
(e) Cyclization of a compound of formula (VII-3),

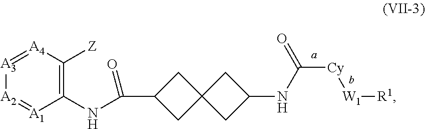

in the presence of a base;
(f) Cyclization of a compound of formula (VII-3) in the presence of DIAD and PPh₃;
(g) Reaction of a compound of formula (I-4),

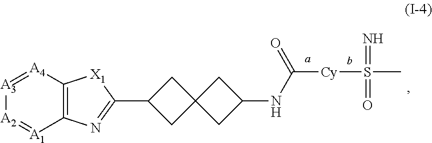

with a cyclized amine (VIII),

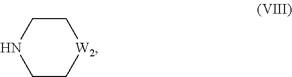

in the presence of a catalyst;
(h) Deprotection of a compound of formula (IX),

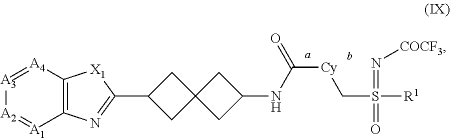

in the presence of a base;
(i) Reaction of a compound of formula (I-8),

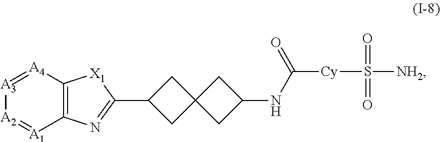

with a halide (X), LG-W₃
(X), in the presence of a base;
   wherein A₁ to A₄, X₁, R^{x}, R^{y}, Cy, L and R¹ are defined above; Z is halogen or OH; LG is halogen or OH; W₁ is S(O), S(O)₂ or S(O)(NH); W₂ is a bond or O; W₃ is C₁₋₆alkyl or C(O)R¹.
   The coupling reagent in step (a) can be for example HATU or T₃P;
   The base in step (a) can be for example TEA or DIPEA;
   The base in step (b) can be for example K₂CO₃;
   The oxidate in step (d) can be for example m-CPBA, or PhI(OAc)₂ and (NH₄)₂CO₃;
   The base in step (e) can be for example K₂CO₃;
   The catalyst in step (g) can be for example thiophene-2-carbonyloxycopper;
   The base in step (h) can be for example K₂CO₃;
   The base in step (i) can be for example K₂CO₃, DMAP or TEA.

A compound of formula (I) or (II) when manufactured according to the above process is also an object of the invention.

The compound of this invention also shows good safety and PK profile.

### Pharmaceutical Compositions and Administration

The invention also relates to a compound of formula (I) or (II) for use as therapeutically active substance. Another embodiment provides pharmaceutical compositions or medicaments containing the compounds of the invention and a therapeutically inert carrier, diluent or excipient, as well as methods of using the compounds of the invention to prepare such compositions and medicaments. In one example, compounds of formula (I) or (II) may be formulated by mixing at ambient temperature at the appropriate pH, and at the desired degree of purity, with physiologically acceptable carriers, i.e., carriers that are non-toxic to recipients at the dosages and concentrations employed into a galenical administration form. The pH of the formulation depends mainly on the particular use and the concentration of compound, but preferably ranges anywhere from about 3 to about 8. In one example, a compound of formula (I) or (II) is formulated in an acetate buffer, at pH 5. In another embodiment, the compounds of formula (I) or (II) are sterile. The compound may be stored, for example, as a solid or amorphous composition, as a lyophilized formulation or as an aqueous solution.

Compositions are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “effective amount” of the compound to be administered will be governed by such considerations, and is the minimum amount necessary to reduction of HBsAg and HBeAg in HBV patients. For example, such amount may be below the amount that is toxic to normal cells, or the mammal as a whole.

In one example, the pharmaceutically effective amount of the compound of the invention administered parenterally per dose will be in the range of about 0.1 to 100 mg/kg, alternatively about 0.1 to 50 mg/kg of patient body weight per day, with the typical initial range of compound used being 0.3 to 15 mg/kg/day. In another embodiment, oral unit dosage forms, such as tablets and capsules, preferably contain from about 25 to about 1000 mg of the compound of the invention.

The compounds of the invention may be administered by any suitable means, including oral, topical (including buccal and sublingual), rectal, vaginal, transdermal, parenteral, subcutaneous, intraperitoneal, intrapulmonary, intradermal, intrathecal and epidural and intranasal, and, if desired for local treatment, intralesional administration. Parenteral infusions include intramuscular, intravenous, intraarterial, intraperitoneal, or subcutaneous administration.

The compounds of the present invention may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents.

A typical formulation is prepared by mixing a compound of the present invention and a carrier or excipient. Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

An example of a suitable oral dosage form is a tablet containing about 25 to 500 mg of the compound of the invention compounded with about 90 to 30 mg anhydrous lactose, about 5 to 40 mg sodium croscarmellose, about 5 to 30 mg polyvinylpyrrolidone (PVP) K30, and about 1 to 10 mg magnesium stearate. The powdered ingredients are first mixed together and then mixed with a solution of the PVP. The resulting composition can be dried, granulated, mixed with the magnesium stearate and compressed to tablet form using conventional equipment. An example of an aerosol formulation can be prepared by dissolving the compound, for example 5 to 400 mg, of the invention in a suitable buffer solution, e.g. a phosphate buffer, adding a tonicifier, e.g. a salt such sodium chloride, if desired. The solution may be filtered, e.g., using a 0.2 micron filter, to remove impurities and contaminants.

An embodiment, therefore, includes a pharmaceutical composition comprising a compound of formula (I) or (II), or a pharmaceutically acceptable salt thereof.

In a further embodiment includes a pharmaceutical composition comprising a compound of formula (I) or (II), or a pharmaceutically acceptable salt thereof, together with a pharmaceutically acceptable carrier or excipient.

Another embodiment includes a pharmaceutical composition comprising a compound of formula (I) or (II), or a pharmaceutically acceptable salt thereof for use in the treatment of HBV infection.

### Indications and Methods of Treatment

The compounds of the invention have anti-HBV activity. Accordingly, the compounds of the invention are useful for the treatment or prophylaxis of HBV infection.

The invention also relates to the use of a compound of formula (I) or (II) for the inhibition of HBeAg.

The invention further relates to the use of a compound of formula (I) or (II) for the inhibition of HBsAg.

The invention relates to the use of a compound of formula (I) or (II) for the inhibition of HBV DNA.

The invention relates to the use of a compound of formula (I) or (II) for use in the treatment or prophylaxis of HBV infection.

The use of a compound of formula (I) or (II) for the preparation of medicaments useful in the treatment or prophylaxis diseases that are related to HBV infection is an object of the invention.

The invention relates in particular to the use of a compound of formula (I) or (II) for the preparation of a medicament for the treatment or prophylaxis of HBV infection.

Another embodiment includes a method for the treatment or prophylaxis of HBV infection, which method comprises administering an effective amount of a compound of formula (I) or (II), or a pharmaceutically acceptable salt thereof.

The invention relates in particular to a compound of formula (I) and (II) for use in the treatment or prophylaxis of HBV infection.

### BRIEF DESCRIPTION OF THE FIGURES

FIG. 1: X-ray crystal structure of Example 15-b

FIG. 2: X-ray crystal structure of Example 52-d

FIG. 3: X-ray crystal structure of Example 53-c

### EXAMPLES

The invention will be more fully understood by reference to the following examples. They should not, however, be construed as limiting the scope of the invention.

Abbreviations used herein are as follows:
ACN: acetonitrile
CDCl₃: deuterated chloroform
CD₃OD: deuterated methanol
DIAD: diisopropyl azodicarboxylate
DIPEA: N,N-diisopropylethylamine
DMAP 4-dimethylaminopyridine
DMF: dimethylformamide
DMPU: 1,3-dimethyl-tetrahydropyrimidin-2(1H)-one
DMSO-d₆: deuterated dimethylsulfoxide
EtOAc: ethyl acetate
HATU O-(7-aza-1H-benzotriazole-1-yl)-N,N,N′,N′-tetramethyluronium hexafluorophosphate
HPLC: high performance liquid chromatography
h: hour
IC₅₀: the half maximal inhibitory concentration
LC/MS: liquid chromatography/mass spectrometry
MeOH: methanol
M: molarity
m-CPBA: 3-chloroperoxybenzoic acid
MHz: megahertz
min: minute
mL: milliliter
mmol: millimole
MS (ESI): mass spectroscopy (electron spray ionization)
NMP: N-methyl pyrrolidone
NMR: nuclear magnetic resonance
obsd. observed
PPh₃: triphenylphosphine
SFC: supercritical fluid chromatography
TEA: triethylamine
TFA: trifluoroacetic acid
THF: tetrahydrofuran
TLC: thin layer chromatography
T₃P: 2,4,6-tripropyl-1,3,5,2,4,6-trioxatriphosphinane 2,4,6-trioxide
δ: chemical shift

### General Experimental Conditions

Intermediates and final compounds were purified by flash chromatography using one of the following instruments: i) Biotage SP1 system and the Quad 12/25 Cartridge module. ii) ISCO combi-flash chromatography instrument. Silica gel Brand and pore size: i) KP-SIL 60 Å, particle size: 40-60 μm; ii) CAS registry NO: Silica Gel: 63231-67-4, particle size: 47-60 micron silica gel; iii) ZCX from Qingdao Haiyang Chemical Co., Ltd, pore: 200-300 or 300-400.

Intermediates and final compounds were purified by preparative HPLC on reversed phase column using X Bridge™ Perp C₁₈ (5 μm, OBD™ 30×100 mm) column or SunFire™ Perp C₁₈ (5 μm, OBD™ 30×100 mm) column.

LC/MS spectra were obtained using an Acquity Ultra Performance LC—3100 Mass Detector or Acquity Ultra Performance LC—SQ Detector. Standard LC/MS conditions were as follows (running time 3 minutes):
Acidic condition: A: 0.1% formic acid in H₂O; B: 0.1% formic acid in acetonitrile;
Basic condition: A: 0.05% NH₃H₂O in H₂O; B: acetonitrile;
Neutral condition: A: H₂O; B: acetonitrile.

Mass spectra (MS): generally only ions which indicate the parent mass are reported, and unless otherwise stated the mass ion quoted is the positive mass ion (M+H)+.

The microwave assisted reactions were carried out in a Biotage Initiator Sixty or CEM Discover.

NMR Spectra were obtained using Bruker Avance 400 MHz.

All reactions involving air-sensitive reagents were performed under an argon atmosphere. Reagents were used as received from commercial suppliers without further purification unless otherwise noted.

### PREPARATIVE EXAMPLES

The invention will be more fully understood by reference to the following examples. They should not, however, be construed as limiting the scope of the invention.

### Intermediate Int-1

### 5-methylsulfanylfuran-2-carboxylic acid

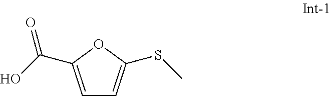

The title compound was prepared according to the following scheme:

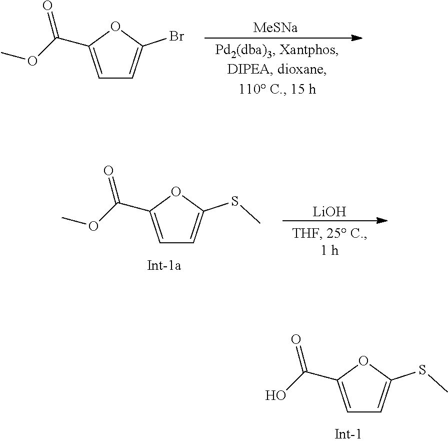

### Step 1: Preparation of methyl 5-methylsulfanylfuran-2-carboxylate (Int-1a)

A mixture of methyl 5-bromo-2-furoate (10 g, 48.78 mmol), sodium thiomethoxide (6.84 g, 97.56 mmol), 9,9-dimethyl-4,5-bis(diphenylphosphino)xanthene (2.82 g, 4.88 mmol), N,N-diisopropylethylamine (25.49 mL, 146.33 mmol) and tris(dibenzylideneacetone)dipalladium (0) (2.23 g, 2.44 mmol) in 1,4-dioxane (200 mL) was stirred at 110° C. for 15 h. The solvent was evaporated in vacuo and the EtOAc (300 mL) was added. The resulting mixture was filtered by a short silica gel column. The filtrate was concentrated and purified by flash column (eluting with EtOAc/PE=3/97) to give methyl 5-methylsulfanylfuran-2-carboxylate as a yellow oil (Int-1a, 7 g, 83.3%). MS obsd. (ESI⁺) [(M+H)⁺]: 173.1.

### Step 2: Preparation of 5-methylsulfanylfuran-2-carboxylic acid (Int-1)

To a solution of methyl 5-methylsulfanylfuran-2-carboxylate (Int-1a, 7 g, 40.65 mmol) in THF (10 mL) was added LiOH (2.5 M, 19.5 mL). After being stirred at 25° C. for 1 h, the reaction mixture was concentrated under reduced pressure to remove the organic solvent. The aqueous layer was extracted with EtOAc (100 mL×2), and then acidified with aqueous HCl until pH=3 to 4. The precipitate was collected by filtration and dried in vacuo to give 5-methylsulfanylfuran-2-carboxylic acid as alight yellow solid (Int-1, 5.6 g, 87.1%). MS obsd. (ESI⁺) [(M+H)⁺]: 159.1. ¹H NMR (400 MHz, CDCl₃) δ ppm: 7.30 (d, J=3.6 Hz, 1H), 6.40 (d, J=3.6 Hz, 1H), 2.55 (s, 3H).

### Intermediate Int-2

### 5-methylsulfinylfuran-2-carboxylic acid

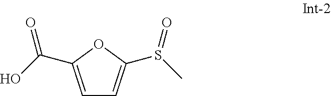

The title compound was prepared according to the following scheme:

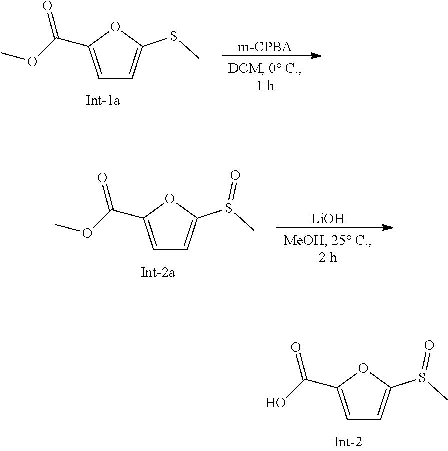

### Step 1: Preparation of methyl 5-methylsulfinylfuran-2-carboxylate (Int-2a)

To a solution of methyl 5-methylsulfanylfuran-2-carboxylate (Int-1a, 4.5 g, 26.1 mmol) in DCM (10 mL) was added m-CPBA (4.5 g, 26.1 mmol). After being stirred at 0° C. for 1 h, the mixture was washed with saturated Na₂CO₃. The organic layer was dried over Na₂SO₄, filtered and concentrated in vacuo. The residue was purified by flash column (eluting with EtOAc/PE=1/2) to give methyl 5-methylsulfinylfuran-2-carboxylate as a light yellow solid (Int-2a, 3.7 g, 75%). MS obsd. (ESI⁺) [(M+H)⁺]: 189.1.

### Step 2: Preparation of 5-methylsulfinylfuran-2-carboxylic acid (Int-2)

To a solution of methyl 5-methylsulfinylfuran-2-carboxylate (Int-2a, 90 mg, 0.48 mmol) in a mixed solvent of MeOH (10 mL) and water (10 mL) was added LiOH·H₂O (134 mg, 2.4 mmol). After being stirred at 25° C. for 2 h, the MeOH was evaporated. The residue was acidified by HCl (1 M) to pH=2 and extracted with DCM (15 mL×3). The combined organic layer was dried over Na₂SO₄, filtered and concentrated in vacuo to give 5-methylsulfinylfuran-2-carboxylic acid as a yellow oil (Int-2, 60 mg, 72%). MS obsd. (ESI⁺) [(M+H)⁺]: 175.1.

### Intermediate Int-3

### 5-methylsulfonylfuran-2-carboxylic acid

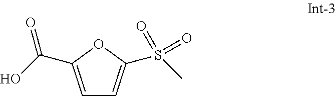

The title compound was prepared according to the following scheme:

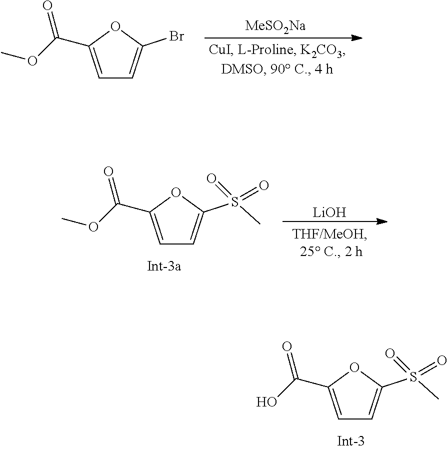

### Step 1: Preparation of methyl 5-methylsulfonylfuran-2-carboxylate (Int-3a)

To a solution of methyl 5-bromo-2-furoate (2.05 g, 10 mmol) in DMSO (25 mL) was added methylsulfinyloxysodium (1.23 g, 12 mmol) followed by copper (I) iodide (380.9 mg, 2 mmol), L-proline (460.5 mg, 4 mmol) and K₂CO₃ (414.6 mg, 3 mmol). The resulting mixture was heated with stirring at 90° C. for 4 h. The reaction was diluted with water (30 mL) and extracted with EtOAc (30 mL×3). The combined organic layer was washed with brine (50 mL×2), dried over Na₂SO₄, filtered and concentrated in vacuo. The residue was purified by flash column (eluting with EtOAc/PE=1/3) to give methyl 5-methylsulfonylfuran-2-carboxylate as a white solid (Int-3a, 750 mg, 36.7%). MS obsd. (ESI⁺) [(M+H)⁺]: 205.1.

### Step 2: Preparation of 5-methylsulfonylfuran-2-carboxylic acid (Int-3)

To a solution of methyl 5-methylsulfonylfuran-2-carboxylate (Int-3a, 750 mg, 3.7 mmol) in a mixed solvent of THF (25 mL) and MeOH (25 mL) was added an aqueous of LiOH·H₂O (2 M, 55 mL). After being stirred at 25° C. for 2 h, most of the solvent was evaporated. The residue was acidified by HCl (2 M) to pH=3 and extracted with EtOAc (20 mL×3). The combined organic layer was dried over Na₂SO₄, filtered and concentrated in vacuo to give 5-methylsulfonylfuran-2-carboxylic acid as a white solid (Int-3, 655 mg, 89.1%). MS obsd. (ESI⁺) [(M+H)⁺]: 191.1.

### Intermediate Int-4

### 5-ethylsulfonylfuran-2-carboxylic acid

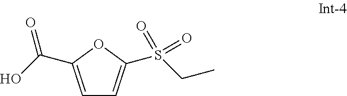

The title compound was prepared according to the following scheme:

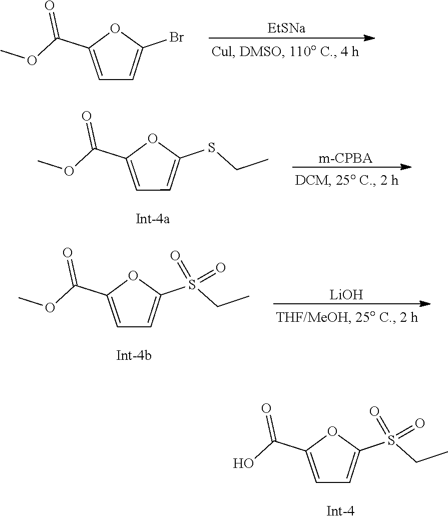

### Step 1: Preparation of methyl 5-ethylsulfanylfuran-2-carboxylate (Int-4a)

To a solution of methyl 5-bromo-2-furoate (610 mg, 3 mmol) in DMSO (6 mL) was added ethylsulfanylsodium (501 mg, 6 mmol) followed by copper (I) iodide (567 mg, 3 mmol). The mixture was then heated with stirring at 110° C. for 4 h. The reaction was diluted with water (30 mL) and extracted with EtOAc (20 mL×3). The combined organic layer was washed with brine (50 mL×2), dried over Na₂SO₄, filtered and concentrated to afford methyl 5-ethylsulfanylfuran-2-carboxylate as a brown oil (Int-4a, 318 mg), which was used for the next step without further purification. MS obsd. (ESI⁺) [(M+H)⁺]: 187.1.

### Step 2: Preparation of methyl 5-ethylsulfonylfuran-2-carboxylate (Int-4b)

To a solution of methyl 5-ethylsulfanylfuran-2-carboxylate (Int-4a, 310 mg, 1.7 mmol) in DCM (10 mL) was added m-CPBA (862 mg, 5 mmol). After being stirred at 25° C. for 2 h, the mixture was washed with saturated NaHCO₃. The organic layer was dried over Na₂SO₄, filtered and concentrated in vacuo. The residue was purified by flash column (eluting with EtOAc/PE=1/3) to give methyl 5-ethylsulfonylfuran-2-carboxylate as a light yellow oil (Int-4b, 240 mg, 55.1%). MS obsd. (ESI⁺) [(M+H)⁺]: 219.1.

### Step 3: Preparation of 5-ethylsulfonylfuran-2-carboxylic acid (Int-4)

To a solution of methyl 5-ethylsulfonylfuran-2-carboxylate (Int-4b, 240 mg, 1.1 mmol) in a mixed solvent of THF (5 mL) and MeOH (5 mL) was added an aqueous of LiOH·H₂O (2 M, 5.5 mL). After being stirred at 25° C. for 2 h, most of the solvent was evaporated. The residue was acidified by HCl (2 M) to pH=3 and extracted with EtOAc (5 mL×3). The combined organic layer was dried over Na₂SO₄, filtered and concentrated in vacuo to give 5-ethylsulfonylfuran-2-carboxylic acid as a light yellow solid (Int-4, 220 mg), which was used for the next step without further purification. MS obsd. (ESI⁺) [(M+H)⁺]: 205.1.

### Intermediate Int-5

### 5-cyclopropylsulfanylfuran-2-carboxylic acid

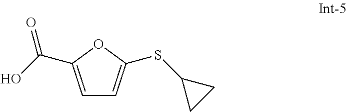 [SMILES: O=C(O)c1ccc(SC2CC2)o1]

The title compound was prepared according to the following scheme:

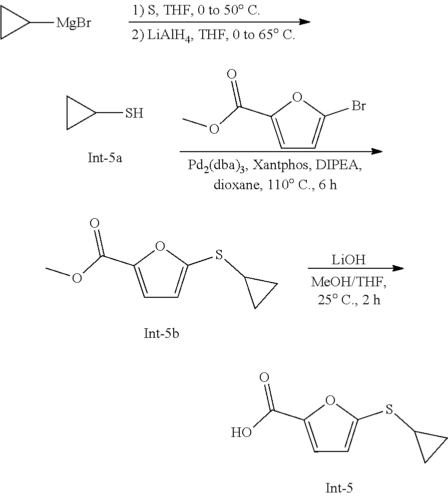

### Step 1: Preparation of cyclopropanethiol (Int-5a)

To a solution of cyclopropylmagnesium bromide (10 mL, 5 mmol) in THF was added sulfur (160 mg, 0.63 mmol) at 0° C. Then the solution was heated at 50° C. with stirring for 3 h. After being cooled in an ice-bath, lithium aluminum hydride (5 mL, 5 mmol) in THF was added. The resulting mixture was stirred at 65° C. for 0.5 h and quenched by H₂O (0.5 mL) at 0° C., then acidified by H₂SO₄ (5% v/v, 20 mL). The organic phase containing cyclopropanethiol (Int-5a) was separated, dried over anhydrous Na₂SO₄ and used for the next step without purification.

### Step 2: Preparation of methyl 5-cyclopropylsulfanylfuran-2-carboxylate (Int-5b)

To a mixture of methyl 5-bromo-2-furoate (410 mg, 2 mmol), tris(dibenzylideneacetone)dipalladium (0) (183 mg, 0.2 mmol), 9,9-dimethyl-4,5-bis(diphenylphosphino)xanthene (116 mg, 0.2 mmol) and N,N-diisopropylethylamine (1.74 mL, mmol) in 1,4-dioxane (15 mL) was added the above solution containing cyclopropanethiol (Int-5a). After being stirred at 110° C. for 6 h, the mixture was filtered with a short silica gel column. The filtration was concentrated and purified by flash column (eluting with EtOAc/PE=1/99) to give methyl 5-cyclopropylsulfanylfuran-2-carboxylate as a colorless oil (Int-5b, 120 mg, 12%). MS obsd. (ESI⁺) [(M+H)⁺]: 199.1.

### Step 3: Preparation of 5-cyclopropylsulfanylfuran-2-carboxylic acid (Int-5)

To a solution of methyl 5-cyclopropylsulfanylfuran-2-carboxylate (Int-5b, 50 mg, 0.25 mmol) in a mixed solvent of THF (2 mL) and MeOH (2 mL) was added a solution of LiOH in water (1.9 mL, 2 M). After being stirred at 25° C. for 2 h, the mixture was acidified by HCl (1 M) to pH=5, then extracted by EtOAc (10 mL×3). The combined organic layer was dried over Na₂SO₄, filtered and concentrated to give 5-cyclopropylsulfanylfuran-2-carboxylic acid as a white solid (Int-5, 46 mg, 99%). MS obsd. (ESI⁺) [(M+H)⁺]: 185.1.

### Intermediate Int-6

### 5-cyclopropylsulfonylfuran-2-carboxylic acid

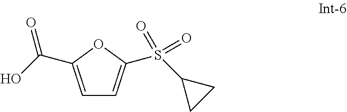 [SMILES: O=C(O)c1ccc(S(=O)(=O)C2CC2)o1]

The title compound was prepared in analogy to the procedure described for the preparation of 5-methylsulfonylfuran-2-carboxylic acid (Int-3), by using cyclopropylsulfinyloxysodium instead of methylsulfinyloxysodium. MS obsd. (ESI⁺) [(M+H)⁺]: 217.1.

### Intermediate Int-7

### 5-(oxetan-3-ylsulfonyl)furan-2-carboxylic acid

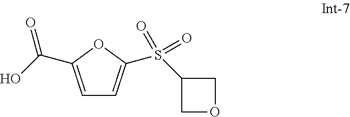 [SMILES: O=C(O)c1ccc(S(=O)(=O)C2COC2)o1]

The title compound was prepared according to the following scheme:

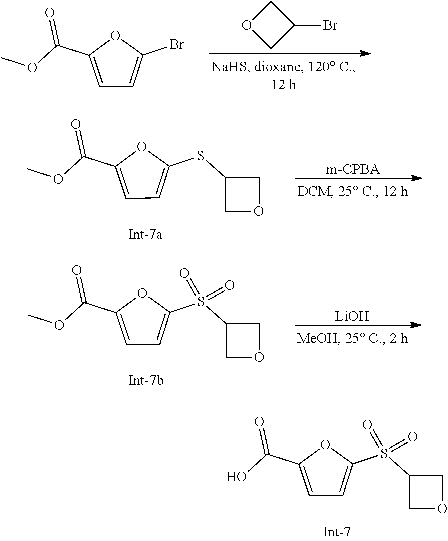

### Step 1: Preparation of methyl 5-(oxetan-3-ylsulfanyl)furan-2-carboxylate (Int-7a)

To a solution of methyl 5-bromo-2-furoate (1 g, 4.88 mmol) in 1,4-dioxane (10 mL) was added sodium hydrosulfide (2.74 g, 48.8 mmol) and 3-bromooxetane (4.68 g, 34.1 mmol). After being stirred at 120° C. for 12 h, the reaction mixture was quenched with H₂O (50 mL) and extracted with DCM (50 mL×3). The combined organic layer was washed with brine, dried over Na₂SO₄ and concentrated in vacuo. The residue was purified by flash column (eluting with EtOAc/PE=0 to 20%) to afford methyl 5-(oxetan-3-ylsulfanyl)furan-2-carboxylate as a colorless oil (Int-7a, 195 mg, 18.7%). MS obsd. (ESI⁺) [(M+H)⁺]: 215.1. ¹H NMR (400 MHz, CDCl₃) δ ppm: 7.16 (d, J=3.4 Hz, 1H), 6.60 (d, J=3.4 Hz, 1H), 4.97 (dd, J=11.0, 3.9 Hz, 2H), 4.67 (t, J=6.7 Hz, 2H), 4.40 (tt, J=7.7, 6.5 Hz, 1H), 3.90 (s, 3H).

### Step 2: Preparation of methyl 5-(oxetan-3-ylsulfonyl)furan-2-carboxylate (Int-7b)

To a solution of methyl 5-(oxetan-3-ylsulfanyl)furan-2-carboxylate (Int-7a, 175 mg, 0.82 mmol) in DCM (10 mL) was added m-CPBA (705 mg, 4.1 mmol). After being stirred at 25° C. for 12 h, the mixture was washed with saturated NaHCO₃. The organic layer was dried over Na₂SO₄, filtered and concentrated in vacuo. The residue was purified by flash column (eluting with EtOAc/PE=0 to 50%) to give methyl 5-(oxetan-3-ylsulfonyl)furan-2-carboxylate as an off-white solid (Int-7b, 124 mg, 62%). MS obsd. (ESI⁺) [(M+H)⁺]: 247.1. ¹H NMR (400 MHz, CDCl₃) δ ppm: 7.31 (d, J=3.6 Hz, 1H), 7.27 (d, J=3.7 Hz, 1H), 5.00 (dd, J=7.6, 6.2 Hz, 2H), 4.91 (t, J=7.8 Hz, 2H), 4.64 (tt, J=8.2, 6.0 Hz, 1H), 3.95 (s, 3H).

### Step 3: Preparation of 5-(oxetan-3-ylsulfonyl)furan-2-carboxylic acid (Int-7)

To a solution of methyl 5-(oxetan-3-ylsulfonyl)furan-2-carboxylate (Int-7b, 124 mg, 0.5 mmol) in a mixed solvent of MeOH (9 mL) and water (3 mL) was added LiOH (60 mg, 2.5 mmol). After being stirred at 25° C. for 2 h, most of the solvent was evaporated. The residue was acidified by HCl (1 M) to pH=2 and extracted with DCM (30 mL×3). The combined organic layer was dried over Na₂SO₄, filtered and concentrated in vacuo to give 5-(oxetan-3-ylsulfonyl)furan-2-carboxylic acid as an off-white solid (Int-7, 103 mg, 88%). MS obsd. (ESI⁺) [(M+Na)⁺]: 255.0.

### Intermediate Int-8

### 4-bromo-5-ethylsulfonyl-furan-2-carboxylic acid

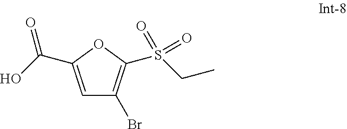

The title compound was prepared in analogy to the procedure described for the preparation of 5-methylsulfonylfuran-2-carboxylic acid (Int-3), by using ethylsulfinyloxysodium instead of methylsulfinyloxysodium and 4,5-dibromofuran-2-carboxylate instead of 5-bromo-2-furoate. MS obsd. (ESI⁺) [(M+H)⁺]: 282.9.

### Intermediate Int-9

### 5-ethylsulfonyl-3-methyl-furan-2-carboxylic acid

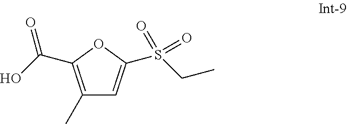

The title compound was prepared in analogy to the procedure described for the preparation of 5-methylsulfonylfuran-2-carboxylic acid (Int-3), by using ethylsulfinyloxysodium instead of methylsulfinyloxysodium and 5-bromo-3-methyl-furan-2-carboxylic acid instead of 5-bromo-2-furoate. MS obsd. (ESI⁺) [(M+H)⁺]: 219.1.

### Intermediate Int-10

### 5-tert-butylsulfanylfuran-2-carboxylic acid

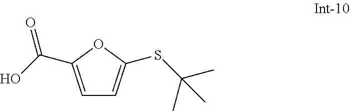

The title compound was prepared according to the following scheme:

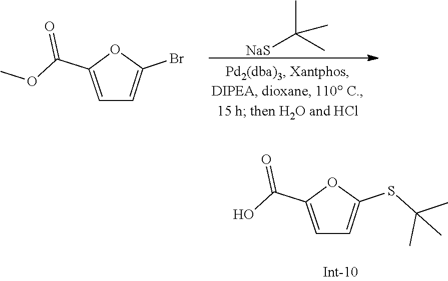

A mixture of methyl 5-bromo-2-furoate (1.0 g, 4.88 mmol), sodium 2-methyl-2-propanethiolate (1.09 g, 9.76 mmol), 9,9-dimethyl-4,5-bis(diphenylphosphino)xanthene (0.56 g, 0.98 mmol), N,N-diisopropylethylamine (2.55 mL, 14.63 mmol) and tris(dibenzylideneacetone)dipalladium (0) (0.45 g, 0.49 mmol) in 1,4-dioxane (20 mL) was stirred at 110° C. for 15 h. After being cooled to 25° C., water (50 mL) was added and the resulting mixture was stirred for 2 h. The solvent was evaporated, and the residue was extracted with EtOAc (100 mL). The aqueous was collected and acidified with HCl (1 M) to pH=1 to 3. The solid was precipitated and collected by filtration to give 5-tert-butylsulfanylfuran-2-carboxylic acid as a light yellow solid (Int-10, 400 mg, 38.27%). MS obsd. (ESI⁺) [(M+H)⁺]: 201.1.

### Intermediate Int-11

### 5-(trifluoromethylsulfanyl)furan-2-carboxylic acid

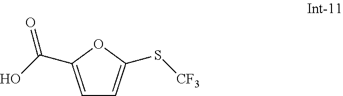 [SMILES: O=C(O)c1ccc(SC(F)(F)F)o1]

The title compound was prepared according to the following scheme:

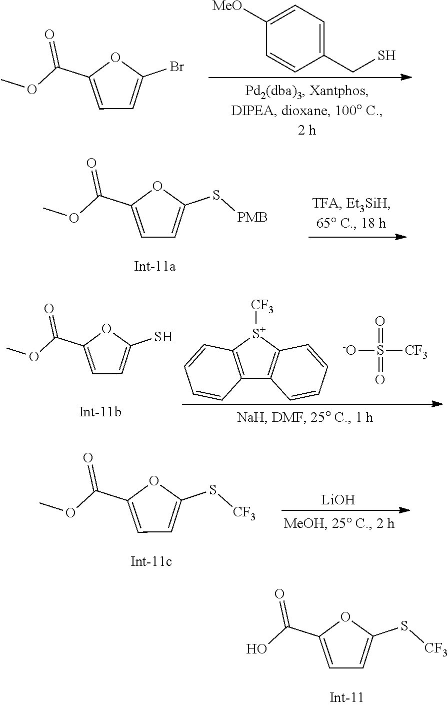

### Step 1: Preparation of methyl 5-[(4-methoxyphenyl)methylsulfanyl]furan-2-carboxylate (Int-11a)

A mixture of methyl 5-bromo-2-furoate (2.24 g, 10.9 mmol), 9,9-dimethyl-4,5-bis(diphenylphosphino)xanthene (631.1 mg, 1.09 mmol), N,N-diisopropylethylamine (3.8 mL, 21.8 mmol), tris(dibenzylideneacetone)dipalladium (0) (499.4 mg, 0.55 mmol) and 4-methoxybenzyl mercaptan (1.68 g, 10.9 mmol) in 1,4-dioxane (50 mL) was stirred at 100° C. for 2 h. The mixture was filtered to remove the solid and the filtrate was concentrated to dryness. The crude product was purified by flash column (eluting with EtOAc/PE=0 to 3%) to afford methyl 5-[(4-methoxyphenyl)methylsulfanyl]furan-2-carboxylate as a yellow oil (Int-11a, 2.7 g, 73.5%). MS obsd. (ESI⁺) [(M+H)⁺]: 279.1.

### Step 2: Preparation of methyl 5-sulfanylfuran-2-carboxylate (Int-11b)

A mixture of methyl 5-[(4-methoxyphenyl)methylsulfanyl]furan-2-carboxylate (Int-11a, 2.7 g, 8.02 mmol), TFA (30 mL, 8.02 mmol) and Et₃SiH (15 mL, 8.02 mmol) was stirred at 65° C. for 16 h. The reaction mixture was concentrated in vacuo to give methyl 5-sulfanylfuran-2-carboxylate as a brown oil (Int-11b, 1.72 g), which was used for the next step without further purification. MS obsd. (ESI⁺) [(M+H)⁺]: 159.1.

### Step 3: Preparation of methyl 5-(trifluoromethylsulfanyl)furan-2-carboxylate (Int-11c)

To a solution of methyl 5-sulfanylfuran-2-carboxylate (Int-11b, 7.5 g, 34.61 mmol) in DMF (90 mL) was added sodium hydride (60% in oil, 2.08 g, 51.92 mmol) at 0° C. After being stirred for 15 min, S-(trifluoromethyl)dibenzothiophenium trifluoromethanesulfonate (15.32 g, 38.07 mmol) was added. The reaction mixture was stirred at 25° C. for 1 h, then quenched with water (100 mL) and extracted with EtOAc (200 mL×2). The organic layer was washed with brine (50 mL), dried over Na₂SO₄ and concentrated in vacuo, the residue was purified by column (eluting with EtOAc/PE=1/100 to 1/15) to give methyl 5-(trifluoromethylsulfanyl)furan-2-carboxylate as a yellow oil (Int-11c, 1.5 g, 19.2%). ¹H NMR (400 MHz, CDCl₃) δ ppm: 7.23 (d, J=3.5 Hz, 1H), 6.99 (d, J=3.5 Hz, 1H), 3.93 (s, 3H).

### Step 4: Preparation of 5-(trifluoromethylsulfanyl)furan-2-carboxylic acid (Int-11)

To a solution of methyl 5-(trifluoromethylsulfanyl)furan-2-carboxylate (Int-11c, 100 mg, 0.44 mmol) in a mixed solvent of MeOH (1.5 mL) and water (0.5 mL) was added LiOH (31.8 mg, 1.3 mmol). After being stirred at 25° C. for 2 h, the mixture was concentrated to give the crude product, which was treated with water (5 mL), washed with DCM (10 mL×3), acidified by HCl (1 M) to pH=3-4, and then extracted with DCM (10 mL). The organic layer was concentrated to give 5-(trifluoromethylsulfanyl)furan-2-carboxylic acid as a white solid (Int-11, 90 mg, 96%). MS obsd. (ESI⁺) [(M+Na)⁺]: 213.0. ¹H NMR (400 MHz, CDCl₃) δ ppm: 7.36 (d, J=3.5 Hz, 1H), 7.03 (d, J=3.5 Hz, 1H).

### Intermediate Int-12

### 5-(cyclopropylmethylsulfanyl)furan-2-carboxylic acid

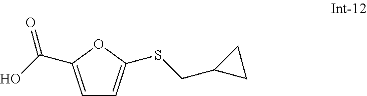 [SMILES: O=C(O)c1ccc(SCC2CC2)o1]

The title compound was prepared according to the following scheme:

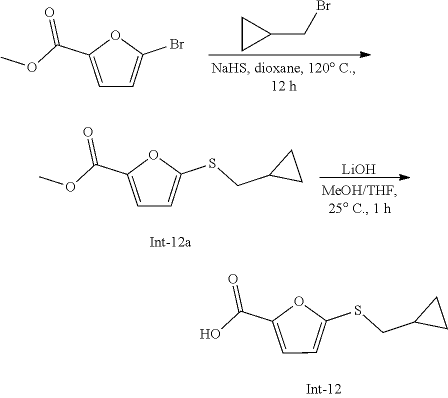

### Step 1: Preparation of methyl 5-(cyclopropylmethylsulfanyl)furan-2-carboxylate (Int-12a)

To a solution of methyl 5-bromo-2-furoate (2 g, 9.76 mmol) in 1,4-dioxane (20 mL) was added sodium hydrosulfide (5.5 g, 97.6 mmol) and (bromomethyl)cyclopropane (3.32 mL, 34.1 mmol). After being stirred at 120° C. for 12 h, the reaction mixture was quenched with H₂O (50 mL) and extracted with DCM (50 mL×3). The combined organic layer was washed with brine, dried over Na₂SO₄ and concentrated in vacuo. The residue was purified by flash column (eluting with 100% PE) to afford methyl 5-(cyclopropylmethylsulfanyl)furan-2-carboxylate as a colorless oil (Int-12a, 580 mg, 28%). MS obsd. (ESI⁺) [(M+H)⁺]: 213.1. ¹H NMR (400 MHz, CDCl₃) δ ppm: 7.14 (d, J=3.5 Hz, 1H), 6.50 (d, J=3.5 Hz, 1H), 3.89 (s, 3H), 2.86 (d, J=7.2 Hz, 2H), 0.95-1.10 (m, 1H), 0.51-0.61 (m, 2H), 0.14-0.24 (m, 2H).

### Step 2: Preparation of 5-(cyclopropylmethylsulfanyl)furan-2-carboxylic acid (Int-12)

To a solution of methyl 5-(cyclopropylmethylsulfanyl)furan-2-carboxylate (Int-12a, 308 mg, 1.45 mmol) in a mixed solvent of THF (2 mL) and methanol (2 mL) was added a solution of LiOH in water (2.2 mL, 2 M). After being stirred at 20° C. for 1 h, the mixture was acidified by HCl (2.5 mL, 2 M). The solvent was evaporated and the residue was separated by EtOAc (20 mL) and water (20 mL). The organic layer was dried over Na₂SO₄, filtered and concentrated to give the 5-(cyclopropylmethylsulfanyl)furan-2-carboxylic acid as a white solid (Int-12, 280 mg, 97%). MS obsd. (ESI⁺) [(M+H)⁺]: 199.1.

### Intermediate Int-13

### 5-(cyclopropylmethylsulfinyl)furan-2-carboxylic acid

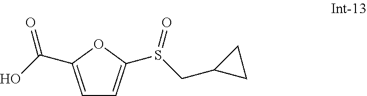 [SMILES: O=C(O)c1ccc(S(=O)CC2CC2)o1]

The title compound was prepared according to the following scheme:

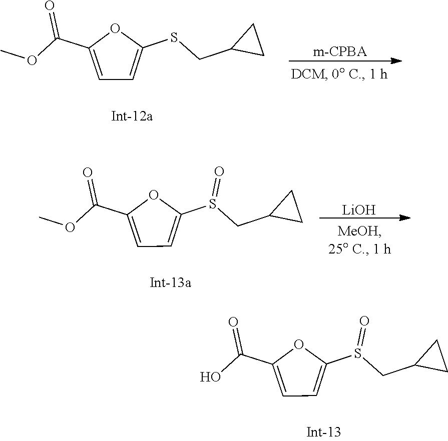

### Step 1: Preparation of methyl 5-(cyclopropylmethylsulfanyl)furan-2-carboxylate (Int-13a)

To a solution of 5-(cyclopropylmethylsulfanyl)furan-2-carboxylate (Int-12a, 382 mg, 1.8 mmol) in DCM (10 mL) was added m-CPBA (310 mg, 1.8 mmol). After being stirred at 0° C. for 1 h, the mixture was washed with saturated NaHCO₃. The organic layer was dried over Na₂SO₄, filtered and concentrated in vacuo. The residue was purified by flash column (eluting with EtOAc/PE=1/2) to give methyl 5-(cyclopropylmethylsulfinyl)furan-2-carboxylate as a light yellow oil (Int-13a, 380 mg, 92.5%). MS obsd. (ESI⁺) [(M+H)⁺]: 229.1.

### Step 2: Preparation of 5-(cyclopropylmethylsulfinyl)furan-2-carboxylic acid (Int-13)

To a solution of methyl 5-(cyclopropylmethylsulfinyl)furan-2-carboxylate (Int-13a, 380 mg, 1.66 mmol) in a mixed solvent of MeOH (2.5 mL) and THF (2.5 mL) was added LiOH·H₂O (2.5 mL, 2 M). After being stirred at 25° C. for 1 h, most of the solvent was evaporated. The residue was acidified by 3 mL of HCl (2 M) and extracted with EtOAc (20 mL). The organic layer was dried over Na₂SO₄, filtered and concentrated in vacuo to give 5-(cyclopropylmethylsulfinyl)furan-2-carboxylic acid as a white solid (Int-13, 320 mg, 89.7%). MS obsd. (ESI⁺) [(M+H)⁺]: 215.1.

### Intermediate Int-14

### 5-(cyclopropylmethylsulfonyl)furan-2-carboxylic acid

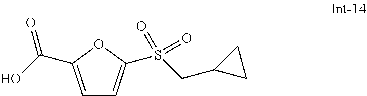 [SMILES: O=C(O)c1ccc(S(=O)(=O)CC2CC2)o1]

The title compound was prepared according to the following scheme:

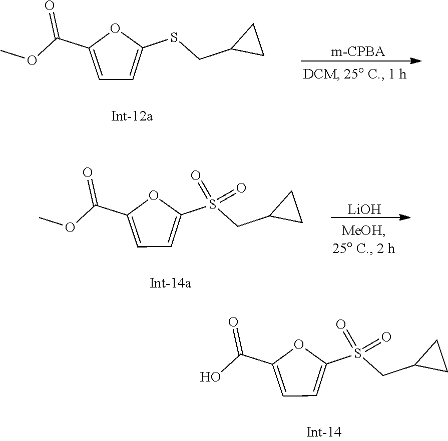

### Step 1: Preparation of methyl 5-(cyclopropylmethylsulfonyl)furan-2-carboxylate (Int-14a)

To a solution of methyl 5-(cyclopropylmethylsulfanyl)furan-2-carboxylate (Int-12a, 272 mg, 0.69 mmol) in DCM (10 mL) was added m-CPBA (358 mg, 2 mmol). After being stirred at 25° C. for 1 h, the mixture was washed with saturated NaHCO₃. The organic layer was dried over Na₂SO₄, filtered and concentrated in vacuo. The residue was purified by flash column (eluting with EtOAc/PE=0 to 20%) to give methyl 5-(cyclopropylmethylsulfonyl)furan-2-carboxylate as a colorless oil (Int-14a, 110 mg, 59.8%). MS obsd. (ESI⁺) [(M+H)⁺]: 245.1. ¹H NMR (400 MHz, CDCl₃) δ ppm: 7.22-7.25 (m, 2H), 3.94 (d, J=4.2 Hz, 3H), 3.21 (d, J=7.3 Hz, 2H), 1.02-1.18 (m, 1H), 0.56-0.68 (m, 2H), 0.14-0.26 (m, 2H).

### Step 2: Preparation of 5-(cyclopropylmethylsulfonyl)furan-2-carboxylic acid (Int-14)

To a solution of methyl 5-(cyclopropylmethylsulfonyl)furan-2-carboxylate (Int-14a, 101 mg, 0.41 mmol) in a mixed solvent of MeOH (9 mL) and water (3 mL) was added LiOH (50 mg, 2 mmol). After being stirred at 25° C. for 2 h, most of the solvent was evaporated. The residue was acidified by HCl (1 M) to pH=2 and extracted with DCM (30 mL×3). The combined organic layer was dried over Na₂SO₄, filtered and concentrated in vacuo to give 5-(cyclopropylmethylsulfonyl)furan-2-carboxylic acid as an off-white solid (Int-14, 85 mg, 84.8%). MS obsd. (ESI⁺) [(M+H)⁺]: 231.0.

### Intermediate Int-15

### 5-isobutylsulfanylfuran-2-carboxylic acid

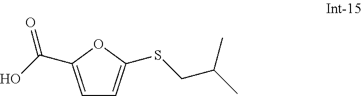

The title compound was prepared in analogy to the procedure described for the preparation of 5-(cyclopropylmethylsulfanyl)furan-2-carboxylic acid (Int-12), by using 1-bromo-2-methylpropane instead of (bromomethyl)cyclopropane. MS obsd. (ESI⁺) [(M+H)⁺]: 201.1.

### Intermediate Int-16

### 5-isobutylsulfinylfuran-2-carboxylic acid

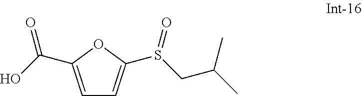

The title compound was prepared in analogy to the procedure described for the preparation of 5-(cyclopropylmethylsulfinyl)furan-2-carboxylic acid (Int-13), by using 1-bromo-2-methylpropane instead of (bromomethyl)cyclopropane. MS obsd. (ESI⁺) [(M+H)⁺]: 217.1.

### Intermediate Int-17

### 5-isobutylsulfonylfuran-2-carboxylic acid

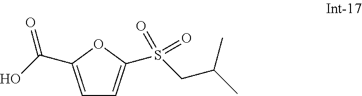

The title compound was prepared in analogy to the procedure described for the preparation of 5-(cyclopropylmethylsulfonyl)furan-2-carboxylic acid (Int-14), by using 1-bromo-2-methylpropane instead of (bromomethyl)cyclopropane. MS obsd. (ESI⁺) [(M+H)⁺]: 233.1.

### Intermediate Int-18

### 5-(cyclobutylmethylsulfonyl)furan-2-carboxylic acid

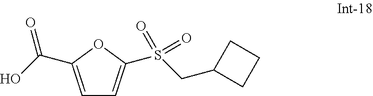 [SMILES: O=C(O)c1ccc(S(=O)(=O)CC2CCC2)o1]

The title compound was prepared in analogy to the procedure described for the preparation of 5-(cyclopropylmethylsulfonyl)furan-2-carboxylic acid (Int-14), by using (bromomethyl)cyclobutane instead of (bromomethyl)cyclopropane. MS obsd. (ESI⁺) [(M+H)⁺]: 245.1.

### Intermediate Int-19

### 5-(oxetan-3-ylmethylsulfanyl)furan-2-carboxylic acid

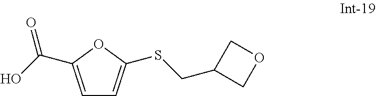 [SMILES: O=C(O)c1ccc(SCC2COC2)o1]

The title compound was prepared according to the following scheme:

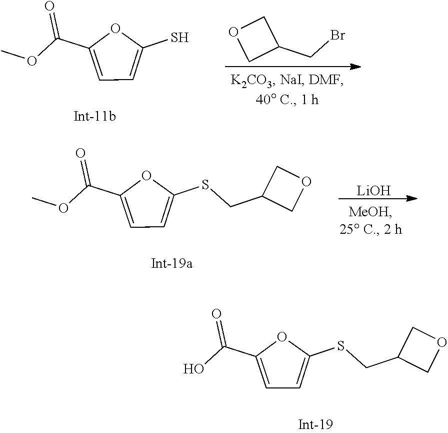

### Step 1: Preparation of methyl 5-(oxetan-3-ylmethylsulfanyl)furan-2-carboxylate (Int-19a)

To a solution of methyl 5-sulfanylfuran-2-carboxylate (Int-11b, 450 mg, 2.84 mmol) in DMF (5 mL) were added K₂CO₃ (2.4 g, 17.07 mmol), NaI (42.64 mg, 0.28 mmol) and 3-(bromomethyl)oxetane (0.55 mL, 7.11 mmol). After being stirred at 40° C. for 1 h, the mixture was quenched by water (50 mL) and extracted with EtOAc (30 mL×3). The combined organic layer was concentrated in vacuo. The crude product was purified by flash column (eluting with EtOAc/PE=0 to 10%) to afford methyl 5-(oxetan-3-ylmethylsulfanyl)furan-2-carboxylate as a light yellow oil (Int-19a, 304 mg, 46.8%). MS obsd. (ESI⁺) [(M+H)⁺]: 229.1. ¹H NMR (400 MHz, CDCl₃) δ ppm: 7.13 (d, J=3.4 Hz, 1H), 6.51 (d, J=3.4 Hz, 1H), 4.78 (dd, J=7.4, 6.5 Hz, 2H), 4.36 (t, J=5.9 Hz, 2H), 3.90 (s, 3H), 3.10-3.28 (m, 3H).

### Step 2: Preparation of 5-(oxetan-3-ylmethylsulfanyl)furan-2-carboxylic acid (Int-19)

To a solution of methyl 5-(oxetan-3-ylmethylsulfanyl)furan-2-carboxylate (Int-19a, 291 mg, 1.27 mmol) in a mixed solvent of MeOH (15 mL) and water (5 mL) was added LiOH (91.6 mg, 3.8 mmol). After being stirred at 25° C. for 2 h, most of the solvent was evaporated. The residue was acidified by HCl (2 M) to pH=5 and extracted with EtOAc (10 mL×3). The combined organic layer was dried over Na₂SO₄, filtered and concentrated in vacuo to give 5-(oxetan-3-ylmethylsulfanyl)furan-2-carboxylic acid as a light yellow oil (Int-19, 254 mg, 93%). MS obsd. (ESI⁺) [(M+H)⁺]: 215.0.

### Intermediate Int-20

### [5-(oxetan-3-ylmethylsulfonyl)furan-2-carbonyl]oxylithium

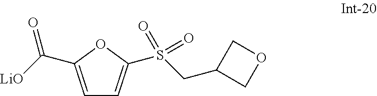

The title compound was prepared according to the following scheme:

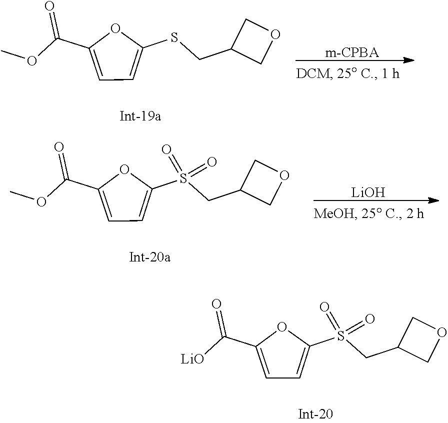

### Step 1: Preparation of methyl 5-(oxetan-3-ylmethylsulfonyl)furan-2-carboxylate (Int-20a)

To a solution of methyl 5-(oxetan-3-ylmethylsulfanyl)furan-2-carboxylate (Int-19a, 150 mg, 0.66 mmol) in DCM (5 mL) was added m-CPBA (340 mg, 1.97 mmol). After being stirred at 25° C. for 2 h, the mixture was concentrated to dryness and purified by column (eluting wih EtOAc/PE=0 to 50%) to afford methyl 5-(oxetan-3-ylmethylsulfonyl)furan-2-carboxylate as an off-white solid (Int-20a, 142 mg, 64.3%). MS obsd. (ESI⁺) [(M+H)⁺]: 261.1.

### Step 2: Preparation of [5-(oxetan-3-ylmethylsulfonyl)furan-2-carbonyl]oxylithium (Int-20)

To a solution of methyl 5-(oxetan-3-ylmethylsulfonyl)furan-2-carboxylate (Int-20a, 142 mg, 0.55 mmol) in a mixed solvent of MeOH (6 mL) and water (2 mL) was added LiOH (39.2 mg, 1.64 mmol). After being stirred at 25° C. for 2 h, the MeOH was removed and the residue was lyophilized to afford [5-(oxetan-3-ylmethylsulfonyl)furan-2-carbonyl]oxylithium as an off-white solid (Int-20, 166 mg, 99.5%). MS obsd. (ESI⁺) [(M+H)⁺]: 247.1.

### Intermediate Int-21

### [5-[(3,3-difluorocyclobutyl)methylsulfonyl]furan-2-carbonyl]oxylithium

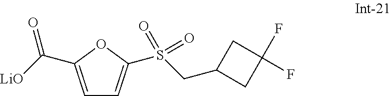

The title compound was prepared in analogy to the procedure described for the preparation of [5-(oxetan-3-ylmethylsulfonyl)furan-2-carbonyl]oxylithium (Int-20), by using 3-(bromomethyl)-1,1-difluoro-cyclobutane instead of 3-(bromomethyl)oxetane. MS obsd. (ESI⁺) [(M+Na)⁺]: 303.0.

### Intermediate Int-22

### 5-[(1-methylazetidin-3-yl)methylsulfonyl]furan-2-carboxylic acid

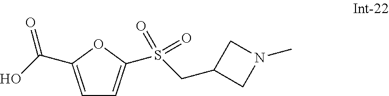 [SMILES: CN1CC(CS(=O)(=O)c2ccc(C(=O)O)o2)C1]

The title compound was prepared according to the following scheme:

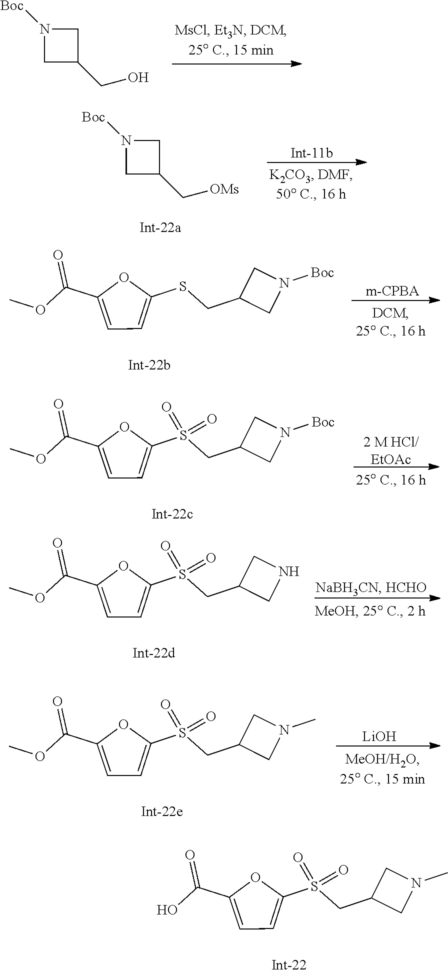

### Step 1: Preparation of tert-butyl 3-(methylsulfonyloxymethyl)azetidine-1-carboxylate (Int-22a)

To a solution of tert-butyl 3-(hydroxymethyl)azetidine-1-carboxylate (9 g, 48.07 mmol) in DCM (90 mL) was added triethylamine (10 mL, 72.1 mmol) and MsCl (4.84 mL, 62.49 mmol).

After being stirred at 25° C. for 15 min, the reaction was quenched with saturated NaHCO₃ and extracted with DCM (100 mL). The organic layer was concentrated to give the crude product, which was purified by flash column (eluting with EtOAc/PE=0 to 50%) to give tert-butyl 3-(methylsulfonyloxymethyl)azetidine-1-carboxylate as a colorless oil (Int-22a, 12.6 g, 98.8%). ¹H NMR (400 MHz, CDCl₃) δ ppm: 4.35 (d, J=6.8 Hz, 2H), 4.05 (t, J=8.7 Hz, 2H), 3.72 (dd, J=9.0, 5.2 Hz, 2H), 3.05 (s, 3H), 2.86-2.97 (m, 1H), 1.44 (s, 9H).

### Step 2: Preparation of tert-butyl 3-[(5-methoxycarbonyl-2-furyl)sulfanylmethyl]azetidine-1-carboxylate (Int-22b)

To a solution of tert-butyl 3-(methylsulfonyloxymethyl)azetidine-1-carboxylate (Int-22a, 7 g, 26.38 mmol) in DMF (70 mL) was added methyl 5-sulfanylfuran-2-carboxylate (Int-11b, 4.17 g, 26.38 mmol) and K₂CO₃ (10.94 g, 79.15 mmol). After being stirred at 50° C. for 16 h, the mixture was extracted with EtOAc (200 mL×2), washed with water (200 mL×3). The combined organic layer was concentrated to give the crude product, which was purified by flash column (eluting with EtOAc/PE=0 to 1/3) to give tert-butyl 3-[(5-methoxycarbonyl-2-furyl)sulfanylmethyl]azetidine-1-carboxylate as a light yellow oil (Int-22b, 6.5 g, 75.25%). MS obsd. (ESI⁺) [(M+Na)⁺]: 350.0. ¹H NMR (400 MHz, CDCl₃) δ ppm: 7.13 (d, J=3.4 Hz, 1H), 6.51 (d, J=3.4 Hz, 1H), 4.02 (t, J=8.6 Hz, 2H), 3.89 (s, 3H), 3.59 (dd, J=9.0, 5.3 Hz, 2H), 3.12 (d, J=7.8 Hz, 2H), 2.71 (pt, J=8.0, 5.4 Hz, 1H), 1.43 (s, 9H).

### Step 3: Preparation of tert-butyl 3-[(5-methoxycarbonyl-2-furyl)sulfonylmethyl]azetidine-1-carboxylate (Int-22c)

To a solution of tert-butyl 3-[(5-methoxycarbonyl-2-furyl)sulfanylmethyl]azetidine-1-carboxylate (Int-22b, 6.5 g, 19.85 mmol) in DCM (80 mL) was added m-CPBA (8.57 g, 49.63 mmol). After being stirred at 25° C. for 16 h, the reaction mixture was washed with saturated Na₂SO₃ (100 mL×2), saturated NaHCO₃ (100 mL×2) and brine (200 mL), dried over Na₂SO₄ and concentrated in vacuo. The residue was purified by flash column (eluting with EtOAc/PE=1/10 to 1/2) to give tert-butyl 3-[(5-methoxycarbonyl-2-furyl)sulfonylmethyl]azetidine-1-carboxylate as a white solid (Int-22c, 6.5 g, 91.1%). MS obsd. (ESI⁺) [(M+Na)⁺]: 382.0. ¹H NMR (400 MHz, CDCl₃) δ ppm: 7.24 (dd, J=8.9, 3.6 Hz, 2H), 4.11 (t, J=8.7 Hz, 2H), 3.95 (s, 3H), 3.70 (dd, J=9.0, 5.6 Hz, 2H), 3.57 (d, J=7.5 Hz, 2H), 3.15-2.99 (m, 1H), 1.43 (s, 9H).

### Step 4: Preparation of methyl 5-(azetidin-3-ylmethylsulfonyl)furan-2-carboxylate (Int-22d)

To a solution of tert-butyl 3-[(5-methoxycarbonyl-2-furyl)sulfonylmethyl]azetidine-1-carboxylate (Int-22c, 1 g, 2.78 mmol) in EtOAc (10 mL) was added HCl/EtOAc (4.17 mL, 8.35 mmol). After being stirred at 25° C. for 16 h, the reaction mixture was concentrated to give the crude product, which was purified by flash column (eluting with DCM/MeOH=20/1 to 5/1) to give methyl 5-(azetidin-3-ylmethylsulfonyl)furan-2-carboxylate as a white solid (Int-22d, 400 mg, 55.4%). MS obsd. (ESI⁺) [(M+H)⁺]: 260.0.

### Step 5: Preparation of methyl 5-[(1-methylazetidin-3-yl)methylsulfonyl]furan-2-carboxylate (Int-22e)

To a solution of methyl 5-(azetidin-3-ylmethylsulfonyl)furan-2-carboxylate (Int-22d, 300 mg, 1.16 mmol) in MeOH (6 mL) was added paraformaldehyde (1.13 g, 11.57 mmol), After being stirred at 25° C. for 15 min, NaBH₃CN (145.4 mg, 2.3 mmol) was added and then the reaction mixture was stirred at 25° C. for 2 h. The resulting mixture was concentrated to give the crude product, which was purified by flash column (eluting with MeOH/DCM=0 to 1/3) to give methyl 5-[(1-methylazetidin-3-yl)methylsulfonyl]furan-2-carboxylate as a white solid (Int-22e, 300 mg, 94.6%). MS obsd. (ESI⁺) [(M+H)⁺]: 274.1.

### Step 6: Preparation of 5-[(1-methylazetidin-3-yl)methylsulfonyl]furan-2-carboxylic acid (Int-22)

A solution of methyl 5-[(1-methylazetidin-3-yl)methylsulfonyl]furan-2-carboxylate (Int-22e, 400 mg, 1.46 mmol) in MeOH (4 mL) was added LiOH (2 M in water, 4.39 mmol), then the reaction mixture was stirred at 25° C. for 0.25 h. The reaction mixture was acidified by 2 M HCl solution to pH=3 and extracted with EtOAc (20 mL). The organic layer was concentrated to give 5-[(1-methylazetidin-3-yl)methylsulfonyl]furan-2-carboxylic acid as a white solid (Int-22, 360 mg, 94.9%). MS obsd. (ESI⁺) [(M+H)⁺]: 260.1.

### Intermediate Int-23

### 5-[(1-benzyloxycarbonylazetidin-3-yl)methylsulfonyl]furan-2-carboxylic acid

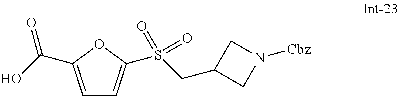 [SMILES: O=C(O)c1ccc(S(=O)(=O)CC2CN(C(=O)OCc3ccccc3)C2)o1]

The title compound was prepared in analogy to the procedure described for the preparation of 5-[(1-methylazetidin-3-yl)methylsulfonyl]furan-2-carboxylic acid (Int-22), by using benzyl 3-(methylsulfonyloxymethyl)azetidine-1-carboxylate instead of tert-butyl 3-(methylsulfonyloxymethyl)azetidine-1-carboxylate, MS obsd. (ESI⁺) [(M+H)⁺]: 380.0.

### Intermediate Int-24

### 5-(2-methoxyethylsulfonyl)furan-2-carboxylic acid

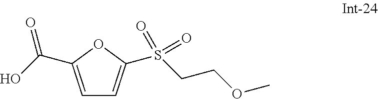

The title compound was prepared in analogy to the procedure described for the preparation of [5-(oxetan-3-ylmethylsulfonyl)furan-2-carbonyl]oxylithium (Int-20), by using 2-bromoethyl methyl ether instead of 3-(bromomethyl)oxetane, MS obsd. (ESI⁺) [(M+H)⁺]: 235.1.

### Intermediate Int-25

### 5-(ethylsulfinylmethyl)furan-2-carboxylic acid

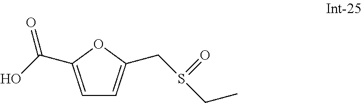

The title compound was prepared according to the following scheme:

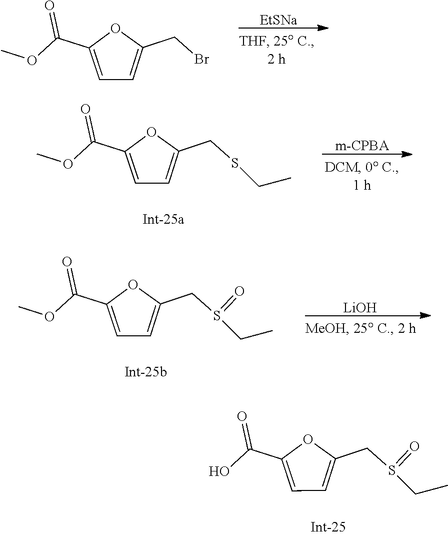

### Step 1: Preparation of methyl 5-(ethylsulfanylmethyl)furan-2-carboxylate (Int-25a)

To a solution of methyl 5-(bromomethyl)furan-2-carboxylate (500 mg, 2.28 mmol) in THF (10 mL) was added ethylsulfanylsodium (576 mg, 6.8 mmol). After being stirred at 25° C. for 2 h, the mixture was quenched with water (50 mL) and extracted with EtOAc (20 mL×3). The combined organic phase was dried over Na₂SO₄ and concentrated in vacuo to give methyl 5-(ethylsulfanylmethyl)furan-2-carboxylate as a yellow oil (Int-25a, 400 mg, 87.5%), which was used for the step without purification. MS obsd. (ESI⁺) [(M+H)⁺]: 201.0.

### Step 2: Preparation of methyl 5-(ethylsulfinylmethyl)furan-2-carboxylate (Int-25b)

To a solution of methyl 5-(ethylsulfanylmethyl)furan-2-carboxylate (Int-25a, 400 mg, 2 mmol) in DCM (5 mL) was added m-CPBA (344 mg, 2 mmol) at 0° C. After being stirred for further 1 h, the reaction was quenched with saturated Na₂CO₃ aqueous solution (10 mL). The organic phase was separated, dried over Na₂SO₄, filtered and concentrate to give a crude product which was purified by flash column (eluting with DCM/MeOH=94/6) to give methyl 5-(ethylsulfinylmethyl)furan-2-carboxylate as a yellow oil (Int-25b, 370 mg, 85.7%). MS obsd. (ESI⁺) [(M+H)⁺]: 217.0.

### Step 3: Preparation of 5-(ethylsulfanylmethyl)furan-2-carboxylic acid (Int-25)

To a solution of methyl 5-(ethylsulfinylmethyl)furan-2-carboxylate (Int-25b, 160 mg, 0.74 mmol) in a mixed solvent of MeOH (5 mL) and water (5 mL) was added LiOH (609 mg, 10.9 mmol) and the reaction was stirred at 25° C. for 2 h. The resulting mixture was concentrated to remove the MeOH and the residue was acidified to pH=2 with 1 M HCl, extracted with DCM (40 mL×3). The combined organic layer was dried by anhydrous Na₂SO₄ and concentrated under vacuo to give 5-(ethylsulfinylmethyl)furan-2-carboxylic acid as a yellow oil (Int-25, 90 mg, 60.2%). MS obsd. (ESI⁺) [(M+H)⁺]: 203.0.

### Intermediate Int-26

### 5-(isopropylsulfanylmethyl)furan-2-carboxylic acid

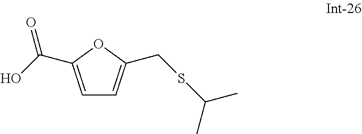

The title compound was prepared according to the following scheme:

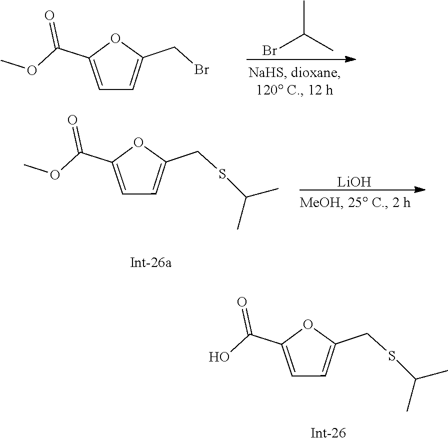

### Step 1: Preparation of methyl 5-(isopropylsulfanylmethyl)furan-2-carboxylate (Int-26a)

To a solution of methyl 5-(bromomethyl)furan-2-carboxylate (500 mg, 2.28 mmol) in 1,4-dioxane (10 mL) was added sodium hydrosulfide (1.28 g, 22.8 mmol) and 2-bromopropane (2.8 g, 22.8 mmol). After being stirred at 120° C. for 5 h, the reaction mixture was quenched with H₂O (30 mL) and extracted with EtOAc (30 mL×3). The combined organic layer was washed with brine, dried over Na₂SO₄ and concentrated in vacuo. The residue was purified by flash column (eluting with EtOAc/PE=9/1) to afford methyl 5-(isopropylsulfanylmethyl)furan-2-carboxylate as a yellow oil (Int-26a, 100 mg, 20.4%). MS obsd. (ESI⁺) [(M+H)⁺]: 215.1.

### Step 2: Preparation of 5-(isopropylsulfanylmethyl)furan-2-carboxylic acid (Int-26)

To a solution of methyl 5-(isopropylsulfanylmethyl)furan-2-carboxylate (Int-26a, 100 mg, 0.47 mmol) in a mixed solvent of MeOH (5 mL) and water (5 mL) was added LiOH (130.8 mg, 2.33 mmol). After being stirred at 25° C. for 2 h, the mixture was concentrated, and the residue was acidified to pH=2 with 1 M HCl, extracted by DCM (10 mL×3). The organic layer was dried over Na₂SO₄ and concentrated in vacuo to give 5-(isopropylsulfanylmethyl)furan-2-carboxylic acid as a yellow oil (Int-26, 70 mg, 74.9%), which was used for the next step without purification. MS obsd. (ESI⁺) [(M+H)⁺]: 201.1.

### Intermediate Int-27

### 5-(cyclopropylsulfanylmethyl)furan-2-carboxylic acid

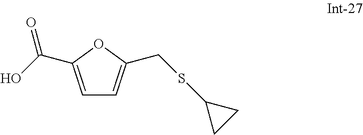 [SMILES: O=C(O)c1ccc(CSC2CC2)o1]

The title compound was prepared in analogy to the procedure described for the preparation of 5-(isopropylsulfanylmethyl)furan-2-carboxylic acid (Int-26), by using cyclopropanethiol instead of sodium hydrosulfide and 2-bromopropane. MS obsd. (ESI⁺) [(M+H)⁺]: 213.1.

### Intermediate Int-28

### 5-(cyclopropylsulfonylmethyl)furan-2-carboxylic acid

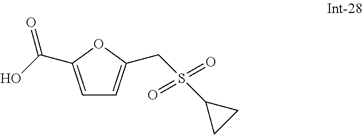 [SMILES: O=C(O)c1ccc(CS(=O)(=O)C2CC2)o1]

The title compound was prepared in analogy to the procedure described for the preparation of 5-methylsulfonylfuran-2-carboxylic acid (Int-3), by using cyclopropylsulfinyloxysodium instead of methylsulfinyloxysodium and 5-(bromomethyl)furan-2-carboxylate instead of 5-bromo-2-furoate. MS obsd. (ESI⁺) [(M+H)⁺]: 231.1.

### Intermediate Int-29

### 5-(2-methylsulfonylethyl)furan-2-carboxylic acid

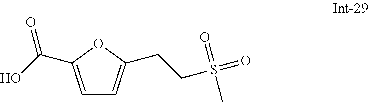

The title compound was prepared according to the following scheme:

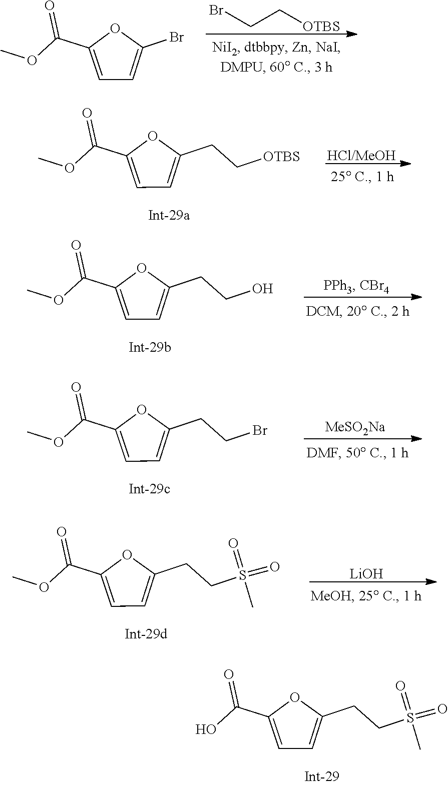

### Step 1: Preparation of methyl 5-[2-[tert-butyl(dimethyl)silyl]oxyethyl]furan-2-carboxylate (Int-29a)

A mixture of methyl 5-bromo-2-furoate (2.05 mg, 10 mmol), (2-bromoethoxy)-tert-butyldimethylsilane (3 g, 12.5 mmol), nickel (II) iodide (312.5 mg, 1 mmol), 4,4′-di-O-tert-butyl-2,2′-bypyridine (268.4 mg, 1 mmol), pyridine (0.08 mL, 1 mmol), NaI (750 mg, 5 mmol) and Zn (1.3 g, 20 mmol) in DMPU (60 mL, 10 mmol) was stirred at 60° C. for 3 h. The mixture was diluted with water (100 mL) and extracted with EtOAc (100 mL). The organic phase was washed with brine (100 mL×3), dried over Na₂SO₄, filtered and concentrated to give a crude product which was purified by flash column (eluting with 100% PE) to give methyl 5-[2-[tert-butyl(dimethyl)silyl]oxyethyl]furan-2-carboxylate as a colorless oil (Int-29a, 520 mg, 18.4%). MS obsd. (ESI⁺) [(M+H)⁺]: 285.2.

### Step 2: Preparation of methyl 5-(2-hydroxyethyl)furan-2-carboxylate (Int-29b)

HCl/MeOH (3.5 mL, 14 mmol) was added into methyl 5-[2-[tert-butyl(dimethyl)silyl]oxyethyl]furan-2-carboxylate (Int-29a, 520 mg, 1.83 mmol). After being stirred at 25° C. for 1 h, the solvent was evaporated to give methyl 5-(2-hydroxyethyl)furan-2-carboxylate (Int-29b, 300 mg), which was used for the next step without further purification. MS obsd. (ESI⁺) [(M+H)⁺]: 171.1.

### Step 3: Preparation of methyl 5-(2-bromoethyl)furan-2-carboxylate (Int-29c)

To a solution of methyl 5-(2-hydroxyethyl)furan-2-carboxylate (Int-29b, 300 mg, 1.76 mmol) in DCM (5 mL) were added CBr₄ (1.2 g, 3.53 mmol) and PPh₃ (694 mg, 2.64 mmol). After being stirred at 20° C. for 2 h, the mixture was purified by flash column (eluting with EtOAc/PE=1/40) to give methyl 5-(2-bromoethyl)furan-2-carboxylate as a colorless oil (Int-29c, 220 mg, 53.5%). MS obsd. (ESI⁺) [(M+H)⁺]: 232.9.

### Step 4: Preparation of methyl 5-(2-methylsulfonylethyl)furan-2-carboxylate (Int-29d)

To a solution of methyl 5-(2-bromoethyl)furan-2-carboxylate (Int-29c, 180 mg, 0.77 mmol) and sodium iodide (116 mg, 0.77 mmol) in DMF (3 mL) was added methylsulfinyloxysodium (236.5 mg, 2.32 mmol). After being stirred at 50° C. for 1 h, the mixture was treated with water (20 mL) and extracted with EtOAc (20 mL). The organic layer was washed with brine (20 mL×3), dried over Na₂SO₄, filtered and concentrated in vacuo. The residue was purified by flash column (eluting with EtOAc/PE=1/3) to give methyl 5-(2-methylsulfonylethyl)furan-2-carboxylate as a light yellow oil (Int-29d, 140 mg, 78%). MS obsd. (ESI⁺) [(M+H)⁺]: 233.0.

### Step 5: Preparation of 5-(2-methylsulfonylethyl)furan-2-carboxylic acid (Int-29)

To a solution of methyl 5-(2-methylsulfonylethyl)furan-2-carboxylate (Int-29d, 80 mg, 0.34 mmol) in a mixed solvent of THF (3 mL) and MeOH (3 mL) was added a solution of LiOH in water (1.72 mL, 2 M). After being stirred at 25° C. for 1 h, most of the solvent was evaporated. The residue was acidified by HCl (1 M) to pH=5 and extracted with EtOAc (10 mL×3). The combined organic layer was dried over Na₂SO₄, filtered and concentrated in vacuo to give 5-(2-methylsulfonylethyl)furan-2-carboxylic acid as a white solid (Int-29, 75 mg, 99.8%). MS obsd. (ESI⁺) [(M+H)⁺]: 219.1.

### Intermediate Int-30

### 5-(sulfamoylmethyl)furan-2-carboxylic acid

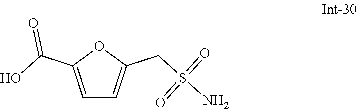 [SMILES: NS(=O)(=O)Cc1ccc(C(=O)O)o1]

The title compound was prepared according to the following scheme:

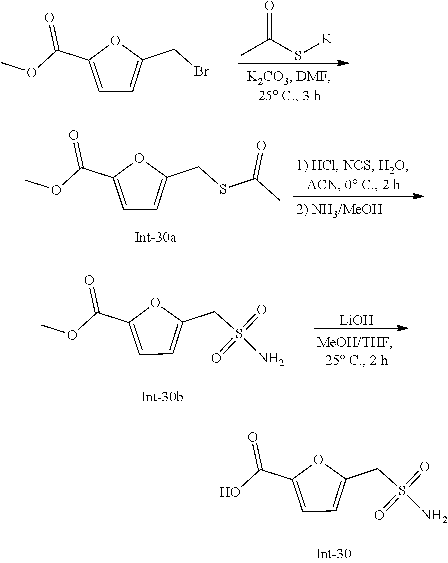

### Step 1: Preparation of methyl 5-(acetylsulfanylmethyl)furan-2-carboxylate (Int-30a)

A mixture of methyl 5-(bromomethyl)furan-2-carboxylate (1.1 g, 5 mmol) and acetylsulfanylpotassium (1.14 g, 10 mmol) in DMF (30 mL) was stirred at 25° C. for 3 h. The solvent was evaporated and the residue was purified by flash column (eluting with EtOAc/PE=1/12) to give methyl 5-(acetylsulfanylmethyl)furan-2-carboxylate as a yellow oil (Int-30a, 550 mg, 51.3%). MS obsd. (ESI⁺) [(M+H)⁺]: 215.1.

### Step 2: Preparation of methyl 5-(sulfamoylmethyl)furan-2-carboxylate (Int-30b)

To a solution of HCl/H₂O (2 M, 4.18 mL, 8.36 mmol) and ACN (4 mL) was added N-chlorosuccinimide (1.37 g, 10.3 mmol) followed by methyl 5-(acetylsulfanylmethyl)furan-2-carboxylate (Int-30a, 550 mg, 2.57 mmol) at 0° C. After being treated with NH₃/MeOH (0.3 M, 20 mL) at 0° C. for further 2 h, the solvent was evaporated and the residue was separated by EtOAc (20 mL) and brine (30 mL). The organic layer was separated, dried over Na₂SO₄, filtered and concentrated to give methyl 5-(sulfamoylmethyl)furan-2-carboxylate as a yellow oil (Int-30b, 230 mg, 40.87%). MS obsd. (ESI⁺) [(M+Na)⁺]: 242.0.

### Step 3: Preparation of 5-(sulfamoylmethyl)furan-2-carboxylic acid (Int-30)

LiOH·H₂O (10 mL, 20 mmol) in water (10 mL) was added into a solution of methyl 5-(sulfamoylmethyl)furan-2-carboxylate (Int-30b, 250 mg, 1.14 mmol) in a mixed solvent of methanol (10 mL) and THF (10 mL). After being stirred at 25° C. for 2 h, the mixture was acidified by 1 M HCl to pH=5. The solvent was evaporated, and the residue was separated by EtOAc (30 mL) and brine (30 mL). The organic layer was dried over Na₂SO₄, filtered and concentrated in vacuo to give 5-(sulfamoylmethyl)furan-2-carboxylic acid as a light yellow solid (Int-30, 170 mg, 72.65%). MS obsd. (ESI⁺) [(M+NH₄)⁺]: 223.1.

### Intermediate Int-31

### 5-(2-sulfamoylethyl)furan-2-carboxylic acid

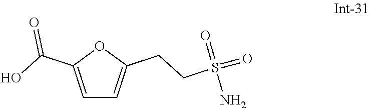 [SMILES: NS(=O)(=O)CCc1ccc(C(=O)O)o1]

The title compound was prepared according to the following scheme:

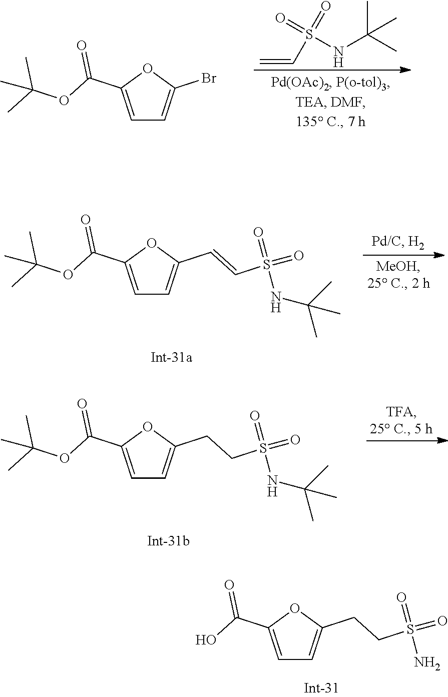

### Step 1: Preparation of tert-butyl 5-[(E)-2-(tert-butylsulfamoyl)vinyl]furan-2-carboxylate (Int-31a)

A mixture of tert-butyl 5-bromofuran-2-carboxylate (1 g, 4.05 mmol), N-tert-butylethenesulfonamide (694 mg, 4.25 mmol), palladium (II) acetate (45.4 mg, 0.2 mmol), tri-o-tolylphosphine (123.2 mg, 0.4 mmol) and triethylamine (1.7 mL, 12.1 mmol) in DMF (10 mL) was heated with stirring at 135° C. for 7 h. The solvent was removed in vacuo and the residue was purified by flash column (ACN/H₂O, with 0.01% NH₄OH) to give tert-butyl 5-[(E)-2-(tert-butylsulfamoyl)vinyl]furan-2-carboxylate as a white solid (Int-31a, 380 mg, 28.5%). MS obsd. (ESI⁺) [(M+Na)⁺]: 352.1.

### Step 2: Preparation of tert-butyl 5-[2-(tert-butylsulfamoyl)ethyl]furan-2-carboxylate (Int-31b)

Under an atmosphere of hydrogen (760 mmHg), a mixture of tert-butyl 5-[(E)-2-(tert-butylsulfamoyl)vinyl]furan-2-carboxylate (Int-31a, 280 mg, 0.85 mmol) and Pd/C (60 mg, 0.85 mmol) in MeOH (10 mL) was stirred at 25° C. for 2 h. The reaction mixture was filtered, the filtration was concentrated to give tert-butyl 5-[2-(tert-butylsulfamoyl)ethyl]furan-2-carboxylate as a white solid (Int-31b, 260 mg, 92.3%). MS obsd. (ESI⁺) [(M+Na)⁺]: 354.1.

### Step 3: Preparation of 5-(2-sulfamoylethyl)furan-2-carboxylic acid (Int-31)

The mixture of tert-butyl 5-[2-(tert-butylsulfamoyl)ethyl]furan-2-carboxylate (Int-31b, 260 mg, 0.78 mmol) and trifluoroacetic acid (5.0 mL, 0.78 mmol) was stirred at 25° C. for 5 h. The solvent was removed under reduced pressure to give 5-(2-sulfamoylethyl)furan-2-carboxylic acid as a light yellow oil (Int-31, 140 mg, 81.4%), which was used for the next step without further purification. MS obsd. (ESI⁺) [(M+NH₄)⁺]: 220.1.

### Intermediate Int-32

### 5-[(sulfamoylamino)methyl]furan-2-carboxylic acid

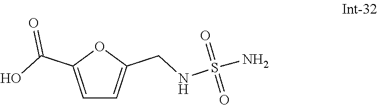 [SMILES: NS(=O)(=O)NCc1ccc(C(=O)O)o1]

The title compound was prepared according to the following scheme:

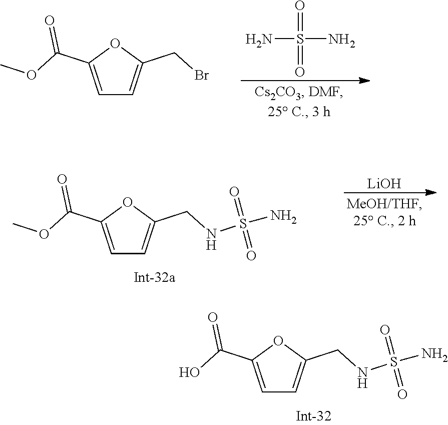

### Step 1: Preparation of methyl 5-[(sulfamoylamino)methyl]furan-2-carboxylate (Int-32a)

To a solution of sulfamide (0.72 mL, 12 mmol) in DMF (15 mL) was added cesium carbonate (1.30 g, 4 mmol), followed by a solution of methyl 5-(bromomethyl)furan-2-carboxylate (876.1 mg, 4 mmol) in DMF (1 mL) dropwise during 10 min. After being stirred at 25° C. for 3 h, the solvent was evaporated and the residue was purified by flash column (eluting with EtOAc/PE=1/4) to give methyl 5-[(sulfamoylamino)methyl]furan-2-carboxylate as a colorless oil (Int-32a, 420 mg, 44.8%). MS obsd. (ESI⁺) [(M+H)⁺]: 235.1.

### Step 2: Preparation of 5-[(sulfamoylamino)methyl]furan-2-carboxylic acid (Int-32)

LiOH·H₂O (14.97 mL, 29.95 mmol) in water (25 mL) was added into a solution of methyl 5-[(sulfamoylamino)methyl]furan-2-carboxylate (Int-32a, 400 mg, 1.71 mmol) in a mixed solvent of MeOH (25 mL) and THF (25 mL). After being stirred at 25° C. for 2 h, the reaction was acidified by 1 M HCl to pH=5. The solvent was evaporated and the residue was extracted by EtOAc (30 mL) and brine (30 mL). The organic layer was separated, dried over Na₂SO₄, filtered and concentrated in vacuo to give 5-[(sulfamoylamino)methyl]furan-2-carboxylic acid as a light yellow solid (Int-32, 340 mg, 90%). MS obsd. (ESI⁺) [(M+H)⁺]: 221.1.

### Intermediate Int-33

### 5-(1-oxo-4,5-dihydro-3H-isothiazol-1-yl)furan-2-carboxylic acid

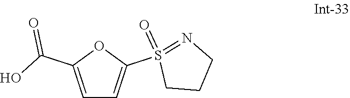 [SMILES: O=C(O)c1ccc(S2(=O)=NCCC2)o1]

The title compound was prepared according to the following scheme:

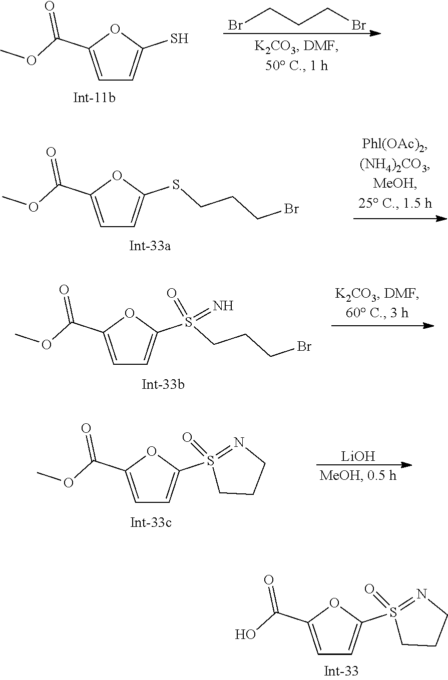

### Step 1: Preparation of methyl 5-(3-bromopropylsulfanyl)furan-2-carboxylate (Int-33a)

To a solution of methyl 5-sulfanylfuran-2-carboxylate (Int-11b, 160 mg, 1.01 mmol) in DMF (1 mL) were added 1,3-dibromopropane (1.23 g, 6.07 mmol) and K₂CO₃ (978.6 mg, 7.08 mmol). After being stirred at 50° C. for 1 h, the mixture was dissolved in EtOAc (80 mL), washed with brine (15 mL×5). The organics were combined, dried over Na₂SO₄ and concentrated to give methyl 5-(3-bromopropylsulfanyl)furan-2-carboxylate as a light yellow oil (Int-33a, 254 mg, yield 72%). MS obsd. (ESI⁺) [(M+H)⁺]: 279.0.

### Step 2: Preparation of methyl 5-(3-bromopropylsulfonimidoyl)furan-2-carboxylate (Int-33b)

To a solution of methyl 5-(3-bromopropylsulfanyl)furan-2-carboxylate (Int-33a, 250 mg, 0.9 mmol) in MeOH (5 mL) were added iodobenzene diacetate (432.7 mg, 1.34 mmol) and ammoniumcarbonate (60.2 mg, 0.63 mmol). After being stirred at 25° C. for 1.5 h, the mixture was taken up in DCM (100 mL), washed with water (20 mL×3) and brine (20 mL). The organic layer was dried over Na₂SO₄ and concentrated in vacuo to give methyl 5-(3-bromopropylsulfonimidoyl)furan-2-carboxylate as a light yellow oil (Int-33b, 305 mg, yield 87.8%), which was used for the next step without further purification. MS obsd. (ESI⁺) [(M+H)⁺]: 310.0.

### Step 3: Preparation of methyl 5-(1-oxo-4,5-dihydro-3H-isothiazol-1-yl)furan-2-carboxylate (Int-33c)

To a solution of methyl 5-(3-bromopropylsulfonimidoyl)furan-2-carboxylate (Int-33b, 270 mg, 0.87 mmol) in DMF (9 mL) was added K₂CO₃ (180.2 mg, 1.31 mmol). After being heated with stirring at 60° C. for 3 h, the reaction mixture was filtered and the filtrate was concentration to dryness. The crude product was purified by flash column (eluting with EtOAc/PE=80%) to give methyl 5-(1-oxo-4,5-dihydro-3H-isothiazol-1-yl)furan-2-carboxylate as a light yellow solid (Int-33c, 50 mg, 19.8%). MS obsd. (ESI⁺) [(M+H)⁺]: 230.1.

### Step 4: Preparation of 5-(1-oxo-4,5-dihydro-3H-isothiazol-1-yl)furan-2-carboxylic acid (Int-33)

To a solution of methyl 5-(1-oxo-4,5-dihydro-3H-isothiazol-1-yl)furan-2-carboxylate (Int-33c, 40 mg, 0.17 mmol) in a mixed solvent of MeOH (2.5 mL) and water (0.8 mL) was added LiOH·H₂O (8.05 mg, 0.19 mmol) and the reaction was stirred at 25° C. for 0.5 h. The mixture was concentrated to remove MeOH. The aqueous solution was diluted with water (5 mL) and acidified to pH=7 with 2 M HCl and lyophilized to give 5-(1-oxo-4,5-dihydro-3H-isothiazol-1-yl)furan-2-carboxylic acid as a light yellow oil (Int-33, 60 mg, 86.3%). MS obsd. (ESI⁺) [(M+H)⁺]: 216.1.

### Intermediate Int-34

### 5-(1-oxo-1lambda6-thia-2-azacyclohexen-1-yl)furan-2-carboxylic acid

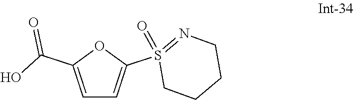 [SMILES: O=C(O)c1ccc(S2(=O)=NCCCC2)o1]

The title compound was prepared in analogy to the procedure described for the preparation of 5-(1-oxo-4,5-dihydro-3H-isothiazol-1-yl)furan-2-carboxylic acid (Int-33), by using 1,4-dibromobutane instead of 1,3-dibromopropane. MS obsd. (ESI⁺) [(M+H)⁺]: 230.1

### Example 1

### N-[6-(5-chloro-1,3-benzoxazol-2-yl)spiro[3.3]heptan-2-yl]-5-(trifluoromethyl)furan-2-carboxamide

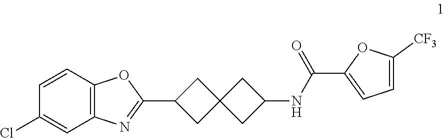 [SMILES: O=C(NC1CC2(C1)CC(c1nc3cc(Cl)ccc3o1)C2)c1ccc(C(F)(F)F)o1]

The title compound was prepared according to the following scheme:

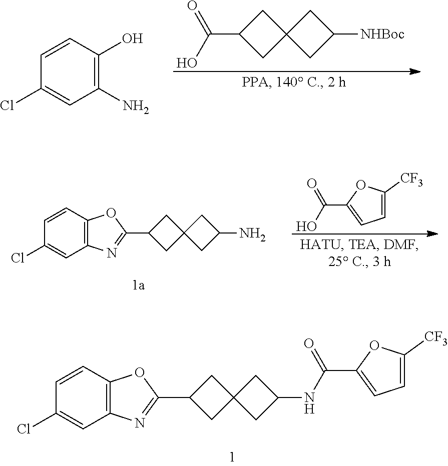 [SMILES: Nc1cc(Cl)ccc1O ; CC(C)(C)OC(=O)NC1CC2(C1)CC(C(=O)O)C2 ; NC1CC2(C1)CC(c1nc3cc(Cl)ccc3o1)C2 ; O=C(O)c1ccc(C(F)(F)F)o1 ; O=C(NC1CC2(C1)CC(c1nc3cc(Cl)ccc3o1)C2)c1ccc(C(F)(F)F)o1]

### Step 1: Preparation of 6-(5-chloro-1,3-benzoxazol-2-yl)spiro[3.3]heptan-2-amine (1a)

The polyphosphoric acid (4.8 g, 20 mmol) in a seal tube was heated with stirring at 110° C. for 10 min, then a mixture of 2-amino-4-chlorophenol (1.44 mg, 10 mmol) and 2-(tert-butoxycarbonylamino)spiro[3.3]heptane-6-carboxylic acid (2.68 g, 10.5 mmol) was added. The resulting mixture was heated with stirring at 140° C. for 2 h. After being poured into ice-water (60 mL) and basified with NH₄OH to pH=8, the resulting mixture was extracted with DCM (100 mL×3). The organic layer was dried over Na₂SO₄ and concentrated in vacuo. The residue was purified by flash column (eluting with 8% NH₄OH in MeOH/DCM=0 to 20%) to give 6-(5-chloro-1,3-benzoxazol-2-yl)spiro[3.3]heptan-2-amine as a light brown oil (1a, 2.5 g), which was used for the next step without further purification. MS obsd. (ESI⁺) [(M+H)⁺]: 263.1. ¹H NMR (400 MHz, CDCl₃) δ ppm: 7.63 (d, J=1.9 Hz, 1H), 7.38 (d, J=8.6 Hz, 1H), 7.27 (d, J=1.7 Hz, 1H), 3.65 (p, J=8.6 Hz, 1H), 3.41 (p, J=7.8 Hz, 1H), 2.49-2.58 (m, 6H), 2.40 (ddd, J=17.3, 10.3, 5.8 Hz, 2H), 1.84 (ddd, J=31.5, 11.2, 8.4 Hz, 2H).

### Step 2: Preparation of 6-(5-chloro-1,3-benzoxazol-2-yl)spiro[3.3]heptan-2-amine (Example 1)

To a solution of 5-(trifluoromethyl)-2-furoic acid (102.8 mg, 0.57 mmol) and 6-(5-chloro-1,3-benzoxazol-2-yl)spiro[3.3]heptan-2-amine (1a, 100 mg, 0.38 mmol) in DMF (5 mL) were added triethylamine (115.5 mg, 1.14 mmol) and HATU (217 mg, 0.57 mmol). After being stirred at 25° C. for 3 h, the mixture was diluted with EtOAc (40 mL), washed with water (20 mL×2) and brine (20 mL). The organic layer was dried over Na₂SO₄, concentrated in vacuo. The residue was purified by flash column (eluting with EtOAc/PE=30/70) and then preparative HPLC to give N-[6-(5-chloro-1,3-benzoxazol-2-yl)spiro[3.3]heptan-2-yl]-5-(trifluoromethyl)furan-2-carboxamide as a white solid (Example 1, 59 mg, 36.1%). MS obsd. (ESI⁺) [(M+H)⁺]: 425.1. ¹H NMR (400 MHz, DMSO-d₆) δ ppm: 8.88 (br d, J=7.58 Hz, 1H), 7.80 (d, J=1.96 Hz, 1H), 7.72 (d, J=8.68 Hz, 1H), 7.33-7.44 (m, 2H), 7.30 (d, J=3.55 Hz, 1H), 4.23-4.36 (m, 1H), 3.74 (t, J=8.50 Hz, 1H), 2.53-2.68 (m, 3H), 2.40-2.47 (m, 2H), 2.12-2.36 (m, 3H).

### Example 2

### N-[6-(5-chloro-1,3-benzoxazol-2-yl)spiro[3.3]heptan-2-yl]-5-cyano-furan-2-carboxamide

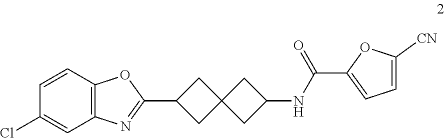 [SMILES: N#Cc1ccc(C(=O)NC2CC3(C2)CC(c2nc4cc(Cl)ccc4o2)C3)o1]

The title compound was prepared in analogy to the procedure described for the preparation of Example 1, by using 5-cyanofuran-2-carboxylic acid instead of 5-(trifluoromethyl)-2-furoic acid. The product was purified by preparative HPLC to afford Example 2 as a white solid. MS obsd. (ESI⁺) [(M+H)⁺]: 382.1. ¹H NMR (400 MHz, CDCl₃) δ ppm: 7.65 (d, J=3.6 Hz, 1H), 7.40 (d, J=8.0 Hz, 1H), 7.27-7.29 (m, 1H), 7.14-7.17 (m, 2H), 6.49 (d, J=8.0 Hz, 1H), 4.44-4.55 (m, 1H), 3.66-3.74 (m, 1H), 2.71-2.77 (m, 1H), 2.43-2.64 (m, 5H), 2.02-2.18 (m, 2H).

### Example 3

### 5-bromo-N-[6-(5-chloro-1,3-benzoxazol-2-yl)spiro[3.3]heptan-2-yl]furan-2-carboxamide

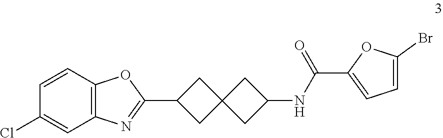 [SMILES: O=C(NC1CC2(C1)CC(c1nc3cc(Cl)ccc3o1)C2)c1ccc(Br)o1]

The title compound was prepared in analogy to the procedure described for the preparation of Example 1, by using 5-bromofuran-2-carboxylic acid instead of 5-(trifluoromethyl)-2-furoic acid. The product was purified by preparative HPLC to afford Example 3 as a white solid. MS obsd. (ESI⁺) [(M+H)⁺]: 435.0. ¹H NMR (400 MHz, CDCl₃) δ ppm: 7.66 (d, J=1.96 Hz, 1H), 7.41 (d, J=8.68 Hz, 1H), 7.29 (m, 1H), 7.07 (d, J=3.42 Hz, 1H), 6.46 (d, J=3.42 Hz, 1H), 6.38 (br d, J=7.46 Hz, 1H), 4.46-4.56 (m, 1H), 3.71 (quin, J=8.56 Hz, 1H), 2.69-2.78 (m, 1H), 2.45-2.68 (m, 5H), 2.03-2.20 (m, 2H).

### Example 4

### 4-bromo-N-[6-(5-chloro-1,3-benzoxazol-2-yl)spiro[3.3]heptan-2-yl]furan-2-carboxamide

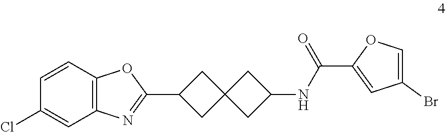 [SMILES: O=C(NC1CC2(C1)CC(c1nc3cc(Cl)ccc3o1)C2)c1cc(Br)co1]

The title compound was prepared in analogy to the procedure described for the preparation of Example 1, by using 4-bromofuran-2-carboxylic acid instead of 5-(trifluoromethyl)-2-furoic acid. The product was purified by preparative HPLC to afford Example 4 as a white solid. MS obsd. (ESI⁺) [(M+H)⁺]: 435.0. ¹H NMR (400 MHz, CDCl₃) δ ppm: 7.65 (d, J=4.0 Hz, 1H), 7.42-7.44 (m, 1H), 7.38-7.41 (m, 1H), 7.25-7.29 (m, 1H), 7.10-7.12 (m, 1H), 6.39 (d, J=8.0 Hz, 1H), 4.53-4.43 (m, 1H), 3.65-3.74 (m, 1H), 2.69-2.75 (m, 1H), 2.43-2.64 (m, 5H), 2.02-2.13 (m, 2H).

### Example 5

### 3-bromo-N-[6-(5-chloro-1,3-benzoxazol-2-yl)spiro[3.3]heptan-2-yl]furan-2-carboxamide

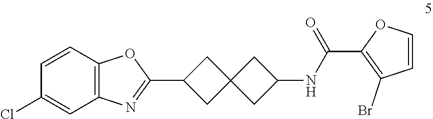 [SMILES: O=C(NC1CC2(C1)CC(c1nc3cc(Cl)ccc3o1)C2)c1occc1Br]

The title compound was prepared in analogy to the procedure described for the preparation of Example 1, by using 3-bromofuran-2-carboxylic acid instead of 5-(trifluoromethyl)-2-furoic acid. The product was purified by preparative HPLC to afford Example 4 as a white solid. MS obsd. (ESI⁺) [(M+H)⁺]: 435.0. ¹H NMR (400 MHz, CDCl₃) δ ppm: 7.64 (d, J=1.83 Hz, 1H), 7.37-7.44 (m, 2H), 7.25-7.28 (m, 1H), 6.57 (d, J=1.83 Hz, 1H), 6.48-6.55 (m, 1H), 4.43-4.54 (m, 1H), 3.69 (quin, J=8.59 Hz, 1H), 2.67-2.79 (m, 1H), 2.43-2.66 (m, 5H), 1.99-2.18 (m, 2H).

### Example 6

### 5-chloro-N-[6-(5-chloro-1,3-benzoxazol-2-yl)spiro[3.3]heptan-2-yl]furan-2-carboxamide

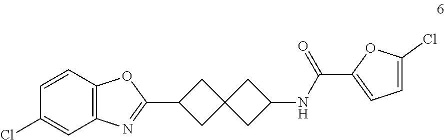 [SMILES: O=C(NC1CC2(C1)CC(c1nc3cc(Cl)ccc3o1)C2)c1ccc(Cl)o1]

The title compound was prepared in analogy to the procedure described for the preparation of Example 1, by using 5-chlorofuran-2-carboxylic acid instead of 5-(trifluoromethyl)-2-furoic acid. The product was purified by preparative HPLC to afford Example 6 as a white solid. MS obsd. (ESI⁺) [(M+H)⁺]: 391.1. ¹H NMR (400 MHz, DMSO-d₆) δ ppm: 8.59 (d, J=7.70 Hz, 1H), 7.80 (d, J=1.96 Hz, 1H), 7.72 (d, J=8.56 Hz, 1H), 7.40 (dd, J=2.20, 8.68 Hz, 1H), 7.16 (d, J=3.55 Hz, 1H), 6.65 (d, J=3.55 Hz, 1H), 4.27 (q, J=8.15 Hz, 1H), 3.73 (t, J=8.50 Hz, 1H), 2.41-2.62 (m, 5H), 2.08-2.31 (m, 3H).

### Example 7

### N-[6-(5-chloro-1,3-benzoxazol-2-yl)spiro[3.3]heptan-2-yl]-5-isopropyl-furan-2-carboxamide

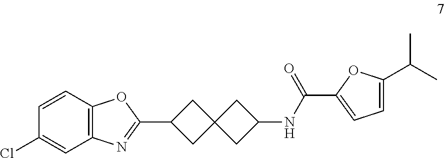 [SMILES: CC(C)c1ccc(C(=O)NC2CC3(C2)CC(c2nc4cc(Cl)ccc4o2)C3)o1]

The title compound was prepared in analogy to the procedure described for the preparation of Example 1, by using 5-isopropylfuran-2-carboxylic acid instead of 5-(trifluoromethyl)-2-furoic acid. The product was purified by preparative HPLC to afford Example 7 as a white solid. MS obsd. (ESI⁺) [(M+H)⁺]: 399.0. ¹H NMR (400 MHz, DMSO-d₆) δ ppm: 8.30 (d, J=7.58 Hz, 1H), 7.80 (d, J=1.96 Hz, 1H), 7.72 (d, J=8.56 Hz, 1H), 7.40 (dd, J=2.20, 8.56 Hz, 1H), 6.96 (d, J=3.42 Hz, 1H), 6.21 (d, J=3.42 Hz, 1H), 4.15-4.39 (m, 1H), 3.74 (quin, J=8.38 Hz, 1H), 2.95 (td, J=7.03, 13.82 Hz, 1H), 2.41-2.65 (m, 5H), 2.09-2.32 (m, 3H), 1.23 (d, J=6.85 Hz, 6H).

### Example 8

### N-[6-(5-chloro-1,3-benzoxazol-2-yl)spiro[3.3]heptan-2-yl]-5-methoxy-furan-2-carboxamide

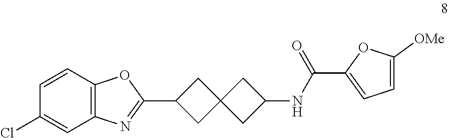 [SMILES: COc1ccc(C(=O)NC2CC3(C2)CC(c2nc4cc(Cl)ccc4o2)C3)o1]

The title compound was prepared in analogy to the procedure described for the preparation of Example 1, by using 5-methoxyfuran-2-carboxylic acid instead of 5-(trifluoromethyl)-2-furoic acid. The product was purified by preparative HPLC to afford Example 8 as a white solid. MS obsd. (ESI⁺) [(M+H)⁺]: 387.1. ¹H NMR (400 MHz, DMSO-d₆) δ ppm: 8.23 (br d, J=7.70 Hz, 1H), 7.80 (d, J=1.96 Hz, 1H), 7.72 (d, J=8.68 Hz, 1H), 7.40 (dd, J=1.90, 8.62 Hz, 1H), 7.02 (d, J=3.42 Hz, 1H), 5.52 (d, J=3.42 Hz, 1H), 4.25 (sxt, J=8.09 Hz, 1H), 3.88 (s, 3H), 3.73 (quin, J=8.50 Hz, 1H), 2.22-2.63 (m, 5H), 2.22-2.30 (m, 1H), 2.18 (br t, J=9.84 Hz, 1H), 2.04-2.13 (m, 1H).

### Example 9

### N-[6-(5-chloro-1,3-benzoxazol-2-yl)spiro[3.3]heptan-2-yl]-5-(methoxymethyl)furan-2-carboxamide

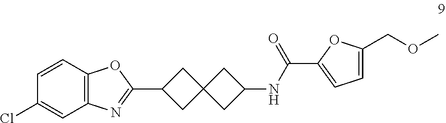 [SMILES: COCc1ccc(C(=O)NC2CC3(C2)CC(c2nc4cc(Cl)ccc4o2)C3)o1]

The title compound was prepared in analogy to the procedure described for the preparation of Example 1, by using 5-(methoxymethyl)furan-2-carboxylic acid instead of 5-(trifluoromethyl)-2-furoic acid. The product was purified by preparative HPLC to afford Example 9 as a white solid. MS obsd. (ESI⁺) [(M+H)⁺]: 401.1. ¹H NMR (400 MHz, DMSO-d₆) δ ppm: 8.49 (d, J=7.82 Hz, 1H), 7.80 (d, J=2.08 Hz, 1H), 7.71 (d, J=8.68 Hz, 1H), 7.40 (dd, J=2.08, 8.68 Hz, 1H), 7.04 (d, J=3.42 Hz, 1H), 6.55 (d, J=3.30 Hz, 1H), 4.39 (s, 2H), 4.29 (q, J=8.40 Hz, 1H), 3.73 (quin, J=8.50 Hz, 1H), 3.26 (s, 3H), 2.39-2.63 (m, 5H), 2.10-2.34 (m, 3H).

### Example 10

### N-[6-(5-chloro-1,3-benzoxazol-2-yl)spiro[3.3]heptan-2-yl]-5-(cyclopropanecarbonyl)furan-2-carboxamide

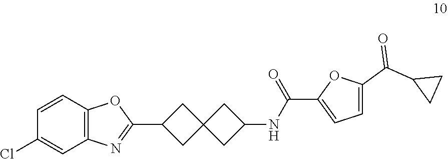 [SMILES: O=C(NC1CC2(C1)CC(c1nc3cc(Cl)ccc3o1)C2)c1ccc(C(=O)C2CC2)o1]

The title compound was prepared in analogy to the procedure described for the preparation of Example 1, by using 5-(cyclopropanecarbonyl)furan-2-carboxylic acid instead of 5-(trifluoromethyl)-2-furoic acid. The product was purified by preparative HPLC to afford Example 10 as a white solid. MS obsd. (ESI⁺) [(M+H)⁺]: 425.3. ¹H NMR (400 MHz, DMSO-d₆) δ ppm: 8.83 (d, J=7.70 Hz, 1H), 7.80 (d, J=2.20 Hz, 1H), 7.72 (d, J=8.68 Hz, 1H), 7.57 (d, J=3.67 Hz, 1H), 7.40 (dd, J=2.20, 8.68 Hz, 1H), 7.26 (d, J=3.67 Hz, 1H), 4.33 (q, J=8.19 Hz, 1H), 3.75 (t, J=8.50 Hz, 1H), 2.84-2.93 (m, 1H), 2.53-2.48 (m, 5H), 2.13-2.38 (m, 3H), 1.01-1.11 (m, 4H).

### Example 11

### N2-[6-(5-Chloro-1,3-benzoxazol-2-yl)spiro[3.3]heptan-2-yl]furan-2,5-dicarboxamide

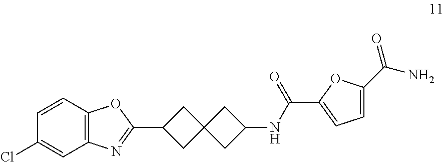 [SMILES: NC(=O)c1ccc(C(=O)NC2CC3(C2)CC(c2nc4cc(Cl)ccc4o2)C3)o1]

The title compound was prepared in analogy to the procedure described for the preparation of Example 1, by using 5-carbamoylfuran-2-carboxylic acid instead of 5-(trifluoromethyl)-2-furoic acid. The product was purified by preparative HPLC to afford Example 11 as a white solid. MS obsd. (ESI⁺) [(M+H)⁺]: 400.1. ¹H NMR (400 MHz, DMSO-d₆) δ ppm: 8.60 (d, J=7.7 Hz, 1H), 8.03 (s, 1H), 7.80 (d, J=2.1 Hz, 1H), 7.71 (t, J=5.8 Hz, 2H), 7.40 (dd, J=8.7, 2.1 Hz, 1H), 7.11 (s, 2H), 4.24-4.39 (m, 1H), 3.72-3.79 (m, 1H), 2.61 (dd, J=8.3, 2.4 Hz, 1H), 2.54-2.59 (m, 2H), 2.46 (dd, J=8.4, 5.4 Hz, 2H), 2.33-2.38 (m, 1H), 2.07-2.23 (m, 2H).

### Example 12

### N-[6-(5-chloro-1,3-benzoxazol-2-yl)spiro[3.3]heptan-2-yl]-3-(trifluoromethyl)-1H-pyrazole-5-carboxamide

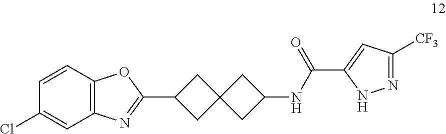 [SMILES: O=C(NC1CC2(C1)CC(c1nc3cc(Cl)ccc3o1)C2)c1cc(C(F)(F)F)n[nH]1]

The title compound was prepared in analogy to the procedure described for the preparation of Example 1, by using 3-(trifluoromethyl)-1H-pyrazole-5-carboxylic acid instead of 5-(trifluoromethyl)-2-furoic acid. The product was purified by preparative HPLC to afford Example 12 as a white solid. MS obsd. (ESI⁺) [(M+H)⁺]: 425.0. ¹H NMR (400 MHz, DMSO-d₆) δ ppm: 14.42 (s, 1H), 8.77 (br d, J=7.21 Hz, 1H), 7.80 (d, J=1.96 Hz, 1H), 7.72 (d, J=8.68 Hz, 1H), 7.40 (dd, J=1.96, 8.68 Hz, 1H), 7.29 (s, 1H), 4.25-4.36 (m, 1H), 3.75 (quin, J=8.44 Hz, 1H), 2.46-2.65 (m, 5H), 2.31-2.40 (m, 1H), 2.13-2.22 (m, 1H), 2.05-2.13 (m, 1H).

### Example 13

### N-[6-(5-chloro-1,3-benzoxazol-2-yl)spiro[3.3]heptan-2-yl]-1-methyl-pyrazole-4-carboxamide

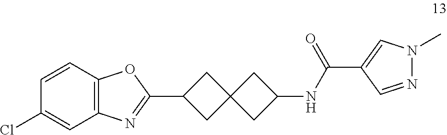 [SMILES: Cn1cc(C(=O)NC2CC3(C2)CC(c2nc4cc(Cl)ccc4o2)C3)cn1]

The title compound was prepared in analogy to the procedure described for the preparation of Example 1, by using 1-methylpyrazole-4-carboxylic acid instead of 5-(trifluoromethyl)-2-furoic acid. The product was purified by preparative HPLC to afford Example 13 as a white solid. MS obsd. (ESI⁺) [(M+H)⁺]: 371.1. ¹H NMR (400 MHz, DMSO-d₆) δ ppm: 8.13 (br d, J=7.46 Hz, 1H), 8.08 (s, 1H), 7.81 (br s, 1H), 7.79 (br s, 1H), 7.71 (d, J=8.68 Hz, 1H), 7.39 (br d, J=8.68 Hz, 1H), 4.26 (sxt, J=7.97 Hz, 1H), 3.83 (s, 3H), 3.73 (quin, J=8.38 Hz, 1H), 2.43-2.63 (m, 5H), 2.25-2.34 (m, 1H), 2.12 (br t, J=9.84 Hz, 1H), 2.03 (br t, J=10.03 Hz, 1H).

### Example 14

### N-[6-(5-chloro-1,3-benzoxazol-2-yl)spiro[3.3]heptan-2-yl]-5-methylsulfinyl-furan-2-carboxamide

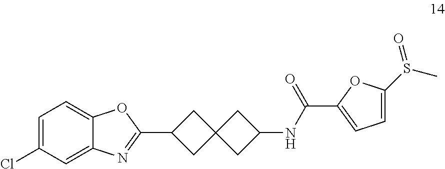 [SMILES: CS(=O)c1ccc(C(=O)NC2CC3(C2)CC(c2nc4cc(Cl)ccc4o2)C3)o1]

The title compound was prepared in analogy to the procedure described for the preparation of Example 1, by using 5-methylsulfinylfuran-2-carboxylic acid (Int-2) instead of 5-(trifluoromethyl)-2-furoic acid. The product was purified by preparative HPLC to afford Example 14 as a white solid. MS obsd. (ESI⁺) [(M+H)⁺]: 419.1. ¹H NMR (400 MHz, CDCl₃) δ ppm: 7.65 (d, J=1.96 Hz, 1H), 7.40 (d, J=8.68 Hz, 1H), 7.28 (dd, J=2.08, 8.0 Hz, 1H), 7.17 (d, J=3.55 Hz, 1H), 6.96 (d, J=3.55 Hz, 1H), 6.66 (br d, J=7.46 Hz, 1H), 4.50 (sxt, J=8.02 Hz, 1H), 3.70 (quin, J=8.56 Hz, 1H), 3.01 (s, 3H), 2.69-2.78 (m, 1H), 2.62-2.66 (m, 2H), 2.44-2.61 (m, 3H), 2.06-2.20 (m, 2H).

### Example 15

### Example 15-a, Example 15-b

### N-[6-(5-chloro-1,3-benzoxazol-2-yl)spiro[3.3]heptan-2-yl]-5-methylsulfonyl-furan-2-carboxamide

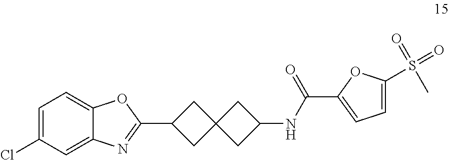 [SMILES: CS(=O)(=O)c1ccc(C(=O)NC2CC3(C2)CC(c2nc4cc(Cl)ccc4o2)C3)o1]

N-[6-(5-Chloro-1,3-benzoxazol-2-yl)spiro[3.3]heptan-2-yl]-5-methylsulfonyl-furan-2-carboxamide (Example 15) was prepared in analogy to the procedure described for the preparation of Example 1, by using 5-methylsulfonylfuran-2-carboxylic acid (Int-3) instead of 5-(trifluoromethyl)-2-furoic acid. MS obsd. (ESI⁺) [(M+H)⁺]: 435.0. ¹H NMR (400 MHz, CD₃Cl) δ ppm: 7.65 (d, J=1.96 Hz, 1H), 7.40 (d, J=8.56 Hz, 1H), 7.29 (d, J=2.08 Hz, 1H), 7.17-7.24 (m, 2H), 6.61 (br d, J=7.70 Hz, 1H), 4.51 (sxt, J=8.17 Hz, 1H), 3.70 (quin, J=8.56 Hz, 1H), 3.21 (s, 3H), 2.70-2.78 (m, 1H), 2.64 (d, J=9.41 Hz, 2H), 2.44-2.61 (m, 3H), 2.06-2.23 (m, 2H).

The two enantiomers (Example 15-a, Example 15-b) were obtained through SFC [Instrument: SFC 80; Column: OJ, 250×20 mm I.D., 5 μm; Mobile phase: A for CO₂ and B for Methanol (0.25% NH₄OH); Gradient: B 15%; Flow rate: 50 mL/min; Back pressure: 100 bar; Column temperature: 35° C.] chiral separation of N-[6-(5-chloro-1,3-benzoxazol-2-yl)spiro[3.3]heptan-2-yl]-5-methylsulfonyl-furan-2-carboxamide (Example 15). Example 15-a was eluted out before Example 15-b. The absolute configuration of Example 15-b was determined by X-ray diffraction study (FIG. 1).

### (Sₐ)—N-[6-(5-chloro-1,3-benzoxazol-2-yl)spiro[3.3]heptan-2-yl]-5-methylsulfonyl-furan-2-carboxamide (Example 15-a)

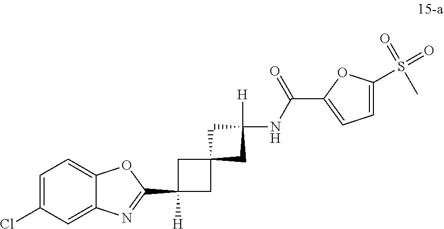

### (Rₐ)—N-[6-(5-chloro-1,3-benzoxazol-2-yl)spiro[3.3]heptan-2-yl]-5-methylsulfonyl-furan-2-carboxamide (Example 15-b)

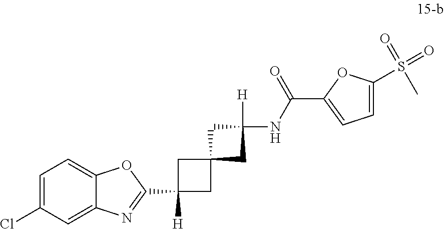

Example 15-a: white solid. [α]²⁰_{D}=+28.621 (C=0.1, MeOH). MS obsd. (ESI⁺) [(M+H)⁺]: 435.2. ¹H NMR (400 MHz, CD₃OD) δ ppm: 7.63 (d, J=2.08 Hz, 1H), 7.55 (d, J=8.68 Hz, 1H), 7.35 (dd, J=1.96, 8.68 Hz, 1H), 7.29 (d, J=3.67 Hz, 1H), 7.21 (d, J=3.79 Hz, 1H), 4.41 (quin, J=8.28 Hz, 1H), 3.75 (quin, J=8.50 Hz, 1H), 3.29 (s, 3H), 2.60-2.72 (m, 3H), 2.41-2.59 (m, 3H), 2.15-2.32 (m, 2H).

Example 15-b: white solid. [α]²⁰_{D}=−28.475 (C=0.1, MeOH). MS obsd. (ESI⁺) [(M+H)⁺]: 435.1. ¹H NMR (400 MHz, CD₃OD) δ ppm: 7.63 (d, J=2.08 Hz, 1H), 7.55 (d, J=8.68 Hz, 1H), 7.35 (dd, J=2.08, 8.68 Hz, 1H), 7.29 (d, J=3.67 Hz, 1H), 7.21 (d, J=3.67 Hz, 1H), 4.41 (quin, J=8.28 Hz, 1H), 3.75 (quin, J=8.50 Hz, 1H), 3.29 (s, 3H), 2.60-2.71 (m, 3H), 2.40-2.60 (m, 3H), 2.14-2.33 (m, 2H).

### Example 16

### N-[6-(5-chloro-1,3-benzoxazol-2-yl)spiro[3.3]heptan-2-yl]-5-ethylsulfonyl-furan-2-carboxamide

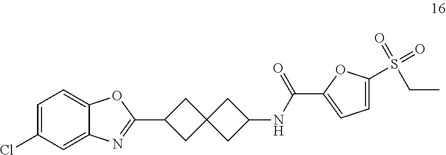 [SMILES: CCS(=O)(=O)c1ccc(C(=O)NC2CC3(C2)CC(c2nc4cc(Cl)ccc4o2)C3)o1]

The title compound was prepared in analogy to the procedure described for the preparation of Example 1, by using 5-ethylsulfonylfuran-2-carboxylic acid (Int-4) instead of 5-(trifluoromethyl)-2-furoic acid. The product was purified by preparative HPLC to afford Example 16 as a white solid. MS obsd. (ESI⁺) [(M+H)⁺]: 449.1. ¹H NMR (400 MHz, DMSO-d₆) δ ppm: 8.87 (d, J=7.70 Hz, 1H), 7.80 (d, J=1.96 Hz, 1H), 7.72 (d, J=8.68 Hz, 1H), 7.38-7.43 (m, 2H), 7.29 (d, J=3.67 Hz, 1H), 4.30 (sxt, J=8.07 Hz, 1H), 3.75 (quin, J=8.47 Hz, 1H), 3.46 (q, J=7.38 Hz, 2H), 2.41-2.74 (m, 5H), 2.29-2.38 (m, 1H), 2.0-2.28 (m, 2H), 1.17 (t, J=7.34 Hz, 3H).

### Example 17

### N-[6-(5-chloro-1,3-benzoxazol-2-yl)spiro[3.3]heptan-2-yl]-5-cyclopropylsulfonyl-furan-2-carboxamide

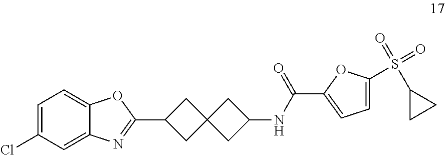 [SMILES: O=C(NC1CC2(C1)CC(c1nc3cc(Cl)ccc3o1)C2)c1ccc(S(=O)(=O)C2CC2)o1]

The title compound was prepared in analogy to the procedure described for the preparation of Example 1, by using 5-cyclopropylsulfonylfuran-2-carboxylic acid (Int-6), instead of 5-(trifluoromethyl)-2-furoic acid. The product was purified by preparative HPLC to afford Example 17 as a white solid. MS obsd. (ESI⁺) [(M+H)⁺]: 461.0. ¹H NMR (400 MHz, CD₃OD) δ ppm: 7.64 (d, J=1.96 Hz, 1H), 7.55 (d, J=8.68 Hz, 1H), 7.35 (dd, J=1.96, 8.68 Hz, 1H), 7.20-7.27 (m, 2H), 4.41 (quin, J=8.16 Hz, 1H), 3.75 (quin, J=8.38 Hz, 1H), 2.80-2.88 (m, 1H), 2.60-2.71 (m, 3H), 2.41-2.60 (m, 3H), 2.17-2.32 (m, 2H), 1.30-1.37 (m, 2H), 1.13-1.19 (m, 2H).

### Example 18

### N-[6-(5-chloro-1,3-benzoxazol-2-yl)spiro[3.3]heptan-2-yl]-5-(oxetan-3-ylsulfonyl)furan-2-carboxamide

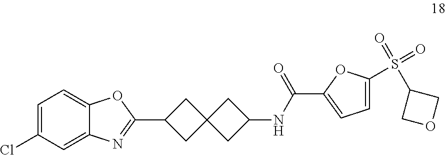 [SMILES: O=C(NC1CC2(C1)CC(c1nc3cc(Cl)ccc3o1)C2)c1ccc(S(=O)(=O)C2COC2)o1]

The title compound was prepared in analogy to the procedure described for the preparation of Example 1, by using 5-(oxetan-3-ylsulfonyl)furan-2-carboxylic acid (Int-7) instead of 5-(trifluoromethyl)-2-furoic acid. The product was purified by preparative HPLC to afford Example 18 as a white solid. MS obsd. (ESI⁺) [(M+H)⁺]: 477.1. ¹H NMR (400 MHz, CDCl₃) δ ppm: 7.65 (s, 1H), 7.40 (d, J=8.68 Hz, 1H), 7.31 (d, J=3.67 Hz, 1H), 7.29 (s, 1H), 7.23 (d, J=3.67 Hz, 1H), 6.59 (br d, J=7.21 Hz, 1H), 4.89-4.99 (m, 4H), 4.60 (tt, J=6.07, 7.93 Hz, 1H), 4.49 (sxt, J=8.14 Hz, 1H), 3.71 (quin, J=8.56 Hz, 1H), 2.69-2.77 (m, 1H), 2.44-2.67 (m, 5H), 2.07-2.20 (m, 2H).

### Example 19

### Example 19-a, Example 19-b, Example 19-c, Example 19-d

### N-[6-(5-chloro-1,3-benzoxazol-2-yl)spiro[3.3]heptan-2-yl]-5-(cyclopropylmethylsulfinyl)furan-2-carboxamide

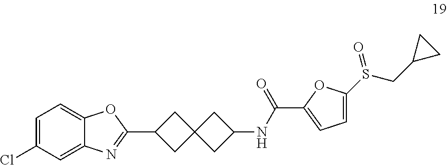 [SMILES: O=C(NC1CC2(C1)CC(c1nc3cc(Cl)ccc3o1)C2)c1ccc(S(=O)CC2CC2)o1]

N-[6-(5-Chloro-1,3-benzoxazol-2-yl)spiro[3.3]heptan-2-yl]-5-(cyclopropylmethylsulfinyl)furan-2-carboxamide (Example 19) was prepared in analogy to the procedure described for the preparation of Example 1, by using 5-(cyclopropylmethylsulfinyl)furan-2-carboxylic acid (Int-13) instead of 5-(trifluoromethyl)-2-furoic acid. MS obsd. (ESI⁺) [(M+H)⁺]: 459.1. ¹H NMR (400 MHz, CD₃OD) δ ppm: 7.64 (d, J=2.20 Hz, 1H), 7.55 (d, J=8.56 Hz, 1H), 7.35 (dd, J=2.08, 8.68 Hz, 1H), 7.19-7.24 (m, 1H), 4.41 (td, J=8.07, 16.63 Hz, 1H), 3.75 (quin, J=8.56 Hz, 1H), 3.35-3.41 (m, 1H), 3.22-3.28 (m, 1H), 2.41-2.72 (m, 6H), 2.15-2.32 (m, 2H), 0.88-1.00 (m, 1H), 0.57-0.72 (m, 2H), 0.42 (dt, J=4.65, 9.29 Hz, 1H), 0.30 (td, J=4.74, 9.35 Hz, 1H).

The four diastereomers (Example 19-a, Example 19-b, Example 19-c, Example 19-d) were obtained through SFC chiral separation of N-[6-(5-chloro-1,3-benzoxazol-2-yl)spiro[3.3]heptan-2-yl]-5-(cyclopropylmethylsulfinyl)furan-2-carboxamide (Example 19).

### (Rₐ)—N-[6-(5-chloro-1,3-benzoxazol-2-yl)spiro[3.3]heptan-2-yl]-5-[(S)-cyclopropylmethylsulfinyl]furan-2-carboxamide

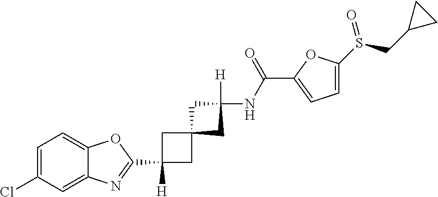

### (Rₐ)—N-[6-(5-chloro-1,3-benzoxazol-2-yl)spiro[3.3]heptan-2-yl]-5-[(R)-cyclopropylmethylsulfinyl]furan-2-carboxamide

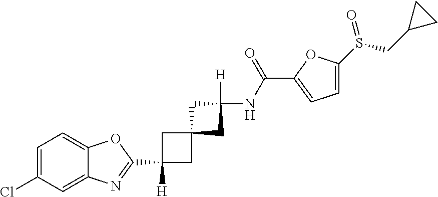

### (Sₐ)—N-[6-(5-Chloro-1,3-benzoxazol-2-yl)spiro[3.3]heptan-2-yl]-5-[(S)-cyclopropylmethylsulfinyl]furan-2-carboxamide

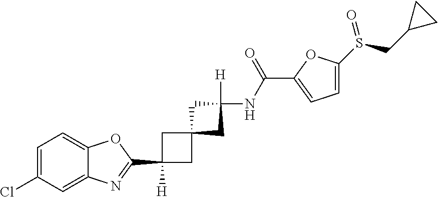

### (Sₐ)—N-[6-(5-chloro-1,3-benzoxazol-2-yl)spiro[3.3]heptan-2-yl]-5-[(R)-cyclopropylmethylsulfinyl]furan-2-carboxamide

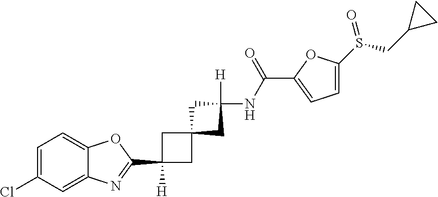

Example 19-a: white solid. SFC analysis: RT=11.010 min (Instrument: Waters Acquity UPCC; Column: Daicel CHIRALPAK IG-3, 3.0*150 mm, 3 μm; Mobile Phase: CO₂/EtOH=80/20; Flow rate: 2.0 mL/min; Column Temp: 37° C.). MS obsd. (ESI⁺) [(M+H)⁺]: 459.1. ¹H NMR (400 MHz, DMSO-d₆) δ ppm: 8.80 (d, J=7.7 Hz, 1H), 7.80 (d, J=2.1 Hz, 1H), 7.72 (d, J=8.7 Hz, 1H), 7.40 (dd, J=8.7, 2.1 Hz, 1H), 7.29 (d, J=3.6 Hz, 1H), 7.22 (d, J=3.6 Hz, 1H), 4.25-4.37 (m, 1H), 3.70-3.79 (m, 1H), 3.37 (d, J=7.1 Hz, 1H), 3.21 (dd, J=13.0, 7.6 Hz, 1H), 2.52-2.69 (m, 3H), 2.39-2.48 (m, 2H), 2.29-2.37 (m, 1H), 2.20-2.28 (m, 1H), 2.13-2.18 (m, 1H), 0.76-0.88 (m, 1H), 0.47-0.64 (m, 2H), 0.34-0.43 (m, 1H), 0.20-0.31 (m, 1H).

Example 19-b: white solid. SFC analysis: RT=9.119 min (Instrument: Waters Acquity UPCC; Column: Daicel CHIRALPAK IG-3, 3.0*150 mm, 3 μm; Mobile Phase: CO₂/EtOH=80/20; Flow rate: 2.0 mL/min; Column Temp: 37° C.). MS obsd. (ESI⁺) [(M+H)⁺]: 459.1. ¹H NMR (400 MHz, DMSO-d₆) δ ppm: 8.80 (d, J=7.7 Hz, 1H), 7.80 (d, J=2.0 Hz, 1H), 7.72 (d, J=8.7 Hz, 1H), 7.40 (dd, J=8.7, 2.1 Hz, 1H), 7.29 (d, J=3.6 Hz, 1H), 7.22 (d, J=3.6 Hz, 1H), 4.26-4.36 (m, 1H), 3.71-3.79 (m, 1H), 3.34-3.44 (m, 1H), 3.21 (dd, J=13.0, 7.6 Hz, 1H), 2.52-2.72 (m, 3H), 2.38-2.48 (m, 2H), 2.29-2.35 (m, 1H), 2.12-2.27 (m, 2H), 0.75-0.87 (m, 1H), 0.47-0.64 (m, 2H), 0.34-0.43 (m, 1H), 0.21-0.31 (m, 1H).

Example 19-c: white solid. SFC analysis: RT=10.021 min (Instrument: Waters Acquity UPCC; Column: Daicel CHIRALPAK IG-3, 3.0*150 mm, 3 μm; Mobile Phase: CO₂/EtOH=80/20; Flow rate: 2.0 mL/min; Column Temp: 37° C.). MS obsd. (ESI⁺) [(M+H)⁺]: 459.0. ¹H NMR (400 MHz, DMSO-d₆) δ ppm: 8.80 (d, J=7.7 Hz, 1H), 7.80 (d, J=2.0 Hz, 1H), 7.72 (d, J=8.7 Hz, 1H), 7.40 (dd, J=8.7, 2.1 Hz, 1H), 7.29 (d, J=3.6 Hz, 1H), 7.22 (d, J=3.6 Hz, 1H), 4.25-4.37 (m, 1H), 3.71-3.79 (m, 1H), 3.34-3.40 (m, 1H), 3.21 (dd, J=13.0, 7.6 Hz, 1H), 2.51-2.71 (m, 3H), 2.39-2.48 (m, 2H), 2.29-2.35 (m, 1H), 2.12-2.37 (m, 2H), 0.75-0.87 (m, 1H), 0.48-0.63 (m, 2H), 0.35-0.43 (m, 1H), 0.21-0.29 (m, 1H).

Example 19-d: white solid. SFC analysis: RT=8.703 min (Instrument: Waters Acquity UPCC; Column: Daicel CHIRALPAK IG-3, 3.0*150 mm, 3 μm; Mobile Phase: CO₂/EtOH=80/20; Flow rate: 2.0 mL/min; Column Temp: 37° C.). MS obsd. (ESI⁺) [(M+H)⁺]: 459.1. ¹H NMR (400 MHz, DMSO-d₆) δ ppm: 8.81 (d, J=7.6 Hz, 1H), 7.80 (d, J=2.1 Hz, 1H), 7.72 (d, J=8.7 Hz, 1H), 7.40 (dd, J=8.7, 2.1 Hz, 1H), 7.29 (d, J=3.6 Hz, 1H), 7.22 (d, J=3.6 Hz, 1H), 4.24-4.39 (m, 1H), 3.70-3.79 (m, 1H), 3.36-3.45 (m, 1H), 3.21 (dd, J=13.0, 7.6 Hz, 1H), 2.52-2.72 (m, 3H), 2.39-2.49 (m, 2H), 2.29-2.38 (m, 1H), 2.20-2.29 (m, 1H), 2.13-2.18 (m, 1H), 0.76-0.88 (m, 1H), 0.48-0.63 (m, 2H), 0.34-0.43 (m, 1H), 0.20-0.31 (m, 1H).

### Example 20

### N-[6-(5-chloro-1,3-benzoxazol-2-yl)spiro[3.3]heptan-2-yl]-5-(cyclopropylmethylsulfonyl)furan-2-carboxamide

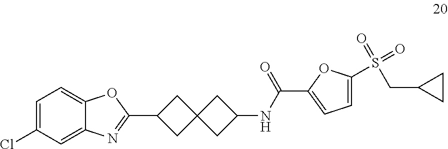 [SMILES: O=C(NC1CC2(C1)CC(c1nc3cc(Cl)ccc3o1)C2)c1ccc(S(=O)(=O)CC2CC2)o1]

The title compound was prepared in analogy to the procedure described for the preparation of Example 1, by using 5-(cyclopropylmethylsulfonyl)furan-2-carboxylic acid (Int-14) instead of 5-(trifluoromethyl)-2-furoic acid. The product was purified by preparative HPLC to afford Example 20 as a white solid. MS obsd. (ESI⁺) [(M+H)⁺]: 475.1. ¹H NMR (400 MHz, CDCl₃) δ ppm: 7.66 (d, J=1.96 Hz, 1H), 7.40 (d, J=8.56 Hz, 1H), 7.28 (dd, J=2.02, 8.62 Hz, 1H), 7.24-7.25 (m, 1H), 7.20-7.22 (m, 1H), 6.63 (br d, J=7.82 Hz, 1H), 4.44-4.55 (m, 1H), 3.71 (quin, J=8.53 Hz, 1H), 3.17 (d, J=7.21 Hz, 2H), 2.69-2.77 (m, 1H), 2.62-2.67 (m, 2 H), 2.45-2.62 (m, 3H), 2.07-2.21 (m, 2H), 1.05-1.14 (m, 1H), 0.62-0.68 (m, 2H), 0.18-0.24 (m, 2H).

### Example 21

### N-[6-(5-chloro-1,3-benzoxazol-2-yl)spiro[3.3]heptan-2-yl]-5-isobutylsulfinyl-furan-2-carboxamide

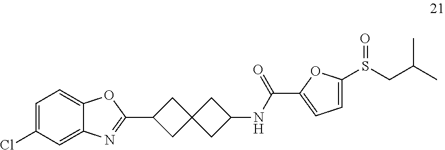 [SMILES: CC(C)CS(=O)c1ccc(C(=O)NC2CC3(C2)CC(c2nc4cc(Cl)ccc4o2)C3)o1]

The title compound was prepared in analogy to the procedure described for the preparation of Example 1, by using 5-isobutylsulfinylfuran-2-carboxylic acid (Int-16) instead of 5-(trifluoromethyl)-2-furoic acid. The product was purified by preparative HPLC to afford Example 21 as a white solid. MS obsd. (ESI⁺) [(M+H)⁺]: 461.2. ¹H NMR (400 MHz, DMSO-d₆) δ ppm: 8.81 (d, J=7.70 Hz, 1H), 7.80 (d, J=2.08 Hz, 1H), 7.72 (d, J=8.56 Hz, 1H), 7.40 (dd, J=2.14, 8.62 Hz, 1H), 7.29 (d, J=3.67 Hz, 1H), 7.23 (d, J=3.67 Hz, 1H), 4.31 (sxt, J=8.12 Hz, 1H), 3.75 (quin, J=8.47 Hz, 1H), 3.36 (dd, J=5.62, 12.84 Hz, 1H), 2.98 (dd, J=8.44, 12.84 Hz, 1H), 2.41-2.65 (m, 5H), 2.28-2.36 (m, 1H), 2.20-2.28 (m, 1H), 2.16 (ddd, J=5.14, 8.99, 11.19 Hz, 1H), 1.98 (quind, J=6.77, 13.87 Hz, 1H), 1.05 (dd, J=3.00, 6.66 Hz, 6H).

### Example 22

### N-[6-(5-chloro-1,3-benzoxazol-2-yl)spiro[3.3]heptan-2-yl]-5-isobutylsulfonyl-furan-2-carboxamide

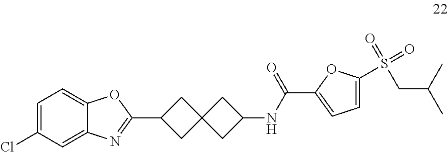 [SMILES: CC(C)CS(=O)(=O)c1ccc(C(=O)NC2CC3(C2)CC(c2nc4cc(Cl)ccc4o2)C3)o1]

The title compound was prepared in analogy to the procedure described for the preparation of Example 1, by using 5-isobutylsulfonylfuran-2-carboxylic acid (Int-17) instead of 5-(trifluoromethyl)-2-furoic acid. The product was purified by preparative HPLC to afford Example 22 as a white solid. MS obsd. (ESI⁺) [(M+H)⁺]: 477.1. ¹H NMR (400 MHz, CDCl₃) δ ppm: 7.64 (s, 1H), 7.39 (dd, J=8.6, 2.0 Hz, 1H), 7.29 (d, J=2.1 Hz, 1H), 7.14-7.22 (m, 2H), 6.62 (d, J=7.4 Hz, 1H), 4.38-4.60 (m, 1H), 3.61-3.80 (m, 1H), 3.14 (dd, J=6.5, 2.1 Hz, 2H), 2.72 (dd, J=11.0, 5.6 Hz, 1H), 2.64 (d, J=8.5 Hz, 2H), 2.52-2.61 (m, 2H), 2.42-2.51 (m, 1H), 2.32 (tt, J=13.2, 5.6 Hz, 1H), 2.14 (dt, J=20.6, 9.5 Hz, 2H), 1.11 (dd, J=6.6, 1.9 Hz, 6H).

### Example 23

### N-[6-(5-chloro-1,3-benzoxazol-2-yl)spiro[3.3]heptan-2-yl]-5-(cyclobutylmethylsulfonyl)furan-2-carboxamide

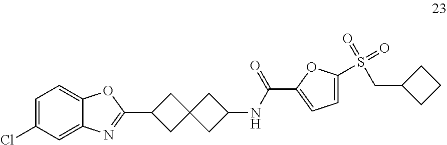 [SMILES: O=C(NC1CC2(C1)CC(c1nc3cc(Cl)ccc3o1)C2)c1ccc(S(=O)(=O)CC2CCC2)o1]

The title compound was prepared in analogy to the procedure described for the preparation of Example 1, by using 5-(cyclobutylmethylsulfonyl)furan-2-carboxylic acid (Int-18) instead of 5-(trifluoromethyl)-2-furoic acid. The product was purified by preparative HPLC to afford Example 23 as a white solid. MS obsd. (ESI⁺) [(M+H)⁺]: 489.2. ¹H NMR (400 MHz, DMSO-d₆) δ ppm: 8.86 (d, J=7.46 Hz, 1H), 7.80 (d, J=2.08 Hz, 1H), 7.72 (d, J=8.68 Hz, 1H), 7.40 (dd, J=2.08, 8.68 Hz, 1H), 7.37 (d, J=3.79 Hz, 1H), 7.28 (d, J=3.79 Hz, 1H), 4.31 (sxt, J=8.12 Hz, 1H), 3.75 (quin, J=8.50 Hz, 1H), 3.60 (d, J=7.21 Hz, 2H), 2.53-2.67 (m, 4H), 2.46 (qd, J=2.69, 8.44 Hz, 2H), 2.28-2.38 (m, 1H), 2.20-2.28 (m, 1H), 2.16 (dd, J=9.05, 11.13 Hz, 1H), 1.91-2.01 (m, 2H), 1.80-1.90 (m, 1H), 1.69-1.80 (m, 3H).

### Example 24

### N-[6-(5-chloro-1,3-benzoxazol-2-yl)spiro[3.3]heptan-2-yl]-5-(oxetan-3-ylmethylsulfonyl)furan-2-carboxamide

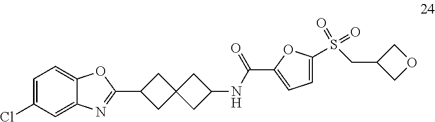 [SMILES: O=C(NC1CC2(C1)CC(c1nc3cc(Cl)ccc3o1)C2)c1ccc(S(=O)(=O)CC2COC2)o1]

The title compound was prepared in analogy to the procedure described for the preparation of Example 1, by using [5-(oxetan-3-ylmethylsulfonyl)furan-2-carbonyl]oxylithium (Int-20) instead of 5-(trifluoromethyl)-2-furoic acid. The product was purified by preparative HPLC to afford Example 24 as a white solid. MS obsd. (ESI⁺) [(M+H)⁺]: 491.1. ¹H NMR (400 MHz, CDCl₃) δ ppm: 7.65 (d, J=1.96 Hz, 1H), 7.40 (d, J=8.68 Hz, 1H), 7.27-7.29 (m, 1H), 7.22 (q, J=3.59 Hz, 2H), 6.59 (br d, J=7.95 Hz, 1H), 4.82-4.87 (m, 2H), 4.49-4.56 (m, 1H), 4.43-4.48 (m, 2H), 3.71 (t, J=8.56 Hz, 1H), 3.62-3.67 (m, 2H), 3.47-3.59 (m, 1H), 2.70-2.79 (m, 1H), 2.55-2.68 (m, 4H), 2.44-2.55 (m, 1H), 2.07-2.22 (m, 2H).

### Example 25

### N-[6-(5-chloro-1,3-benzoxazol-2-yl)spiro[3.3]heptan-2-yl]-5-[(1-methylazetidin-3-yl)methylsulfonyl]furan-2-carboxamide

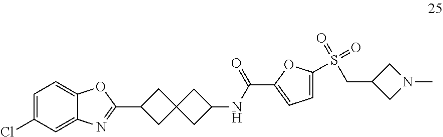 [SMILES: CN1CC(CS(=O)(=O)c2ccc(C(=O)NC3CC4(C3)CC(c3nc5cc(Cl)ccc5o3)C4)o2)C1]

The title compound was prepared in analogy to the procedure described for the preparation of Example 1, by using 5-[(1-methylazetidin-3-yl)methylsulfonyl]furan-2-carboxylic acid (Int-22) instead of 5-(trifluoromethyl)-2-furoic acid. The product was purified by preparative HPLC to afford Example 25 as an off-white solid. MS obsd. (ESI⁺) [(M+H)⁺]: 504.1. ¹H NMR (400 MHz, DMSO-d₆) δ ppm: 8.88 (d, J=7.58 Hz, 1H), 7.80 (d, J=2.08 Hz, 1H), 7.72 (d, J=8.68 Hz, 1H), 7.38-7.42 (m, 2H), 7.29 (d, J=3.79 Hz, 1H), 4.31 (sxt, J=8.09 Hz, 1H), 3.70-3.79 (m, 3H), 3.27 (t, J=7.21 Hz, 2H), 2.71-2.77 (m, 2H), 2.42-2.69 (m, 6H), 2.33 (qd, J=5.49, 7.50 Hz, 1H), 2.15-2.27 (m, 2H), 2.13 (s, 3H).

### Example 26

### benzyl 3-[[5-[[6-(5-chloro-1,3-benzoxazol-2-yl)spiro[3.3]heptan-2-yl]carbamoyl]-2-furyl]sulfonylmethyl]azetidine-1-carboxylate

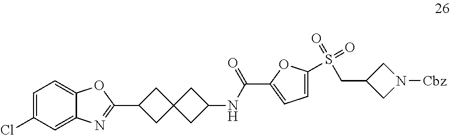 [SMILES: O=C(NC1CC2(C1)CC(c1nc3cc(Cl)ccc3o1)C2)c1ccc(S(=O)(=O)CC2CN(C(=O)OCc3ccccc3)C2)o1]

The title compound was prepared in analogy to the procedure described for the preparation of Example 1, by using 5-[(1-benzyloxycarbonylazetidin-3-yl)methylsulfonyl]furan-2-carboxylic acid (Int-23) instead of 5-(trifluoromethyl)-2-furoic acid. The product was purified by preparative HPLC to afford Example 26 as an off-white solid. MS obsd. (ESI⁺) [(M+H)⁺]: 624.1. ¹H NMR (400 MHz, CD₃OD) δ ppm: 7.64 (d, J=1.83 Hz, 1H), 7.55 (d, J=8.68 Hz, 1H), 7.35 (dd, J=2.08, 8.68 Hz, 1H), 7.27-7.34 (m, 6H), 7.23 (d, J=3.79 Hz, 1H), 5.05 (s, 2H), 4.36-4.45 (m, 1H), 4.12 (br s, 2H), 3.68-3.82 (m, 5H), 3.03-3.15 (m, 1H), 2.40-2.71 (m, 6H), 2.14-2.31 (m, 2H).

### Example 27

### N-[6-(5-chloro-1,3-benzoxazol-2-yl)spiro[3.3]heptan-2-yl]-5-[(3,3-difluorocyclobutyl)methylsulfonyl]furan-2-carboxamide

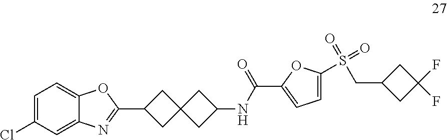 [SMILES: O=C(NC1CC2(C1)CC(c1nc3cc(Cl)ccc3o1)C2)c1ccc(S(=O)(=O)CC2CC(F)(F)C2)o1]

The title compound was prepared in analogy to the procedure described for the preparation of Example 1, by using [5-[(3,3-difluorocyclobutyl)methylsulfonyl]furan-2-carbonyl]oxylithium (Int-21) instead of 5-(trifluoromethyl)-2-furoic acid. The product was purified by preparative HPLC to afford Example 27 as a white solid. MS obsd. (ESI⁺) [(M+H)⁺]: 525.1. ¹H NMR (400 MHz, CDCl₃) δ ppm: 7.65 (d, J=1.96 Hz, 1H), 7.40 (d, J=8.68 Hz, 1H), 7.27-7.30 (m, 1H), 7.23-7.25 (m, 1H), 7.21-7.23 (m, 1H), 6.60 (br d, J=7.82 Hz, 1H), 4.50 (sxt, J=8.19 Hz, 1H), 3.71 (quin, J=8.56 Hz, 1H), 3.44 (d, J=7.34 Hz, 2H), 2.79-2.92 (m, 2H), 2.70-2.78 (m, 1H), 2.53-2.67 (m, 5H), 2.34-2.52 (m, 3H), 2.08-2.21 (m, 2H).

### Example 28

### N-[6-(5-chloro-1,3-benzoxazol-2-yl)spiro[3.3]heptan-2-yl]-5-(2-methoxyethylsulfonyl)furan-2-carboxamide

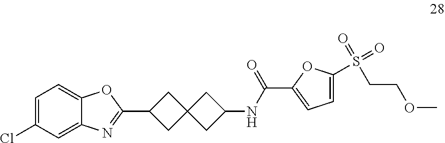 [SMILES: COCCS(=O)(=O)c1ccc(C(=O)NC2CC3(C2)CC(c2nc4cc(Cl)ccc4o2)C3)o1]

The title compound was prepared in analogy to the procedure described for the preparation of Example 1, by using 5-(2-methoxyethylsulfonyl)furan-2-carboxylic acid (Int-24) instead of 5-(trifluoromethyl)-2-furoic acid. The product was purified by preparative HPLC to afford Example 28 as a white solid. MS obsd. (ESI⁺) [(M+H)⁺]: 479.1. ¹H NMR (400 MHz, DMSO-d₆) δ ppm: 8.86 (d, J=7.58 Hz, 1H), 7.80 (d, J=2.08 Hz, 1H), 7.72 (d, J=8.68 Hz, 1H), 7.40 (dd, J=2.14, 8.62 Hz, 1H), 7.35 (d, J=3.67 Hz, 1H), 7.26 (d, J=3.67 Hz, 1H), 4.31 (sxt, J=8.14 Hz, 1H), 3.72-3.78 (m, 3H), 3.65-3.71 (m, 2H), 3.12 (s, 3H), 2.38-2.66 (m, 5H), 2.28-2.36 (m, 1H), 2.19-2.27 (m, 1H), 2.15 (dd, J=9.11, 11.06 Hz, 1H).

### Example 29

### Example 29-a, Example 29-b

### N-[6-(5-chloro-1,3-benzoxazol-2-yl)spiro[3.3]heptan-2-yl]-5-sulfamoyl-furan-2-carboxamide

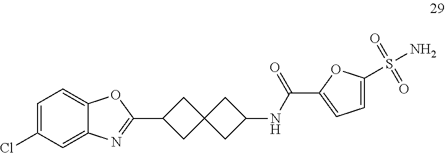 [SMILES: NS(=O)(=O)c1ccc(C(=O)NC2CC3(C2)CC(c2nc4cc(Cl)ccc4o2)C3)o1]

N-[6-(5-Chloro-1,3-benzoxazol-2-yl)spiro[3.3]heptan-2-yl]-5-sulfamoyl-furan-2-carboxamide (Example 29) was prepared in analogy to the procedure described for the preparation of Example 1, by using 5-sulfamoylfuran-2-carboxylic acid instead of 5-(trifluoromethyl)-2-furoic acid. MS obsd. (ESI⁺) [(M+H)⁺]: 436.2. ¹H NMR (400 MHz, DMSO-d₆) δ ppm: 8.65 (br d, J=7.46 Hz, 1H), 7.80 (d, J=2.08 Hz, 1H), 7.71 (d, J=8.68 Hz, 1H), 7.40 (dd, J=2.20, 8.68 Hz, 1H), 7.18 (d, J=3.30 Hz, 1H), 6.95 (br s, 1H), 4.29 (sxt, J=8.12 Hz, 1H), 3.74 (quin, J=8.50 Hz, 1H), 2.40-2.65 (m, 5H), 2.26-2.37 (m, 1H), 2.16-2.25 (m, 1H), 2.04-2.16 (m, 1H).

The two enantiomers (Example 29-a, Example 29-b) were obtained through SFC [Instrument: SFC 80; Column: AD, 250×30 mm I.D., 5 μm; Mobile phase: A for CO₂ and B for Ethanol (0.10% NH₄OH); Gradient: B 30%; Flow rate: 70 mL/min; Back pressure: 100 bar; Column temperature: 40° C.] chiral separation of N-[6-(5-chloro-1,3-benzoxazol-2-yl)spiro[3.3]heptan-2-yl]-5-sulfamoyl-furan-2-carboxamide (Example 29). Example 29-a was eluted out before Example 29-b.

### (Sₐ)—N-[6-(5-chloro-1,3-benzoxazol-2-yl)spiro[3.3]heptan-2-yl]-5-sulfamoyl-furan-2-carboxamide

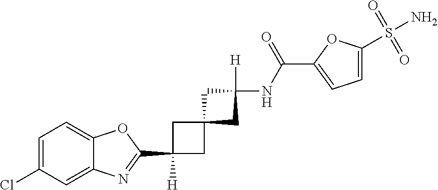

### (Rₐ)—N-[6-(5-chloro-1,3-benzoxazol-2-yl)spiro[3.3]heptan-2-yl]-5-sulfamoyl-furan-2-carboxamide

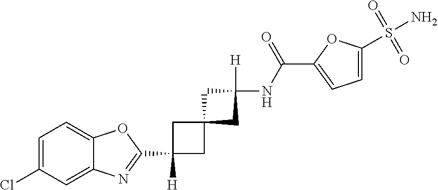

Example 29-a: white solid. [α]²⁰_{D}=+25.276 (C=0.1, MeOH). MS obsd. (ESI⁺) [(M+H)⁺]: 436.3. ¹H NMR (400 MHz, DMSO-d₆) δ ppm: 8.69 (d, J=7.6 Hz, 1H), 7.89 (s, 2H), 7.80 (d, J=2.1 Hz, 1H), 7.71 (d, J=8.7 Hz, 1H), 7.40 (dd, J=8.7, 2.2 Hz, 1H), 7.22 (d, J=3.5 Hz, 1H), 7.04 (d, J=3.5 Hz, 1H), 4.29 (sxt, J=8.2 Hz, 1H), 3.74 (quin, J=8.5 Hz, 1H), 2.52-2.68 (m, 3H), 2.29-2.47 (m, 3H), 2.08-2.24 (m, 2H).

Example 29-b: white solid. [α]²⁰_{D}=−25.921 (C=0.1, MeOH). MS obsd. (ESI⁺) [(M+H)⁺]: 436.3. ¹H NMR (400 MHz, DMSO-d₆) δ ppm: 8.69 (d, J=7.6 Hz, 1H), 7.88 (s, 2H), 7.80 (d, J=2.1 Hz, 1H), 7.72 (d, J=8.7 Hz, 1H), 7.40 (dd, J=8.7, 2.2 Hz, 1H), 7.22 (d, J=3.7 Hz, 1H), 7.04 (d, J=3.5 Hz, 1H), 4.29 (sxt, J=8.1 Hz, 1H), 3.74 (quin, J=8.5 Hz, 1H), 2.52-2.68 (m, 3H), 2.29-2.47 (m, 3H), 2.08-2.24 (m, 2H).

### Example 30

### N-[6-(5-chloro-1,3-benzoxazol-2-yl)spiro[3.3]heptan-2-yl]-5-(methylsulfonylmethyl)furan-2-carboxamide

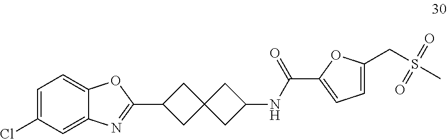 [SMILES: CS(=O)(=O)Cc1ccc(C(=O)NC2CC3(C2)CC(c2nc4cc(Cl)ccc4o2)C3)o1]

The title compound was prepared in analogy to the procedure described for the preparation of Example 1, by using 5-(methylsulfonylmethyl)furan-2-carboxylic acid instead of 5-(trifluoromethyl)-2-furoic acid. The product was purified by preparative HPLC to afford Example 30 as a white solid. MS obsd. (ESI⁺) [(M+H)⁺]: 449.1. ¹H NMR (400 MHz, DMSO-d₆) δ ppm: 8.48 (d, J=7.58 Hz, 1H), 7.80 (d, J=2.20 Hz, 1H), 7.72 (d, J=8.80 Hz, 1H), 7.40 (dd, J=2.08, 8.68 Hz, 1H), 7.14 (d, J=3.42 Hz, 1H), 6.65 (d, J=3.67 Hz, 1H), 4.70 (s, 2H), 4.28 (qd, J=8.04, 16.23 Hz, 1H), 3.74 (quin, J=8.50 Hz, 1H), 3.04 (s, 3H), 2.39-2.64 (m, 5H), 2.26-2.35 (m, 1H), 2.16-2.24 (m, 1H), 2.11 (dd, J=9.17, 11.13 Hz, 1H).

### Example 31

### N-[6-(5-chloro-1,3-benzoxazol-2-yl)spiro[3.3]heptan-2-yl]-5-(cyclopropylsulfonylmethyl)furan-2-carboxamide

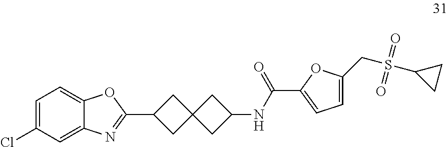 [SMILES: O=C(NC1CC2(C1)CC(c1nc3cc(Cl)ccc3o1)C2)c1ccc(CS(=O)(=O)C2CC2)o1]

The title compound was prepared in analogy to the procedure described for the preparation of Example 1, by using 5-(cyclopropylsulfonylmethyl)furan-2-carboxylic acid (Int-28) instead of 5-(trifluoromethyl)-2-furoic acid. The product was purified by preparative HPLC to afford Example 31 as an off-white solid. MS obsd. (ESI⁺) [(M+H)⁺]: 475.1. ¹H NMR (400 MHz, DMSO-d₆) δ ppm: 8.46 (d, J=7.70 Hz, 1H), 7.80 (d, J=1.96 Hz, 1H), 7.72 (d, J=8.68 Hz, 1H), 7.40 (dd, J=2.20, 8.68 Hz, 1H), 7.14 (d, J=3.42 Hz, 1H), 6.64 (d, J=3.42 Hz, 1H), 4.73 (s, 2H), 4.28 (sxt, J=8.19 Hz, 1H), 3.74 (quin, J=8.47 Hz, 1H), 2.65-2.74 (m, 1H), 2.42-2.64 (m, 5H), 2.25-2.35 (m, 1H), 2.16-2.24 (m, 1H), 2.11 (dd, J=9.05, 11.13 Hz, 1H), 0.98-1.06 (m, 2H), 0.85-0.93 (m, 2H).

### Example 32

### N-[6-(5-chloro-1,3-benzoxazol-2-yl)spiro[3.3]heptan-2-yl]-5-(2-methylsulfonylethyl)furan-2-carboxamide

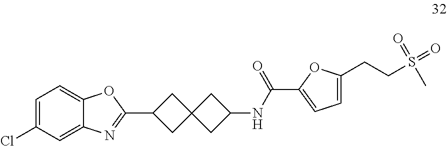 [SMILES: CS(=O)(=O)CCc1ccc(C(=O)NC2CC3(C2)CC(c2nc4cc(Cl)ccc4o2)C3)o1]

The title compound was prepared in analogy to the procedure described for the preparation of Example 1, by using 5-(2-methylsulfonylethyl)furan-2-carboxylic acid (Int-29) instead of 5-(trifluoromethyl)-2-furoic acid. The product was purified by preparative HPLC to afford Example 32 as a white solid. MS obsd. (ESI⁺) [(M+H)⁺]: 463.0. ¹H NMR (400 MHz, CD₃OD) δ ppm: 7.63 (d, J=1.83 Hz, 1H), 7.55 (d, J=8.68 Hz, 1H), 7.35 (dd, J=2.08, 8.68 Hz, 1H), 7.02 (d, J=3.42 Hz, 1H), 6.38 (d, J=3.42 Hz, 1H), 4.34-4.44 (m, 1H), 3.75 (quin, J=8.53 Hz, 1H), 3.50-3.58 (m, 2H), 3.20-3.26 (m, 2H), 2.95 (s, 3H), 2.38-2.72 (m, 6H), 2.13-2.30 (m, 2H).

### Example 33

### N-[6-(5-chloro-1,3-benzoxazol-2-yl)spiro[3.3]heptan-2-yl]-5-(sulfamoylmethyl)furan-2-carboxamide

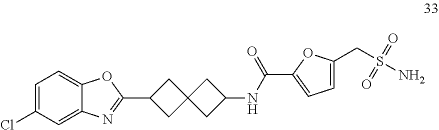 [SMILES: NS(=O)(=O)Cc1ccc(C(=O)NC2CC3(C2)CC(c2nc4cc(Cl)ccc4o2)C3)o1]

The title compound was prepared in analogy to the procedure described for the preparation of Example 1, by using 5-(sulfamoylmethyl)furan-2-carboxylic acid (Int-30) instead of 5-(trifluoromethyl)-2-furoic acid. The product was purified by preparative HPLC to afford Example 33 as a white solid. MS obsd. (ESI⁺) [(M+H)⁺]: 450.1. ¹H NMR (400 MHz, CD₃OD) δ ppm: 7.63 (d, J=2.08 Hz, 1H), 7.55 (d, J=8.68 Hz, 1H), 7.35 (dd, J=2.08, 8.68 Hz, 1H), 7.09 (d, J=3.55 Hz, 1H), 6.61 (d, J=3.42 Hz, 1H), 4.51 (s, 2H), 4.39 (quin, J=8.25 Hz, 1H), 3.75 (quin, J=8.53 Hz, 1H), 2.38-2.73 (m, 6H), 2.25 (dd, J=8.93, 10.88 Hz, 1H), 2.17 (dd, J=8.93, 11.37 Hz, 1H).

### Example 34

### N-[6-(5-chloro-1,3-benzoxazol-2-yl)spiro[3.3]heptan-2-yl]-5-(2-sulfamoylethyl)furan-2-carboxamide

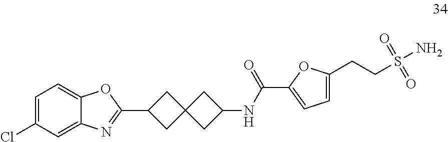 [SMILES: NS(=O)(=O)CCc1ccc(C(=O)NC2CC3(C2)CC(c2nc4cc(Cl)ccc4o2)C3)o1]

The title compound was prepared in analogy to the procedure described for the preparation of Example 1, by using 5-(sulfamoylethyl)furan-2-carboxylic acid (Int-31) instead of 5-(trifluoromethyl)-2-furoic acid. The product was purified by preparative HPLC to afford Example 34 as a white solid. MS obsd. (ESI⁺) [(M+H)⁺]: 464.1. ¹H NMR (400 MHz, DMSO-d₆) δ ppm: 8.39 (d, J=7.70 Hz, 1H), 7.80 (d, J=2.08 Hz, 1H), 7.72 (d, J=8.68 Hz, 1H), 7.40 (dd, J=2.14, 8.62 Hz, 1H), 6.98 (d, J=3.42 Hz, 1H), 6.96 (s, 2H), 6.39 (d, J=3.30 Hz, 1H), 4.28 (sxt, J=8.17 Hz, 1H), 3.74 (quin, J=8.50 Hz, 1H), 3.31-3.39 (m, 2H), 3.01-3.13 (m, 2H), 2.40-2.66 (m, 5H), 2.24-2.36 (m, 1H), 2.15-2.23 (m, 1H), 2.06-2.15 (m, 1H).

### Example 35

### N-[6-(5-chloro-1,3-benzoxazol-2-yl)spiro[3.3]heptan-2-yl]-5-[(sulfamoylamino)methyl]furan-2-carboxamide

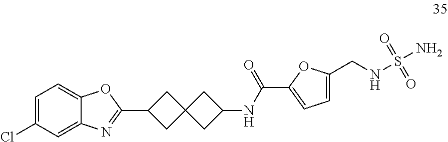 [SMILES: NS(=O)(=O)NCc1ccc(C(=O)NC2CC3(C2)CC(c2nc4cc(Cl)ccc4o2)C3)o1]

The title compound was prepared in analogy to the procedure described for the preparation of Example 1, by using 5-[(sulfamoylamino)methyl]furan-2-carboxylic acid (Int-32) instead of 5-(trifluoromethyl)-2-furoic acid. The product was purified by preparative HPLC to afford Example 35 as a white solid. MS obsd. (ESI⁺) [(M+H)⁺]: 465.1. ¹H NMR (400 MHz, CD₃OD) δ ppm: 7.64 (d, J=1.8 Hz, 1H), 7.56 (d, J=8.7 Hz, 1H), 7.36 (dd, J=8.7, 2.1 Hz, 1H), 7.04 (d, J=3.4 Hz, 1H), 6.47 (d, J=3.4 Hz, 1H), 4.40 (p, J=8.8 Hz, 1H), 4.26 (s, 2H), 3.75 (p, J=8.5 Hz, 1H), 2.38-2.69 (m, 6H), 2.11-2.27 (m, 2H).

### Example 36

### 4-bromo-N-[6-(5-chloro-1,3-benzoxazol-2-yl)spiro[3.3]heptan-2-yl]-5-ethylsulfonyl-furan-2-carboxamide

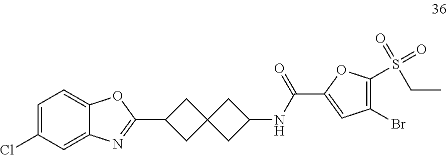 [SMILES: CCS(=O)(=O)c1oc(C(=O)NC2CC3(C2)CC(c2nc4cc(Cl)ccc4o2)C3)cc1Br]

The title compound was prepared in analogy to the procedure described for the preparation of Example 1, by using 4-bromo-5-ethylsulfonyl-furan-2-carboxylic acid (Int-8) instead of 5-(trifluoromethyl)-2-furoic acid. The product was purified by preparative HPLC to afford Example 36 as a white solid. MS obsd. (ESI⁺) [(M+H)⁺]: 527.1. ¹H NMR (400 MHz, CDCl₃) δ ppm: 7.66 (d, J=1.96 Hz, 1H), 7.41 (d, J=8.56 Hz, 1H), 7.29 (dd, J=1.96, 8.56 Hz, 1H), 7.24 (s, 1H), 6.70 (br d, J=8.56 Hz, 1H), 4.41-4.54 (m, 1H), 3.72 (quin, J=8.56 Hz, 1H), 3.32 (q, J=7.34 Hz, 2H), 2.68-2.77 (m, 1H), 2.42-2.67 (m, 5H), 2.06-2.23 (m, 2H), 1.38 (t, J=7.46 Hz, 3H).

### Example 37

### N-[6-(5-chloro-1,3-benzoxazol-2-yl)spiro[3.3]heptan-2-yl]-5-ethylsulfonyl-3-methyl-furan-2-carboxamide

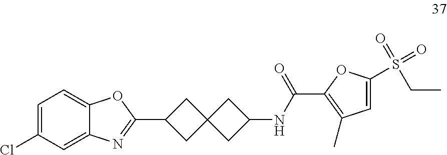 [SMILES: CCS(=O)(=O)c1cc(C)c(C(=O)NC2CC3(C2)CC(c2nc4cc(Cl)ccc4o2)C3)o1]

The title compound was prepared in analogy to the procedure described for the preparation of Example 1, by using 5-ethylsulfonyl-3-methyl-furan-2-carboxylic acid (Int-9) instead of 5-(trifluoromethyl)-2-furoic acid. The product was purified by preparative HPLC to afford Example 37 as an off-white solid. MS obsd. (ESI⁺) [(M+H)⁺]: 463.1. ¹H NMR (400 MHz, DMSO-d₆) δ ppm: 8.68 (br d, J=7.58 Hz, 1H), 7.80 (d, J=1.83 Hz, 1H), 7.71 (d, J=8.56 Hz, 1H), 7.40 (dd, J=2.08, 8.68 Hz, 1H), 7.31 (s, 1H), 4.30 (sxt, J=8.12 Hz, 1H), 3.74 (quin, J=8.44 Hz, 1H), 3.48 (q, J=7.34 Hz, 2H), 2.39-2.67 (m, 5H), 2.29 (s, 3H), 2.13-2.35 (m, 3H), 1.16 (t, J=7.34 Hz, 3H).

### Example 38

### 5-tert-butylsulfinyl-N-[6-(5-chloro-1,3-benzoxazol-2-yl)spiro[3.3]heptan-2-yl]furan-2-carboxamide

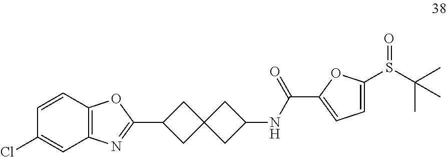 [SMILES: CC(C)(C)S(=O)c1ccc(C(=O)NC2CC3(C2)CC(c2nc4cc(Cl)ccc4o2)C3)o1]

The title compound was prepared according to the following scheme:

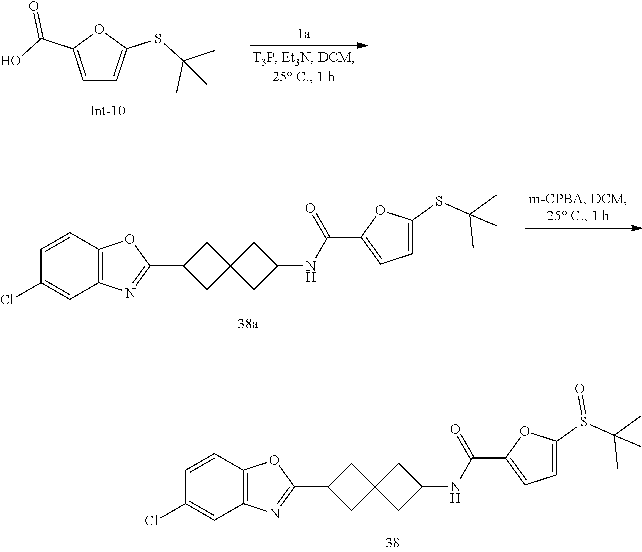 [SMILES: CC(C)(C)Sc1ccc(C(=O)O)o1 ; CC(C)(C)Sc1ccc(C(=O)NC2CC3(C2)CC(c2nc4cc(Cl)ccc4o2)C3)o1 ; CC(C)(C)S(=O)c1ccc(C(=O)NC2CC3(C2)CC(c2nc4cc(Cl)ccc4o2)C3)o1]

### Step 1: Preparation of 5-tert-butylsulfanyl-N-[6-(5-chloro-1,3-benzoxazol-2-yl)spiro[3.3]heptan-2-yl]furan-2-carboxamide (38a)

To a solution of 5-tert-butylsulfanylfuran-2-carboxylic acid (Int-10, 251.5 mg, 1.26 mmol) in DCM (10 mL) were added triethylamine (0.16 mL, 1.14 mmol) and 2,4,6-tripropyl-1,3,5,2,4,6-trioxatriphosphinane 2,4,6-trioxide (1.09 g, 1.71 mmol). After being stirred at 25° C. for 0.5 h, 6-(5-chloro-1,3-benzoxazol-2-yl)spiro[3.3]heptan-2-amine (1a, 300 mg, 1.14 mmol) was added and the reaction was stirred at 25° C. for 1 h. The resulting mixture was extracted with EtOAc (50 mL×3) and water (30 mL×2). The combined organic layer was dried over Na₂SO₄ and concentrated in vacuo. The residue was purified by flash column (eluting with EtOAc/PE=1/4) to give 5-tert-butylsulfanyl-N-[6-(5-chloro-1,3-benzoxazol-2-yl)spiro[3.3]heptan-2-yl]furan-2-carboxamide as a light yellow solid (38a, 300 mg, 59.04%). MS obsd. (ESI⁺) [(M+H)⁺]: 445.1.

### Step 2: Preparation of 5-tert-butylsulfinyl-N-[6-(5-chloro-1,3-benzoxazol-2-yl)spiro[3.3]heptan-2-yl]furan-2-carboxamide (Example 38)

To a solution of m-CPBA (31 mg, 0.18 mmol) in DCM (1.18 mL) was added 5-tert-butylsulfanyl-N-[6-(5-chloro-1,3-benzoxazol-2-yl)spiro[3.3]heptan-2-yl]furan-2-carboxamide (38a, 80 mg, 0.18 mmol). After being stirred at 25° C. for 2 h, the mixture was extracted with EtOAc (30 mL×3) and water (20 mL×2). The organic layer was dried over Na₂SO₄ and concentrated in vacuo. The residue was purified by preparative HPLC to give 5-tert-butylsulfinyl-N-[6-(5-chloro-1,3-benzoxazol-2-yl)spiro[3.3]heptan-2-yl]furan-2-carboxamide as a white solid (Example 38, 29.8 mg, 35.8%). MS obsd. (ESI⁺) [(M+H)⁺]: 461.1. ¹H NMR (400 MHz, DMSO-d₆) δ ppm: 8.69 (d, J=7.58 Hz, 1H), 7.80 (d, J=2.08 Hz, 1H), 7.72 (d, J=8.68 Hz, 1H), 7.40 (dd, J=2.08, 8.68 Hz, 1H), 7.28 (d, J=3.67 Hz, 1H), 7.15 (d, J=3.55 Hz, 1H), 4.29 (sxt, J=8.14 Hz, 1H), 3.74 (quin, J=8.47 Hz, 1H), 2.41-2.66 (m, 5H), 2.27-2.35 (m, 1H), 2.19-2.26 (m, 1H), 2.11-2.18 (m, 1H), 1.19 (s, 9H).

### Example 39

### 5-tert-butylsulfonyl-N-[6-(5-chloro-1,3-benzoxazol-2-yl)spiro[3.3]heptan-2-yl]furan-2-carboxamide

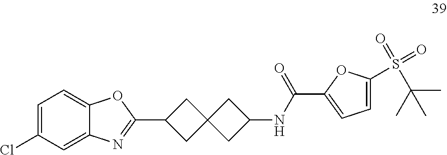 [SMILES: CC(C)(C)S(=O)(=O)c1ccc(C(=O)NC2CC3(C2)CC(c2nc4cc(Cl)ccc4o2)C3)o1]

The title compound was prepared according to the following scheme:

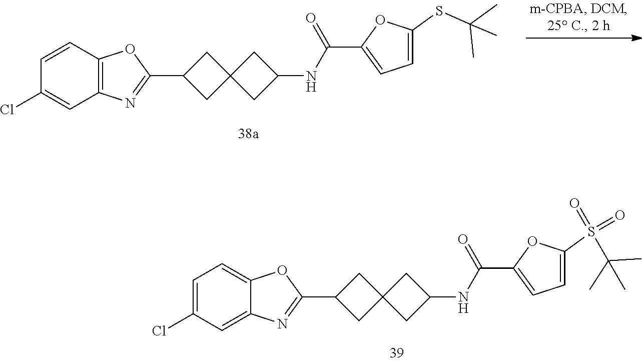 [SMILES: CC(C)(C)Sc1ccc(C(=O)NC2CC3(C2)CC(c2nc4cc(Cl)ccc4o2)C3)o1 ; CC(C)(C)S(=O)(=O)c1ccc(C(=O)NC2CC3(C2)CC(c2nc4cc(Cl)ccc4o2)C3)o1]

To a solution of m-CPBA (104.7 mg, 0.61 mmol) in DCM (1.33 mL) was added 5-tert-butylsulfanyl-N-[6-(5-chloro-1,3-benzoxazol-2-yl)spiro[3.3]heptan-2-yl]furan-2-carboxamide (38a, 90.0 mg, 0.2 mmol). After being stirred at 25° C. for 2 h, the mixture was extracted with EtOAc (30 mL×3) and water (20 mL×2). The combined organic layer was dried over Na₂SO₄ and concentrated in vacuo. The residue was purified by preparative HPLC to give 5-tert-butylsulfonyl-N-[6-(5-chloro-1,3-benzoxazol-2-yl)spiro[3.3]heptan-2-yl]furan-2-carboxamide as a white solid (Example 39, 41.9 mg, 43.27%). MS obsd. (ESI⁺) [(M+H)⁺]: 477.1. ¹H NMR (400 MHz, DMSO-d₆) δ ppm: 8.86 (br d, J=7.34 Hz, 1H), 7.80 (d, J=2.08 Hz, 1H), 7.72 (d, J=8.68 Hz, 1H), 7.44 (d, J=3.79 Hz, 1H), 7.40 (dd, J=2.08, 8.68 Hz, 1H), 7.35 (d, J=3.67 Hz, 1H), 4.30 (sxt, J=8.07 Hz, 1H), 3.74 (quin, J=8.47 Hz, 1H), 2.41-2.66 (m, 5H), 2.28-2.36 (m, 1H), 2.20-2.28 (m, 1H), 2.11-2.20 (m, 1H), 1.30 (s, 9H).

### Example 40

### N-[6-(5-chloro-1,3-benzoxazol-2-yl)spiro[3.3]heptan-2-yl]-5-cyclopropylsulfinyl-furan-2-carboxamide

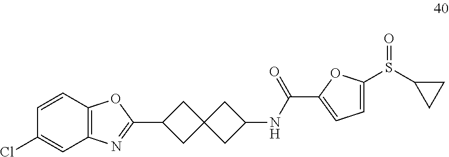 [SMILES: O=C(NC1CC2(C1)CC(c1nc3cc(Cl)ccc3o1)C2)c1ccc(S(=O)C2CC2)o1]

The title compound was prepared in analogy to the procedure described for the preparation of Example 38, by using 5-cyclopropylsulfanylfuran-2-carboxylic acid (Int-5) instead of 5-tert-butylsulfanylfuran-2-carboxylic acid (Int-10). The product was purified by preparative HPLC to afford Example 40 as a white solid. MS obsd. (ESI⁺) [(M+H)⁺]: 445.1. ¹H NMR (400 MHz, CD₃OD) (ppm: 7.64 (d, J=1.96 Hz, 1H), 7.55 (d, J=8.80 Hz, 1H), 7.35 (dd, J=2.08, 8.68 Hz, 1H), 7.16-7.22 (m, 2H), 4.41 (quin, J=8.28 Hz, 1H), 3.75 (quin, J=8.50 Hz, 1H), 2.86-2.95 (m, 1H), 2.41-2.72 (m, 6H), 2.14-2.33 (m, 2H), 1.27-1.36 (m, 1H), 1.18-1.27 (m, 1H), 1.05-1.15 (m, 1H), 0.89-0.99 (m, 1H).

### Example 41

### N-[6-(5-chloro-1,3-benzoxazol-2-yl)spiro[3.3]heptan-2-yl]-5-(trifluoromethylsulfanyl)furan-2-carboxamide

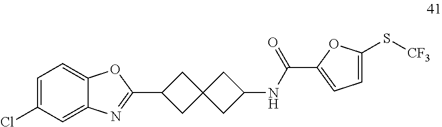 [SMILES: O=C(NC1CC2(C1)CC(c1nc3cc(Cl)ccc3o1)C2)c1ccc(SC(F)(F)F)o1]

The title compound was prepared in analogy to the procedure described for the preparation of 38a, by using 5-(trifluoromethylsulfanyl)furan-2-carboxylic acid (Int-11) instead of 5-tert-butylsulfanylfuran-2-carboxylic acid (Int-10). The product was purified by preparative HPLC to afford Example 41 as a white solid. MS obsd. (ESI⁺) [(M+H)⁺]: 457.0. ¹H NMR (400 MHz, CD₃OD) δ ppm: 7.63 (d, J=1.9 Hz, 1H), 7.55 (d, J=8.7 Hz, 1H), 7.35 (dd, J=8.7, 2.1 Hz, 1H), 7.19 (dd, J=12.2, 3.5 Hz, 2H), 4.40 (s, 1H), 3.74 (t, J=8.5 Hz, 1H), 2.38-2.73 (m, 6H), 2.15-2.35 (m, 2H).

### Example 42

### N-[6-(5-chloro-1,3-benzoxazol-2-yl)spiro[3.3]heptan-2-yl]-5-(trifluoromethylsulfinyl)furan-2-carboxamide

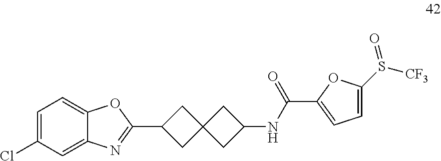 [SMILES: O=C(NC1CC2(C1)CC(c1nc3cc(Cl)ccc3o1)C2)c1ccc(S(=O)C(F)(F)F)o1]

The title compound was prepared in analogy to the procedure described for the preparation of Example 38, by using 5-(trifluoromethylsulfanyl)furan-2-carboxylic acid (Int-11) instead of 5-tert-butylsulfanylfuran-2-carboxylic acid (Int-10). The product was purified by preparative HPLC to afford Example 42 as a white solid. MS obsd. (ESI⁺) [(M+H)⁺]: 473.0. ¹H NMR (400 MHz, CD₃OD) δ ppm: 7.64 (d, J=2.0 Hz, 1H), 7.55 (t, J=6.6 Hz, 2H), 7.38-7.29 (m, 2H), 4.46-4.35 (m, 1H), 3.75 (p, J=8.5 Hz, 1H), 2.40-2.72 (m, 6H), 2.15-2.32 (m, 2H).

### Example 43

### N-[6-(5-chloro-1,3-benzoxazol-2-yl)spiro[3.3]heptan-2-yl]-5-(trifluoromethylsulfonyl)furan-2-carboxamide

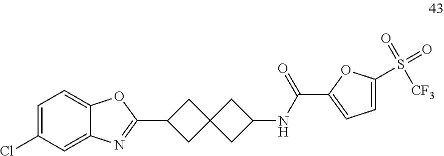 [SMILES: O=C(NC1CC2(C1)CC(c1nc3cc(Cl)ccc3o1)C2)c1ccc(S(=O)(=O)C(F)(F)F)o1]

The title compound was prepared in analogy to the procedure described for the preparation of Example 39, by using 5-(trifluoromethylsulfanyl)furan-2-carboxylic acid (Int-11) instead of 5-tert-butylsulfanylfuran-2-carboxylic acid (Int-10). The product was purified by preparative HPLC to afford Example 43 as a white solid. MS obsd. (ESI⁺) [(M+H)⁺]: 489.0. ¹H NMR (400 MHz, CD₃OD) δ ppm: 7.86 (d, J=3.8 Hz, 1H), 7.63 (d, J=1.8 Hz, 1H), 7.55 (d, J=8.7 Hz, 1H), 7.30-7.45 (m, 2H), 4.35-4.48 (m, 1H), 3.74 (p, J=8.5 Hz, 1H), 2.39-2.73 (m, 6H), 2.16-2.35 (m, 2H).

### Example 44

### N-[6-(5-chloro-1,3-benzoxazol-2-yl)spiro[3.3]heptan-2-yl]-5-(isopropylsulfonylmethyl)furan-2-carboxamide

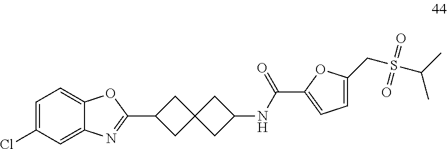 [SMILES: CC(C)S(=O)(=O)Cc1ccc(C(=O)NC2CC3(C2)CC(c2nc4cc(Cl)ccc4o2)C3)o1]

The title compound was prepared in analogy to the procedure described for the preparation of Example 39, by using 5-(isopropylsulfanylmethyl)furan-2-carboxylic acid (Int-26) instead of 5-tert-butylsulfanylfuran-2-carboxylic acid (Int-10). The product was purified by preparative HPLC to afford Example 44 as a white solid. MS obsd. (ESI⁺) [(M+H)⁺]: 477.1. ¹H NMR (400 MHz, DMSO-d₆) δ ppm: 8.47 (d, J=7.70 Hz, 1H), 7.80 (d, J=1.96 Hz, 1H), 7.72 (d, J=8.56 Hz, 1H), 7.40 (dd, J=2.14, 8.62 Hz, 1H), 7.14 (d, J=3.55 Hz, 1H), 6.63 (d, J=3.42 Hz, 1H), 4.68 (s, 2H), 4.28 (sxt, J=8.12 Hz, 1H), 3.74 (quin, J=8.50 Hz, 1H), 3.21-3.32 (m, 1H), 2.41-2.65 (m, 5H), 2.26-2.36 (m, 1H), 2.16-2.24 (m, 1H), 2.11 (dd, J=9.05, 11.13 Hz, 1H), 1.28 (d, J=6.85 Hz, 6H).

### Example 45

### N-[6-(5-chloro-1,3-benzoxazol-2-yl)spiro[3.3]heptan-2-yl]-5-methylsulfonyl-thiophene-2-carboxamide

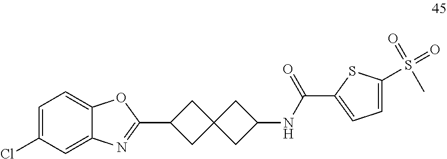 [SMILES: CS(=O)(=O)c1ccc(C(=O)NC2CC3(C2)CC(c2nc4cc(Cl)ccc4o2)C3)s1]

The title compound was prepared in analogy to the procedure described for the preparation of Example 1, by using 5-methylsulfonylthiophene-2-carboxylic acid instead of 5-(trifluoromethyl)-2-furoic acid. The product was purified by preparative HPLC to afford Example 45 as a white solid. MS obsd. (ESI⁺) [(M+H)⁺]: 451.0. ¹H NMR (400 MHz, DMSO-d₆) δ ppm: 8.96 (br d, J=7.21 Hz, 1H), 7.77-7.86 (m, 3H), 7.72 (d, J=8.68 Hz, 1H), 7.35-7.44 (m, 1H), 4.28 (sxt, J=7.97 Hz, 1H), 3.75 (quin, J=8.41 Hz, 1H), 3.38 (s, 3H), 2.46-2.67 (m, 5H), 2.29-2.39 (m, 1H), 2.16-2.25 (m, 1H), 2.05-2.15 (m, 1H).

### Example 46

### N-[6-(6-chloro-1,3-benzoxazol-2-yl)spiro[3.3]heptan-2-yl]-5-(trifluoromethyl)furan-2-carboxamide

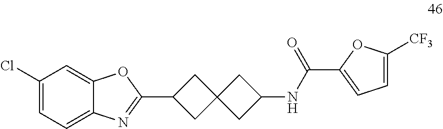 [SMILES: O=C(NC1CC2(C1)CC(c1nc3ccc(Cl)cc3o1)C2)c1ccc(C(F)(F)F)o1]

The title compound was prepared in analogy to the procedure described for the preparation of Example 1, by using 2-amino-5-chloro-phenol instead of 2-amino-4-chloro-phenol. The product was purified by preparative HPLC to afford Example 46 as an orange solid. MS obsd. (ESI⁺) [(M+H)⁺]: 425.0. ¹H NMR (400 MHz, CDCl₃) δ ppm: 7.56 (d, J=8.44 Hz, 1H), 7.48 (d, J=1.71 Hz, 1H), 7.28 (dd, J=1.77, 8.50 Hz, 1H), 7.14 (d, J=3.30 Hz, 1H), 6.88 (d, J=2.93 Hz, 1H), 6.73 (br d, J=7.58 Hz, 1H), 4.52 (sxt, J=8.12 Hz, 1H), 3.69 (quin, J=8.53 Hz, 1H), 2.69-2.77 (m, 1H), 2.60-2.66 (m, 2H), 2.51-2.60 (m, 2H), 2.41-2.50 (m, 1H), 2.07-2.23 (m, 2H).

### Example 47

### N-[6-(6-fluoro-1,3-benzoxazol-2-yl)spiro[3.3]heptan-2-yl]-5-(trifluoromethyl)furan-2-carboxamide

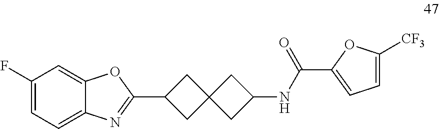 [SMILES: O=C(NC1CC2(C1)CC(c1nc3ccc(F)cc3o1)C2)c1ccc(C(F)(F)F)o1]

The title compound was prepared in analogy to the procedure described for the preparation of Example 1, by using 2-amino-5-fluoro-phenol instead of 2-amino-4-chloro-phenol. The product was purified by preparative HPLC to afford Example 47 as a white solid. MS obsd. (ESI⁺) [(M+H)⁺]: 409.1. ¹H NMR (400 MHz, CDCl₃) δ ppm: 7.60 (dd, J=4.89, 8.80 Hz, 1H), 7.21 (dd, J=2.32, 7.95 Hz, 1H), 7.14 (d, J=3.06 Hz, 1H), 7.06 (dt, J=2.45, 9.17 Hz, 1H), 6.87-6.90 (m, 1H), 6.47 (br d, J=7.46 Hz, 1H), 4.51 (sxt, J=8.14 Hz, 1H), 3.71 (quin, J=8.59 Hz, 1H), 2.70-2.79 (m, 1H), 2.64 (d, J=9.29 Hz, 2H), 2.5-2.62 (m, 2H), 2.43-2.52 (m, 1H), 2.07-2.21 (m, 2H).

### Example 48

### N-[6-(6-chlorooxazolo[5,4-b]pyridin-2-yl)spiro[3.3]heptan-2-yl]-5-methylsulfonyl-furan-2-carboxamide

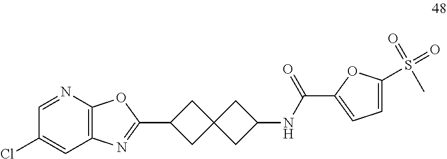 [SMILES: CS(=O)(=O)c1ccc(C(=O)NC2CC3(C2)CC(c2nc4cc(Cl)cnc4o2)C3)o1]

The title compound was prepared according to the following scheme:

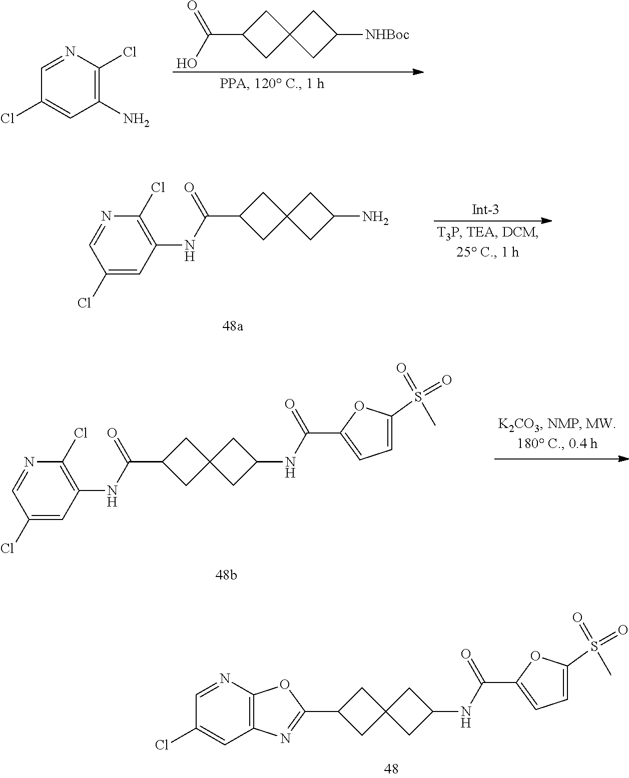 [SMILES: Nc1cc(Cl)cnc1Cl ; CC(C)(C)OC(=O)NC1CC2(C1)CC(C(=O)O)C2 ; NC1CC2(C1)CC(C(=O)Nc1cc(Cl)cnc1Cl)C2 ; CS(=O)(=O)c1ccc(C(=O)NC2CC3(C2)CC(C(=O)Nc2cc(Cl)cnc2Cl)C3)o1 ; CS(=O)(=O)c1ccc(C(=O)NC2CC3(C2)CC(c2nc4cc(Cl)cnc4o2)C3)o1]

### Step 1: Preparation of 2-amino-N-(2,5-dichloro-3-pyridyl)spiro[3.3]heptane-6-carboxamide (48a)

The polyphosphoric acid (471 mg, 1.96 mmol) was heated at 110° C. and stirred for 10 min. A mixture of 2-(tert-butoxycarbonylamino)spiro[3.3]heptane-6-carboxylic acid (275 mg, 1.08 mmol) and 3-amino-2,5-dichloropyridine (160 mg, 0.98 mmol) was added and stirred at 120° C. for 1 h. The mixture was poured into ice-water (300 mL), adjusted pH to 8 by NH₄OH and extracted with DCM (300 mL×3). The combined organic layer was separated, dried over Na₂SO₄ and concentrated in vacuo to afford 2-amino-N-(2,5-dichloro-3-pyridyl)spiro[3.3]heptane-6-carboxamide as a light yellow solid (48a, 150 mg, 50.9%). MS obsd. (ESI⁺) [(M+H)⁺]: 300.1.

### Step 2: Preparation of N-[6-[(2,5-dichloro-3-pyridyl)carbamoyl]spiro[3.3]heptan-2-yl]-5-methylsulfonyl-furan-2-carboxamide (48b)

To a solution of 5-methylsulfonylfuran-2-carboxylic acid (Int-3, 97.1 mg, 0.51 mmol) in DCM (2 mL) were added 2,4,6-tripropyl-1,3,5,2,4,6-trioxatriphosphinane 2,4,6-trioxide (0.36 mL, 0.6 mmol) and triethylamine (0.21 mL, 1.5 mmol). After being stirred at 25° C. for 30 min, 2-amino-N-(2,5-dichloro-3-pyridyl)spiro[3.3]heptane-6-carboxamide (48a, 150 mg, 0.5 mmol) was added, and the reaction was stirred for further 1 h. The mixture was diluted with water (20 mL) and extracted with EtOAc (30 mL×3). The combined organic layer was dried with Na₂SO₄ and concentrated in vacuo. The residue was purified by preparative TLC (EtOAc/PE=3/1) to give a crude product. The crude product was further purified by preparative HPLC to give N-[6-[(2,5-dichloro-3-pyridyl)carbamoyl]spiro[3.3]heptan-2-yl]-5-methylsulfonyl-furan-2-carboxamide as an off-white solid (48b, 100 mg, 42.4%). MS obsd. (ESI⁺) [(M+H)⁺]: 472.1.

### Step 3: Preparation of N-[6-(6-chlorooxazolo[5,4-b]pyridin-2-yl)spiro[3.3]heptan-2-yl]-5-methylsulfonyl-furan-2-carboxamide (Example 48)

To a solution of N-[6-[(2,5-dichloro-3-pyridyl)carbamoyl]spiro[3.3]heptan-2-yl]-5-methylsulfonyl-furan-2-carboxamide (48b, 35 mg, 0.07 mmol) in NMP (2 mL) was added K₂CO₃ (12.4 mg, 0.09 mmol). After being irradiated in a microwave reactor at 180° C. for 0.4 h, the mixture was quenched by water (40 mL) and extracted with EtOAc (30 mL×3). The combined organic layer was dried over Na₂SO₄ and concentrated in vacuo. The residue was purified by preparative HPLC to give N-[6-(6-chlorooxazolo[5,4-b]pyridin-2-yl)spiro[3.3]heptan-2-yl]-5-methylsulfonyl-furan-2-carboxamide as a light yellow solid (Example 48, 11.5 mg, 34.4%). MS obsd. (ESI⁺) [(M+H)⁺]: 436.1. ¹H NMR (400 MHz, CD₃OD) δ ppm: 8.29 (d, J=2.32 Hz, 1H), 8.12 (d, J=2.32 Hz, 1H), 7.27-7.33 (m, 1H), 7.19-7.23 (m, 1H), 4.34-4.47 (m, 1H), 3.78 (quin, J=8.44 Hz, 1H), 3.28 (s, 3H), 2.42-2.73 (m, 6H), 2.16-2.33 (m, 2H).

### Example 49

### Example 49-a, Example 49-b

### N-[6-(6-chlorooxazolo[5,4-b]pyridin-2-yl)spiro[3.3]heptan-2-yl]-5-cyclopropylsulfonyl-furan-2-carboxamide

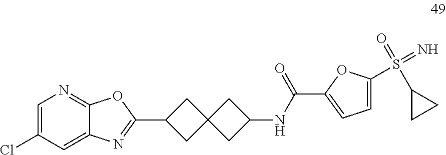 [SMILES: N=S(=O)(c1ccc(C(=O)NC2CC3(C2)CC(c2nc4cc(Cl)cnc4o2)C3)o1)C1CC1]

N-[6-(6-Chlorooxazolo[5,4-b]pyridin-2-yl)spiro[3.3]heptan-2-yl]-5-cyclopropylsulfonyl-furan-2-carboxamide (Example 49) was prepared in analogy to the procedure described for the preparation of Example 48, by using 5-cyclopropylsulfonylfuran-2-carboxylic acid (Int-6) instead of 5-methylsulfonylfuran-2-carboxylic acid (Int-3). MS obsd. (ESI⁺) [(M+H)⁺]: 462.1. ¹H NMR (400 MHz, DMSO-d₆) δ ppm: 8.87 (d, J=7.46 Hz, 1H), 8.36-8.42 (m, 2H), 7.36 (d, J=3.67 Hz, 1H), 7.29 (d, J=3.67 Hz, 1H), 4.30 (sxt, J=8.12 Hz, 1H), 3.78 (quin, J=8.53 Hz, 1H), 2.92-3.03 (m, 1H), 2.43-2.66 (m, 5H), 2.29-2.38 (m, 1H), 2.20-2.27 (m, 1H), 2.11-2.20 (m, 1H), 1.19-1.27 (m, 2H), 1.12-1.19 (m, 2H).

The two enantiomers (Example 49-a, Example 49-b) were obtained through SFC chiral separation of N-[6-(6-chlorooxazolo[5,4-b]pyridin-2-yl)spiro[3.3]heptan-2-yl]-5-cyclopropylsulfonyl-furan-2-carboxamide (Example 49).

### (Rₐ)—N-[6-(6-chlorooxazolo[5,4-b]pyridin-2-yl)spiro[3.3]heptan-2-yl]-5-cyclopropylsulfonyl-furan-2-carboxamide

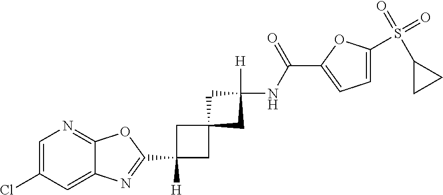

### (Sₐ)—N-[6-(6-chlorooxazolo[5,4-b]pyridin-2-yl)spiro[3.3]heptan-2-yl]-5-cyclopropylsulfonyl-furan-2-carboxamide

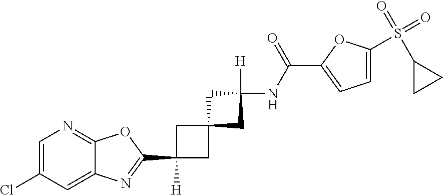

Example 49-a: white solid. SFC analysis: RT=5.049 min (Instrument: Waters Acquity UPCC; Column: Daicel CHIRALPAK IG-3, 3.0*150 mm, 3 μm; Mobile Phase: CO₂/EtOH=85/15; Flow rate: 2.0 mL/min; Column Temp: 37° C.). MS obsd. (ESI⁺) [(M+H)⁺]: 462.1. ¹H NMR (400 MHz, DMSO-d₆) δ ppm: 8.87 (d, J=7.46 Hz, 1H), 8.36-8.41 (m, 2H), 7.26-7.39 (m, 2H), 4.31 (sxt, J=8.12 Hz, 1H), 3.78 (quin, J=8.47 Hz, 1H), 2.88-3.04 (m, 1H), 2.41-2.70 (m, 5H), 2.29-2.38 (m, 1H), 2.11-2.28 (m, 2H), 1.19-1.27 (m, 2H), 1.11-1.19 (m, 2H).

Example 49-b: white solid. SFC analysis: RT=7.167 min (Instrument: Waters Acquity UPCC; Column: Daicel CHIRALPAK IG-3, 3.0*150 mm, 3 μm; Mobile Phase: CO₂/EtOH=85/15; Flow rate: 2.0 mL/min; Column Temp: 37° C.). MS obsd. (ESI⁺) [(M+H)⁺]: 462.0. ¹H NMR (400 MHz, DMSO-d₆) δ ppm: 8.87 (d, J=7.46 Hz, 1H), 8.38 (s, 2H), 7.25-7.41 (m, 2H), 4.31 (sxt, J=8.09 Hz, 1H), 3.78 (quin, J=8.50 Hz, 1H), 2.90-3.02 (m, 1H), 2.40-2.69 (m, 5H), 2.30-2.39 (m, 1H), 2.12-2.29 (m, 2H), 1.19-1.27 (m, 2H), 1.11-1.18 (m, 2H).

### Example 50

### N-[6-(6-chlorooxazolo[5,4-b]pyridin-2-yl)spiro[3.3]heptan-2-yl]-5-cyclopropylsulfinyl-furan-2-carboxamide

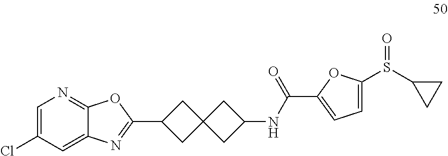 [SMILES: O=C(NC1CC2(C1)CC(c1nc3cc(Cl)cnc3o1)C2)c1ccc(S(=O)C2CC2)o1]

The title compound was prepared according to the following scheme:

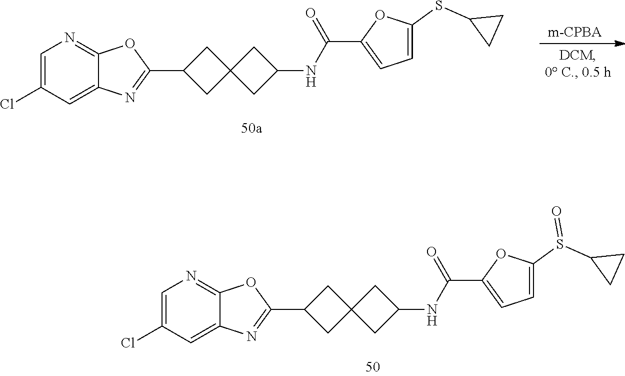 [SMILES: O=C(NC1CC2(C1)CC(c1nc3cc(Cl)cnc3o1)C2)c1ccc(SC2CC2)o1 ; O=C(NC1CC2(C1)CC(c1nc3cc(Cl)cnc3o1)C2)c1ccc(S(=O)C2CC2)o1]

The mixture of N-[6-(6-chlorooxazolo[5,4-b]pyridin-2-yl)spiro[3.3]heptan-2-yl]-5-cyclopropylsulfanyl-furan-2-carboxamide [50a, 50 mg, 0.12 mmol, prepared in analogy to the procedure described for the preparation of Example 48, by using 5-cyclopropylsulfanylfuran-2-carboxylic acid (Int-5) instead of 5-methylsulfonylfuran-2-carboxylic acid (Int-3)] and m-CPBA (20 mg, 0.12 mmol) in DCM (3 mL) was stirred at 0° C. for 0.5 h. The reaction was quenched by saturated NaHCO₃ and extracted with DCM (15 mL×3). The combined organic layer was washed with brine (10 mL×3), dried over MgSO4, filtered, and concentrated in vacuo. The residue was purified by preparative HPLC to give N-[6-(6-chlorooxazolo[5,4-b]pyridin-2-yl)spiro[3.3]heptan-2-yl]-5-cyclopropylsulfinyl-furan-2-carboxamide as a white solid (Example 50, 14.5 mg, 28%). MS obsd. (ESI⁺) [(M+H)⁺]: 446.1. ¹H NMR (400 MHz, DMSO-d₆) δ ppm: 8.80 (d, J=7.58 Hz, 1H), 8.33-8.42 (m, 2H), 7.17-7.28 (m, 2H), 4.31 (sxt, J=8.12 Hz, 1H), 3.78 (quin, J=8.44 Hz, 1H), 2.85-3.00 (m, 1H), 2.40-2.66 (m, 5H), 2.28-2.38 (m, 1H), 2.24 (br t, J=9.66 Hz, 1H), 2.12-2.20 (m, 1H), 1.08-1.22 (m, 2H), 0.96-1.07 (m, 1H), 0.78 (tdd, J=5.04, 6.43, 10.13 Hz, 1H).

### Example 51

### N-[6-(6-chlorooxazolo[4,5-c]pyridin-2-yl)spiro[3.3]heptan-2-yl]-5-cyclopropylsulfonyl-furan-2-carboxamide

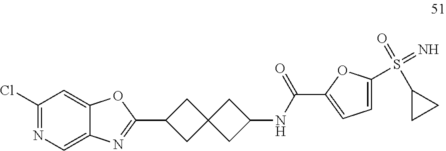 [SMILES: N=S(=O)(c1ccc(C(=O)NC2CC3(C2)CC(c2nc4cnc(Cl)cc4o2)C3)o1)C1CC1]

The title compound was prepared according to the following scheme:

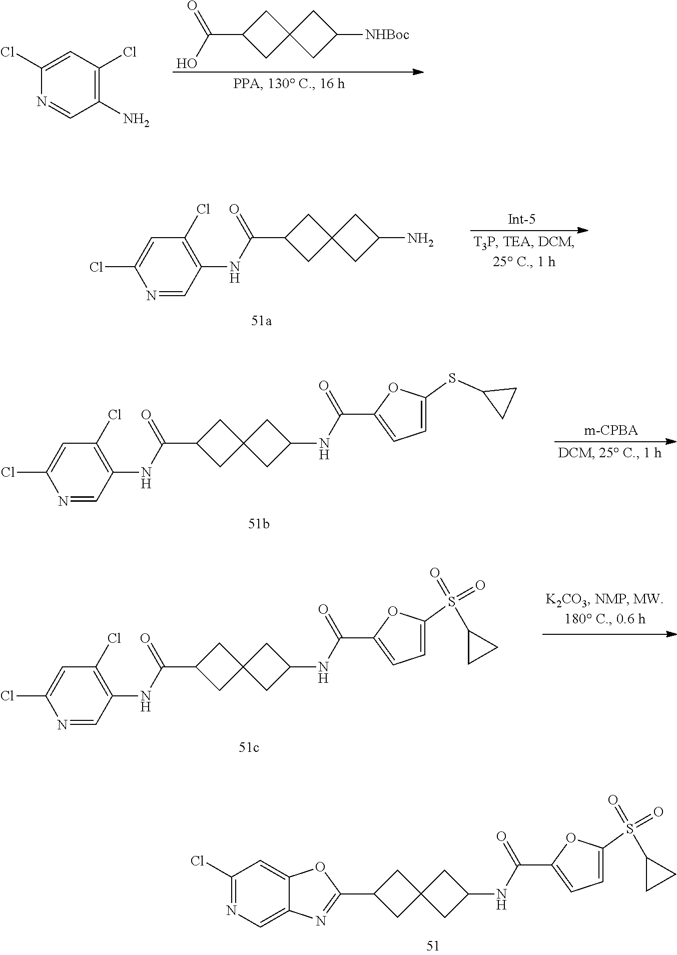 [SMILES: Nc1cnc(Cl)cc1Cl ; CC(C)(C)OC(=O)NC1CC2(C1)CC(C(=O)O)C2 ; NC1CC2(C1)CC(C(=O)Nc1cnc(Cl)cc1Cl)C2 ; O=C(NC1CC2(C1)CC(C(=O)Nc1cnc(Cl)cc1Cl)C2)c1ccc(SC2CC2)o1 ; O=C(NC1CC2(C1)CC(C(=O)Nc1cnc(Cl)cc1Cl)C2)c1ccc(S(=O)(=O)C2CC2)o1 ; O=C(NC1CC2(C1)CC(c1nc3cnc(Cl)cc3o1)C2)c1ccc(S(=O)(=O)C2CC2)o1]

### Step 1: Preparation of 2-amino-N-(4,6-dichloro-3-pyridyl)spiro[3.3]heptane-6-carboxamide (51a)

A solution of 4,6-dichloropyridin-3-amine (300 mg, 1.84 mmol,) and 2-(tert-butoxycarbonylamino)spiro[3.3]heptane-6-carboxylic acid (469.9 mg, 1.84 mmol) in polyphosphoric acid (6.2 g, 18.4 mmol) was stirred at 130° C. for 16 h. The resulting mixture was quenched with cooled NH₄OH (100 mL) and extracted with EtOAc (100 mL×2). The combined organic layer was concentrated to give the crude product, which was purified by flash column (eluting with MeOH/DCM=1/50 to 1/6) to give 2-amino-N-(4,6-dichloro-3-pyridyl)spiro[3.3]heptane-6-carboxamide as a yellow solid (51a, 360 mg, 65.2%). MS obsd. (ESI⁺) [(M+H)⁺]: 300.1.

### Step 2: Preparation of 5-cyclopropylsulfanyl-N-[6-[(4,6-dichloro-3-pyridyl)carbamoyl]spiro[3.3]heptan-2-yl]furan-2-carboxamide (51b)

To a solution of 2-amino-N-(4,6-dichloro-3-pyridyl)spiro[3.3]heptane-6-carboxamide (51a, 310 mg, 1.03 mmol) and 5-cyclopropylsulfanylfuran-2-carboxylic acid (Int-5, 209.3 mg, 1.14 mmol) in DCM (5 mL) were added 2,4,6-tripropyl-1,3,5,2,4,6-trioxatriphosphinane 2,4,6-trioxide (394 mg, 1.2 mmol) and triethylamine (0.43 mL, 3.1 mmol). After being stirred at 25° C. for 1 h, the solvent was removed in vacuo, and the residue was purified by flash column (eluting with EtOAc/PE=1/20 to 1/1) to give 5-cyclopropylsulfanyl-N-[6-[(4,6-dichloro-3-pyridyl)carbamoyl]spiro[3.3]heptan-2-yl]furan-2-carboxamide as a colorless oil (51b, 280 mg, 58.1%). MS obsd. (ESI⁺) [(M+H)⁺]: 466.0.

### Step 3: Preparation of 5-cyclopropylsulfonyl-N-[6-[(4,6-dichloro-3-pyridyl)carbamoyl]spiro[3.3]heptan-2-yl]furan-2-carboxamide (51c)

To a solution of 5-cyclopropylsulfanyl-N-[6-[(4,6-dichloro-3-pyridyl)carbamoyl]spiro[3.3]heptan-2-yl]furan-2-carboxamide (51b, 280 mg, 0.6 mmol) in DCM (5 mL) was added m-CPBA (311 mg, 1.8 mmol). After being stirred at 25° C. for 1 h, the solvent was removed in vacuo, and the residue was purified by flash column (eluting with EtOAc/PE=0 to 50%) to give 5-cyclopropylsulfonyl-N-[6-[(4,6-dichloro-3-pyridyl)carbamoyl]spiro[3.3]heptan-2-yl]furan-2-carboxamide as a white solid (51c, 250 mg, 83.6%). MS obsd. (ESI⁺) [(M+H)⁺]: 500.1.

### Step 4: Preparation of N-[6-(6-chlorooxazolo[4,5-c]pyridin-2-yl)spiro[3.3]heptan-2-yl]-5-cyclopropylsulfonyl-furan-2-carboxamide (Example 51)

To a solution of 5-cyclopropylsulfonyl-N-[6-[(4,6-dichloro-3-pyridyl)carbamoyl]spiro[3.3]heptan-2-yl]furan-2-carboxamide (51c, 200 mg, 0.4 mmol) in NMP (2 mL) was added K₂CO₃ (83.2 mg, 0.6 mmol). After being irradiated in a microwave reactor at 180° C. for 0.6 h, the mixture was purified by preparative HPLC to give N-[6-(6-chlorooxazolo[4,5-c]pyridin-2-yl)spiro[3.3]heptan-2-yl]-5-cyclopropylsulfonyl-furan-2-carboxamide as a light-grey solid (Example 51, 30.6 mg, 16.5%). MS obsd. (ESI⁺) [(M+H)⁺]: 462.0. ¹H NMR (400 MHz, CD₃OD) δ ppm: 8.68 (s, 1H), 7.76 (s, 1H), 7.19-7.26 (m, 2H), 4.41 (quin, J=8.28 Hz, 1H), 3.79 (quin, J=8.47 Hz, 1H), 2.84 (tt, J=4.72, 7.93 Hz, 1H), 2.39-2.74 (m, 6H), 2.16-2.34 (m, 2H), 1.27-1.38 (m, 2H), 1.12-1.22 (m, 2H).

### Example 52

### Example 52-a, Example 52-b, Example 52-c, Example 52-d

### N-[6-(5-chloro-1,3-benzoxazol-2-yl)spiro[3.3]heptan-2-yl]-5-(methylsulfonimidoyl)furan-2-carboxamide

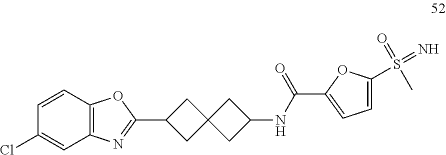 [SMILES: CS(=N)(=O)c1ccc(C(=O)NC2CC3(C2)CC(c2nc4cc(Cl)ccc4o2)C3)o1]

N-[6-(5-Chloro-1,3-benzoxazol-2-yl)spiro[3.3]heptan-2-yl]-5-(methylsulfonimidoyl)furan-2-carboxamide (Example 52) was prepared according to the following scheme:

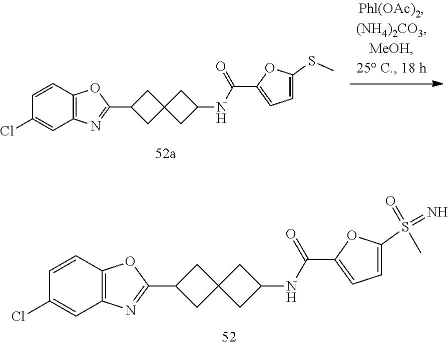 [SMILES: CSc1ccc(C(=O)NC2CC3(C2)CC(c2nc4cc(Cl)ccc4o2)C3)o1 ; CS(=N)(=O)c1ccc(C(=O)NC2CC3(C2)CC(c2nc4cc(Cl)ccc4o2)C3)o1]

A mixture of iodobenzene diacetate (2.98 g, 9.24 mmol) and ammonium carbonate (289.6 mg, 3.01 mmol) in methanol (50 mL) was stirred at 25° C. for 5 min. And then N-[6-(5-chloro-1,3-benzoxazol-2-yl)spiro[3.3]heptan-2-yl]-5-methylsulfanyl-furan-2-carboxamide {52a, 1.62 g, 4.02 mmol, prepared in analogy to the procedure described for the preparation of Example 1, by using 5-methylsulfanylfuran-2-carboxylic acid (Int-1) instead of 5-(trifluoromethyl)-2-furoic acid. MS obsd. (ESI⁺) [(M+H)⁺]: 403.1.} was added. After being stirred at 25° C. for 18 h, the mixture was quenched by water, concentrated in vacuo to remove the organic solvent. The residue was extracted with DCM (50 mL×3). The combined organic layers were washed with brine (50 mL×3), dried over Na₂SO₄, filtered and concentrated under reduced pressure. The residue was purified by preparative HPLC to give N-[6-(5-chloro-1,3-benzoxazol-2-yl)spiro[3.3]heptan-2-yl]-5-(methylsulfonimidoyl)furan-2-carboxamide as a white solid (Example 52, 819 mg, 46.75%). MS obsd. (ESI⁺) [(M+H)⁺]: 434.1. ¹H NMR (400 MHz, DMSO-d₆) δ ppm: 8.78 (br d, J=7.58 Hz, 1H), 7.79 (d, J=1.96 Hz, 1H), 7.71 (d, J=8.68 Hz, 1H), 7.39 (dd, J=2.02, 8.62 Hz, 1H), 7.20 (d, J=3.55 Hz, 1H), 7.13 (d, J=3.55 Hz, 1H), 4.86 (br s, 1H), 4.30 (sxt, J=8.09 Hz, 1H), 3.74 (quin, J=8.44 Hz, 1H), 3.19 (s, 3H), 2.41-2.67 (m, 5H), 2.28-2.37 (m, 1H), 2.18-2.27 (m, 1H), 2.09-2.18 (m, 1H).

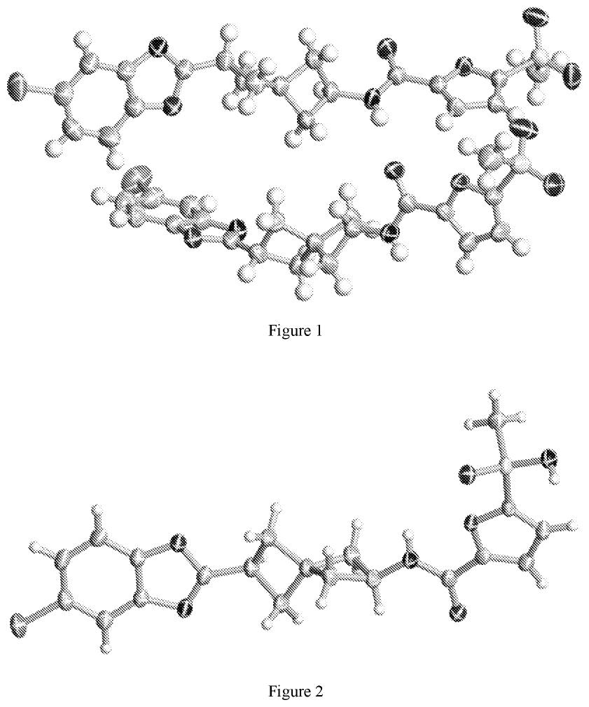

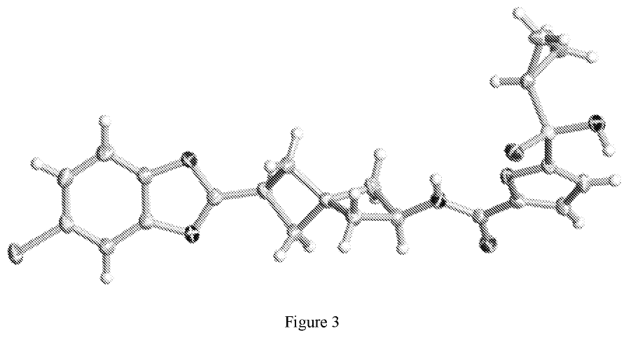

The four diastereomers (Example 52-a, Example 52-b, Example 52-c, Example 52-d) were obtained through chiral separation of N-[6-(5-chloro-1,3-benzoxazol-2-yl)spiro[3.3]heptan-2-yl]-5-(methylsulfonimidoyl)furan-2-carboxamide (Example 52). The absolute configuration of Example 52-d was determined by X-ray diffraction study (FIG. 2).

### (Rₐ)—N-[6-(5-chloro-1,3-benzoxazol-2-yl)spiro[3.3]heptan-2-yl]-5-[(S)-methylsulfonimidoyl]furan-2-carboxamide

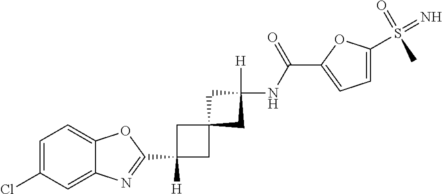

### (Rₐ)—N-[6-(5-chloro-1,3-benzoxazol-2-yl)spiro[3.3]heptan-2-yl]-5-[(R)-methylsulfonimidoyl]furan-2-carboxamide (Example 52-d)

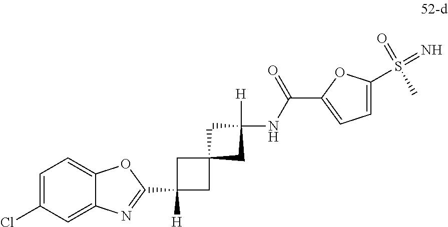

### (Sₐ)—N-[6-(5-chloro-1,3-benzoxazol-2-yl)spiro[3.3]heptan-2-yl]-5-[(S)-methylsulfonimidoyl]furan-2-carboxamide

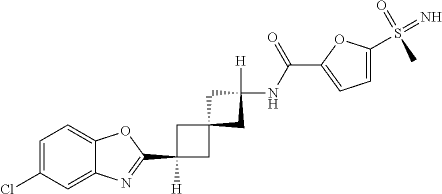

### (Sₐ)—N-[6-(5-chloro-1,3-benzoxazol-2-yl)spiro[3.3]heptan-2-yl]-5-[(R)-methylsulfonimidoyl]furan-2-carboxamide

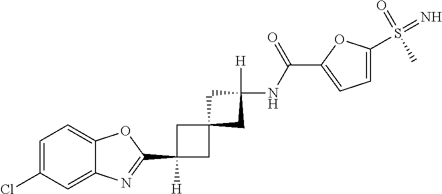

Example 52-a: (141.3 mg, white solid). Chiral analysis: RT=7.558 min [HPLC equipment: Shimadzu LC-20AT; Column: CHIRALPAK IG(IG00CE-UE011); Column size: 0.46 cm I.D.×25 cm L; Injection: 5.0 μl; Mobile phase: MeOH/ACN=90/10(V/); Flow rate: 1.0 mL/min; Temperature: 35° C.]. MS obsd. (ESI⁺) [(M+H)⁺]: 434.1. ¹H NMR (400 MHz, DMSO-d₆) δ ppm: 8.78 (d, J=7.6 Hz, 1H), 7.80 (d, J=2.1 Hz, 1H), 7.72 (d, J=8.7 Hz, 1H), 7.40 (dd, J=8.7, 2.1 Hz, 1H), 7.20 (d, J=3.6 Hz, 1H), 7.14 (d, J=3.6 Hz, 1H), 4.87 (s, 1H), 4.24-4.38 (m, 1H), 3.70-3.79 (m, 1H), 3.20 (d, J=0.7 Hz, 3H), 2.39-2.71 (m, 5H), 2.28-2.36 (m, 1H), 2.20-2.26 (m, 1H), 2.12-2.18 (m, 1H).

Example 52-b: (87 mg, white solid). Chiral analysis: RT=8.855 min [HPLC equipment: Shimadzu LC-20AT; Column: CHIRALPAK IG(IG00CE-UE011); Column size: 0.46 cm I.D.×25 cm L; Injection: 5.0 μl; Mobile phase: MeOH/ACN=90/10(V/); Flow rate: 1.0 mL/min; Temperature: 35° C.]. MS obsd. (ESI⁺) [(M+H)⁺]: 434.0. ¹H NMR (400 MHz, DMSO-d₆) δ ppm: 8.78 (d, J=7.6 Hz, 1H), 7.80 (d, J=2.1 Hz, 1H), 7.72 (d, J=8.7 Hz, 1H), 7.40 (dd, J=8.7, 2.1 Hz, 1H), 7.20 (d, J=3.6 Hz, 1H), 7.14 (d, J=3.6 Hz, 1H), 4.87 (s, 1H), 4.24-4.37 (m, 1H), 3.70-3.79 (m, 1H), 3.20 (d, J=1.1 Hz, 3H), 2.39-2.68 (m, 5H), 2.28-2.37 (m, 1H), 2.19-2.27 (m, 1H), 2.12-2.19 (m, 1H).

Example 52-c: (68 mg, white solid). Chiral analysis: RT=9.328 min [HPLC equipment: Shimadzu LC-20AT; Column: CHIRALPAK IG(IG00CE-UE011); Column size: 0.46 cm I.D.×25 cm L; Injection: 5.0 μl; Mobile phase: MeOH/ACN=90/10(V/); Flow rate: 1.0 mL/min; Temperature: 35° C.]. MS obsd. (ESI⁺) [(M+H)⁺]: 434.1. ¹H NMR (400 MHz, DMSO-d₆) δ ppm: 8.78 (d, J=7.6 Hz, 1H), 7.80 (d, J=2.1 Hz, 1H), 7.72 (d, J=8.7 Hz, 1H), 7.40 (dd, J=8.7, 2.1 Hz, 1H), 7.20 (d, J=3.6 Hz, 1H), 7.13 (d, J=3.6 Hz, 1H), 4.86 (s, 1H), 4.24-4.37 (m, 1H), 3.70-3.79 (m, 1H), 3.20 (d, J=0.7 Hz, 3H), 2.39-2.71 (m, 5H), 2.28-2.37 (m, 1H), 2.20-2.27 (m, 1H), 2.12-2.18 (m, 1H).

Example 52-d: (140.3 mg, off-white solid). Chiral analysis: RT=17.011 min [HPLC equipment: Shimadzu LC-20AT; Column: CHIRALPAK IG(IG00CE-UE011); Column size: 0.46 cm I.D.×25 cm L; Injection: 5.0 μl; Mobile phase: MeOH/ACN=90/10(V/); Flow rate: 1.0 mL/min; Temperature: 35° C.]. MS obsd. (ESI⁺) [(M+H)⁺]: 434.1. ¹H NMR (400 MHz, DMSO-d₆) δ ppm: 8.78 (d, J=7.6 Hz, 1H), 7.80 (d, J=2.1 Hz, 1H), 7.72 (d, J=8.7 Hz, 1H), 7.40 (dd, J=8.7, 2.1 Hz, 1H), 7.20 (d, J=3.6 Hz, 1H), 7.13 (d, J=3.6 Hz, 1H), 4.87 (s, 1H), 4.24-4.37 (m, 1H), 3.70-3.79 (m, 1H), 3.20 (s, 3H), 2.40-2.70 (m, 5H), 2.28-2.36 (m, 1H), 2.20-2.27 (m, 1H), 2.12-2.19 (m, 1H).

### Example 53

### Example 53-a, Example 53-b, Example 53-c, Example 53-d

### N-[6-(5-chloro-1,3-benzoxazol-2-yl)spiro[3.3]heptan-2-yl]-5-(cyclopropylsulfonimidoyl)furan-2-carboxamide

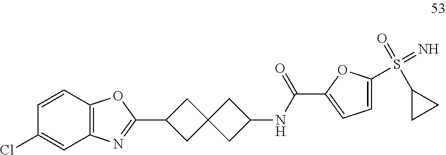 [SMILES: N=S(=O)(c1ccc(C(=O)NC2CC3(C2)CC(c2nc4cc(Cl)ccc4o2)C3)o1)C1CC1]

N-[6-(5-Chloro-1,3-benzoxazol-2-yl)spiro[3.3]heptan-2-yl]-5-(cyclopropylsulfonimidoyl)furan-2-carboxamide (Example 53) was prepared in analogy to the procedure described for the preparation of Example 52, by using 5-cyclopropylsulfanylfuran-2-carboxylic acid (Int-5) instead of 5-methylsulfanylfuran-2-carboxylic acid (Int-1). MS obsd. (ESI⁺) [(M+H)⁺]: 460.1. ¹H NMR (400 MHz, CD₃OD) δ ppm: 7.64 (d, J=2.08 Hz, 1H), 7.55 (d, J=8.68 Hz, 1H), 7.35 (dd, J=2.08, 8.68 Hz, 1H), 7.16-7.22 (m, 2H), 4.41 (quin, J=8.28 Hz, 1H), 3.75 (quin, J=8.47 Hz, 1H), 2.80-2.89 (m, 1H), 2.40-2.72 (m, 6H), 2.27 (dd, J=9.23, 10.82 Hz, 1H), 2.19 (dd, J=8.99, 11.31 Hz, 1H), 1.29-1.38 (m, 1H), 1.25 (dt, J=4.65, 10.58 Hz, 1H), 1.12-1.20 (m, 1H), 1.01-1.12 (m, 1H).

The four diastereomers (Example 53-a, Example 53-b, Example 53-c and Example 53-d) were obtained through SFC [Condition I, Instrument: SFC 80, Column: AD, 250×30 mm I.D., 5 μm; Mobile phase: A for CO₂ and B for Ethanol (0.1% NH₄OH); Gradient: B 20%; Flow rate: 50 mL/min; Back pressure: 100 bar; Column temperature: 40° C.; elution order was a mixture of Example 53-a and Example 53-b, Example 53-c, Example 53-d. Condition II, Instrument: Waters Acquity UPCC; Column: Daicel CHIRALPAK IC_3, 3.0*150 mm, 3 μm; Mobile Phase: CO₂/EtOH=75/25; Flow rate: 2.0 mL/min; Column Temp,: 37° C.; Example 53-a was eluted out before Example 53-b] chiral separation of N-[6-(5-chloro-1,3-benzoxazol-2-yl)spiro[3.3]heptan-2-yl]-5-(cyclopropylsulfonimidoyl)furan-2-carboxamide (Example 53). The absolute configuration of Example 53-c was determined by X-ray diffraction study (FIG. 3).

### (Rₐ)—N-[6-(5-chloro-1,3-benzoxazol-2-yl)spiro[3.3]heptan-2-yl]-5-[(S)-cyclopropylsulfonimidoyl]furan-2-carboxamide

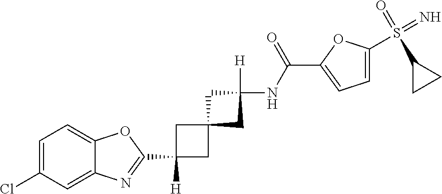

### (Rₐ)—N-[6-(5-chloro-1,3-benzoxazol-2-yl)spiro[3.3]heptan-2-yl]-5-[(R)-cyclopropylsulfonimidoyl]furan-2-carboxamide (Example 53-c)

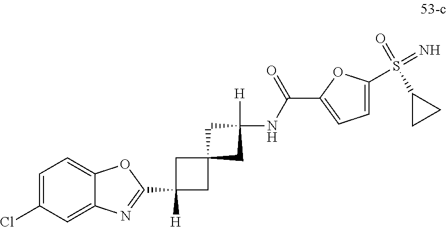

### (Sₐ)—N-[6-(5-chloro-1,3-benzoxazol-2-yl)spiro[3.3]heptan-2-yl]-5-[(S)-cyclopropylsulfonimidoyl]furan-2-carboxamide

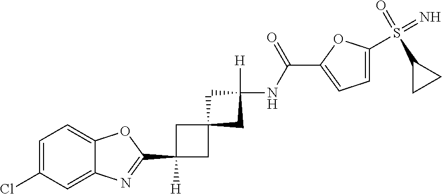

### (Sₐ)—N-[6-(5-chloro-1,3-benzoxazol-2-yl)spiro[3.3]heptan-2-yl-5-[(R)-cyclopropylsulfonimidoyl]furan-2-carboxamide

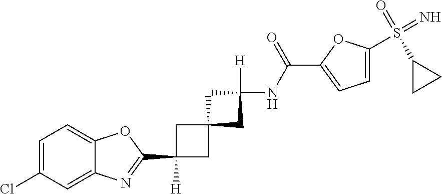

Example 53-a: MS obsd. (ESI⁺) [(M+H)⁺]: 460.1. ¹H NMR (400 MHz, CD₃OD) δ ppm: 7.64 (d, J=1.96 Hz, 1H), 7.55 (d, J=8.80 Hz, 1H), 7.35 (dd, J=2.08, 8.68 Hz, 1H), 7.19 (q, J=3.59 Hz, 2H), 4.32-4.48 (m, 1H), 3.75 (quin, J=8.56 Hz, 1H), 2.84 (tt, J=4.68, 7.92 Hz, 1H), 2.43-2.71 (m, 6H), 2.04-2.33 (m, 2H), 1.12-1.36 (m, 3H), 0.98-1.10 (m, 1H).

Example 53-b: MS obsd. (ESI⁺) [(M+H)⁺]: 460.1. ¹H NMR (400 MHz, CD₃OD) δ ppm: 7.64 (d, J=1.96 Hz, 1H), 7.56 (d, J=8.56 Hz, 1H), 7.35 (dd, J=2.08, 8.68 Hz, 1H), 7.19 (q, J=3.67 Hz, 2H), 4.31-4.49 (m, 1H), 3.75 (quin, J=8.50 Hz, 1H), 2.84 (tt, J=4.74, 7.86 Hz, 1H), 2.43-2.71 (m, 6H), 2.12-2.36 (m, 2H), 1.13-1.40 (m, 3H), 1.03-1.11 (m, 1H).

Example 53-c: MS obsd. (ESI⁺) [(M+H)⁺]: 460.3. ¹H NMR (400 MHz, DMSO-d₆) δ ppm: 8.75 (d, J=7.58 Hz, 1H), 7.80 (d, J=2.08 Hz, 1H), 7.72 (d, J=8.68 Hz, 1H), 7.40 (dd, J=2.14, 8.62 Hz, 1H), 7.22 (d, J=3.55 Hz, 1H), 7.10 (d, J=3.55 Hz, 1H), 4.92 (s, 1H), 4.25-4.35 (m, 1H), 3.74 (t, J=8.50 Hz, 1H), 2.73-2.81 (m, 1H), 2.40-2.67 (m, 5H), 2.12-2.37 (m, 3H), 0.96-1.20 (m, 4H).

Example 53-d: MS obsd. (ESI⁺) [(M+H)⁺]: 460.3. ¹H NMR (400 MHz, DMSO-d₆) δ ppm: 8.75 (d, J=7.58 Hz, 1H), 7.80 (d, J=2.08 Hz, 1H), 7.72 (d, J=8.56 Hz, 1H), 7.40 (dd, J=2.14, 8.62 Hz, 1H), 7.22 (d, J=3.67 Hz, 1H), 7.10 (d, J=3.55 Hz, 1H), 4.92 (s, 1H), 4.25-4.36 (m, 1H), 3.74 (t, J=8.50 Hz, 1H), 2.73-2.80 (m, 1H), 2.40-2.67 (m, 5H), 2.12-2.37 (m, 3H), 0.96-1.20 (m, 4H).

### Example 54

### Example 54-a, Example 54-b, Example 54-c, Example 54-d

### N-[6-(5-chloro-1,3-benzoxazol-2-yl)spiro[3.3]heptan-2-yl]-5-(cyclopropylmethylsulfonimidoyl)furan-2-carboxamide

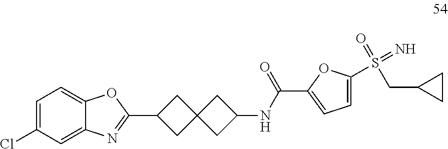 [SMILES: N=S(=O)(CC1CC1)c1ccc(C(=O)NC2CC3(C2)CC(c2nc4cc(Cl)ccc4o2)C3)o1]

N-[6-(5-Chloro-1,3-benzoxazol-2-yl)spiro[3.3]heptan-2-yl]-5-(cyclopropylmethylsulfonimidoyl)furan-2-carboxamide (Example 54) was prepared in analogy to the procedure described for the preparation of Example 52, by using 5-(cyclopropylmethylsulfanyl)furan-2-carboxylic acid (Int-12) instead of 5-methylsulfanylfuran-2-carboxylic acid (Int-1). The product was purified by preparative HPLC to afford Example 54 as a white solid. MS obsd. (ESI⁺) [(M+H)⁺]: 474.1. ¹H NMR (400 MHz, CD₃OD) δ ppm: 7.64 (d, J=2.0 Hz, 1H), 7.56 (d, J=8.7 Hz, 1H), 7.36 (dd, J=8.7, 2.1 Hz, 1H), 7.24 (dd, J=15.1, 3.6 Hz, 2H), 4.41 (p, J=8.4 Hz, 1H), 3.76 (p, J=8.5 Hz, 1H), 3.33 (s, 2H), 2.41-2.73 (m, 6H), 2.11-2.31 (m, 2H), 0.99-1.18 (m, 1H), 0.47-0.62 (m, 2H), 0.15 (tt, J=9.7, 4.9 Hz, 2H).

The four diastereomers (Example 54-a, Example 54-b, Example 54-c, Example 54-d) were obtained through SFC [Instrument: SFC 80, Column: AD, 250×20 mm I.D., 5 μm; Mobile phase: A for C₀₂ and B for Methanol (0.1% NH₄OH); Gradient: B 40%; Flow rate: 40 mL/min; Back pressure: 100 bar; Column temperature: 35° C.; elution order was Example 54-a, Example 54-b, Example 54-c, Example 54-d] chiral separation of N-[6-(5-chloro-1,3-benzoxazol-2-yl)spiro[3.3]heptan-2-yl]-5-(cyclopropylmethylsulfonimidoyl)furan-2-carboxamide (Example 54).

### (Rₐ)—N-[6-(5-chloro-1,3-benzoxazol-2-yl)spiro[3.3]heptan-2-yl]-5-[(S)-cyclopropylmethylsulfonimidoyl]furan-2-carboxamide

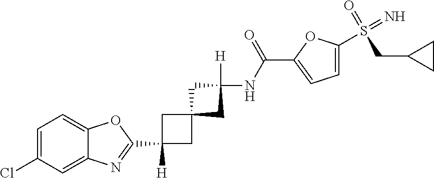

### (Rₐ)—N-[6-(5-Chloro-1,3-benzoxazol-2-yl)spiro[3.3]heptan-2-yl]-5-[(R)-cyclopropylmethylsulfonimidoyl]furan-2-carboxamide

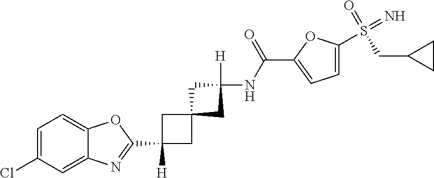

### (Sₐ)—N-[6-(5-Chloro-1,3-benzoxazol-2-yl)spiro[3.3]heptan-2-yl]-5-[(S)-cyclopropylmethylsulfonimidoyl]furan-2-carboxamide

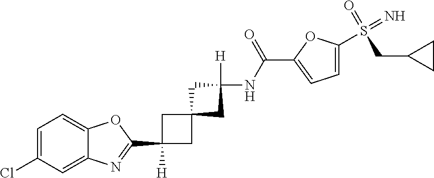

### (Sₐ)—N-[6-(5-Chloro-1,3-benzoxazol-2-yl)spiro[3.3]heptan-2-yl]-5-[(R)-cyclopropylmethylsulfonimidoyl]furan-2-carboxamide

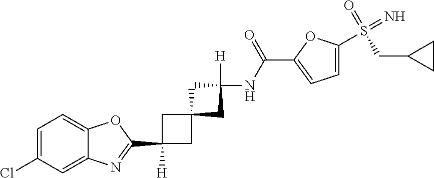

Example 54-a: MS obsd. (ESI⁺) [(M+H)⁺]: 474.2. Example 54-b: MS obsd. (ESI⁺) [(M+H)⁺]: 474.3. Example 54-c: MS obsd. (ESI⁺) [(M+H)⁺]: 474.3. Example 54-d: MS obsd. (ESI⁺) [(M+H)⁺]: 474.3.

### Example 55

### N-[6-(5-chloro-1,3-benzoxazol-2-yl)spiro[3.3]heptan-2-yl]-5-(isobutylsulfonimidoyl)furan-2-carboxamide

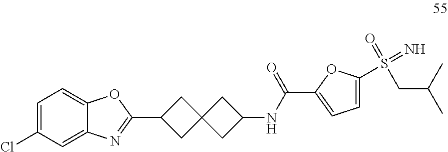 [SMILES: CC(C)CS(=N)(=O)c1ccc(C(=O)NC2CC3(C2)CC(c2nc4cc(Cl)ccc4o2)C3)o1]

The title compound was prepared in analogy to the procedure described for the preparation of Example 52, by using 5-isobutylsulfanylfuran-2-carboxylic acid (Int-15) instead of 5-methylsulfanylfuran-2-carboxylic acid (Int-1). The product was purified by preparative HPLC to afford Example 55 as a white solid. MS obsd. (ESI⁺) [(M+H)⁺]: 476.2. ¹H NMR (400 MHz, DMSO-d₆) δ ppm: 8.77 (d, J=7.58 Hz, 1H), 7.80 (d, J=2.08 Hz, 1H), 7.72 (d, J=8.68 Hz, 1H), 7.40 (dd, J=2.14, 8.62 Hz, 1H), 7.13-7.24 (m, 2H), 4.87 (s, 1H), 4.31 (sxt, J=8.17 Hz, 1H), 3.74 (quin, J=8.47 Hz, 1H), 3.09-3.25 (m, 2H), 2.38-2.66 (m, 3H), 2.28-2.36 (m, 1H), 2.19-2.27 (m, 1H), 2.09-2.19 (m, 2H), 0.96 (dd, J=2.93, 6.72 Hz, 6H).

### Example 56

### N-[6-(5-chloro-1,3-benzoxazol-2-yl)spiro[3.3]heptan-2-yl]-5-(oxetan-3-ylmethylsulfonimidoyl)furan-2-carboxamide

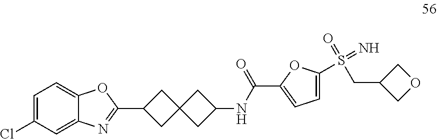 [SMILES: N=S(=O)(CC1COC1)c1ccc(C(=O)NC2CC3(C2)CC(c2nc4cc(Cl)ccc4o2)C3)o1]

The title compound was prepared in analogy to the procedure described for the preparation of Example 52, by using 5-(oxetan-3-ylmethylsulfanyl)furan-2-carboxylic acid (Int-19) instead of 5-methylsulfanylfuran-2-carboxylic acid (Int-1). The product was purified by preparative HPLC to afford Example 56 as a white solid. MS obsd. (ESI⁺) [(M+H)⁺]: 490.1. ¹H NMR (400 MHz, CDCl₃) δ ppm: 7.65 (d, J=1.8 Hz, 1H), 7.40 (d, J=8.7 Hz, 1H), 7.27-7.31 (m, 1H), 7.16 (dd, J=21.3, 3.6 Hz, 2H), 6.68 (d, J=7.7 Hz, 1H), 4.78-4.88 (m, 2H), 4.49 (ddd, J=15.9, 11.4, 7.4 Hz, 3H), 3.53-3.76 (m, 4H), 2.43-2.80 (m, 6H), 2.06-2.21 (m, 2H).

### Example 57

### N-[6-(5-chloro-1,3-benzoxazol-2-yl)spiro[3.3]heptan-2-yl]-5-[(3,3-difluorocyclobutyl)methylsulfonimidoyl]furan-2-carboxamide

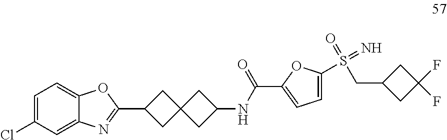 [SMILES: N=S(=O)(CC1CC(F)(F)C1)c1ccc(C(=O)NC2CC3(C2)CC(c2nc4cc(Cl)ccc4o2)C3)o1]

The title compound was prepared in analogy to the procedure described for the preparation of Example 56, by using 3-(bromomethyl)-1,1-difluoro-cyclobutane instead of 3-(bromomethyl)oxetane. The product was purified by preparative HPLC to afford Example 57 as a white solid. MS obsd. (ESI⁺) [(M+H)⁺]: 524.1. ¹H NMR (400 MHz, CDCl₃) δ ppm: 7.65 (d, J=2.0 Hz, 1H), 7.40 (d, J=8.6 Hz, 1H), 7.29 (d, J=2.1 Hz, 1H), 7.14-7.22 (m, 2H), 6.74 (s, 1H), 4.50 (d, J=7.1 Hz, 1H), 3.65-3.76 (m, 1H), 3.44-3.57 (m, 2H), 2.31-2.93 (m, 11H), 2.08-2.23 (m, 2H).

### Example 58

### N-[6-(5-chloro-1,3-benzoxazol-2-yl)spiro[3.3]heptan-2-yl]-5-[(ethylsulfonimidoyl)methyl]furan-2-carboxamide

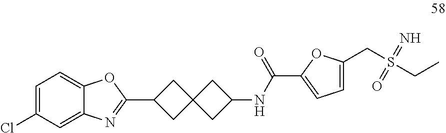 [SMILES: CCS(=N)(=O)Cc1ccc(C(=O)NC2CC3(C2)CC(c2nc4cc(Cl)ccc4o2)C3)o1]

The title compound was prepared according to the following scheme:

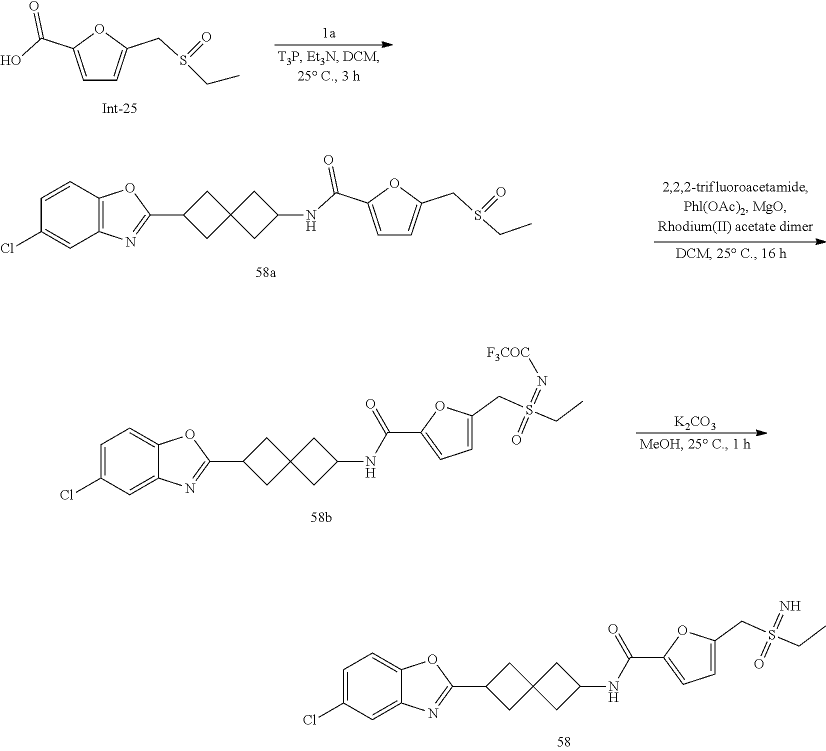

### Step 1: Preparation of N-[6-(5-chloro-1,3-benzoxazol-2-yl)spiro[3.3]heptan-2-yl]-5-(ethylsulfinylmethyl)furan-2-carboxamide (58a)

A mixture of 5-(ethylsulfinylmethyl)furan-2-carboxylic acid (Int-25, 140 mg, 0.69 mmol), 6-(5-chloro-1,3-benzoxazol-2-yl)spiro[3.3]heptan-2-amine (1a, 181.9 mg, 0.69 mmol), 2,4,6-tripropyl-1,3,5,2,4,6-trioxatriphosphorinane-2,4,6-trioxide (660.8 mg, 1.04 mmol) and triethylamine (0.29 mL, 2.08 mmol) in DMF (5 mL) was stirred at 25° C. for 3 h. Then the mixture was purified by flash column (eluting with EtOAc/PE=1/1) to give N-[6-(5-chloro-1,3-benzoxazol-2-yl)spiro[3.3]heptan-2-yl]-5-(ethylsulfinylmethyl)furan-2-carboxamide as a white solid (58a, 120 mg, 38.8%). MS obsd. (ESI⁺) [(M+H)⁺]: 447.0.

### Step 2: Preparation of N-[6-(5-chloro-1,3-benzoxazol-2-yl)spiro[3.3]heptan-2-yl]-5-[[S-ethyl-N-(2,2,2-trifluoroacetyl)sulfonimidoyl]methyl]furan-2-carboxamide (58b)

A mixture of N-[6-(5-chloro-1,3-benzoxazol-2-yl)spiro[3.3]heptan-2-yl]-5-(ethylsulfinylmethyl)furan-2-carboxamide (55a, 120 mg, 0.27 mmol), 2,2,2-trifluoroacetamide (136.6 mg, 1.21 mmol), magnesium oxide (64.4 mg, 1.61 mmol), rhodium(II) acetate dimer (11.8 mg, 0.03 mmol), and iodobenzene diacetate (259.4 mg, 0.81 mmol) in DCM (2 mL) was stirred at 25° C. for 16 h. The resulting mixture was concentrated and purified by flash column (eluting with EtOAc/PE=1/2) to give N-[6-(5-chloro-1,3-benzoxazol-2-yl)spiro[3.3]heptan-2-yl]-5-[[S-ethyl-N-(2,2,2-trifluoroacetyl)sulfonimidoyl]methyl]furan-2-carboxamide as a dark brown oil (58b, 90 mg, 60%). MS obsd. (ESI⁺) [(M+H)⁺]: 558.0.

### Step 3: Preparation of give N-[6-(5-chloro-1,3-benzoxazol-2-yl)spiro[3.3]heptan-2-yl]-5-[(ethylsulfonimidoyl)methyl]furan-2-carboxamide (Example 58)

To a stirring solution of N-[6-(5-chloro-1,3-benzoxazol-2-yl)spiro[3.3]heptan-2-yl]-5-[[S-ethyl-N-(2,2,2-trifluoroacetyl)sulfonimidoyl]methyl]furan-2-carboxamide (58b, 90 mg, 0.16 mmol) in MeOH (3 mL) was added K₂CO₃ (66.9 mg, 0.48 mmol). After being stirred at 25° C. for 1 h, the precipitate was filtered off. The filtration was concentrated to give a crude product which was purified by preparative HPLC to give N-[6-(5-chloro-1,3-benzoxazol-2-yl)spiro[3.3]heptan-2-yl]-5-[(ethylsulfonimidoyl)methyl]furan-2-carboxamide as a white solid (Example 58, 9.1 mg, 12%). MS obsd. (ESI⁺) [(M+H)⁺]: 462.0. ¹H NMR (400 MHz, CD₃OD) δ ppm: 7.64 (s, 1H), 7.54 (s, 1H), 7.36 (d, J=8.6 Hz, 1H), 7.10 (d, J=3.4 Hz, 1H), 6.67 (d, J=3.4 Hz, 1H), 4.78-4.86 (m, 2H), 4.30-4.49 (m, 1H), 3.74 (dd, J=8.2, 16.4 Hz, 1H), 3.15 (s, 2H), 2.38-2.72 (m, 6H), 2.21 (dt, J=9.8, 20.2 Hz, 2H), 1.38 (t, J=7.4 Hz, 3H).

### Example 59

### N-[6-(5-chloro-1,3-benzoxazol-2-yl)spiro[3.3]heptan-2-yl]-5-[(isopropylsulfonimidoyl)methyl]furan-2-carboxamide

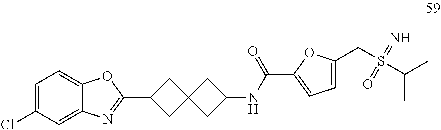 [SMILES: CC(C)S(=N)(=O)Cc1ccc(C(=O)NC2CC3(C2)CC(c2nc4cc(Cl)ccc4o2)C3)o1]

The title compound was prepared in analogy to the procedure described for the preparation of Example 58, by using methyl 5-(isopropylsulfanylmethyl)furan-2-carboxylate (Int-26a) instead of methyl 5-(ethylsulfanylmethyl)furan-2-carboxylate (Int-25a). The product was purified by preparative HPLC to afford Example 59 as a white solid. MS obsd. (ESI⁺) [(M+H)⁺]: 476.0. ¹H NMR (400 MHz, DMSO-d₆) δ ppm: 8.37 (d, J=7.7 Hz, 1H), 7.73 (d, J=2.1 Hz, 1H), 7.65 (d, J=8.7 Hz, 1H), 7.33 (dd, J=2.1, 8.7 Hz, 1H), 7.04 (d, J=3.4 Hz, 1H), 6.53 (d, J=3.4 Hz, 1H), 4.34-4.52 (m, 2H), 4.22 (dt, J=8.3, 16.1 Hz, 1H), 3.77 (s, 1H), 3.66 (dd, J=8.5, 17.0 Hz, 1H), 3.06 (dt, J=6.8, 13.5, Hz, 1H), 2.47-2.57 (m, 2H), 2.33-2.40 (m, 2H), 2.18-2.28 (m, 1H), 2.00-2.16 (m, 2H), 1.93 (dd, J=7.1, 14.7 Hz, 1H), 1.20 (dd, J=6.8, 2.3 Hz, 6H).

### Example 60

### N-[6-(5-chloro-1,3-benzoxazol-2-yl)spiro[3.3]heptan-2-yl]-5-[(cyclopropylsulfonimidoyl)methyl]furan-2-carboxamide

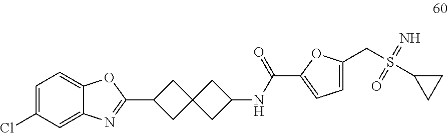 [SMILES: N=S(=O)(Cc1ccc(C(=O)NC2CC3(C2)CC(c2nc4cc(Cl)ccc4o2)C3)o1)C1CC1]

The title compound was prepared in analogy to the procedure described for the preparation of Example 58, by using N-[6-(5-chloro-1,3-benzoxazol-2-yl)spiro[3.3]heptan-2-yl]-5-(cyclopropylsulfinylmethyl)furan-2-carboxamide [prepared in analogy to the procedure described for the preparation of Example 38, by using 5-(cyclopropylsulfanylmethyl)furan-2-carboxylic acid (Int-27) instead of 5-tert-butylsulfanylfuran-2-carboxylic acid (Int-10)] instead of N-[6-(5-chloro-1,3-benzoxazol-2-yl)spiro[3.3]heptan-2-yl]-5-(ethylsulfinylmethyl)furan-2-carboxamide (58a). The product was purified by preparative HPLC to afford Example 60 as an off-white solid (11.6 mg, 11.3%). MS obsd. (ESI⁺) [(M+H)⁺]: 474.0. ¹H NMR (400 MHz, DMSO-d₆) δ ppm: 8.43 (d, J=7.70 Hz, 1H), 7.80 (d, J=2.08 Hz, 1H), 7.72 (d, J=8.68 Hz, 1H), 7.40 (dd, J=2.08, 8.68 Hz, 1H), 7.11 (d, J=3.42 Hz, 1H), 6.60 (d, J=3.42 Hz, 1H), 4.52 (s, 2H), 4.28 (sxt, J=8.14 Hz, 1H), 3.74 (quin, J=8.47 Hz, 1H), 2.53-2.67 (m, 4H), 2.44 (qd, J=2.71, 8.39 Hz, 2H), 2.25-2.35 (m, 1H), 2.16-2.24 (m, 1H), 2.11 (dd, J=9.17, 11.00 Hz, 1H), 0.83-1.00 (m, 3H), 0.68-0.82 (m, 1H).

### Example 61

### N-[6-(5-chloro-1,3-benzoxazol-2-yl)spiro[3.3]heptan-2-yl]-5-(1-oxo-4,5-dihydro-3H-isothiazol-1-yl)furan-2-carboxamide

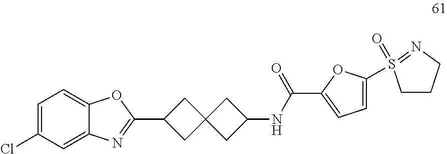 [SMILES: O=C(NC1CC2(C1)CC(c1nc3cc(Cl)ccc3o1)C2)c1ccc(S2(=O)=NCCC2)o1]

The title compound was prepared in analogy to the procedure described for the preparation of Example 1, by using 5-(1-oxo-4,5-dihydro-3H-isothiazol-1-yl)furan-2-carboxylic acid (Int-33) instead of 5-(trifluoromethyl)-2-furoic acid. The product was purified by preparative HPLC to afford Example 61 as a white solid. MS obsd. (ESI⁺) [(M+H)⁺]: 460.1. ¹H NMR (400 MHz, DMSO-d₆) δ ppm: 8.76 (d, J=7.46 Hz, 1H), 7.80 (d, J=2.08 Hz, 1H), 7.72 (d, J=8.68 Hz, 1H), 7.40 (dd, J=2.08, 8.68 Hz, 1H), 7.25 (q, J=3.67 Hz, 2H), 4.29 (sxt, J=8.17 Hz, 1H), 3.67-3.83 (m, 2H), 3.52-3.66 (m, 2H), 3.41 (ddd, J=5.81, 7.70, 13.51 Hz, 1H), 2.38-2.66 (m, 5H), 2.11-2.38 (m, 5H).

### Example 62

### N-[6-(5-chloro-1,3-benzoxazol-2-yl)spiro[3.3]heptan-2-yl]-5-(1-oxo-1lambda6-thia-2-azacyclohexen-1-yl)furan-2-carboxamide

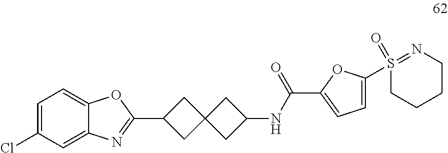 [SMILES: O=C(NC1CC2(C1)CC(c1nc3cc(Cl)ccc3o1)C2)c1ccc(S2(=O)=NCCCC2)o1]

The title compound was prepared in analogy to the procedure described for the preparation of Example 1, by using 5-(1-oxo-1lambda6-thia-2-azacyclohexen-1-yl)furan-2-carboxylic acid (Int-34) instead of 5-(trifluoromethyl)-2-furoic acid. The product was purified by preparative HPLC to afford Example 62 as a white solid. MS obsd. (ESI⁺) [(M+H)⁺]: 474.1. ¹H NMR (400 MHz, DMSO-d₆) δ ppm: 8.80 (d, J=7.58 Hz, 1H), 7.80 (d, J=2.08 Hz, 1H), 7.72 (d, J=8.56 Hz, 1H), 7.40 (dd, J=2.08, 8.68 Hz, 1H), 7.18-7.31 (m, 2H), 4.30 (sxt, J=8.14 Hz, 1H), 3.75 (quin, J=8.47 Hz, 1H), 3.49-3.59 (m, 1H), 3.41-3.49 (m, 1H), 3.23-3.31 (m, 1H), 3.03 (ddd, J=4.22, 9.26, 13.30 Hz, 1H), 2.45-2.66 (m, 5H), 2.28-2.38 (m, 1H), 2.07-2.28 (m, 3H), 1.70-1.88 (m, 1H), 1.44-1.66 (m, 2H).

### Example 63

### N-[6-(6-chlorooxazolo[5,4-b]pyridin-2-yl)spiro[3.3]heptan-2-yl]-5-(methylsulfonimidoyl)furan-2-carboxamide

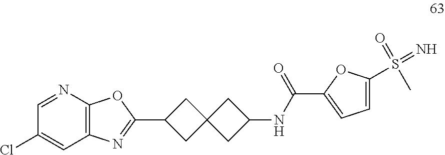 [SMILES: CS(=N)(=O)c1ccc(C(=O)NC2CC3(C2)CC(c2nc4cc(Cl)cnc4o2)C3)o1]

The title compound was prepared according to the following scheme:

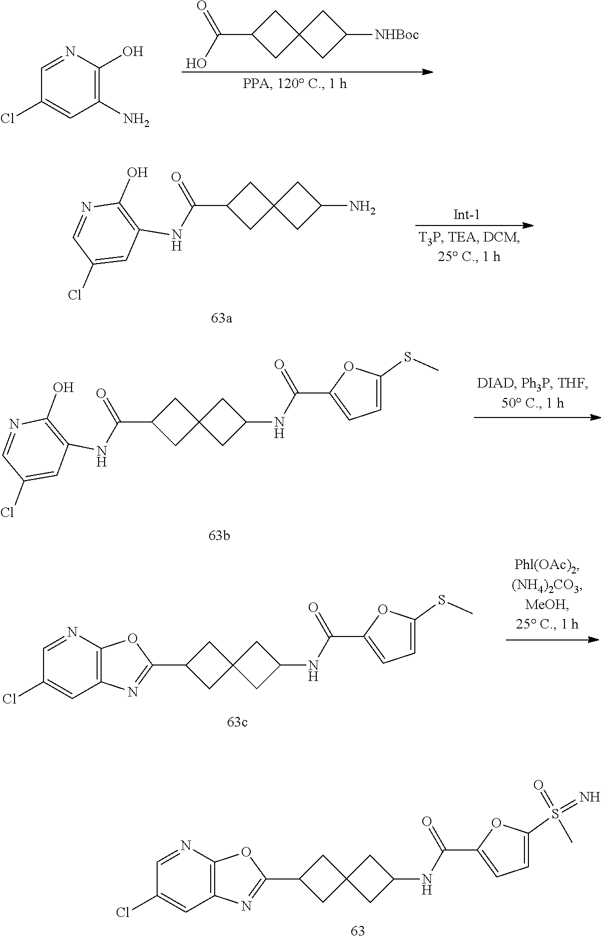 [SMILES: Nc1cc(Cl)cnc1O ; CC(C)(C)OC(=O)NC1CC2(C1)CC(C(=O)O)C2 ; NC1CC2(C1)CC(C(=O)Nc1cc(Cl)cnc1O)C2 ; CSc1ccc(C(=O)NC2CC3(C2)CC(C(=O)Nc2cc(Cl)cnc2O)C3)o1 ; CSc1ccc(C(=O)NC2CC3(C2)CC(c2nc4cc(Cl)cnc4o2)C3)o1 ; CS(=N)(=O)c1ccc(C(=O)NC2CC3(C2)CC(c2nc4cc(Cl)cnc4o2)C3)o1]

### Step 1: Preparation of 2-amino-N-(5-chloro-2-hydroxy-3-pyridyl)spiro[3.3]heptane-6-carboxamide (63a)

The polyphosphoric acid (12 g, 35 mmol) was heated at 110° C. and stirred for 10 min. A mixture of 2-(tert-butoxycarbonylamino)spiro[3.3]heptane-6-carboxylic acid (883 mg, 3.5 mmol) and 3-amino-5-chloro-pyridin-2-ol (500 mg, 3.5 mmol) was added and stirred at 140° C. for 18 h. The mixture was poured into ice-water, adjusted pH to 8 by NH₄OH and extracted with MeOH (50 mL×5). The combined organic layer was dried over Na₂SO₄ and concentrated in vacuo to afford 2-amino-N-(5-chloro-2-hydroxy-3-pyridyl)spiro[3.3]heptane-6-carboxamide as a green solid (63a, 555 mg, 57%), which was used for the next step without further purification. MS obsd. (ESI⁺) [(M+H)⁺]: 282.1.

### Step 2: Preparation of N-[6-[(5-chloro-2-hydroxy-3-pyridyl)carbamoyl]spiro[3.3]heptan-2-yl]-5-methylsulfanyl-furan-2-carboxamide (63b)

To a solution of 5-methylsulfanylfuran-2-carboxylic acid (Int-1, 421.8 mg, 2.67 mmol) in DCM (2 mL) were added 2,4,6-tripropyl-1,3,5,2,4,6-trioxatriphosphinane 2,4,6-trioxide (1.86 mL, 3.13 mmol) and triethylamine (1.09 mL, 7.83 mmol). After being stirred at 25° C. for 30 min, 2-amino-N-(5-chloro-2-hydroxy-3-pyridyl)spiro[3.3]heptane-6-carboxamide (63a, 735 mg, 2.61 mmol) was added, and the reaction was stirred at 25° C. for 1 h. The resulting mixture was diluted with EtOAc (30 mL×3) and washed with water (20 mL×2). The combined organic layer was dried over Na₂SO₄ and concentrated in vacuo. The residue was purified by flash column (eluting with EtOAc/PE=1/1 to 3/2) to give N-[6-[(5-chloro-2-hydroxy-3-pyridyl)carbamoyl]spiro[3.3]heptan-2-yl]-5-methylsulfanyl-furan-2-carboxamide as a light grey solid (63b, 450 mg, 40.9%). MS obsd. (ESI⁺) [(M+H)⁺]: 422.1.

### Step 3: Preparation of N-[6-(6-chlorooxazolo[5,4-b]pyridin-2-yl)spiro[3.3]heptan-2-yl]-5-methylsulfanyl-furan-2-carboxamide (63c)

A mixture of PPh₃ (310.8 mg, 1.19 mmol) and DIAD (0.28 mL, 1.42 mmol) in THF (4 mL) was added N-[6-[(5-chloro-2-hydroxy-3-pyridyl)carbamoyl]spiro[3.3]heptan-2-yl]-5-methylsulfanyl-furan-2-carboxamide (63b, 200 mg, 0.47 mmol). After being stirred at 50° C. for 1 h, the mixture was diluted with EtOAc (30 mL×3) and washed with water (20 mL×2). The combined organic layer was dried over Na₂SO₄ and concentrated under vacuo. The residue was purified by flash column (eluting with EtOAc/PE=1/3 to 1/2) to give N-[6-(6-chlorooxazolo[5,4-b]pyridin-2-yl)spiro[3.3]heptan-2-yl]-5-methylsulfanyl-furan-2-carboxamide as alight yellow oil (63c, 156 mg, 81.5%). MS obsd. (ESI⁺) [(M+H)⁺]: 404.1.

### Step 4: Preparation of N-[6-(6-chlorooxazolo[5,4-b]pyridin-2-yl)spiro[3.3]heptan-2-yl]-5-(methylsulfonimidoyl)furan-2-carboxamide (Example 63)

To a solution of N-[6-(6-chlorooxazolo[5,4-b]pyridin-2-yl)spiro[3.3]heptan-2-yl]-5-methylsulfanyl-furan-2-carboxamide (63c, 154 mg, 0.38 mmol) in MeOH (3 mL) were added ammonium carbonate (55 mg, 0.57 mmol) and iodobenzene diacetate (282.5 mg, 0.88 mmol). After being stirred at 20° C. for 1 h, the mixture was extracted with EtOAc (30 mL×3) and washed with water (20 mL×2). The combined organic layer was dried over Na₂SO₄ and concentrated under vacuo. The residue was purified by preparative HPLC to give N-[6-(6-chlorooxazolo[5,4-b]pyridin-2-yl)spiro[3.3]heptan-2-yl]-5-(methylsulfonimidoyl)furan-2-carboxamide as a white solid (Example 63, 80 mg, 48.2%). MS obsd. (ESI⁺) [(M+H)⁺]: 435.1. ¹H NMR (400 MHz, DMSO-d₆) δ ppm: 8.78 (d, J=7.70 Hz, 1H), 8.34-8.44 (m, 2H), 7.20 (d, J=3.55 Hz, 1H), 7.13 (d, J=3.67 Hz, 1H), 4.86 (d, J=1.10 Hz, 1H), 4.31 (sxt, J=8.12 Hz, 1H), 3.78 (quin, J=8.50 Hz, 1H), 3.20 (d, J=1.22 Hz, 3H), 2.39-2.71 (m, 5H), 2.28-2.38 (m, 1H), 2.20-2.28 (m, 1H), 2.11-2.19 (m, 1H).

### Example 64

### Example 64-a, Example 64-b, Example 64-c, Example 64-d

### N-[6-(6-chlorooxazolo[5,4-b]pyridin-2-yl)spiro[3.3]heptan-2-yl]-5-(cyclopropylsulfonimidoyl)furan-2-carboxamide

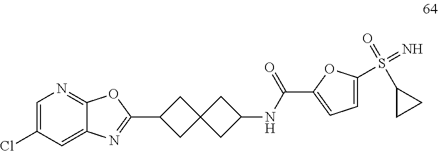 [SMILES: N=S(=O)(c1ccc(C(=O)NC2CC3(C2)CC(c2nc4cc(Cl)cnc4o2)C3)o1)C1CC1]

N-[6-(6-Chlorooxazolo[5,4-b]pyridin-2-yl)spiro[3.3]heptan-2-yl]-5-(cyclopropylsulfonimidoyl)furan-2-carboxamide (Example 64) was prepared in analogy to the procedure described for the preparation of Example 63, by using 5-cyclopropylsulfanylfuran-2-carboxylic acid (Int-5), instead of 5-methylsulfanylfuran-2-carboxylic acid (Int-1). MS obsd. (ESI⁺) [(M+H)⁺]: 461.1. ¹H NMR (400 MHz, CD₃OD) δ ppm: 8.29 (d, J=2.32 Hz, 1H), 8.12 (d, J=2.32 Hz, 1H), 7.19 (q, J=3.67 Hz, 2H), 4.41 (quin, J=8.25 Hz, 1H), 3.78 (quin, J=8.44 Hz, 1H), 2.76-2.89 (m, 1H), 2.42-2.74 (m, 6H), 2.27 (dd, J=9.05, 11.13 Hz, 1H), 2.20 (dd, J=8.99, 11.43 Hz, 1H), 1.29-1.37 (m, 1H), 1.21-1.29 (m, 1H), 1.11-1.21 (m, 1H), 1.01-1.11 (m, 1H).

The four diastereomers (Example 64-a, Example 64-b, Example 64-c, Example 64-d) were obtained through chiral separation of N-[6-(6-chlorooxazolo[5,4-b]pyridin-2-yl)spiro[3.3]heptan-2-yl]-5-(cyclopropylsulfonimidoyl)furan-2-carboxamide (Example 64).

### (Rₐ)—N-[6-(6-chlorooxazolo[5,4-b]pyridin-2-yl)spiro[3.3]heptan-2-yl]-5-[(S)-cyclopropylsulfonimidoyl]furan-2-carboxamide

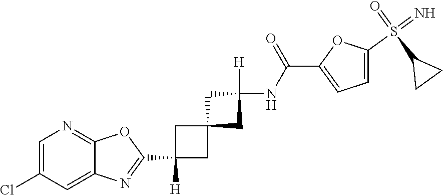

### (Rₐ)—N-[6-(6-chlorooxazolo[5,4-b]pyridin-2-yl)spiro[3.3]heptan-2-yl]-5-[(R)-cyclopropylsulfonimidoyl]furan-2-carboxamide

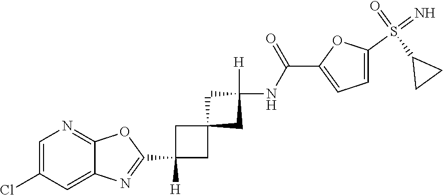

### (Sₐ)—N-[6-(6-chlorooxazolo[5,4-b]pyridin-2-yl)spiro[3.3]heptan-2-yl]-5-[(S)-cyclopropylsulfonimidoyl]furan-2-carboxamide

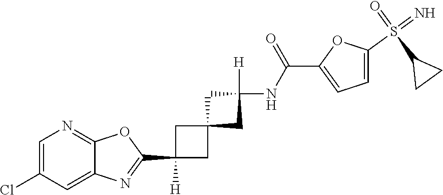

### (Sₐ)—N-[6-(6-chlorooxazolo[5,4-b]pyridin-2-yl)spiro[3.3]heptan-2-yl]-5-[(R)-cyclopropylsulfonimidoyl]furan-2-carboxamide

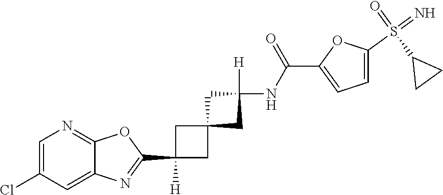

Example 64-a: Chiral HPLC analysis: RT=20.728 min [Instrument: Shimadzu LC-20A; Column: Daicel IC-3 4.6 mm×150 mmL, 3 μm; Mobile Phase: Hex/EtOH (0.1% DEA)]. MS obsd. (ESI⁺) [(M+H)⁺]: 461.1. ¹H NMR (400 MHz, CD₃OD) δ ppm: 8.29 (d, J=2.32 Hz, 1H), 8.12 (d, J=2.20 Hz, 1H), 7.19 (q, J=3.67 Hz, 2H), 4.41 (quin, J=8.28 Hz, 1H), 3.78 (quin, J=8.25 Hz, 1H), 2.80-2.89 (m, 1H), 2.63-2.73 (m, 3H), 2.41-2.63 (m, 3H), 2.15-2.32 (m, 2H), 1.29-1.39 (m, 1H), 1.21-1.29 (m, 1H), 1.21-1.29 (m, 1H), 1.12-1.21 (m, 1H), 1.01-1.11 (m, 1H).

Example 64-b: Chiral HPLC analysis: RT=21.841 min [Instrument: Shimadzu LC-20A; Column: Daicel IC-3 4.6 mm×150 mmL, 3 μm; Mobile Phase: Hex/EtOH (0.1% DEA)]. MS obsd. (ESI⁺) [(M+H)⁺]: 461.1. ¹H NMR (400 MHz, CD₃OD) δ ppm: 8.28-8.30 (m, 1H), 8.10-8.14 (m, 1H), 7.18 (q, J=3.67 Hz, 2H), 4.41 (quin, J=8.22 Hz, 1H), 3.78 (quin, J=8.41 Hz, 1H), 2.79-2.88 (m, 1H), 2.63-2.74 (m, 3H), 2.40-2.63 (m, 3H), 2.12-2.33 (m, 2H), 1.29-1.38 (m, 1H), 1.21-1.29 (m, 1H), 1.12-1.20 (m, 1H), 0.96-1.11 (m, 1H).

Example 64-c: Chiral HPLC analysis: RT=19.724 min [Instrument: Shimadzu LC-20A; Column: Daicel IC-3 4.6 mm×150 mmL, 3 μm; Mobile Phase: Hex/EtOH (0.1% DEA)]. MS obsd. (ESI⁺) [(M+H)⁺]: 461.1. ¹H NMR (400 MHz, CD₃OD) δ ppm: 8.29 (d, J=2.20 Hz, 1H), 8.12 (d, J=2.32 Hz, 1H), 7.19 (q, J=3.67 Hz, 2H), 4.41 (quin, J=8.25 Hz, 1H), 3.78 (quin, J=8.47 Hz, 1H), 2.80-2.89 (m, 1H), 2.63-2.74 (m, 3H), 2.42-2.63 (m, 3H), 2.14-2.33 (m, 2H), 1.29-1.37 (m, 1H), 1.21-1.29 (m, 1H), 1.13-1.21 (m, 1H), 1.02-1.11 (m, 1H).

Example 64-d: Chiral HPLC analysis: RT=18.550 min [Instrument: Shimadzu LC-20A; Column: Daicel IC-3 4.6 mm×150 mmL, 3 μm; Mobile Phase: Hex/EtOH (0.1% DEA)]. MS obsd. (ESI⁺) [(M+H)⁺]: 461.1. ¹H NMR (400 MHz, CD₃OD) δ ppm: 8.29 (d, J=2.20 Hz, 1H), 8.11 (d, J=2.32 Hz, 1H), 7.18 (q, J=3.55 Hz, 2H), 4.41 (quin, J=8.25 Hz, 1H), 3.78 (quin, J=8.47 Hz, 1H), 2.84 (tt, J=4.72, 7.87 Hz, 1H), 2.63-2.73 (m, 3H), 2.41-2.62 (m, 3H), 2.15-2.32 (m, 2H), 1.29-1.38 (m, 1H), 1.21-1.29 (m, 1H), 1.12-1.21 (m, 1H), 1.01-1.12 (m, 1H).

### Example 65

### N-[6-(6-chlorooxazolo[4,5-c]pyridin-2-yl)spiro[3.3]heptan-2-yl]-5-(cyclopropylsulfonimidoyl)furan-2-carboxamide

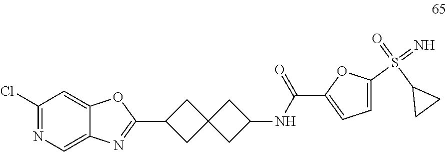 [SMILES: N=S(=O)(c1ccc(C(=O)NC2CC3(C2)CC(c2nc4cnc(Cl)cc4o2)C3)o1)C1CC1]

The title compound was prepared in analogy to the procedure described for the preparation of Example 63, by using 2-amino-N-(4,6-dichloro-3-pyridyl)spiro[3.3]heptane-6-carboxamide (51a) instead of 2-amino-N-(5-chloro-2-hydroxy-3-pyridyl)spiro[3.3]heptane-6-carboxamide (63a). The product was purified by preparative HPLC to afford Example 65 as a white solid. MS obsd. (ESI⁺) [(M+H)⁺]: 461.0. ¹H NMR (400 MHz, DMSO-d₆) δ ppm: 8.81 (d, J=0.61 Hz, 1H), 8.76 (d, J=7.58 Hz, 1H), 8.03 (d, J=0.61 Hz, 1H), 7.22 (d, J=3.55 Hz, 1H), 7.10 (d, J=3.55 Hz, 1H), 4.93 (s, 1H), 4.24-4.36 (m, 1H), 3.80 (quin, J=8.41 Hz, 1H), 2.73-2.81 (m, 1H), 2.41-2.69 (m, 5H), 2.28-2.38 (m, 1H), 2.10-2.28 (m, 2H), 1.13-1.21 (m, 1H), 0.94-1.12 (m, 3H).

### Example 66

### N-[6-(5-chloro-1,3-benzoxazol-2-yl)spiro[3.3]heptan-2-yl]-5-(pyrrolidin-1-ylsulfonimidoyl)furan-2-carboxamide

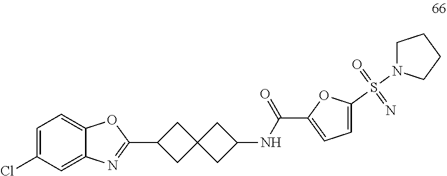

The title compound was prepared according to the following scheme:

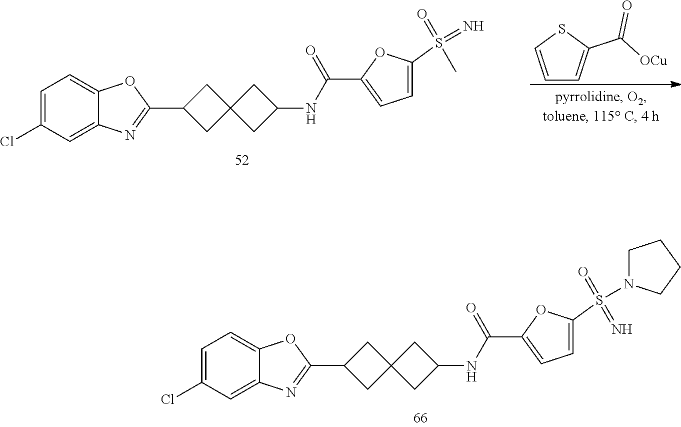

A sealed tube (25 mL) equipped with a stir bar was charged with N-[6-(5-chloro-1,3-benzoxazol-2-yl)spiro[3.3]heptan-2-yl]-5-(methylsulfonimidoyl)furan-2-carboxamide (Example 52, 54 mg, 0.12 mmol), pyrrolidine (71 mg, 1 mmol) and thiophene-2-carbonyloxycopper (4.8 mg, 0.03 mmol), followed by addition of dry toluene (2 mL). The tube was flushed with dioxygen for 1 min and then sealed with a Teflon lined cap. The reaction mixture was stirred at 115° C. for 4 h. After cooling to ambient temperature, the mixture was diluted with DCM (5 mL×2) and filtered. The combined organic layer was concentrated. The residue was purified by preparative HPLC to give N-[6-(5-chloro-1,3-benzoxazol-2-yl)spiro[3.3]heptan-2-yl]-5-(pyrrolidin-1-ylsulfonimidoyl)furan-2-carboxamide as an off-white solid (Example 66, 6 mg, 9.5%). MS obsd. (ESI⁺) [(M+H)⁺]: 489.1. ¹H NMR (400 MHz, DMSO-d₆) δ ppm: 8.69 (d, J=7.58 Hz, 1H), 7.80 (d, J=2.08 Hz, 1H), 7.72 (d, J=8.68 Hz, 1H), 7.40 (dd, J=2.08, 8.68 Hz, 1H), 7.22 (d, J=3.67 Hz, 1H), 7.07 (d, J=3.55 Hz, 1H), 5.01 (s, 1H), 4.30 (sxt, J=8.22 Hz, 1H), 3.74 (quin, J=8.44 Hz, 1H), 3.15-3.26 (m, 4H), 2.37-2.65 (m, 5H), 2.27-2.35 (m, 1H), 2.20-2.27 (m, 1H), 2.11-2.19 (m, 1H), 1.66 (td, J=3.27, 6.42 Hz, 4H).

### Example 67

### N-[6-(5-chloro-1,3-benzoxazol-2-yl)spiro[3.3]heptan-2-yl]-5-(morpholinosulfonimidoyl)furan-2-carboxamide

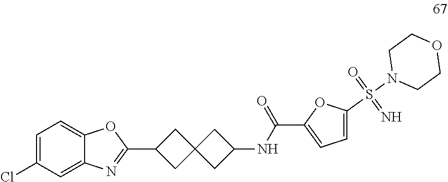 [SMILES: N=S(=O)(c1ccc(C(=O)NC2CC3(C2)CC(c2nc4cc(Cl)ccc4o2)C3)o1)N1CCOCC1]

The title compound was prepared in analogy to the procedure described for the preparation of Example 66, by using morpholine instead of pyrrolidine. The product was purified by preparative HPLC to afford Example 67 as a light yellow solid. MS obsd. (ESI⁺) [(M+H)⁺]: 505.0. ¹H NMR (400 MHz, DMSO-d₆) δ ppm: 8.70 (d, J=7.46 Hz, 1H), 7.80 (d, J=2.08 Hz, 1H), 7.72 (d, J=8.68 Hz, 1H), 7.40 (dd, J=2.14, 8.62 Hz, 1H), 7.25 (d, J=3.67 Hz, 1H), 7.09 (d, J=3.67 Hz, 1H), 5.22 (s, 1H), 4.24-4.35 (m, 1H), 3.74 (quin, J=8.53 Hz, 1H), 3.61 (t, J=4.65 Hz, 4H), 3.02 (q, J=4.36 Hz, 4H), 2.45-2.65 (m, 5H), 2.27-2.36 (m, 1H), 2.20-2.26 (m, 1H), 2.11-2.19 (m, 1H).

### Example 68

### (Sₐ)—N-[6-(5-chloro-1,3-benzoxazol-2-yl)spiro[3.3]heptan-2-yl]-5-(propanoylsulfamoyl)furan-2-carboxamide, or (Rₐ)—N-[6-(5-chloro-1,3-benzoxazol-2-yl)spiro[3.3]heptan-2-yl]-5-(propanoylsulfamoyl)furan-2-carboxamide

The title compound was prepared according to the following scheme:

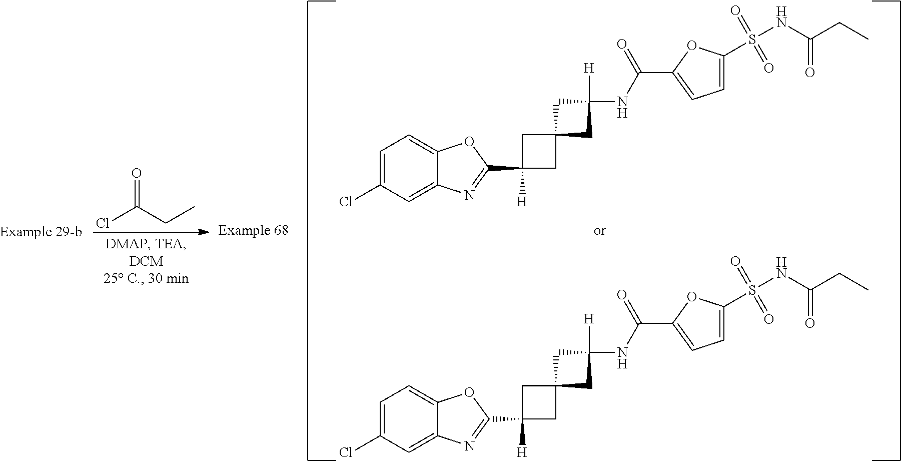

To a solution of Example 29-b [100 mg, 0.23 mmol, (Sₐ)—N-[6-(5-chloro-1,3-benzoxazol-2-yl)spiro[3.3]heptan-2-yl]-5-(propanoylsulfamoyl)furan-2-carboxamide, or (Rₐ)—N-[6-(5-chloro-1,3-benzoxazol-2-yl)spiro[3.3]heptan-2-yl]-5-(propanoylsulfamoyl)furan-2-carboxamide)], DMAP (1 mg, 8.19 μmol) and triethylamine (46.4 mg, 0.46 mmol) in DCM (5 mL) was added propionyl chloride (31.8 mg, 0.34 μmol) dropwise at 0° C. Then the reaction was stirred at 25° C. for 30 min. The reaction solution was diluted with DCM (15 mL), washed with HCl (1 M, 10 mL). The organic layer was dried over Na₂SO₄ and purified by flash column (eluting with MeOH/DCM=1/50 to 1/30) to give Example 68 as a white solid [75 mg, 65.8%, (Sₐ)—N-[6-(5-chloro-1,3-benzoxazol-2-yl)spiro[3.3]heptan-2-yl]-5-(propanoylsulfamoyl)furan-2-carboxamide, or (Rₐ)—N-[6-(5-chloro-1,3-benzoxazol-2-yl)spiro[3.3]heptan-2-yl]-5-(propanoylsulfamoyl)furan-2-carboxamide]. (MS obsd. (ESI⁺) [(M+H)⁺]: 492.6. ¹H NMR (400 MHz, DMSO-d₆) δ ppm: 12.44 (br s, 1H), 8.80 (d, J=7.3 Hz, 1H), 7.80 (d, J=2.0 Hz, 1H), 7.71 (d, J=8.7 Hz, 1H), 7.40 (dd, J=8.7, 2.1 Hz, 1H), 7.35 (d, J=3.7 Hz, 1H), 7.29 (d, J=3.8 Hz, 1H), 4.27 (sxt, J=8.0 Hz, 1H), 3.74 (quin, J=8.5 Hz, 1H), 2.52-2.68 (m, 3H), 2.40-2.48 (m, 2H), 2.09-2.35 (m, 5H), 0.92 (t, J=7.4 Hz, 3H).

### Example 69

### (Sₐ)—N-(butanoylsulfamoyl)-N-[6-(5-chloro-1,3-benzoxazol-2-yl)spiro[3.3]heptan-2-yl]furan-2-carboxamide, or (Rₐ)—N-(butanoylsulfamoyl)-N-[6-(5-chloro-1,3-benzoxazol-2-yl)spiro[3.3]heptan-2-yl]furan-2-carboxamide

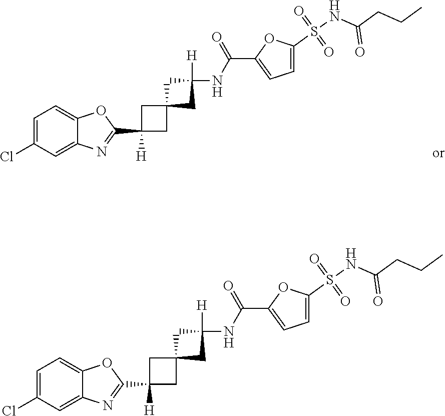

The title compound was prepared in analogy to the procedure described for the preparation of Example 68, by using butyryl chloride instead of propionyl chloride. The product was purified by flash column to afford Example 69 as a white solid. MS obsd. (ESI⁺) [(M+H)⁺]: 506.0. ¹H NMR (400 MHz, DMSO-d₆) δ ppm: 12.44 (br s, 1H), 8.80 (d, J=7.5 Hz, 1H), 7.80 (d, J=2.1 Hz, 1H), 7.71 (d, J=8.7 Hz, 1H), 7.40 (dd, J=8.7, 2.2 Hz, 1H), 7.36 (d, J=3.7 Hz, 1H), 7.29 (d, J=3.7 Hz, 1H), 4.27 (sxt, J=8.1 Hz, 1H), 3.74 (quin, J=8.5 Hz, 1H), 2.53-2.67 (m, 3H), 2.40-2.48 (m, 2H), 2.09-2.35 (m, 5H), 1.39-1.52 (m, 2H), 0.79 (t, J=7.4 Hz, 3H).

### Example 70

### (Sₐ)—N-[6-(5-chloro-1,3-benzoxazol-2-yl)spiro[3.3]heptan-2-yl]-5-(2-methylpropanoylsulfamoyl)furan-2-carboxamide, or (Rₐ)—N-[6-(5-chloro-1,3-benzoxazol-2-yl)spiro[3.3]heptan-2-yl]-5-(2-methylpropanoylsulfamoyl)furan-2-carboxamide

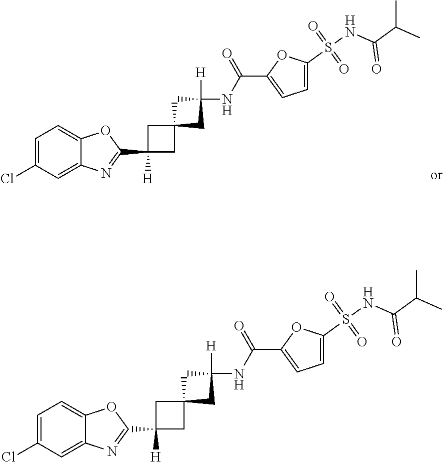

The title compound was prepared in analogy to the procedure described for the preparation of Example 68, by using isobutyryl chloride instead of propionyl chloride. The product was purified by flash column to afford Example 70 as a white solid. MS obsd. (ESI⁺) [(M+H)⁺]: 506.3. ¹H NMR (400 MHz, DMSO-d₆) δ ppm: 12.42 (br s, 1H), 8.80 (br d, J=7.2 Hz, 1H), 7.80 (s, 1H), 7.71 (d, J=8.6 Hz, 1H), 7.33-7.43 (m, 2H), 7.30 (br d, J=3.3 Hz, 1H), 4.22-4.33 (m, 1H), 3.74 (quin, J=8.4 Hz, 1H), 2.56-2.68 (m, 3H), 2.39-2.47 (m, 3H), 2.32 (br s, 1H), 2.05-2.27 (m, 2H), 0.98 (br d, J=6.8 Hz, 6H).

### Example 71

### (Sₐ)—N-[6-(5-chloro-1,3-benzoxazol-2-yl)spiro[3.3]heptan-2-yl]-5-(cyclopropanecarbonylsulfamoyl)furan-2-carboxamide, or (Rₐ)—N-[6-(5-chloro-1,3-benzoxazol-2-yl)spiro[3.3]heptan-2-yl]-5-(cyclopropanecarbonylsulfamoyl)furan-2-carboxamide

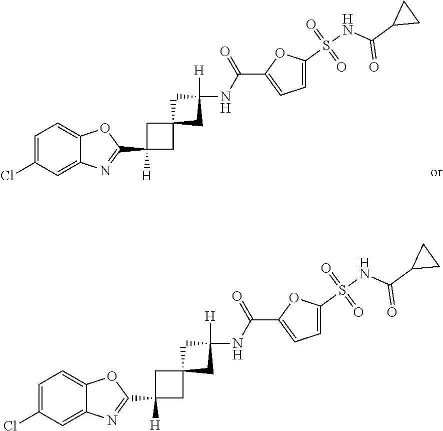

The title compound was prepared in analogy to the procedure described for the preparation of Example 68, by using cyclopropanecarbonyl chloride instead of propionyl chloride. The product was purified by flash column to afford Example 71 as a white solid. MS obsd. (ESI⁺) [(M+H)⁺]: 504.2. ¹H NMR (400 MHz, DMSO-d₆) δ ppm: 12.77 (br s, 1H), 8.82 (d, J=7.5 Hz, 1H), 7.80 (d, J=2.0 Hz, 1H), 7.71 (d, J=8.7 Hz, 1H), 7.27-7.42 (m, 3H), 4.28 (sxt, J=7.9 Hz, 1H), 3.74 (quin, J=8.5 Hz, 1H), 2.53-2.67 (m, 3H), 2.29-2.47 (m, 3H), 2.06-2.26 (m, 2H), 1.70-1.80 (m, 1H), 0.72-0.92 (m, 4H).

### Example 72

### (Sₐ)—N-[6-(5-chloro-1,3-benzoxazol-2-yl)spiro[3.3]heptan-2-yl]-5-(cyclobutanecarbonylsulfamoyl)furan-2-carboxamide, or (Rₐ)—N-[6-(5-chloro-1,3-benzoxazol-2-yl)spiro[3.3]heptan-2-yl]-5-(cyclobutanecarbonylsulfamoyl)furan-2-carboxamide

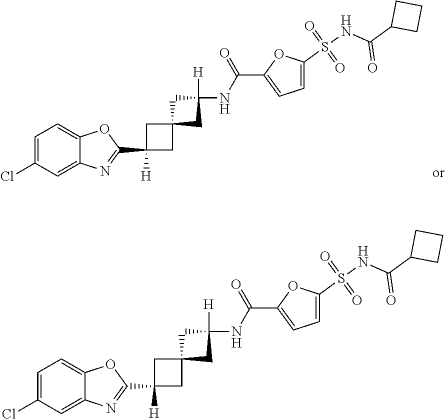

The title compound was prepared in analogy to the procedure described for the preparation of Example 68, by using cyclobutanecarbonyl chloride instead of propionyl chloride. The product was purified by flash column to afford Example 72 as a white solid. MS obsd. (ESI⁺) [(M+H)⁺]: 518.2. ¹H NMR (400 MHz, DMSO-d₆) δ ppm: 12.34 (br s, 1H), 8.80 (d, J=7.5 Hz, 1H), 7.80 (d, J=2.1 Hz, 1H), 7.71 (d, J=8.7 Hz, 1H), 7.34-7.42 (m, 2H), 7.29 (d, J=3.8 Hz, 1H), 4.27 (sxt, J=8.0 Hz, 1H), 3.74 (quin, J=8.5 Hz, 1H), 3.10-3.28 (m, 1H), 2.52-2.68 (m, 3H), 2.29-2.48 (m, 3H), 1.99-2.23 (m, 6H), 1.81-1.95 (m, 1H), 1.66-1.79 (m, 1H).

### Example 73

### (Sₐ)—N-[6-(5-chloro-1,3-benzoxazol-2-yl)spiro[3.3]heptan-2-yl]-5-[(2-methoxyacetyl)sulfamoyl]furan-2-carboxamide, or (Rₐ)—N-[6-(5-chloro-1,3-benzoxazol-2-yl)spiro[3.3]heptan-2-yl]-5-[(2-methoxyacetyl)sulfamoyl]furan-2-carboxamide

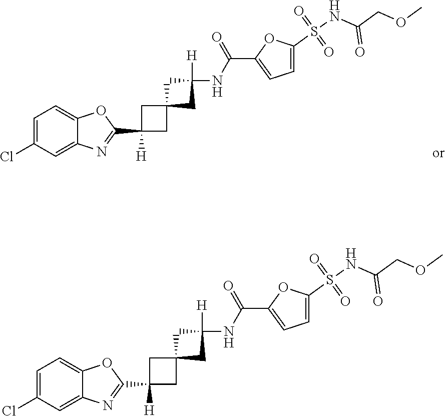

The title compound was prepared in analogy to the procedure described for the preparation of Example 68, by using 2-methoxyacetyl chloride instead of propionyl chloride. The product was purified by flash column to afford Example 73 as a white solid. MS obsd. (ESI⁺) [(M+H)⁺]: 508.4. ¹H NMR (400 MHz, DMSO-d₆) δ ppm: 8.62 (br d, J=7.6 Hz, 1H), 7.80 (d, J=2.1 Hz, 1H), 7.71 (d, J=8.7 Hz, 1H), 7.40 (dd, J=8.7, 2.2 Hz, 1H), 7.13 (d, J=3.4 Hz, 1H), 6.92 (br s, 1H), 4.28 (sxt, J=8.1 Hz, 1H), 3.68-3.78 (m, 3H), 3.21 (s, 3H), 2.52-2.68 (m, 3H), 2.38-2.48 (m, 2H), 2.10-2.34 (m, 3H).

### Example 74

### (Sₐ)-ethyl N-[[5-[[6-(5-chloro-1,3-benzoxazol-2-yl)spiro[3.3]heptan-2-yl]carbamoyl]-2-furyl]sulfonyl]carbamate, or (Rₐ)-ethyl N-[[5-[[6-(5-chloro-1,3-benzoxazol-2-yl)spiro[3.3]heptan-2-yl]carbamoyl]-2-furyl]sulfonyl]carbamate

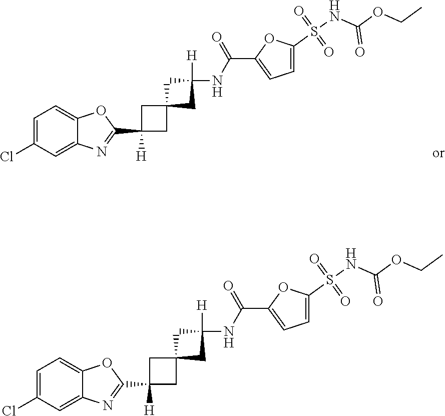

The title compound was prepared in analogy to the procedure described for the preparation of Example 68, by using ethyl carbonochloridate instead of propionyl chloride. The product was purified by flash column to afford Example 74 as a white solid. MS obsd. (ESI⁺) [(M+H)⁺]: 508.4. ¹H NMR (400 MHz, DMSO-d₆) δ ppm: 8.82 (d, J=7.3 Hz, 1H), 7.80 (d, J=2.1 Hz, 1H), 7.71 (d, J=8.7 Hz, 1H), 7.28-7.42 (m, 3H), 4.23-4.33 (m, 1H), 4.06 (q, J=7.1 Hz, 2H), 3.74 (quin, J=8.5 Hz, 1H), 2.52-2.68 (m, 3H), 2.29-2.48 (m, 3H), 2.09-2.25 (m, 2H), 1.14 (t, J=7.1 Hz, 3H).

### Example 75

### (Sₐ)-methyl 4-[[5-[[6-(5-chloro-1,3-benzoxazol-2-yl)spiro[3.3]heptan-2-yl]carbamoyl]-2-furyl]sulfonylamino]-4-oxo-butanoate, or (Rₐ)-methyl 4-[[5-[[6-(5-chloro-1,3-benzoxazol-2-yl)spiro[3.3]heptan-2-yl]carbamoyl]-2-furyl]sulfonylamino]-4-oxo-butanoate

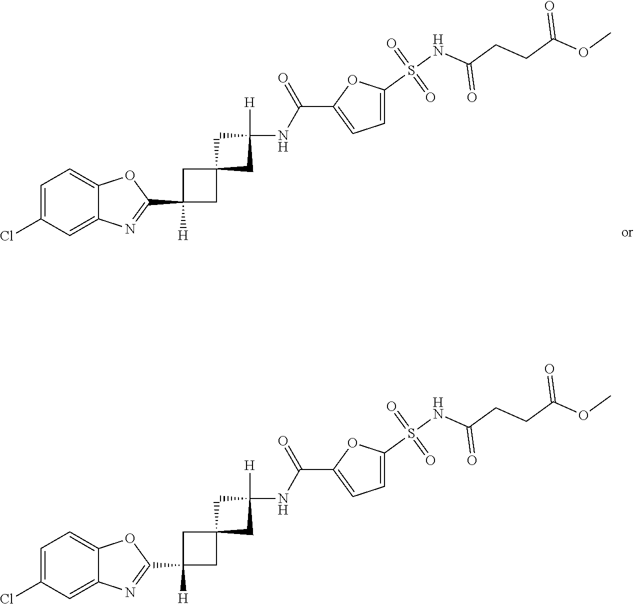

The title compound was prepared in analogy to the procedure described for the preparation of Example 68, by using 4-methoxy-4-oxobutanoic acid instead of propionyl chloride. The product was purified by flash column to afford Example 75 as a white solid. MS obsd. (ESI⁺) [(M+H)⁺]: 550.5. ¹H NMR (400 MHz, DMSO-d₆) δ ppm: 12.59 (br s, 1H), 8.78 (d, J=7.5 Hz, 1H), 7.80 (d, J=2.1 Hz, 1H), 7.71 (d, J=8.7 Hz, 1H), 7.40 (dd, J=8.7, 2.2 Hz, 1H), 7.32 (d, J=3.7 Hz, 1H), 7.27 (d, J=3.8 Hz, 1H), 4.28 (sxt, J=8.1 Hz, 1H), 3.74 (quin, J=8.5 Hz, 1H), 3.53 (s, 3H), 2.53-2.68 (m, 5H), 2.29-2.48 (m, 5H), 2.08-2.24 (m, 2H).

### Example 76

### (Sₐ)-5-(bicyclo[1.1.1]pentane-1-carbonylsulfamoyl)-N-[6-(5-chloro-1,3-benzoxazol-2-yl)spiro[3.3]heptan-2-yl]furan-2-carboxamide, or (Rₐ)-5-(bicyclo[1.1.1]pentane-1-carbonylsulfamoyl)-N-[6-(5-chloro-1,3-benzoxazol-2-yl)spiro[3.3]heptan-2-yl]furan-2-carboxamide

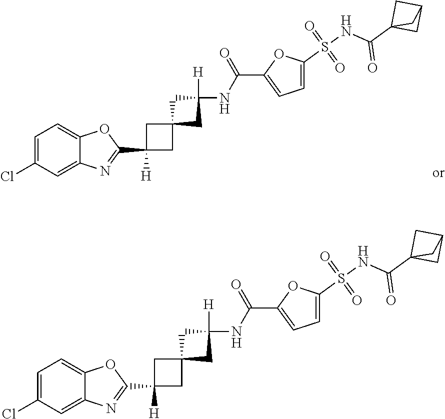

The title compound was prepared in analogy to the procedure described for the preparation of Example 68, by using bicyclo[1.1.1]pentane-1-carboxylic acid instead of propionyl chloride. The product was purified by flash column to afford Example 76 as a white solid. MS obsd. (ESI⁺) [(M+H)⁺]: 530.1. ¹H NMR (DMSO-d₆, 400 MHz) δ: 12.51 (br s, 1H), 8.81 (d, J=7.3 Hz, 1H), 7.80 (d, J=2.1 Hz, 1H), 7.71 (d, J=8.7 Hz, 1H), 7.29-7.42 (m, 3H), 4.27 (sxt, J=8.1 Hz, 1H), 3.74 (quin, J=8.5 Hz, 1H), 2.52-2.68 (m, 3H), 2.41-2.49 (m, 2H), 2.40 (s, 1H), 2.30-2.36 (m, 1H), 2.06-2.23 (m, 2H), 2.03 (s, 6H).

### Example 77

### (Sₐ)—N-[6-(5-chloro-1,3-benzoxazol-2-yl)spiro[3.3]heptan-2-yl]-5-(tetrahydrofuran-3-carbonylsulfamoyl)furan-2-carboxamide, or (Rₐ)—N-[6-(5-chloro-1,3-benzoxazol-2-yl)spiro[3.3]heptan-2-yl]-5-(tetrahydrofuran-3-carbonylsulfamoyl)furan-2-carboxamide

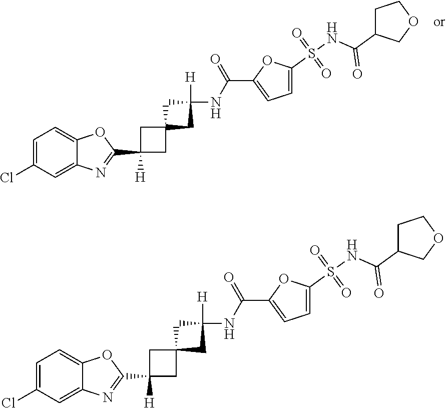

The title compound was prepared in analogy to the procedure described for the preparation of Example 68, by using tetrahydrofuran-3-carboxylic acid instead of propionyl chloride. The product was purified by flash column to afford Example 77 as a white solid. MS obsd. (ESI⁺) [(M+H)⁺]: 534.5. ¹H NMR (400 MHz, DMSO-d₆) δ ppm: 12.35-12.92 (m, 1H), 8.72 (br d, J=7.5 Hz, 1H), 7.80 (d, J=2.1 Hz, 1H), 7.71 (d, J=8.7 Hz, 1H), 7.40 (dd, J=8.6, 2.1 Hz, 1H), 7.13-7.27 (m, 2H), 4.23-4.33 (m, 1H), 3.56-3.82 (m, 5H), 2.95-3.06 (m, 1H), 2.52-2.63 (m, 3H), 2.39-2.48 (m, 2H), 2.09-2.35 (m, 3H), 1.86-2.04 (m, 2H).

### Example 78

### (Sₐ)—N-[6-(5-chloro-1,3-benzoxazol-2-yl)spiro[3.3]heptan-2-yl]-5-[[2-(2-methoxyethoxy)acetyl]sulfamoyl]furan-2-carboxamide, or (Rₐ)—N-[6-(5-chloro-1,3-benzoxazol-2-yl)spiro[3.3]heptan-2-yl]-5-[[2-(2-methoxyethoxy)acetyl]sulfamoyl]furan-2-carboxamide

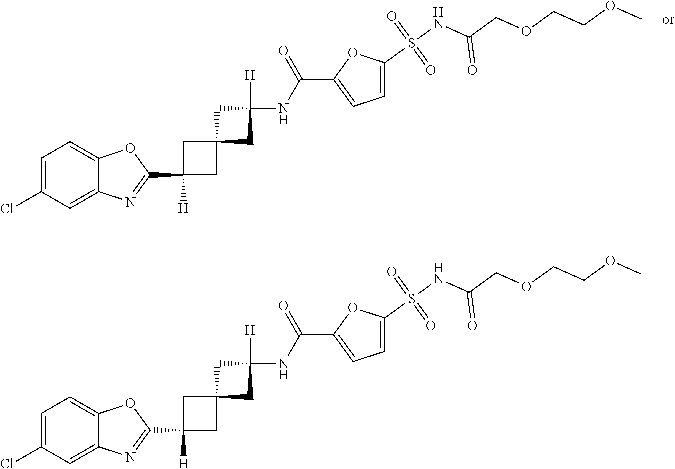

The title compound was prepared in analogy to the procedure described for the preparation of Example 68, by using 2-(2-methoxyethoxy)acetic acid instead of propionyl chloride. The product was purified by flash column to afford Example 78 as an off-white solid. MS obsd. (ESI⁺) [(M+H)⁺]: 552.2. ¹H NMR (400 MHz, DMSO-d₆) δ ppm: 8.71 (d, J=7.6 Hz, 1H), 7.80 (d, J=2.1 Hz, 1H), 7.71 (d, J=8.7 Hz, 1H), 7.40 (dd, J=8.7, 2.2 Hz, 1H), 7.20 (d, J=3.7 Hz, 1H), 7.10-7.16 (m, 1H), 4.28 (sxt, J=8.1 Hz, 1H), 3.93 (s, 2H), 3.65-3.83 (m, 1H), 3.51-3.54 (m, 2H), 3.40-3.42 (m, 2H), 3.22 (s, 3H), 2.52-2.68 (m, 3H), 2.39-2.49 (m, 2H), 2.09-2.34 (m, 3H).

### Example 79

### (Sₐ)—N-[6-(5-chloro-1,3-benzoxazol-2-yl)spiro[3.3]heptan-2-yl]-5-[[2-[2-(2-methoxyethoxy)ethoxy]acetyl]sulfamoyl]furan-2-carboxamide, or (Rₐ)—N-[6-(5-chloro-1,3-benzoxazol-2-yl)spiro[3.3]heptan-2-yl]-5-[[2-[2-(2-methoxyethoxy)ethoxy]acetyl]sulfamoyl]furan-2-carboxamide

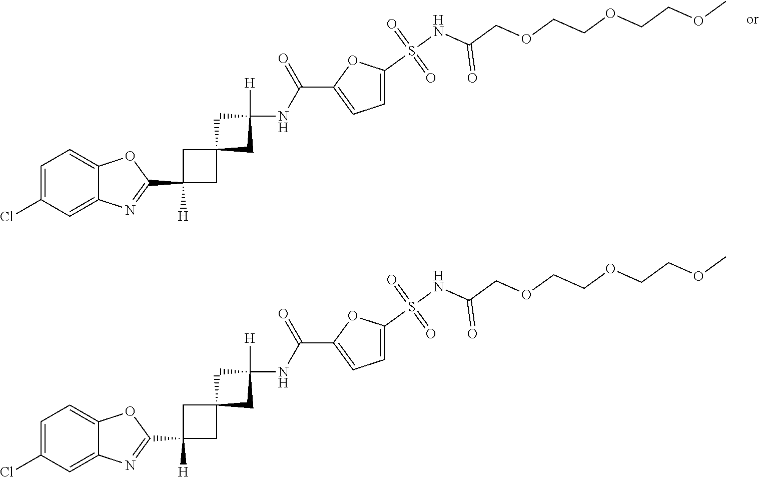

The title compound was prepared in analogy to the procedure described for the preparation of Example 68, by using 2-(2-(2-methoxyethoxy)ethoxy)acetic acid instead of propionyl chloride. The product was purified by flash column to afford Example 79 as a white solid. MS obsd. (ESI⁺) [(M+H)⁺]: 596.2. ¹H NMR (400 MHz, DMSO-d₆) δ ppm: 8.81 (d, J=7.5 Hz, 1H), 7.79 (d, J=2.1 Hz, 1H), 7.71 (d, J=8.7 Hz, 1H), 7.37-7.42 (m, 2H), 7.30 (d, J=3.7 Hz, 1H), 4.11-4.34 (m, 1H), 4.07 (s, 2H), 3.74 (quin, J=8.5 Hz, 1H), 3.52-3.58 (m, 2H), 3.47-3.51 (m, 4H), 3.40-3.43 (m, 2H), 3.23 (s, 3H), 2.53-2.68 (m, 3H), 2.29-2.49 (m, 3H), 1.98-2.25 (m, 2H).

### Example 80

### 5-(acetylsulfamoyl)-N-[6-(5-chloro-1,3-benzoxazol-2-yl)spiro[3.3]heptan-2-yl]furan-2-carboxamide

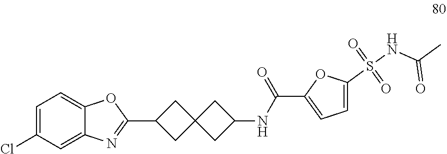 [SMILES: CC(=O)NS(=O)(=O)c1ccc(C(=O)NC2CC3(C2)CC(c2nc4cc(Cl)ccc4o2)C3)o1]

The title compound was prepared in analogy to the procedure described for the preparation of Example 68, by using N-(6-(5-chlorobenzo[d]oxazol-2-yl)spiro[3.3]heptan-2-yl)-5-sulfamoylfuran-2-carboxamide (Example 29) instead of Example 29-b and acetyl chloride instead of propionyl chloride. The product was purified by flash column to afford Example 80 as a white solid. MS obsd. (ESI⁺) [(M+H)⁺]: 478.2. ¹H NMR (400 MHz, DMSO-d₆) δ ppm: 12.51 (br s, 1H), 8.82 (d, J=7.3 Hz, 1H), 7.80 (d, J=2.0 Hz, 1H), 7.72 (d, J=8.7 Hz, 1H), 7.35-7.42 (m, 2H), 7.29 (d, J=3.7 Hz, 1H), 4.28 (sxt, J=8.1 Hz, 1H), 3.74 (quin, J=8.5 Hz, 1H), 2.53-2.68 (m, 3H), 2.29-2.49 (m, 3H), 2.08-2.25 (m, 2H), 1.99 (s, 3H).

### Example 81

### N-[6-(5-chloro-1,3-benzoxazol-2-yl)spiro[3.3]heptan-2-yl]-5-(cyclopentanecarbonylsulfamoyl)furan-2-carboxamide

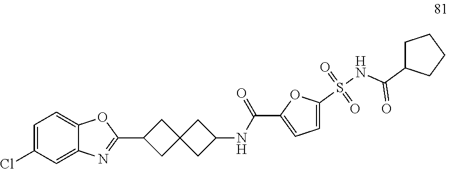 [SMILES: O=C(NC1CC2(C1)CC(c1nc3cc(Cl)ccc3o1)C2)c1ccc(S(=O)(=O)NC(=O)C2CCCC2)o1]

The title compound was prepared in analogy to the procedure described for the preparation of Example 80, by using cyclopentanecarboxylic acid instead of propionyl chloride. The product was purified by flash column to afford Example 81 as a white solid. MS obsd. (ESI⁺) [(M+H)⁺]: 532.4. ¹H NMR (400 MHz, CD₃OD) δ ppm: 7.54 (d, J=2.1 Hz, 1H), 7.46 (d, J=8.7 Hz, 1H), 7.25 (dd, J=2.1, 8.7 Hz, 1H), 7.03 (d, J=3.5 Hz, 1H), 6.97 (d, J=3.7 Hz, 1H), 4.2-4.3 (m, 1H), 3.6-3.7 (m, 1H), 2.3-2.6 (m, 7H), 2.0-2.2 (m, 2H), 1.7-1.8 (m, 2H), 1.4-1.6 (m, 6H).

### Example 82

### N-[6-(5-chloro-1,3-benzoxazol-2-yl)spiro[3.3]heptan-2-yl]-5-(cyclohexanecarbonylsulfamoyl)furan-2-carboxamide

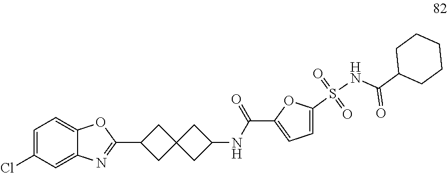 [SMILES: O=C(NC1CC2(C1)CC(c1nc3cc(Cl)ccc3o1)C2)c1ccc(S(=O)(=O)NC(=O)C2CCCCC2)o1]

The title compound was prepared in analogy to the procedure described for the preparation of Example 80, by using cyclohexanecarboxylic acid instead of propionyl chloride. The product was purified by flash column to afford Example 82 as a white solid. MS obsd. (ESI⁺) [(M+H)⁺]: 546.5. ¹H NMR (400 MHz, CD₃OD) δ ppm: 7.54 (d, J=2.0 Hz, 1H), 7.46 (d, J=8.6 Hz, 1H), 7.25 (dd, J=2.1, 8.6 Hz, 1H), 7.01 (d, J=3.5 Hz, 1H), 6.92 (d, J=3.5 Hz, 1H), 4.29 (t, J=8.3 Hz, 1H), 3.65 (t, J=8.5 Hz, 1H), 2.3-2.6 (m, 6H), 2.0-2.2 (m, 3H), 1.6-1.8 (m, 4H), 1.2-1.3 (m, 6H).

### Example 83

### N-[6-(5-chloro-1,3-benzoxazol-2-yl)spiro[3.3]heptan-2-yl]-5-[(1,1-dioxothietane-3-carbonyl)sulfamoyl]furan-2-carboxamide

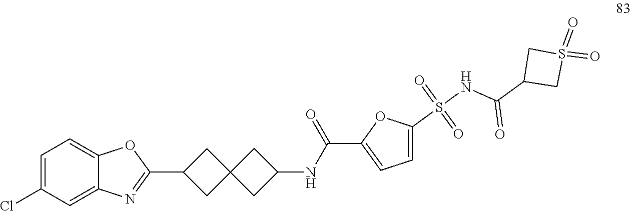 [SMILES: O=C(NC1CC2(C1)CC(c1nc3cc(Cl)ccc3o1)C2)c1ccc(S(=O)(=O)NC(=O)C2CS(=O)(=O)C2)o1]

The title compound was prepared in analogy to the procedure described for the preparation of Example 80, by using 1,1-dioxothietane-3-carboxylic acid instead of propionyl chloride. The product was purified by flash column to afford Example 83 as a white solid. MS obsd. (ESI⁺) [(M+H)⁺]: 568.1. ¹H NMR (400 MHz, CD₃OD) δ ppm: 7.65 (d, J=2.0 Hz, 1H), 7.56 (d, J=8.7 Hz, 1H), 7.36 (dd, J=2.1, 8.7 Hz, 1H), 7.17 (d, J=3.5 Hz, 1H), 7.05 (d, J=3.5 Hz, 1H), 4.3-4.4 (m, 4H), 4.2-4.3 (m, 2H), 3.75 (t, J=8.5 Hz, 1H), 3.2-3.3 (m, 1H), 2.6-2.7 (m, 3H), 2.5-2.6 (m, 2H), 2.2-2.3 (m, 2H).

### Example 84

### N-[6-(5-chloro-1,3-benzoxazol-2-yl)spiro[3.3]heptan-2-yl]-5-[(1,1-dioxothiolane-3-carbonyl)sulfamoyl]furan-2-carboxamide

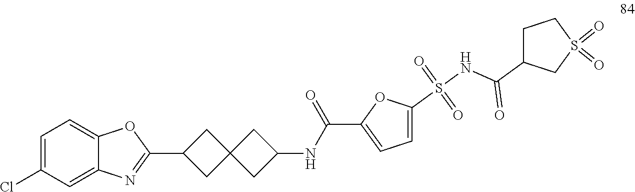 [SMILES: O=C(NC1CC2(C1)CC(c1nc3cc(Cl)ccc3o1)C2)c1ccc(S(=O)(=O)NC(=O)C2CCS(=O)(=O)C2)o1]

The title compound was prepared in analogy to the procedure described for the preparation of Example 80, by using 1,1-dioxothiolane-3-carboxylic acid instead of propionyl chloride. The product was purified by flash column to afford Example 84 as a white solid. MS obsd. (ESI⁺) [(M+H)⁺]: 582.5. ¹H NMR (400 MHz, CD₃OD) δ ppm: 7.54 (d, J=2.0 Hz, 1H), 7.46 (d, J=8.6 Hz, 1H), 7.25 (dd, J=2.1, 8.7 Hz, 1H), 6.99 (d, J=3.7 Hz, 1H), 6.86 (d, J=3.5 Hz, 1H), 4.29 (t, J=7.9 Hz, 1H), 3.65 (t, J=8.5 Hz, 1H), 3.0-3.1 (m, 3H), 2.9-2.9 (m, 2H), 2.5-2.6 (m, 3H), 2.4-2.5 (m, 2H), 2.3-2.4 (m, 2H), 2.1-2.2 (m, 3H).

### Example 85

### N-[6-(5-chloro-1,3-benzoxazol-2-yl)spiro[3.3]heptan-2-yl]-5-[(1,1-dioxothiane-3-carbonyl)sulfamoyl]furan-2-carboxamide

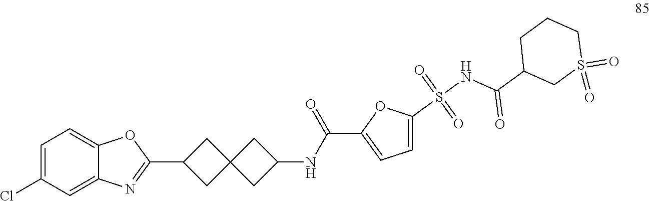 [SMILES: O=C(NC1CC2(C1)CC(c1nc3cc(Cl)ccc3o1)C2)c1ccc(S(=O)(=O)NC(=O)C2CCCS(=O)(=O)C2)o1]

The title compound was prepared in analogy to the procedure described for the preparation of Example 80, by using 1,1-dioxothiane-3-carboxylic acid instead of propionyl chloride. The product was purified by flash column to afford Example 85 as a white solid. MS obsd. (ESI⁺) [(M+H)⁺]: 596.2. ¹H NMR (400 MHz, CD₃OD) δ ppm: 7.65 (d, J=2.1 Hz, 1H), 7.57 (d, J=8.7 Hz, 1H), 7.37 (dd, J=2.1, 8.6 Hz, 1H), 7.10 (d, J=3.5 Hz, 1H), 6.97 (d, J=3.5 Hz, 1H), 4.4-4.4 (m, 1H), 3.77 (t, J=8.6 Hz, 1H), 3.23 (t, J=6.5 Hz, 3H), 3.1-3.2 (m, 2H), 3.0-3.1 (m, 3H), 2.6-2.7 (m, 3H), 2.5-2.6 (m, 3H), 2.29 (dd, J=8.9, 11.1 Hz, 1H), 2.1-2.2 (m, 3H), 1.9-1.9 (m, 2H).

### Example 86

### N-[6-(5-chloro-1,3-benzoxazol-2-yl)spiro[3.3]heptan-2-yl]-5-(oxetane-3-carbonylsulfamoyl)furan-2-carboxamide

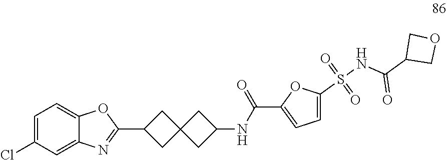 [SMILES: O=C(NC1CC2(C1)CC(c1nc3cc(Cl)ccc3o1)C2)c1ccc(S(=O)(=O)NC(=O)C2COC2)o1]

The title compound was prepared in analogy to the procedure described for the preparation of Example 80, by using oxetane-3-carboxylic acid instead of propionyl chloride. The product was purified by flash column to afford Example 86 as a white solid. MS obsd. (ESI⁺) [(M+H)⁺]: 520.2. ¹H NMR (400 MHz, CD₃OD) δ ppm: 7.54 (d, J=2.1 Hz, 1H), 7.46 (d, J=8.7 Hz, 1H), 7.25 (dd, J=2.1, 8.7 Hz, 1H), 6.99 (d, J=3.5 Hz, 1H), 6.87 (d, J=3.5 Hz, 1H), 4.65 (dd, J=2.3, 7.7 Hz, 4H), 4.29 (t, J=8.2 Hz, 1H), 3.6-3.7 (m, 2H), 2.5-2.6 (m, 4H), 2.3-2.4 (m, 2H), 2.1-2.2 (m, 2H).

### Example 87

### N-[[5-[[6-(5-chloro-1,3-benzoxazol-2-yl)spiro[3.3]heptan-2-yl]carbamoyl]-2-furyl]sulfonyl]tetrahydropyran-4-carboxamide

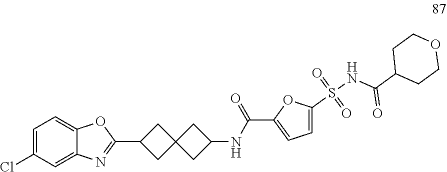 [SMILES: O=C(NC1CC2(C1)CC(c1nc3cc(Cl)ccc3o1)C2)c1ccc(S(=O)(=O)NC(=O)C2CCOCC2)o1]

The title compound was prepared in analogy to the procedure described for the preparation of Example 80, by using tetrahydropyran-4-carboxylic acid instead of propionyl chloride. The product was purified by flash column to afford Example 87 as a white solid. MS obsd. (ESI⁺) [(M+H)⁺]: 548.2. ¹H NMR (400 MHz, CD₃OD) δ ppm: 7.65 (d, J=2.1 Hz, 1H), 7.57 (d, J=8.7 Hz, 1H), 7.37 (dd, J=2.1, 8.7 Hz, 1H), 7.12 (d, J=3.7 Hz, 1H), 7.01 (d, J=3.5 Hz, 1H), 4.40 (t, J=7.9 Hz, 1H), 3.9-4.0 (m, 2H), 3.77 (t, J=8.5 Hz, 1H), 3.42 (dt, J=2.9, 11.3 Hz, 2H), 3.24 (t, J=6.4 Hz, 2H), 2.6-2.7 (m, 3H), 2.4-2.6 (m, 4H), 2.29 (dd, J=8.9, 11.1 Hz, 1H), 2.20 (dd, J=8.9, 11.5 Hz, 1H), 1.9-1.9 (m, 2H).

### Example 88 and Example 89

### N-[6-(5-chloro-1,3-benzoxazol-2-yl)spiro[3.3]heptan-2-yl]-5-(methylsulfamoyl)furan-2-carboxamide (Example 88), and N-[6-(5-chloro-1,3-benzoxazol-2-yl)spiro[3.3]heptan-2-yl]-5-(dimethylsulfamoyl)furan-2-carboxamide (Example 89)

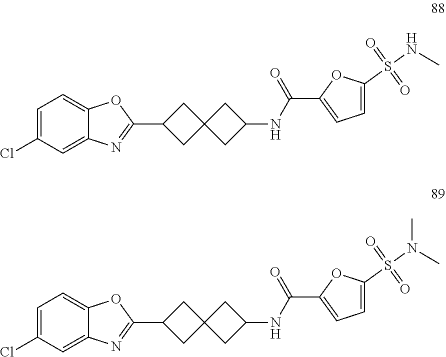 [SMILES: CNS(=O)(=O)c1ccc(C(=O)NC2CC3(C2)CC(c2nc4cc(Cl)ccc4o2)C3)o1 ; CN(C)S(=O)(=O)c1ccc(C(=O)NC2CC3(C2)CC(c2nc4cc(Cl)ccc4o2)C3)o1]

The title compounds were prepared according to the following scheme:

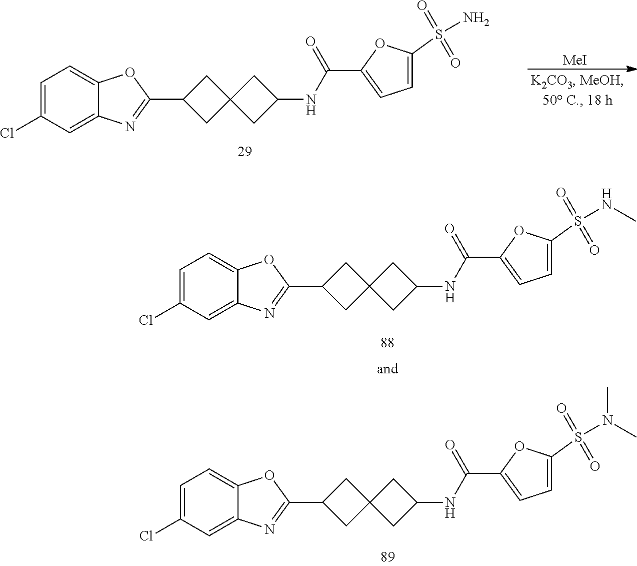 [SMILES: "MeI" ; NS(=O)(=O)c1ccc(C(=O)NC2CC3(C2)CC(c2nc4cc(Cl)ccc4o2)C3)o1 ; CNS(=O)(=O)c1ccc(C(=O)NC2CC3(C2)CC(c2nc4cc(Cl)ccc4o2)C3)o1 ; CN(C)S(=O)(=O)c1ccc(C(=O)NC2CC3(C2)CC(c2nc4cc(Cl)ccc4o2)C3)o1]

To a solution of N-(6-(5-chlorobenzo[d]oxazol-2-yl)spiro[3.3]heptan-2-yl)-5-sulfamoylfuran-2-carboxamide (Example 29, 200 mg, 0.46 mmol) and K₂CO₃ (127 mg, 0.92 mmol) in MeOH (3 mL) was added iodomethane (130 mg, 0.92 mmol). After being stirred at 50° C. for 18 h, the reaction solution was concentrated in vacuo. The residue was extracted with DCM (15 mL×3) and water (15 mL). The organic layers were washed with brine (10 mL), dried over Na₂SO₄ and concentrated in vacuo. The residue was purified by preparative HPLC to give N-[6-(5-chloro-1,3-benzoxazol-2-yl)spiro[3.3]heptan-2-yl]-5-(methylsulfamoyl)furan-2-carboxamide (Example 88, 35 mg, 16.8%) and N-[6-(5-chloro-1,3-benzoxazol-2-yl)spiro[3.3]heptan-2-yl]-5-(dimethylsulfamoyl)furan-2-carboxamide (Example 89, 75 mg, 34.9%).

Example 88: white solid, MS obsd. (ESI⁺) [(M+H)⁺]: 450.3. ¹H NMR (400 MHz, DMSO-d₆) δ ppm: 8.74 (d, J=7.6 Hz, 1H), 7.99 (q, J=4.8 Hz, 1H), 7.80 (d, J=2.1 Hz, 1H), 7.72 (d, J=8.7 Hz, 1H), 7.40 (dd, J=8.7, 2.2 Hz, 1H), 7.26 (d, J=3.7 Hz, 1H), 7.17 (d, J=3.5 Hz, 1H), 4.29 (sxt, J=8.1 Hz, 1H), 3.74 (quin, J=8.5 Hz, 1H), 2.52-2.68 (m, 6H), 2.29-2.47 (m, 3H), 2.07-2.24 (m, 2H).

Example 89: white solid, MS obsd. (ESI⁺) [(M+H)⁺]: 464.3. ¹H NMR (400 MHz, DMSO-d₆) δ ppm: 8.78 (d, J=7.6 Hz, 1H), 7.80 (d, J=2.1 Hz, 1H), 7.72 (d, J=8.7 Hz, 1H), 7.40 (dd, J=8.7, 2.1 Hz, 1H), 7.28 (q, J=3.6 Hz, 2H), 4.30 (sxt, J=8.1 Hz, 1H), 3.74 (quin, J=8.5 Hz, 1H), 2.77 (s, 6H), 2.52-2.68 (m, 3H), 2.40-2.48 (m, 2H), 2.13-2.35 (m, 3H).

### BIOLOGICAL EXAMPLES

### Example 90

### PHH Natural Infection Assay

Detailed procedures regarding primary human hepatocyte (PHH) HBV natural infection assay are described as below. One tube of frozen PHH (10 million cells) is thawed in 37° C. water bath and then transferred to 20 mL of PHH thawing medium (Sigma, InVitroGRO HT Medium, Cat. S03319) with gently mixing. The cells were then centrifuged at 80 g/min for 5 min, the supernatant was discarded and the tube was refilled with 25 mL of PHH plating medium (Sigma, InVitroGRO CP Medium, Cat. S03317). The tube was shaken very gently to re-suspend all cells, and then 50 μl of cells were transferred to each well 384-well collagen I coated plate with appropriate liquid handling equipment, e.g. Integra VIAFLO384 or Agilent Bravo. The cells were then cultured for 24 hours in a cell incubator. For HBV infection, after PHH attachment on the culture plate, the plating medium was removed and replenished with PHH culture medium containing HBV virus. The PHH culture medium was prepared with Dulbecco's Modified Eagle Medium (DMEM)/F12 (1:1 in volume ratio) containing 10% fetal bovine serum (Gibco, Cat. 10099141), 5 ng/mL human epidermal growth factor (Gibco, Cat.PHG0311L), 20 ng/mL dexamethasone (Sigma, Cat.D4902-100 mg), 250 ng/mL human recombinant insulin (Gibco, Cat.41400045) and 100 U/mL penicillin. HBV virus at 200 genome equivalent (GE) per cell with 4% PEG8000 (Sigma, Cat.P1458) containing culture medium were added to the PHH culture medium for infection. The cells were then cultured for 24 hours in cell incubator. Then the cell culture supernatant was removed. The HBV-infected PHH were cultured with sandwich culture method with PHH culture medium containing 1% DMSO and 0.25 mg/mL matrix gel for 72 hours. The supernatant was then refreshed with PHH culture medium containing different concentrations of testing compounds for two times with 72-hour interval. At the end of treatment, the supernatant was collected for viral markers measurements, including HBsAg, HBeAg, HBV DNA and cytotoxicity. HBsAg and HBeAg were detected using alphalisa method using their specific antibodies. For HBV DNA detection, HBV DNA Quantitative Fluorescence Diagnostic Kit (Sansure Biotech Inc.) was used following the manufacture's protocol. Cytotoxicity was determined using Cell Counting Kit-8 (CCK8, Dojindo Molecular Technologies, Inc.).

The compounds of the present invention were tested for their capacity to inhibit HBsAg and HBeAg as described herein. The Examples were tested in the above assay and found to have IC₅₀ below 10 μM. Results of PHH assay are given in Table 1.

TABLE 1
Activity data of compounds of this invention

| | HBsAg | HbeAg | |
|---|---|---|---|
| | IC₅₀ | IC₅₀ | CC₅₀ |
| Example No. | (μM) | (μM) | (μM) |
| Example 1 | 3.633 | 2.291 | 96.803 |
| Example 2 | 5.864 | 4.061 | 61.553 |
| Example 3 | 9.794 | 7.640 | 96.177 |
| Example 4 | 5.568 | 2.940 | 55.986 |
| Example 5 | 8.191 | 6.285 | 73.162 |
| Example 6 | 2.768 | 2.083 | >100 |
| Example 7 | 3.570 | 1.337 | 85.852 |
| Example 8 | 7.965 | 5.838 | 94.206 |
| Example 9 | 1.575 | 1.015 | 90.966 |
| Example 10 | 3.347 | 2.941 | 96.108 |
| Example 11 | 5.882 | 5.221 | >10 |
| Example 12 | 3.962 | 6.855 | 30.186 |
| Example 13 | 9.815 | 6.202 | >100 |
| Example 14 | 1.615 | 0.854 | 76.539 |
| Example 15 | 1.947 | 1.165 | 78.966 |
| Example 15-a | 5.194 | 3.942 | 84.037 |
| Example 15-b | 0.896 | 0.694 | 35.792 |
| Example 16 | 1.528 | 1.026 | 73.966 |
| Example 17 | 0.275 | 0.177 | 89.917 |
| Example 18 | 3.274 | 1.812 | 72.126 |
| Example 19 | 0.036 | 0.045 | 94.537 |
| Example 19-a | 0.088 | 0.067 | >10 |
| Example 19-b | 0.071 | 0.053 | >10 |
| Example 19-c | 2.422 | 1.948 | >10 |
| Example 19-d | 2.859 | 2.499 | >10 |
| Example 20 | 0.445 | 0.180 | 89.225 |
| Example 21 | 0.372 | 0.314 | 92.365 |
| Example 22 | 0.506 | 0.264 | 75.571 |
| Example 23 | 0.321 | 0.883 | >100 |
| Example 24 | 2.44 | 2.334 | 95.284 |
| Example 25 | 4.193 | 3.728 | >10 |
| Example 26 | 0.365 | 0.329 | >100 |
| Example 27 | 0.152 | 0.116 | >100 |
| Example 28 | 0.758 | 0.509 | 94.195 |
| Example 29 | 0.534 | 0.509 | 94.195 |
| Example 29-a | 5.125 | 2.282 | >100 |
| Example 29-b | 1.547 | 0.909 | >100 |
| Example 30 | 3.971 | 2.172 | >100 |
| Example 31 | 7.559 | 6.982 | >100 |
| Example 32 | 4.939 | 3.153 | >100 |
| Example 33 | 1.408 | 1.387 | >100 |
| Example 34 | 8.778 | 5.841 | >100 |
| Example 35 | 7.814 | 6.529 | 98.270 |
| Example 36 | 0.885 | 0.815 | >100 |
| Example 37 | 9.381 | 5.723 | >100 |
| Example 38 | 0.526 | 0.317 | 96.334 |
| Example 39 | 0.960 | 0.603 | >10 |
| Example 40 | 0.084 | 0.067 | >10 |
| Example 41 | 8.471 | 6.620 | >100 |
| Example 42 | 1.515 | 1.414 | 96.215 |
| Example 43 | 1.136 | 0.839 | >100 |
| Example 44 | 1.801 | 1.550 | 95.817 |
| Example 45 | 2.211 | 1.896 | >100 |
| Example 46 | 5.827 | 2.813 | 89.811 |
| Example 47 | 9.587 | 10.622 | >100 |
| Example 48 | 2.403 | 1.816 | >100 |
| Example 49 | 2.401 | 1.738 | 84.516 |
| Example 49-a | 4.170 | 4.247 | 81.051 |
| Example 49-b | 0.647 | 0.626 | 91.527 |
| Example 50 | 0.540 | 0.350 | >100 |
| Example 51 | 1.803 | 1.145 | >100 |
| Example 52 | 1.704 | 1.203 | 71.962 |
| Example 52-a | 4.264 | 1.769 | >100 |
| Example 52-b | 0.939 | 0.898 | >100 |
| Example 52-c | 5.036 | 3.609 | >100 |
| Example 52-d | 0.966 | 0.876 | >100 |
| Example 53 | 0.494 | 0.394 | >10 |
| Example 53-a | 1.887 | 1.270 | >10 |
| Example 53-b | 6.830 | 4.650 | >10 |
| Example 53-c | 0.650 | 0.621 | 91.927 |
| Example 53-d | 1.119 | 1.339 | 92.767 |
| Example 54 | 0.470 | 0.542 | 88.763 |
| Example 54-a | 6.064 | 5.695 | >10 |
| Example 54-b | 0.285 | 0.166 | >100 |
| Example 54-c | 5.841 | 4.592 | >10 |
| Example 54-d | 0.703 | 0.694 | >10 |
| Example 55 | 0.802 | 0.988 | 83.323 |
| Example 56 | 4.409 | 2.897 | >100 |
| Example 57 | 0.884 | 0.754 | 81.866 |
| Example 58 | 8.790 | 5.875 | >100 |
| Example 59 | 3.806 | 2.598 | >100 |
| Example 60 | 1.961 | 1.909 | >100 |
| Example 61 | 3.651 | 3.140 | >100 |
| Example 62 | 5.006 | 4.851 | >100 |
| Example 63 | 6.694 | 4.944 | >100 |
| Example 64 | 2.931 | 2.333 | >100 |
| Example 64-a | 8.448 | 4.809 | >10 |
| Example 64-b | 9.456 | 9.126 | >10 |
| Example 64-d | 1.060 | 0.669 | >10 |
| Example 64-d | 2.969 | 1.456 | >10 |
| Example 65 | 6.376 | 5.828 | >100 |
| Example 66 | 8.184 | 3.814 | >10 |
| Example 67 | 6.238 | 3.651 | >10 |
| Example 68 | 0.626 | 0.371 | >100 |
| Example 69 | 0.329 | 0.223 | >100 |
| Example 70 | 0.361 | 0.376 | >100 |
| Example 71 | 3.663 | 3.279 | >100 |
| Example 72 | 0.314 | 0.386 | >100 |
| Example 73 | 0.416 | 0.256 | >100 |
| Example 74 | 6.668 | 5.917 | >10 |
| Example 75 | 2.492 | 1.691 | >10 |
| Example 76 | 2.844 | 1.991 | >10 |
| Example 77 | 1.157 | 0.563 | >100 |
| Example 78 | 2.452 | 0.929 | >100 |
| Example 79 | 6.129 | 4.652 | >10 |
| Example 80 | 2.774 | 1.796 | >100 |
| Example 81 | 1.736 | 0.796 | 11.342 |
| Example 82 | 0.461 | 0.224 | 85.653 |
| Example 83 | 3.301 | 1.965 | >10 |
| Example 84 | 9.001 | 6.317 | >10 |
| Example 85 | 2.519 | 1.665 | >10 |
| Example 86 | 2.989 | 2.189 | >10 |
| Example 87 | 1.304 | 0.981 | >10 |
| Example 88 | 1.003 | 0.687 | >10 |
| Example 89 | 1.036 | 0.723 | >10 |
| Example 90 | 2.964 | 2.074 | >100 |
| Example 91 | 0.475 | 0.360 | 94.920 |

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

It is to be understood that the invention is not limited to the particular embodiments and aspects of the disclosure described above, as variations of the particular embodiments and aspects may be made and still fall within the scope of the appended claims. All documents cited to or relied upon herein are expressly incorporated by reference.

1. A compound of the formula (I), 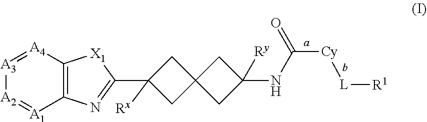
wherein
R¹ is selected from the group consisting of H, halogen, amino, CN, C₁₋₆alkyl, haloC₁₋₆alkyl, C₁₋₆alkoxy, C₁₋₆alkoxycarbonylC₁₋₆alkyl, C₁₋₆alkoxyC₂₋₆alkoxyC₁₋₆alkyl, C₁₋₆alkoxyC₂₋₆alkoxyC₂₋₆alkoxyC₁₋₆alkyl, C₁₋₆alkylamino, (C₁₋₆alkyl)₂amino, C₁₋₆alkoxyC₁₋₆alkyl, C₃₋₇cycloalkyl, C₃₋₇cycloalkylC₁₋₆alkyl, heterocyclyl, heterocyclylC₁₋₆alkyl, C₁₋₆ alkylheterocyclylC₁₋₆alkyl and phenylC₁₋₆alkoxycarbonylheterocyclylC₁₋₆alkyl; wherein C₃₋₇cycloalkyl, C₃₋₇cycloalkylC₁₋₆alkyl, heterocyclyl, heterocyclylC₁₋₆alkyl, C₁₋₆ alkylheterocyclylC₁₋₆alkyl or phenylC₁₋₆alkoxycarbonylheterocyclylC₁₋₆alkyl are unsubstituted or substituted one or two or three times by halogen;
R^{x} is H, halogen, hydroxy or C₁₋₆alkyl;
R^{y} is H, halogen, hydroxy or C₁₋₆alkyl;
A₁ is N or CR²; wherein R² is H or halogen;
A₂ is N or CR³; wherein R³ is H or halogen;
A₃ is N or CR⁴; wherein R⁴ is selected from H and halogen;
A₄ is N or CR⁵; wherein R⁵ is H or halogen;
X₁ is NR^{z} or O; wherein R^{z} is C₁₋₆alkyl;
Cy is 5 membered heteroaryl; wherein 5 membered heteroaryl is unsubstituted or substituted by one or two or three substituents independently selected from halogen, C₁₋₆alkyl, haloC₁₋₆alkyl, C₁₋₆alkoxy or C₁₋₆alkoxyC₁₋₆alkyl;
L is a bond, carbonyl, sulfinyl, sulfonyl, sulfonylC₁₋₆alkyl, sulfonylaminoC₁₋₆alkyl, sulfanyl, sulfonimidoyl, sulfonimidoylC₁₋₆alkyl or carbonylaminosulfonyl;
or a pharmaceutically acceptable salt thereof.

2. A compound according to claim 1, wherein
R¹ is selected from H, halogen, amino, CN, C₁₋₆alkyl, haloC₁₋₆alkyl, C₁₋₆alkoxy, C₁₋₆alkoxycarbonylC₁₋₆alkyl, C₁₋₆alkoxyC₂₋₆alkoxyC₁₋₆alkyl, C₁₋₆alkoxyC₂₋₆alkoxyC₂₋₆alkoxyC₁₋₆alkyl, C₁₋₆alkylamino, (C₁₋₆alkyl)₂amino, C₁₋₆alkoxyC₁₋₆alkyl, C₃₋₇cycloalkyl, C₃₋₇cycloalkylC₁₋₆alkyl, oxetanyl, pyrrolidinyl, morpholino, tetrahydrofuranyl, 1,1-dioxothietanyl, 1,1-dioxothiolanyl, 1,1-dioxothianyl, tetrahydropyranyl, 1-oxo-4,5-dihydro-3H-isothiazol-1-yl, 1-oxo-1lambda6-thia-2-azacyclohexen-1-yl, oxetanylC₁₋₆alkyl, C₁₋₆alkylazetidinylC₁₋₆alkyl or phenylC₁₋₆alkoxycarbonylazetidinylC₁₋₆alkyl; wherein C₃₋₇cycloalkylC₁₋₆alkyl is unsubstituted or substituted one or two or three times by halogen;
R^{x} is H;
R^{y} is H;
A₁ is CH;
A₂ is N or CR³; wherein R³ is H or halogen;
A₃ is CR⁴; wherein R⁴ is H or halogen;
A₄ is N or CH;
X₁ is O;
Cy is formula (Cy1) 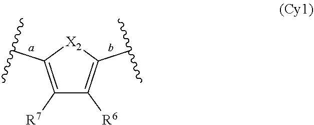
wherein
R⁶ is H or halogen;
R⁷ is H, halogen or C₁₋₆alkyl;
X₂ is O or S;
or formula (Cy2) 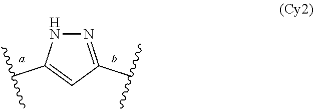 [SMILES: *c1cc(*)[nH]n1]
or formula (Cy3) 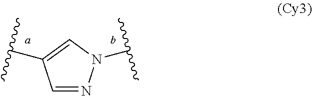 [SMILES: *c1cnn(*)c1]
L is a bond, carbonyl, sulfinyl, sulfonyl, sulfonylC₁₋₆alkyl, sulfonylaminoC₁₋₆alkyl, sulfanyl, sulfonimidoyl, sulfonimidoylC₁₋₆alkyl or carbonylaminosulfonyl;
or a pharmaceutically acceptable salt thereof.

3. A compound according to claim 1 wherein:
R¹ is selected from H, Cl, Br, amino, CN, methyl, ethyl, propyl, isopropyl, isobutyl, tert-butyl, CF₃, methoxy, ethoxy, methoxycarbonylethyl, methoxyethoxymethyl, methoxyethoxyethoxymethyl, methylamino, dimethylamino, methoxymethyl, methoxyethyl, cyclopropyl, cyclobutyl, cyclopentyl, cyclohexyl, bicyclo[1.1.1]pentanyl, cyclopropylmethyl, cyclobutylmethyl, difluorocyclobutylmethyl, oxetanyl, pyrrolidinyl, morpholino, tetrahydrofuranyl, 1,1-dioxothietanyl, 1,1-dioxothiolanyl, 1,1-dioxothianyl, tetrahydropyranyl, 1-oxo-4,5-dihydro-3H-isothiazol-1-yl, 1-oxo-1-lambda6-thia-2-azacyclohexen-1-yl, oxetanylmethyl, methylazetidinylmethyl or phenylmethoxycarbonylazetidinylmethyl;
R^{x} is H;
R^{y} is H;
A₁ is CH;
A₂ is N or CR³; wherein R³ is H or Cl;
A₃ is CR⁴; wherein R⁴ is H, F or Cl;
A₄ is selected N and CH;
X₁ is O;
Cy is formula (Cy1) 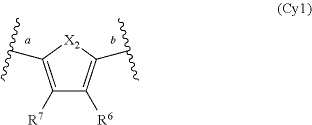
wherein
R⁶ is H or Br;
R⁷ is H, Br or methyl;
X₂ is O or S;
or formula (Cy2) 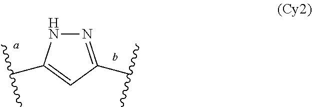 [SMILES: *c1cc(*)[nH]n1]
or formula (Cy3) 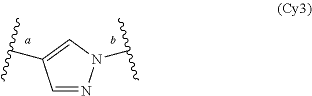 [SMILES: *c1cnn(*)c1]
L is a bond, carbonyl, sulfinyl, sulfonyl, sulfonylmethyl, sulfonylethyl, sulfonylaminomethyl, sulfanyl, sulfonimidoyl, sulfonimidoylmethyl or carbonylaminosulfonyl;
or a pharmaceutically acceptable salt thereof.

4. A compound according to claim 1, or a pharmaceutically acceptable salt thereof, wherein R¹ is amino, C₁₋₆alkyl, haloC₁₋₆alkyl, C₁₋₆alkylamino, C₁₋₆alkoxyC₁₋₆alkyl, C₃₋₇cycloalkyl, C₃₋₇cycloalkylC₁₋₆alkyl or phenylC₁₋₆alkoxycarbonylazetidinylC₁₋₆alkyl; wherein C₃₋₇cycloalkylC₁₋₆alkyl is unsubstituted or substituted one or two times independently by halogen.

5. A compound according to claim 4, or a pharmaceutically acceptable salt thereof, wherein R¹ is m amino, methyl, ethyl, propyl, isopropyl, isobutyl, tert-butyl, CF₃, methylamino, methoxymethyl, methoxyethyl, cyclopropyl, cyclobutyl, cyclohexyl, cyclopropylmethyl, cyclobutylmethyl, difluorocyclobutylmethyl or phenylmethoxycarbonylazetidinylmethyl.

6. A compound according to claim 1, or a pharmaceutically acceptable salt thereof, wherein A₂ is CR³; wherein R³ is halogen.

7. A compound according to claim 6, or a pharmaceutically acceptable salt thereof, wherein A₂ is CCl.

8. A compound according to claim 1, or a pharmaceutically acceptable salt thereof, wherein
Cy is formula (Cy1) 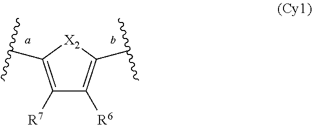
wherein
R⁶ is H or halogen;
R⁷ is H;
X₂ is O.

9. A compound according to claim 8, or a pharmaceutically acceptable salt thereof, wherein R⁶ is m H or Br.

10. A compound according to claim 1, or a pharmaceutically acceptable salt thereof, wherein L is selected from sulfinyl, sulfonyl, sulfonylC₁₋₆alkyl, sulfonimidoyl and carbonylaminosulfonyl.

11. A compound according to claim 10, or a pharmaceutically acceptable salt thereof, wherein L is sulfinyl, sulfonyl, sulfonylmethyl, sulfonimidoyl or carbonylaminosulfonyl.

12. A compound according to claim 1 having the formula (II), 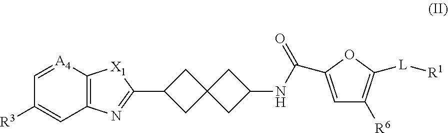
wherein
R¹ is amino, C₁₋₆alkyl, haloC₁₋₆alkyl, C₁₋₆alkylamino, C₁₋₆alkoxyC₁₋₆alkyl, C₃₋₇cycloalkyl, C₃₋₇cycloalkylC₁₋₆alkyl or phenylC₁₋₆alkoxycarbonylazetidinylC₁₋₆alkyl; wherein C₃₋₇cycloalkylC₁₋₆alkyl is unsubstituted or substituted one or two times independently by halogen;
R³ is halogen;
A₄ is N or CH;
X₁ is O;
R⁶ is H or halogen;
L is sulfinyl, sulfonyl, sulfonylC₁₋₆alkyl, sulfonimidoyl or carbonylaminosulfonyl;
or a pharmaceutically acceptable salt thereof.

13. A compound according to claim 12, wherein
R¹ is amino, methyl, ethyl, propyl, isopropyl, isobutyl, tert-butyl, CF₃, methylamino, methoxymethyl, methoxyethyl, cyclopropyl, cyclobutyl, cyclohexyl, cyclopropylmethyl, cyclobutylmethyl, difluorocyclobutylmethyl or phenylmethoxycarbonylazetidinylmethyl;
R³ is Cl;
A₄ is N or CH;
X₁ is O;
R⁶ is H or Br;
L is sulfinyl, sulfonyl, sulfonylmethyl, sulfonimidoyl or carbonylaminosulfonyl;
or a pharmaceutically acceptable salt thereof.

14. A compound according to claim 12, or a pharmaceutically acceptable salt thereof, wherein A₄ is CH.

15. A compound according to claim 1, selected from the group consisting of:
N-[6-(5-chloro-1,3-benzoxazol-2-yl)spiro[3.3]heptan-2-yl]-5-(trifluoromethyl)furan-2-carboxamide;
N-[6-(5-chloro-1,3-benzoxazol-2-yl)spiro[3.3]heptan-2-yl]-5-cyano-furan-2-carboxamide;
5-bromo-N-[6-(5-chloro-1,3-benzoxazol-2-yl)spiro[3.3]heptan-2-yl]furan-2-carboxamide;
4-bromo-N-[6-(5-chloro-1,3-benzoxazol-2-yl)spiro[3.3]heptan-2-yl]furan-2-carboxamide;
3-bromo-N-[6-(5-chloro-1,3-benzoxazol-2-yl)spiro[3.3]heptan-2-yl]furan-2-carboxamide;
5-chloro-N-[6-(5-chloro-1,3-benzoxazol-2-yl)spiro[3.3]heptan-2-yl]furan-2-carboxamide;
N-[6-(5-chloro-1,3-benzoxazol-2-yl)spiro[3.3]heptan-2-yl]-5-isopropyl-furan-2-carboxamide;
N-[6-(5-chloro-1,3-benzoxazol-2-yl)spiro[3.3]heptan-2-yl]-5-methoxy-furan-2-carboxamide;
N-[6-(5-chloro-1,3-benzoxazol-2-yl)spiro[3.3]heptan-2-yl]-5-(methoxymethyl)furan-2-carboxamide;
N-[6-(5-chloro-1,3-benzoxazol-2-yl)spiro[3.3]heptan-2-yl]-5-(cyclopropanecarbonyl)furan-2-carboxamide;
N2-[6-(5-chloro-1,3-benzoxazol-2-yl)spiro[3.3]heptan-2-yl]furan-2,5-dicarboxamide;
N-[6-(5-chloro-1,3-benzoxazol-2-yl)spiro[3.3]heptan-2-yl]-3-(trifluoromethyl)-1H-pyrazole-5-carboxamide;
N-[6-(5-chloro-1,3-benzoxazol-2-yl)spiro[3.3]heptan-2-yl]-1-methyl-pyrazole-4-carboxamide;
N-[6-(5-chloro-1,3-benzoxazol-2-yl)spiro[3.3]heptan-2-yl]-5-methylsulfinyl-furan-2-carboxamide;
N-[6-(5-chloro-1,3-benzoxazol-2-yl)spiro[3.3]heptan-2-yl]-5-methylsulfonyl-furan-2-carboxamide;
(Sₐ)—N-[6-(5-chloro-1,3-benzoxazol-2-yl)spiro[3.3]heptan-2-yl]-5-methylsulfonyl-furan-2-carboxamide;
(Rₐ)—N-[6-(5-chloro-1,3-benzoxazol-2-yl)spiro[3.3]heptan-2-yl]-5-methylsulfonyl-furan-2-carboxamide;
N-[6-(5-chloro-1,3-benzoxazol-2-yl)spiro[3.3]heptan-2-yl]-5-ethylsulfonyl-furan-2-carboxamide;
N-[6-(5-chloro-1,3-benzoxazol-2-yl)spiro[3.3]heptan-2-yl]-5-cyclopropylsulfonyl-furan-2-carboxamide;
N-[6-(5-chloro-1,3-benzoxazol-2-yl)spiro[3.3]heptan-2-yl]-5-(oxetan-3-ylsulfonyl)furan-2-carboxamide;
N-[6-(5-Chloro-1,3-benzoxazol-2-yl)spiro[3.3]heptan-2-yl]-5-(cyclopropylmethylsulfinyl)furan-2-carboxamide;
(Rₐ)—N-[6-(5-chloro-1,3-benzoxazol-2-yl)spiro[3.3]heptan-2-yl]-5-[(S)-cyclopropylmethylsulfinyl]furan-2-carboxamide;
(Rₐ)—N-[6-(5-chloro-1,3-benzoxazol-2-yl)spiro[3.3]heptan-2-yl]-5-[(R)-cyclopropylmethylsulfinyl]furan-2-carboxamide;
(Sₐ)—N-[6-(5-Chloro-1,3-benzoxazol-2-yl)spiro[3.3]heptan-2-yl]-5-[(S)-cyclopropylmethylsulfinyl]furan-2-carboxamide;
(Sₐ)—N-[6-(5-chloro-1,3-benzoxazol-2-yl)spiro[3.3]heptan-2-yl]-5-[(R)-cyclopropylmethylsulfinyl]furan-2-carboxamide;
N-[6-(5-chloro-1,3-benzoxazol-2-yl)spiro[3.3]heptan-2-yl]-5-(cyclopropylmethylsulfonyl)furan-2-carboxamide;
N-[6-(5-chloro-1,3-benzoxazol-2-yl)spiro[3.3]heptan-2-yl]-5-isobutylsulfinyl-furan-2-carboxamide;
N-[6-(5-chloro-1,3-benzoxazol-2-yl)spiro[3.3]heptan-2-yl]-5-isobutylsulfonyl-furan-2-carboxamide;
N-[6-(5-chloro-1,3-benzoxazol-2-yl)spiro[3.3]heptan-2-yl]-5-(cyclobutylmethylsulfonyl)furan-2-carboxamide;
N-[6-(5-chloro-1,3-benzoxazol-2-yl)spiro[3.3]heptan-2-yl]-5-(oxetan-3-ylmethylsulfonyl)furan-2-carboxamide;
N-[6-(5-chloro-1,3-benzoxazol-2-yl)spiro[3.3]heptan-2-yl]-5-[(1-methylazetidin-3-yl)methylsulfonyl]furan-2-carboxamide;
benzyl 3-[[5-[[6-(5-chloro-1,3-benzoxazol-2-yl)spiro[3.3]heptan-2-yl]carbamoyl]-2-furyl]sulfonylmethyl]azetidine-1-carboxylate;
N-[6-(5-chloro-1,3-benzoxazol-2-yl)spiro[3.3]heptan-2-yl]-5-[(3,3-difluorocyclobutyl)methylsulfonyl]furan-2-carboxamide;
N-[6-(5-chloro-1,3-benzoxazol-2-yl)spiro[3.3]heptan-2-yl]-5-(2-methoxyethylsulfonyl)furan-2-carboxamide;
N-[6-(5-chloro-1,3-benzoxazol-2-yl)spiro[3.3]heptan-2-yl]-5-sulfamoyl-furan-2-carboxamide;
(Sₐ)—N-[6-(5-chloro-1,3-benzoxazol-2-yl)spiro[3.3]heptan-2-yl]-5-sulfamoyl-furan-2-carboxamide;
(Rₐ)—N-[6-(5-chloro-1,3-benzoxazol-2-yl)spiro[3.3]heptan-2-yl]-5-sulfamoyl-furan-2-carboxamide;
N-[6-(5-chloro-1,3-benzoxazol-2-yl)spiro[3.3]heptan-2-yl]-5-(methylsulfonylmethyl)furan-2-carboxamide;
N-[6-(5-chloro-1,3-benzoxazol-2-yl)spiro[3.3]heptan-2-yl]-5-(cyclopropylsulfonylmethyl)furan-2-carboxamide;
N-[6-(5-chloro-1,3-benzoxazol-2-yl)spiro[3.3]heptan-2-yl]-5-(2-methylsulfonylethyl)furan-2-carboxamide;
N-[6-(5-chloro-1,3-benzoxazol-2-yl)spiro[3.3]heptan-2-yl]-5-(sulfamoylmethyl)furan-2-carboxamide;
N-[6-(5-chloro-1,3-benzoxazol-2-yl)spiro[3.3]heptan-2-yl]-5-(2-sulfamoylethyl)furan-2-carboxamide;
N-[6-(5-chloro-1,3-benzoxazol-2-yl)spiro[3.3]heptan-2-yl]-5-[(sulfamoylamino)methyl]furan-2-carboxamide;
4-bromo-N-[6-(5-chloro-1,3-benzoxazol-2-yl)spiro[3.3]heptan-2-yl]-5-ethylsulfonyl-furan-2-carboxamide;
N-[6-(5-chloro-1,3-benzoxazol-2-yl)spiro[3.3]heptan-2-yl]-5-ethylsulfonyl-3-methyl-furan-2-carboxamide;
5-tert-butylsulfinyl-N-[6-(5-chloro-1,3-benzoxazol-2-yl)spiro[3.3]heptan-2-yl]furan-2-carboxamide;
5-tert-butylsulfonyl-N-[6-(5-chloro-1,3-benzoxazol-2-yl)spiro[3.3]heptan-2-yl]furan-2-carboxamide;
N-[6-(5-chloro-1,3-benzoxazol-2-yl)spiro[3.3]heptan-2-yl]-5-cyclopropylsulfinyl-furan-2-carboxamide;
N-[6-(5-chloro-1,3-benzoxazol-2-yl)spiro[3.3]heptan-2-yl]-5-(trifluoromethylsulfanyl)furan-2-carboxamide;
N-[6-(5-chloro-1,3-benzoxazol-2-yl)spiro[3.3]heptan-2-yl]-5-(trifluoromethylsulfinyl)furan-2-carboxamide;
N-[6-(5-chloro-1,3-benzoxazol-2-yl)spiro[3.3]heptan-2-yl]-5-(trifluoromethylsulfonyl)furan-2-carboxamide;
N-[6-(5-chloro-1,3-benzoxazol-2-yl)spiro[3.3]heptan-2-yl]-5-(isopropylsulfonylmethyl)furan-2-carboxamide;
N-[6-(5-chloro-1,3-benzoxazol-2-yl)spiro[3.3]heptan-2-yl]-5-methylsulfonyl-thiophene-2-carboxamide;
N-[6-(6-Chloro-1,3-benzoxazol-2-yl)spiro[3.3]heptan-2-yl]-5-(trifluoromethyl)furan-2-carboxamide;
N-[6-(6-Fluoro-1,3-benzoxazol-2-yl)spiro[3.3]heptan-2-yl]-5-(trifluoromethyl)furan-2-carboxamide;
N-[6-(6-chlorooxazolo[5,4-b]pyridin-2-yl)spiro[3.3]heptan-2-yl]-5-methylsulfonyl-furan-2-carboxamide;
N-[6-(6-chlorooxazolo[5,4-b]pyridin-2-yl)spiro[3.3]heptan-2-yl]-5-cyclopropylsulfonyl-furan-2-carboxamide;
(Rₐ)—N-[6-(6-chlorooxazolo[5,4-b]pyridin-2-yl)spiro[3.3]heptan-2-yl]-5-cyclopropylsulfonyl-furan-2-carboxamide;
(Sₐ)—N-[6-(6-chlorooxazolo[5,4-b]pyridin-2-yl)spiro[3.3]heptan-2-yl]-5-cyclopropylsulfonyl-furan-2-carboxamide;
N-[6-(6-chlorooxazolo[5,4-b]pyridin-2-yl)spiro[3.3]heptan-2-yl]-5-cyclopropylsulfonyl-furan-2-carboxamide;
N-[6-(6-chlorooxazolo[4,5-c]pyridin-2-yl)spiro[3.3]heptan-2-yl]-5-cyclopropylsulfonyl-furan-2-carboxamide;
N-[6-(5-chloro-1,3-benzoxazol-2-yl)spiro[3.3]heptan-2-yl]-5-(methylsulfonimidoyl)furan-2-carboxamide;
(Rₐ)—N-[6-(5-chloro-1,3-benzoxazol-2-yl)spiro[3.3]heptan-2-yl]-5-[(S)-methylsulfonimidoyl]furan-2-carboxamide;
(Sₐ)—N-[6-(5-chloro-1,3-benzoxazol-2-yl)spiro[3.3]heptan-2-yl]-5-[(S)-methylsulfonimidoyl]furan-2-carboxamide;
(Rₐ)—N-[6-(5-chloro-1,3-benzoxazol-2-yl)spiro[3.3]heptan-2-yl]-5-[(R)-methylsulfonimidoyl]furan-2-carboxamide;
(Sₐ)—N-[6-(5-chloro-1,3-benzoxazol-2-yl)spiro[3.3]heptan-2-yl]-5-[(R)-methylsulfonimidoyl]furan-2-carboxamide;
N-[6-(5-chloro-1,3-benzoxazol-2-yl)spiro[3.3]heptan-2-yl]-5-(cyclopropylsulfonimidoyl)furan-2-carboxamide;
(Rₐ)—N-[6-(5-chloro-1,3-benzoxazol-2-yl)spiro[3.3]heptan-2-yl]-5-[(S)-cyclopropylsulfonimidoyl]furan-2-carboxamide;
(Sₐ)—N-[6-(5-chloro-1,3-benzoxazol-2-yl)spiro[3.3]heptan-2-yl]-5-[(S)-cyclopropylsulfonimidoyl]furan-2-carboxamide;
(Rₐ)—N-[6-(5-chloro-1,3-benzoxazol-2-yl)spiro[3.3]heptan-2-yl]-5-[(R)-cyclopropylsulfonimidoyl]furan-2-carboxamide;
(Sₐ)—N-[6-(5-chloro-1,3-benzoxazol-2-yl)spiro[3.3]heptan-2-yl]-5-[(R)-cyclopropylsulfonimidoyl]furan-2-carboxamide;
N-[6-(5-chloro-1,3-benzoxazol-2-yl)spiro[3.3]heptan-2-yl]-5-(cyclopropylmethylsulfonimidoyl)furan-2-carboxamide;
(Rₐ)—N-[6-(5-chloro-1,3-benzoxazol-2-yl)spiro[3.3]heptan-2-yl]-5-[(S)-cyclopropylmethylsulfonimidoyl]furan-2-carboxamide;
(Rₐ)—N-[6-(5-Chloro-1,3-benzoxazol-2-yl)spiro[3.3]heptan-2-yl]-5-[(R)-cyclopropylmethylsulfonimidoyl]furan-2-carboxamide;
(Sₐ)—N-[6-(5-Chloro-1,3-benzoxazol-2-yl)spiro[3.3]heptan-2-yl]-5-[(S)-cyclopropylmethylsulfonimidoyl]furan-2-carboxamide;
(Sₐ)—N-[6-(5-Chloro-1,3-benzoxazol-2-yl)spiro[3.3]heptan-2-yl]-5-[(R)-cyclopropylmethylsulfonimidoyl]furan-2-carboxamide;
N-[6-(5-chloro-1,3-benzoxazol-2-yl)spiro[3.3]heptan-2-yl]-5-(isobutylsulfonimidoyl)furan-2-carboxamide;
N-[6-(5-chloro-1,3-benzoxazol-2-yl)spiro[3.3]heptan-2-yl]-5-(oxetan-3-ylmethylsulfonimidoyl)furan-2-carboxamide;
N-[6-(5-chloro-1,3-benzoxazol-2-yl)spiro[3.3]heptan-2-yl]-5-[(3,3-difluorocyclobutyl)methylsulfonimidoyl]furan-2-carboxamide;
N-[6-(5-chloro-1,3-benzoxazol-2-yl)spiro[3.3]heptan-2-yl]-5-[(ethylsulfonimidoyl)methyl]furan-2-carboxamide;
N-[6-(5-chloro-1,3-benzoxazol-2-yl)spiro[3.3]heptan-2-yl]-5-[(isopropylsulfonimidoyl)methyl]furan-2-carboxamide;
N-[6-(5-chloro-1,3-benzoxazol-2-yl)spiro[3.3]heptan-2-yl]-5-[(cyclopropylsulfonimidoyl)methyl]furan-2-carboxamide;
N-[6-(5-chloro-1,3-benzoxazol-2-yl)spiro[3.3]heptan-2-yl]-5-(1-oxo-4,5-dihydro-3H-isothiazol-1-yl)furan-2-carboxamide;
N-[6-(5-chloro-1,3-benzoxazol-2-yl)spiro[3.3]heptan-2-yl]-5-(1-oxo-1lambda6-thia-2-azacyclohexen-1-yl)furan-2-carboxamide;
N-[6-(6-chlorooxazolo[5,4-b]pyridin-2-yl)spiro[3.3]heptan-2-yl]-5-(methylsulfonimidoyl)furan-2-carboxamide;
N-[6-(6-chlorooxazolo[5,4-b]pyridin-2-yl)spiro[3.3]heptan-2-yl]-5-(cyclopropylsulfonimidoyl)furan-2-carboxamide;
(Rₐ)—N-[6-(6-chlorooxazolo[5,4-b]pyridin-2-yl)spiro[3.3]heptan-2-yl]-5-[(S)-cyclopropylsulfonimidoyl]furan-2-carboxamide;
(Rₐ)—N-[6-(6-chlorooxazolo[5,4-b]pyridin-2-yl)spiro[3.3]heptan-2-yl]-5-[(R)-cyclopropylsulfonimidoyl]furan-2-carboxamide;
(Sₐ)—N-[6-(6-chlorooxazolo[5,4-b]pyridin-2-yl)spiro[3.3]heptan-2-yl]-5-[(S)-cyclopropylsulfonimidoyl]furan-2-carboxamide;
(Sₐ)—N-[6-(6-chlorooxazolo[5,4-b]pyridin-2-yl)spiro[3.3]heptan-2-yl]-5-[(R)-cyclopropylsulfonimidoyl]furan-2-carboxamide;
N-[6-(6-chlorooxazolo[4,5-c]pyridin-2-yl)spiro[3.3]heptan-2-yl]-5-(cyclopropylsulfonimidoyl)furan-2-carboxamide;
N-[6-(5-chloro-1,3-benzoxazol-2-yl)spiro[3.3]heptan-2-yl]-5-(pyrrolidin-1-ylsulfonimidoyl)furan-2-carboxamide;
N-[6-(5-chloro-1,3-benzoxazol-2-yl)spiro[3.3]heptan-2-yl]-5-(morpholinosulfonimidoyl)furan-2-carboxamide;
N-[6-(5-chloro-1,3-benzoxazol-2-yl)spiro[3.3]heptan-2-yl]-5-(propanoylsulfamoyl)furan-2-carboxamide;
(Sₐ)—N-[6-(5-chloro-1,3-benzoxazol-2-yl)spiro[3.3]heptan-2-yl]-5-(propanoylsulfamoyl)furan-2-carboxamide;
(Rₐ)—N-[6-(5-chloro-1,3-benzoxazol-2-yl)spiro[3.3]heptan-2-yl]-5-(propanoylsulfamoyl)furan-2-carboxamide;
N-(butanoylsulfamoyl)-N-[6-(5-chloro-1,3-benzoxazol-2-yl)spiro[3.3]heptan-2-yl]furan-2-carboxamide;
(Sₐ)—N-(butanoylsulfamoyl)-N-[6-(5-chloro-1,3-benzoxazol-2-yl)spiro[3.3]heptan-2-yl]furan-2-carboxamide;
(Rₐ)—N-(butanoylsulfamoyl)-N-[6-(5-chloro-1,3-benzoxazol-2-yl)spiro[3.3]heptan-2-yl]furan-2-carboxamide;
N-[6-(5-chloro-1,3-benzoxazol-2-yl)spiro[3.3]heptan-2-yl]-5-(2-methylpropanoylsulfamoyl)furan-2-carboxamide;
(Sₐ)—N-[6-(5-chloro-1,3-benzoxazol-2-yl)spiro[3.3]heptan-2-yl]-5-(2-methylpropanoylsulfamoyl)furan-2-carboxamide;
(Rₐ)—N-[6-(5-chloro-1,3-benzoxazol-2-yl)spiro[3.3]heptan-2-yl]-5-(2-methylpropanoylsulfamoyl)furan-2-carboxamide;
N-[6-(5-chloro-1,3-benzoxazol-2-yl)spiro[3.3]heptan-2-yl]-5-(cyclopropanecarbonylsulfamoyl)furan-2-carboxamide;
(Sₐ)—N-[6-(5-chloro-1,3-benzoxazol-2-yl)spiro[3.3]heptan-2-yl]-5-(cyclopropanecarbonylsulfamoyl)furan-2-carboxamide;
(Rₐ)—N-[6-(5-chloro-1,3-benzoxazol-2-yl)spiro[3.3]heptan-2-yl]-5-(cyclopropanecarbonylsulfamoyl)furan-2-carboxamide;
N-[6-(5-chloro-1,3-benzoxazol-2-yl)spiro[3.3]heptan-2-yl]-5-(cyclobutanecarbonylsulfamoyl)furan-2-carboxamide;
(Sₐ)—N-[6-(5-chloro-1,3-benzoxazol-2-yl)spiro[3.3]heptan-2-yl]-5-(cyclobutanecarbonylsulfamoyl)furan-2-carboxamide;
(Rₐ)—N-[6-(5-chloro-1,3-benzoxazol-2-yl)spiro[3.3]heptan-2-yl]-5-(cyclobutanecarbonylsulfamoyl)furan-2-carboxamide;
N-[6-(5-chloro-1,3-benzoxazol-2-yl)spiro[3.3]heptan-2-yl]-5-[(2-methoxyacetyl)sulfamoyl]furan-2-carboxamide;
(Sₐ)—N-[6-(5-chloro-1,3-benzoxazol-2-yl)spiro[3.3]heptan-2-yl]-5-[(2-methoxyacetyl)sulfamoyl]furan-2-carboxamide;
(Rₐ)—N-[6-(5-chloro-1,3-benzoxazol-2-yl)spiro[3.3]heptan-2-yl]-5-[(2-methoxyacetyl)sulfamoyl]furan-2-carboxamide;
ethyl N-[[5-[[6-(5-chloro-1,3-benzoxazol-2-yl)spiro[3.3]heptan-2-yl]carbamoyl]-2-furyl]sulfonyl]carbamate;
(Sₐ)-ethyl N-[[5-[[6-(5-chloro-1,3-benzoxazol-2-yl)spiro[3.3]heptan-2-yl]carbamoyl]-2-furyl]sulfonyl]carbamate;
(Rₐ)-ethyl N-[[5-[[6-(5-chloro-1,3-benzoxazol-2-yl)spiro[3.3]heptan-2-yl]carbamoyl]-2-furyl]sulfonyl]carbamate;
methyl 4-[[5-[[6-(5-chloro-1,3-benzoxazol-2-yl)spiro[3.3]heptan-2-yl]carbamoyl]-2-furyl]sulfonylamino]-4-oxo-butanoate;
(Sₐ)-methyl 4-[[5-[[6-(5-chloro-1,3-benzoxazol-2-yl)spiro[3.3]heptan-2-yl]carbamoyl]-2-furyl]sulfonylamino]-4-oxo-butanoate;
(Rₐ)-methyl 4-[[5-[[6-(5-chloro-1,3-benzoxazol-2-yl)spiro[3.3]heptan-2-yl]carbamoyl]-2-furyl]sulfonylamino]-4-oxo-butanoate;
5-(bicyclo[1.1.1]pentane-1-carbonylsulfamoyl)-N-[6-(5-chloro-1,3-benzoxazol-2-yl)spiro[3.3]heptan-2-yl]furan-2-carboxamide;
(Sₐ)-5-(bicyclo[1.1.1]pentane-1-carbonylsulfamoyl)-N-[6-(5-chloro-1,3-benzoxazol-2-yl)spiro[3.3]heptan-2-yl]furan-2-carboxamide;
(Rₐ)-5-(bicyclo[1.1.1]pentane-1-carbonylsulfamoyl)-N-[6-(5-chloro-1,3-benzoxazol-2-yl)spiro[3.3]heptan-2-yl]furan-2-carboxamide;
N-[6-(5-chloro-1,3-benzoxazol-2-yl)spiro[3.3]heptan-2-yl]-5-(tetrahydrofuran-3-carbonylsulfamoyl)furan-2-carboxamide;
(Sₐ)—N-[6-(5-chloro-1,3-benzoxazol-2-yl)spiro[3.3]heptan-2-yl]-5-(tetrahydrofuran-3-carbonylsulfamoyl)furan-2-carboxamide;
(Rₐ)—N-[6-(5-chloro-1,3-benzoxazol-2-yl)spiro[3.3]heptan-2-yl]-5-(tetrahydrofuran-3-carbonylsulfamoyl)furan-2-carboxamide;
N-[6-(5-chloro-1,3-benzoxazol-2-yl)spiro[3.3]heptan-2-yl]-5-[[2-(2-methoxyethoxy)acetyl]sulfamoyl]furan-2-carboxamide;
(Sₐ)—N-[6-(5-chloro-1,3-benzoxazol-2-yl)spiro[3.3]heptan-2-yl]-5-[[2-(2-methoxyethoxy)acetyl]sulfamoyl]furan-2-carboxamide;
(Rₐ)—N-[6-(5-chloro-1,3-benzoxazol-2-yl)spiro[3.3]heptan-2-yl]-5-[[2-(2-methoxyethoxy)acetyl]sulfamoyl]furan-2-carboxamide;
N-[6-(5-chloro-1,3-benzoxazol-2-yl)spiro[3.3]heptan-2-yl]-5-[[2-[2-(2-methoxyethoxy)ethoxy]acetyl]sulfamoyl]furan-2-carboxamide;
(Sₐ)—N-[6-(5-chloro-1,3-benzoxazol-2-yl)spiro[3.3]heptan-2-yl]-5-[[2-[2-(2-methoxyethoxy)ethoxy]acetyl]sulfamoyl]furan-2-carboxamide;
(Rₐ)—N-[6-(5-chloro-1,3-benzoxazol-2-yl)spiro[3.3]heptan-2-yl]-5-[[2-[2-(2-methoxyethoxy)ethoxy]acetyl]sulfamoyl]furan-2-carboxamide;
5-(acetylsulfamoyl)-N-[6-(5-chloro-1,3-benzoxazol-2-yl)spiro[3.3]heptan-2-yl]furan-2-carboxamide;
N-[6-(5-chloro-1,3-benzoxazol-2-yl)spiro[3.3]heptan-2-yl]-5-(cyclopentanecarbonylsulfamoyl)furan-2-carboxamide;
N-[6-(5-chloro-1,3-benzoxazol-2-yl)spiro[3.3]heptan-2-yl]-5-(cyclohexanecarbonylsulfamoyl)furan-2-carboxamide;
N-[6-(5-chloro-1,3-benzoxazol-2-yl)spiro[3.3]heptan-2-yl]-5-[(1,1-dioxothietane-3-carbonyl)sulfamoyl]furan-2-carboxamide;
N-[6-(5-chloro-1,3-benzoxazol-2-yl)spiro[3.3]heptan-2-yl]-5-[(1,1-dioxothiolane-3-carbonyl)sulfamoyl]furan-2-carboxamide;
N-[6-(5-chloro-1,3-benzoxazol-2-yl)spiro[3.3]heptan-2-yl]-5-[(1,1-dioxothiane-3-carbonyl)sulfamoyl]furan-2-carboxamide;
N-[6-(5-chloro-1,3-benzoxazol-2-yl)spiro[3.3]heptan-2-yl]-5-(oxetane-3-carbonylsulfamoyl)furan-2-carboxamide;
N-[[5-[[6-(5-chloro-1,3-benzoxazol-2-yl)spiro[3.3]heptan-2-yl]carbamoyl]-2-furyl]sulfonyl]tetrahydropyran-4-carboxamide;
N-[6-(5-chloro-1,3-benzoxazol-2-yl)spiro[3.3]heptan-2-yl]-5-(methylsulfamoyl)furan-2-carboxamide; and, N-[6-(5-chloro-1,3-benzoxazol-2-yl)spiro[3.3]heptan-2-yl]-5-(dimethylsulfamoyl)furan-2-carboxamide;
or a pharmaceutically acceptable salt thereof.

16. A compound according to claim 1, selected from the group consisting of:
(Rₐ)—N-[6-(5-chloro-1,3-benzoxazol-2-yl)spiro[3.3]heptan-2-yl]-5-methylsulfonyl-furan-2-carboxamide;
N-[6-(5-chloro-1,3-benzoxazol-2-yl)spiro[3.3]heptan-2-yl]-5-cyclopropylsulfonyl-furan-2-carboxamide;
N-[6-(5-Chloro-1,3-benzoxazol-2-yl)spiro[3.3]heptan-2-yl]-5-(cyclopropylmethylsulfinyl)furan-2-carboxamide;
(Rₐ)—N-[6-(5-chloro-1,3-benzoxazol-2-yl)spiro[3.3]heptan-2-yl]-5-[(S)-cyclopropylmethylsulfinyl]furan-2-carboxamide;
(Rₐ)—N-[6-(5-chloro-1,3-benzoxazol-2-yl)spiro[3.3]heptan-2-yl]-5-[(R)-cyclopropylmethylsulfinyl]furan-2-carboxamide;
(Sₐ)—N-[6-(5-Chloro-1,3-benzoxazol-2-yl)spiro[3.3]heptan-2-yl]-5-[(S)-cyclopropylmethylsulfinyl]furan-2-carboxamide;
(Sₐ)—N-[6-(5-chloro-1,3-benzoxazol-2-yl)spiro[3.3]heptan-2-yl]-5-[(R)-cyclopropylmethylsulfinyl]furan-2-carboxamide;
N-[6-(5-chloro-1,3-benzoxazol-2-yl)spiro[3.3]heptan-2-yl]-5-(cyclopropylmethylsulfonyl)furan-2-carboxamide;
N-[6-(5-chloro-1,3-benzoxazol-2-yl)spiro[3.3]heptan-2-yl]-5-isobutylsulfinyl-furan-2-carboxamide;
N-[6-(5-chloro-1,3-benzoxazol-2-yl)spiro[3.3]heptan-2-yl]-5-isobutylsulfonyl-furan-2-carboxamide;
N-[6-(5-chloro-1,3-benzoxazol-2-yl)spiro[3.3]heptan-2-yl]-5-(cyclobutylmethylsulfonyl)furan-2-carboxamide;
benzyl 3-[[5-[[6-(5-chloro-1,3-benzoxazol-2-yl)spiro[3.3]heptan-2-yl]carbamoyl]-2-furyl]sulfonylmethyl]azetidine-1-carboxylate;
N-[6-(5-chloro-1,3-benzoxazol-2-yl)spiro[3.3]heptan-2-yl]-5-[(3,3-difluorocyclobutyl)methylsulfonyl]furan-2-carboxamide;
N-[6-(5-chloro-1,3-benzoxazol-2-yl)spiro[3.3]heptan-2-yl]-5-(2-methoxyethylsulfonyl)furan-2-carboxamide;
N-[6-(5-chloro-1,3-benzoxazol-2-yl)spiro[3.3]heptan-2-yl]-5-sulfamoyl-furan-2-carboxamide;
(Sₐ)—N-[6-(5-chloro-1,3-benzoxazol-2-yl)spiro[3.3]heptan-2-yl]-5-sulfamoyl-furan-2-carboxamide;
(Rₐ)—N-[6-(5-chloro-1,3-benzoxazol-2-yl)spiro[3.3]heptan-2-yl]-5-sulfamoyl-furan-2-carboxamide;
N-[6-(5-chloro-1,3-benzoxazol-2-yl)spiro[3.3]heptan-2-yl]-5-(sulfamoylmethyl)furan-2-carboxamide;
4-bromo-N-[6-(5-chloro-1,3-benzoxazol-2-yl)spiro[3.3]heptan-2-yl]-5-ethylsulfonyl-furan-2-carboxamide;
5-tert-butylsulfinyl-N-[6-(5-chloro-1,3-benzoxazol-2-yl)spiro[3.3]heptan-2-yl]furan-2-carboxamide;
5-tert-butylsulfonyl-N-[6-(5-chloro-1,3-benzoxazol-2-yl)spiro[3.3]heptan-2-yl]furan-2-carboxamide;
N-[6-(5-chloro-1,3-benzoxazol-2-yl)spiro[3.3]heptan-2-yl]-5-cyclopropylsulfinyl-furan-2-carboxamide;
N-[6-(5-chloro-1,3-benzoxazol-2-yl)spiro[3.3]heptan-2-yl]-5-(trifluoromethylsulfonyl)furan-2-carboxamide;
N-[6-(6-chlorooxazolo[5,4-b]pyridin-2-yl)spiro[3.3]heptan-2-yl]-5-cyclopropylsulfonyl-furan-2-carboxamide;
(Rₐ)—N-[6-(6-chlorooxazolo[5,4-b]pyridin-2-yl)spiro[3.3]heptan-2-yl]-5-cyclopropylsulfonyl-furan-2-carboxamide;
(Sₐ)—N-[6-(6-chlorooxazolo[5,4-b]pyridin-2-yl)spiro[3.3]heptan-2-yl]-5-cyclopropylsulfonyl-furan-2-carboxamide;
N-[6-(6-chlorooxazolo[5,4-b]pyridin-2-yl)spiro[3.3]heptan-2-yl]-5-cyclopropylsulfinyl-furan-2-carboxamide;
N-[6-(5-chloro-1,3-benzoxazol-2-yl)spiro[3.3]heptan-2-yl]-5-(methylsulfonimidoyl)furan-2-carboxamide;
(Rₐ)—N-[6-(5-chloro-1,3-benzoxazol-2-yl)spiro[3.3]heptan-2-yl]-5-[(S)-methylsulfonimidoyl]furan-2-carboxamide;
(Sₐ)—N-[6-(5-chloro-1,3-benzoxazol-2-yl)spiro[3.3]heptan-2-yl]-5-[(S)-methylsulfonimidoyl]furan-2-carboxamide;
(Rₐ)—N-[6-(5-chloro-1,3-benzoxazol-2-yl)spiro[3.3]heptan-2-yl]-5-[(R)-methylsulfonimidoyl]furan-2-carboxamide;
(Sₐ)—N-[6-(5-chloro-1,3-benzoxazol-2-yl)spiro[3.3]heptan-2-yl]-5-[(R)-methylsulfonimidoyl]furan-2-carboxamide;
N-[6-(5-chloro-1,3-benzoxazol-2-yl)spiro[3.3]heptan-2-yl]-5-(cyclopropylsulfonimidoyl)furan-2-carboxamide;
(Rₐ)—N-[6-(5-chloro-1,3-benzoxazol-2-yl)spiro[3.3]heptan-2-yl]-5-[(S)-cyclopropylsulfonimidoyl]furan-2-carboxamide;
(Sₐ)—N-[6-(5-chloro-1,3-benzoxazol-2-yl)spiro[3.3]heptan-2-yl]-5-[(S)-cyclopropylsulfonimidoyl]furan-2-carboxamide;
(Rₐ)—N-[6-(5-chloro-1,3-benzoxazol-2-yl)spiro[3.3]heptan-2-yl]-5-[(R)-cyclopropylsulfonimidoyl]furan-2-carboxamide;
(Sₐ)—N-[6-(5-chloro-1,3-benzoxazol-2-yl)spiro[3.3]heptan-2-yl]-5-[(R)-cyclopropylsulfonimidoyl]furan-2-carboxamide;
N-[6-(5-chloro-1,3-benzoxazol-2-yl)spiro[3.3]heptan-2-yl]-5-(cyclopropylmethylsulfonimidoyl)furan-2-carboxamide;
(Rₐ)—N-[6-(5-chloro-1,3-benzoxazol-2-yl)spiro[3.3]heptan-2-yl]-5-[(S)-cyclopropylmethylsulfonimidoyl]furan-2-carboxamide;
(Rₐ)—N-[6-(5-Chloro-1,3-benzoxazol-2-yl)spiro[3.3]heptan-2-yl]-5-[(R)-cyclopropylmethylsulfonimidoyl]furan-2-carboxamide;
(Sₐ)—N-[6-(5-Chloro-1,3-benzoxazol-2-yl)spiro[3.3]heptan-2-yl]-5-[(S)-cyclopropylmethylsulfonimidoyl]furan-2-carboxamide;
(Sₐ)—N-[6-(5-Chloro-1,3-benzoxazol-2-yl)spiro[3.3]heptan-2-yl]-5-[(R)-cyclopropylmethylsulfonimidoyl]furan-2-carboxamide;
N-[6-(5-chloro-1,3-benzoxazol-2-yl)spiro[3.3]heptan-2-yl]-5-(isobutylsulfonimidoyl)furan-2-carboxamide;
N-[6-(5-chloro-1,3-benzoxazol-2-yl)spiro[3.3]heptan-2-yl]-5-[(3,3-difluorocyclobutyl)methylsulfonimidoyl]furan-2-carboxamide;
N-[6-(6-chlorooxazolo[5,4-b]pyridin-2-yl)spiro[3.3]heptan-2-yl]-5-(cyclopropylsulfonimidoyl)furan-2-carboxamide;
(Rₐ)—N-[6-(6-chlorooxazolo[5,4-b]pyridin-2-yl)spiro[3.3]heptan-2-yl]-5-[(S)-cyclopropylsulfonimidoyl]furan-2-carboxamide;
(Rₐ)—N-[6-(6-chlorooxazolo[5,4-b]pyridin-2-yl)spiro[3.3]heptan-2-yl]-5-[(R)-cyclopropylsulfonimidoyl]furan-2-carboxamide;
(Sₐ)—N-[6-(6-chlorooxazolo[5,4-b]pyridin-2-yl)spiro[3.3]heptan-2-yl]-5-[(S)-cyclopropylsulfonimidoyl]furan-2-carboxamide;
(Sₐ)—N-[6-(6-chlorooxazolo[5,4-b]pyridin-2-yl)spiro[3.3]heptan-2-yl]-5-[(R)-cyclopropylsulfonimidoyl]furan-2-carboxamide;
N-[6-(5-chloro-1,3-benzoxazol-2-yl)spiro[3.3]heptan-2-yl]-5-(propanoylsulfamoyl)furan-2-carboxamide;
(Sₐ)—N-[6-(5-chloro-1,3-benzoxazol-2-yl)spiro[3.3]heptan-2-yl]-5-(propanoylsulfamoyl)furan-2-carboxamide;
(Rₐ)—N-[6-(5-chloro-1,3-benzoxazol-2-yl)spiro[3.3]heptan-2-yl]-5-(propanoylsulfamoyl)furan-2-carboxamide;
N-(butanoylsulfamoyl)-N-[6-(5-chloro-1,3-benzoxazol-2-yl)spiro[3.3]heptan-2-yl]furan-2-carboxamide;
(Sₐ)—N-(butanoylsulfamoyl)-N-[6-(5-chloro-1,3-benzoxazol-2-yl)spiro[3.3]heptan-2-yl]furan-2-carboxamide;
(Rₐ)—N-(butanoylsulfamoyl)-N-[6-(5-chloro-1,3-benzoxazol-2-yl)spiro[3.3]heptan-2-yl]furan-2-carboxamide;
N-[6-(5-chloro-1,3-benzoxazol-2-yl)spiro[3.3]heptan-2-yl]-5-(2-methylpropanoylsulfamoyl)furan-2-carboxamide;
(Sₐ)—N-[6-(5-chloro-1,3-benzoxazol-2-yl)spiro[3.3]heptan-2-yl]-5-(2-methylpropanoylsulfamoyl)furan-2-carboxamide;
(Rₐ)—N-[6-(5-chloro-1,3-benzoxazol-2-yl)spiro[3.3]heptan-2-yl]-5-(2-methylpropanoylsulfamoyl)furan-2-carboxamide;
N-[6-(5-chloro-1,3-benzoxazol-2-yl)spiro[3.3]heptan-2-yl]-5-(cyclobutanecarbonylsulfamoyl)furan-2-carboxamide;
(Sₐ)—N-[6-(5-chloro-1,3-benzoxazol-2-yl)spiro[3.3]heptan-2-yl]-5-(cyclobutanecarbonylsulfamoyl)furan-2-carboxamide;
(Rₐ)—N-[6-(5-chloro-1,3-benzoxazol-2-yl)spiro[3.3]heptan-2-yl]-5-(cyclobutanecarbonylsulfamoyl)furan-2-carboxamide;
N-[6-(5-chloro-1,3-benzoxazol-2-yl)spiro[3.3]heptan-2-yl]-5-[(2-methoxyacetyl)sulfamoyl]furan-2-carboxamide;
(Sₐ)—N-[6-(5-chloro-1,3-benzoxazol-2-yl)spiro[3.3]heptan-2-yl]-5-[(2-methoxyacetyl)sulfamoyl]furan-2-carboxamide;
(Rₐ)—N-[6-(5-chloro-1,3-benzoxazol-2-yl)spiro[3.3]heptan-2-yl]-5-[(2-methoxyacetyl)sulfamoyl]furan-2-carboxamide;
N-[6-(5-chloro-1,3-benzoxazol-2-yl)spiro[3.3]heptan-2-yl]-5-(cyclohexanecarbonylsulfamoyl)furan-2-carboxamide; and,
N-[6-(5-chloro-1,3-benzoxazol-2-yl)spiro[3.3]heptan-2-yl]-5-(methylsulfamoyl)furan-2-carboxamide;
or a pharmaceutically acceptable salt thereof.

17. A process for the preparation of a compound according to claim 1 comprising at least one of the following steps,
(a) Reaction of a compound of formula (IV), 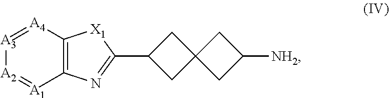
with a compound of formula (V), 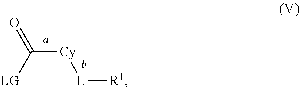
in the presence of a coupling reagent and a base;
(b) Cyclization of a compound of formula (VII-1), 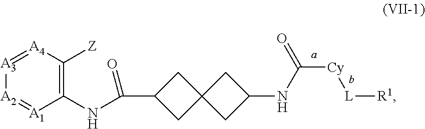
in the presence of a base;
(c) Cyclization of a compound of formula (VII-1) in the presence of DIAD and PPh₃;
(d) Oxidation of a compound of formula (I-2), 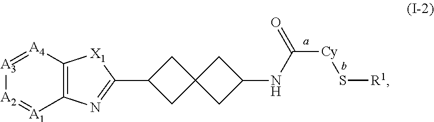
in the presence of an oxidate;
(e) Cyclization of a compound of formula (VII-3), 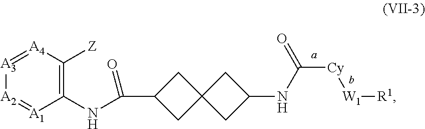
in the presence of a base;
(f) Cyclization of a compound of formula (VII-3) in the presence of DIAD and PPh₃;
(g) Reaction of a compound of formula (I-4), 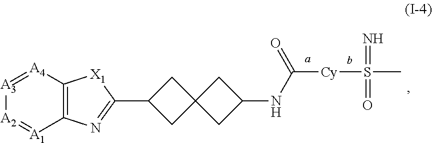
with a cyclized amine (VIII), 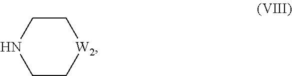
in the presence of a catalyst;
(h) Deprotection of a compound of formula (IX), 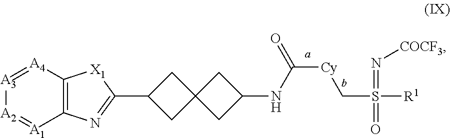
in the presence of a base;
(i) Reaction of a compound of formula (1-8), 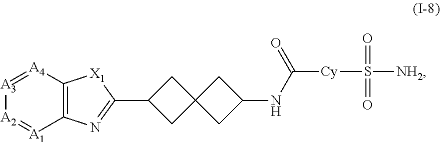
with a halide (X), LG-W₃ (X), in the presence of a base;
wherein A₁ to A₄, X₁, R^{x}, R^{y}, Cy, L and R¹ are defined as any one of claims 1 to 14; Z is halogen or OH; LG is halogen or OH; W₁ is S(O), S(O)₂ or S(O)(NH); W₂ is a bond or O; W₃ is C₁₋₆alkyl or C(O)R¹.

18. A compound according to claim 1 for use as therapeutically active substance.

19. A pharmaceutical composition comprising a compound according to claim 1 and a therapeutically inert carrier.

20. The use of a according to claim 1 for the treatment of HBV infection.

21. The use of a compound according to claim 1 for the preparation of a medicament for the treatment of HBV infection.

22. The use of a compound according to claim 1 for the inhibition of HBeAg.

23. The use of a compound according to claim 1 for the inhibition of HBsAg.

24. The use of a compound according to claim 1 for the inhibition of HBV DNA.

25. A compound according to claim 1 for use in the treatment of HBV infection.

26. A method for the treatment of HBV infection, which method comprises administering an effective amount of a compound according to claim 1.
